(19) United States
(12) Patent Application Publication
(10) Pub. No.: US 2011/0124634 A1
(43) Pub. Date: May 26, 2011

(54) BIOACTIVE COMPOUNDS FOR TREATMENT OF CANCER AND NEURODEGENERATIVE DISEASES

(75) Inventors: Connie L. Sun, Palo Alto, CA (US); Xiaoyuan Li, Los Altos, CA (US)

(73) Assignee: PONIARD PHARMACEUTICALS, INC.

(21) Appl. No.: 12/992,233

(22) PCT Filed: May 8, 2009
(86) PCT No.: PCT/US09/02871

§ 371 Date: Feb. 8, 2011

Related U.S. Application Data
(60) Provisional application No. 61/052,822, filed on May 13, 2008.
(60) Provisional application No. 61/149,268, filed on Feb. 2, 2009.

(51) Int. Cl. A61K 31/541; A61K 31/5377; A61K 31/497; A61K 31/519; C07D 487/04; A61P 25/28; A61P 35/00

(52) U.S. Cl. 514/228.5; 514/234.2; 514/252.16; 514/265.1; 544/61; 544/117; 544/280

(57) ABSTRACT

The invention provides bioactive compounds for the treatment of various malconditions such as cancer and neurodegenerative diseases including Alzheimer's disease. The chemical compounds as disclosed herein are found to show bioactivity in bioassays related to these conditions. Pharmaceutical compositions, combinations and methods of synthesis are provided, as are methods of using the compound, compositions and combinations in the treatment of the diseases.

### CROSS-REFERENCE TO RELATED APPLICATIONS

This application claims the priority of U.S. Ser. No. 61/052,822, filed May 13, 2008, and of U.S. Ser. No. 61/149,268, filed Feb. 2, 2009, the disclosures of which are incorporated herein by reference in their entireties.

### BACKGROUND

HSP90, also known as “heat shock protein 90” is a protein that is believed to act as a molecular chaperone, assisting other proteins in achieving correct folding and conformation in the cellular environment. The protein, having a molecular weight of about 90 kDa, is widely distributed in eukaryotes and is essential for cellular survival. The protein is termed a heat shock protein as it us up-regulated in response to stress. In humans, it exists in at least four highly homologous isoforms. In all the isoforms, there is an N-terminal ATP binding site that can be allosterically regulated. It is believed that HSP90 inhibitors can be useful in the treatment of malconditions such as cancer because HSP90 may delay apoptosis in cells that would otherwise undergo programmed cell death, and HSP90 may stabilize mutant proteins.

### SUMMARY

The present invention is directed to molecular entities having structures as defined herein adapted for use as inhibitors of the protein HSP90 that can be used for the treatment of cancers, neurodegenerative diseases, Alzheimers disease, and other malconditions wherein inhibition of HSP90 is medically indicated.

Various embodiments of the invention provide a compound of formula (I):

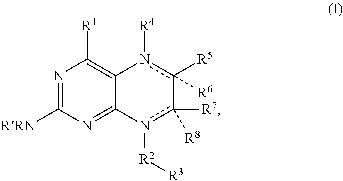

wherein

a dotted line indicates a bond that can be present or absent;

R and R′ are independently at each occurrence H, or (C₁-C₈)alkyl wherein any alkyl is optionally mono- or independently pluri-substituted with OR, halo, cyano, OC(O)R, OC(O)OR, CO₂R, NRR′, C(O)NRR′, N(R)C(O)R′, oxo, OC(O)NRR′, SR, S(O)R, S(O)₂R, S(O)₂OR, or SO₂NRR′ alkyl, acyl, alkoxy, cycloalkyl, heterocyclyl, aryl, or heteroaryl, wherein any alkyl, acyl, alkoxy, cycloalkyl, heterocyclyl, aryl, or heteroaryl is substituted with 0-4 Z; wherein any carbon atom of the alkyl, acyl, alkoxy, cycloalkyl, heterocyclyl, aryl, or heteroaryl, can be replaced by a heteroatom selected from the group consisting of NR, O, S, S(O), and S(O)₂; or, R and R′ can together with a nitrogen atom to which they are bonded form a heterocyclic or heteroaryl ring, wherein the heterocyclic or heteroaryl ring can further comprise 1-3 O, N, S, S(O), or S(O)₂ heteroatoms therewithin and is substituted with 0-4 Z groups wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R¹ comprises H, halo, NRR′, or OR; or comprises (C₁-C₆)alkyl, haloalkyl, cycloalkyl, or heterocyclyl, any of which can be optionally substituted by oxo, CO₂R, heterocylyl, aryl, OR, NRR′, or SR;

R² comprises (C(R″)₂)ₙ wherein n is 0-3, wherein R″ is independently at each occurrence H, alkyl, halo, haloalkyl, OR, or NRR′, or two R″ together with a carbon atom to which they are bonded comprise a carbonyl;

R³ comprises alkyl, cycloalkyl, heterocyclyl, aryl, or heteroaryl, wherein any alkyl, cycloalkyl, heterocyclyl, aryl, or heteroaryl is substituted with 0-5 Z groups,

R⁴ comprises H, alkyl, aryl, or heteroaryl, wherein any alkyl, aryl, heteroaryl, is substituted with 0-4 Z; provided that R⁴ is absent when a double bond is present between the C-6 carbon atom bearing R⁵ or R⁶ and the adjacent N-5 nitrogen atom; and

wherein R⁵ and R⁶ together with the carbon atom to which they are bonded can form a carbonyl group or R⁷ and R⁸ together with the carbon atom to which they are bonded can form a carbonyl group, or both, or neither R⁵ and R⁶ and the carbon atom to which they are bonded nor R⁷ and R⁸ and the carbon atom to which they are bonded forms a carbonyl group; wherein R⁵, R⁶, R⁷, or R⁸, when present, each independently comprises hydrogen or (C₁-C₆)alkyl wherein any alkyl group is substituted with 0-4 Z; wherein a carbon atom that is not within a carbonyl group can bear a single substituent and be double bonded to the adjacent nitrogen atom or can bear two substituents and be single bonded to the adjacent nitrogen atom; or,

a compound of formula (II):

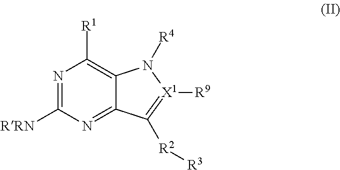

wherein

R and R′ are independently at each occurrence H, or (C₁-C₈)alkyl wherein any alkyl is optionally mono- or independently pluri-substituted with OR, halo, cyano, OC(O)R, OC(O)OR, CO₂R, NRR′, C(O)NRR′, N(R)C(O)R′, oxo, OC(O)NRR′, SR, S(O)R, S(O)₂R, S(O)₂OR, or SO₂NRR′ alkyl, acyl, alkoxy, cycloalkyl, heterocyclyl, aryl, or heteroaryl, wherein any alkyl, acyl, alkoxy, cycloalkyl, heterocyclyl, aryl, or heteroaryl is substituted with 0-4 Z; wherein any carbon atom of the alkyl, acyl, alkoxy, cycloalkyl, heterocyclyl, aryl, or heteroaryl, can be replaced by a heteroatom selected from the group consisting of NR, O, S, S(O), and S(O)₂; or, R and R′ can together with a nitrogen atom to which they are bonded form a heterocyclic or heteroaryl ring, wherein the heterocyclic or heteroaryl ring can further comprise 1-3 O, N, S, S(O), or S(O)₂ heteroatoms therewithin and is substituted with 0-4 Z groups wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R¹ comprises H, halo, NRR′, or hydroxyl; or comprises (C₁-C₆)alkyl, haloalkyl, cycloalkyl, or heterocyclyl, any of which can be optionally substituted by oxo, CO₂R, heterocylyl, aryl, OR, NRR′, or SR;

R² comprises (C(R″)₂)ₙ wherein n is 0-3, wherein R″ is independently at each occurrence H, alkyl, halo, haloalkyl, OR, or NRR′, or two R″ together with a carbon atom to which they are bonded comprise a carbonyl;

R³ comprises alkyl, cycloalkyl, heterocyclyl, aryl, or heteroaryl, wherein any alkyl, cycloalkyl, heterocyclyl, aryl, or heteroaryl is substituted with 0-5 Z groups,

R⁴ comprises H, alkyl, aryl, or heteroaryl, wherein any alkyl, aryl, heteroaryl, is substituted with 0-4 Z; provided that R⁴ is absent when a double bond is present between the carbon atom bearing R⁵ or R⁶ and the adjacent nitrogen atom;

X¹ comprises C or N, provided that when X¹ is N, R⁹ is absent; and

R⁹ comprises H, aryl, or alkyl, wherein any aryl or alkyl is substituted with 0-4 Z, wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′; or,

a compound of formula (III):

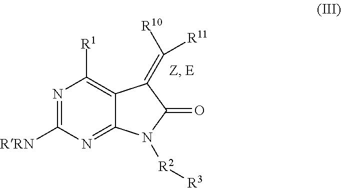

wherein

Y¹ is O or S;

R and R′ are independently at each occurrence H, or (C₁-C₈)alkyl wherein any alkyl is optionally mono- or independently pluri-substituted with OR, halo, cyano, OC(O)R, OC(O)OR, CO₂R, NRR′, C(O)NRR′, N(R)C(O)R′, oxo, OC(O)NRR′, SR, S(O)R, S(O)₂R, S(O)₂OR, or SO₂NRR′ alkyl, acyl, alkoxy, cycloalkyl, heterocyclyl, aryl, or heteroaryl, wherein any alkyl, acyl, alkoxy, cycloalkyl, heterocyclyl, aryl, or heteroaryl is substituted with 0-4 Z; wherein any carbon atom of the alkyl, acyl, alkoxy, cycloalkyl, heterocyclyl, aryl, or heteroaryl, can be replaced by a heteroatom selected from the group consisting of NR, O, S, S(O), and S(O)₂; or, R and R′ can together with a nitrogen atom to which they are bonded form a heterocyclic or heteroaryl ring, wherein the heterocyclic or heteroaryl ring can further comprise 1-3 O, N, S, S(O), or S(O)₂ heteroatoms therewithin and is substituted with 0-4 Z groups wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R¹ comprises H, halo, NRR′, or hydroxyl; or comprises (C₁-C₆)alkyl, haloalkyl, cycloalkyl, or heterocyclyl, any of which can be optionally substituted by oxo, CO₂R, heterocylyl, aryl, OR, NRR′, or SR;

R² comprises (C(R″)₂)ₙ wherein n is 0-3, wherein R″ is independently at each occurrence H, alkyl, halo, haloalkyl, OR, or NRR′, or two R″ together with a carbon atom to which they are bonded comprise a carbonyl;

R³ comprises alkyl, cycloalkyl, heterocyclyl, aryl, or heteroaryl, wherein any alkyl, cycloalkyl, heterocyclyl, aryl, or heteroaryl is substituted with 0-5 Z groups;

R¹⁰ and R¹¹ are each independently H, alkyl, aryl, cycloalkyl, heterocyclyl, or heteroaryl, wherein any alkyl, aryl, cycloalkyl, heterocyclyl, or heteroaryl is substituted with 0-4 Z; wherein any aryl, cycloalkyl, heterocyclyl, or heteroaryl can be fused to a five to seven-membered carbocyclic or heterocyclic ring which is substituted with 0-4 Z; or, R¹⁰ and R¹¹ together with a carbon atom to which they are bonded form an optionally substituted carbocyclic or heterocyclic ring which is substituted with 0-4 Z; wherein a double bond marked Z, E can be in either a Z or an E configuration, or a mixture thereof; or,

a compound of formula (IV):

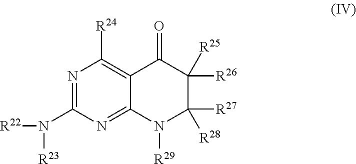

wherein

R²² and R²³ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z, wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²² and R²³ can together with the nitrogen atom to which they are bonded form a heterocyclic ring substituted with 0-4 Z; wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R²⁴ is H, halo, alkyl, haloalkyl, OR, NRR′, nitro, or cyano;

R²⁵ and R²⁶ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁵ and R²⁶ can together with a carbon atom to which they are bonded form a cycloalkyl ring substituted with 0-4 Z;

R²⁷ and R²⁸ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁷ and R²⁸ can together with a carbon atom to which they are bonded form a cycloalkyl ring substituted with 0-4 Z;

R²⁹ is H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁹ comprises a group of the formula R²-R³; or,

a compound of formula (V)

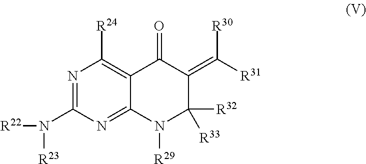

wherein

R²² and R²³ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z, wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²² and R²³ can together with the nitrogen atom to which they are bonded form a heterocyclic ring substituted with 0-4 Z; wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R²⁴ is H, halo, alkyl, haloalkyl, OR, NRR′, nitro, or cyano;

R²⁹ is H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁹ comprises a group of the formula R²-R³;

R³⁰ and R³¹ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R³⁰ and R³¹ can together with a carbon atom to which they are bonded form a cycloalkyl ring substituted with 0-4 Z;

R³² and R³³ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R³² and R³³ can together with a carbon atom to which they are bonded form a cycloalkyl ring substituted with 0-4 Z; or,

a compound of formula (VI)

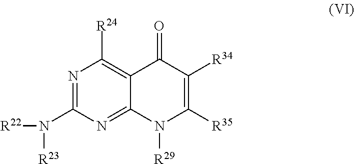

wherein

R²² and R²³ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z, wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²² and R²³ can together with the nitrogen atom to which they are bonded form a heterocyclic ring substituted with 0-4 Z; wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R²⁴ is H, halo, alkyl, haloalkyl, OR, NRR′, nitro, or cyano;

R²⁹ is H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁹ comprises a group of the formula R²-R³;

R³⁴ and R³⁵ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R³⁴ and R³⁵ can together with a carbon atom to which they are bonded form a cycloalkyl ring substituted with 0-4 Z; or,

a compound of formula (VII)

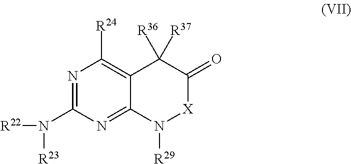

wherein

R²² and R²³ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z, wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²² and R²³ can together with the nitrogen atom to which they are bonded form a heterocyclic ring substituted with 0-4 Z; wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R²⁴ is H, halo, alkyl, haloalkyl, OR, NRR′, nitro, or cyano;

R²⁹ is H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁹ comprises a group of the formula R²-R³;

R³⁶ and R³⁷ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R³⁶ and R³⁷ can together with a carbon atom to which they are bonded form a cycloalkyl ring substituted with 0-4 Z;

X is CR³⁸R³⁹ or NR⁴⁰ wherein R³⁸ and R³⁹ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R³⁸ and R³⁹ can together with a carbon atom to which they are bonded form a cycloalkyl ring substituted with 0-4 Z; and

R⁴⁰ is H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected froth the group consisting of NR, O, S, S(O), and S(O)₂; or

a compound of formula (VIII)

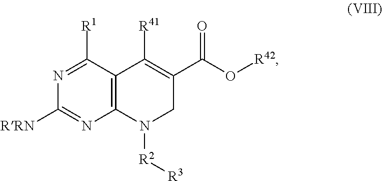

R and R′ are independently at each occurrence H, or (C₁-C₈)alkyl wherein any alkyl is optionally mono- or independently pluri-substituted with OR, halo, cyano, OC(O)R, OC(O)OR, CO₂R, NRR′, C(O)NRR′, N(R)C(O)R′, oxo, OC(O)NRR′, SR, S(O)R, S(O)₂R, S(O)₂OR, or SO₂NRR′ alkyl, acyl, alkoxy, cycloalkyl, heterocyclyl, aryl, or heteroaryl, wherein any alkyl, acyl, alkoxy, cycloalkyl, heterocyclyl, aryl, or heteroaryl is substituted with 0-4 Z; wherein any carbon atom of the alkyl, acyl, alkoxy, cycloalkyl, heterocyclyl, aryl, or heteroaryl, can be replaced by a heteroatom selected from the group consisting of NR, O, S, S(O), and S(O)₂; or, R and R′ can together with a nitrogen atom to which they are bonded form a heterocyclic or heteroaryl ring, wherein the heterocyclic or heteroaryl ring can further comprise 1-3 O, N, S, S(O), or S(O)₂ heteroatoms therewithin and is substituted with 0-4 Z groups wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R¹ comprises H, halo, NRR′, or hydroxyl; or comprises (C₁-C₆)alkyl, haloalkyl, cycloalkyl, or heterocyclyl, any of which can be optionally substituted by oxo, CO₂R, heterocylyl, aryl, OR, NRR′, or SR;

R² comprises (C(R″)₂)ₙ wherein n is 0-3, wherein R″ is independently at each occurrence H, alkyl, halo, haloalkyl, OR, or NRR′, or two R″ together with a carbon atom to which they are bonded comprise a carbonyl;

R³ comprises alkyl, cycloalkyl, heterocyclyl, aryl, or heteroaryl, wherein any alkyl, cycloalkyl, heterocyclyl, aryl, or heteroaryl is substituted with 0-5 Z groups;

R⁴¹ and R⁴² are each independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂;

or any salt, solvate, hydrate, tautomer, stereoisomer, or prodrug of any of the compounds of formulas (I), (II), (III), (IV), (V), (VI), (VII), or (VIII).

In various embodiments, the invention provides a compound of formula (I), a compound of formula (II), a compound of formula (III), a compound of formula (IV), a compound of formula (V), a compound of formula (VI), a compound of formula (VII), a compound of formula (VIII), or any salt, solvate, hydrate, tautomer, stereoisomer, or prodrug thereof.

In various embodiments a pharmaceutical composition comprising a compound of the invention and a suitable excipient is provided.

In various embodiments, a pharmaceutical combination comprising a compound of the invention, optionally including a suitable excipient, and a second medicament, is provided. The second medicament is adapted for treatment of the same condition or a related condition for which administration of a compound of the invention is medically indicated.

In various embodiments, methods of synthesis of the inventive compounds are provided.

Various embodiments of the invention provide methods of use of an inventive compound, composition, or combination, comprising administering the compound, composition, or combination to a patient in need thereof for treatment of a malcondition in a dose, at a frequency, and for a duration of time sufficient to provide a beneficial effect to the patient. In various embodiments inhibition of HSP90 is medically indicated for treatment of the malcondition. The malcondition can comprise cancer, neurodegenerative diseases, or Alzheimer's disease.

Various embodiments provide uses of a compound, composition, or combination of the invention in preparing a medicament for the treatment of a malcondition in a human patient.

### DETAILED DESCRIPTION

The term “treatment” is defined as the management and care of a patient for the purpose of combating the disease, condition, or disorder, for example one of the many types of conditions collectively referred to as “cancer”, and includes administering a compound of the present invention to prevent the onset of the symptoms or complications, or alleviating the symptoms or complications, or curing or eliminating the disease, condition, or disorder.

By “inhibition” of HSP90, what is meant is interference with any normal function of HSP90 in the mammalian body. An “HSP90 inhibitor” or “inhibitory compound” is a substance that interferes with any normal function of HSP90 in the mammalian body.

“Treating” within the context of the instant invention means an alleviation of symptoms associated with a disorder or disease, or inhibition of further progression or worsening of those symptoms, or prevention or prophylaxis of the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result by binding to HSP90 or inhibition of HSP90 activity. A therapeutically effective amount is also one in which any toxic or detrimental effects of compounds of the invention are outweighed by the therapeutically beneficial effects.

“Cancer” as the term is used herein refers to any malignancy, solid tumor, metastasizing neoplasm, or other condition wherein cell division is uncontrolled and cells lose differentiation.

“Neurodegenerative diseases” include without limitation such conditions as amyotrophic lateral sclerosis (ALS), dementias such as vascular dementia, HIV-associated dementia, and Alzheimer's disease, Kreutzfeld-Jacob disease, Huntington's disease, multiple sclerosis (MS), Parkinson's disease, schizophrenia, and the like.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim.

When a substituent is specified to be an atom or atoms of specified identity, “or a bond”, a configuration is referred to when the substituent is “a bond” that the groups that are immediately adjacent to the specified substituent are directly connected to each other by a chemically feasible bonding configuration.

All chiral, diastereomeric, racemic forms of a structure are intended, unless a particular stereochemistry or isomeric form is specifically indicated. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

In general, “substituted” refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted. When a substituent is monovalent, such as, for example, F or Cl, it is bonded to the atom it is substituting by a single bond. When a substituent is more than monovalent, such as 0, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. Alternatively, a divalent substituent such as O, S, C(O), S(O), or S(O)₂ can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups can be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic” and “carbocycle” denote a ring structure wherein the atoms of the ring are carbon. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N-1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂CC(CH₃), and —CH₂C≡C(CH₂CH₃) among others.

Aryl groups are cyclic aromatic hydrocarbons that do not contain heteroatoms. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups include aromatic and non-aromatic ring compounds containing 3 or more ring members, of which, one or more is a heteroatom such as, but not limited to, N, O, and S. In some embodiments, heterocyclyl groups include 3 to 20 ring members, whereas other such groups have 3 to 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

More specifically, aryl and heteroaryl groups can include phenyl, isoindolidinyl, imidazolyl, oxazolyl, benzimidazolyl, and benzoxazolyl; wherein any aryl or heteroaryl can be unsubstituted, mono-substituted, or independently pluri-substituted, for example with J groups as defined herein.

Heterocyclylalkyl groups are alkyl groups as defined above in which a hydrogen or carbon bond of an alkyl group is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like.

“Halo” as the term is used herein includes fluoro, chloro, bromo, and iodo. A “haloalkyl” group includes mono-halo alkyl groups, and poly-halo alkyl groups wherein all halo atoms can be the same or different. Partially halogenated alkyl groups are “haloalkyl” within the meaning herein. Examples of haloalkyl include trifluoromethyl, 1,1-dichloroethyl, 1,2-dichloroethyl, 1,3-dibromo-3,3-difluoropropyl and the like.

The terms “aryloxy” and “arylalkoxy” refer to, respectively, an aryl group bonded to an oxygen atom and an aralkyl group bonded to the oxygen atom at the alkyl moiety. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” includes primary, secondary, and tertiary amines having, e.g., the formula N(group)₃ wherein each group can independently be H or non-H, such as alkyl, aryl, and the like. Amines include but are not limited to R—NH₂, alkylamines, arylamines, alkylarylamines, R₂NH wherein each R is independently selected, such as dialkylamines, diarylamines, aralkylamines, heterocyclylamines and the like, and R₃N wherein each R is independently selected, such as trialkylamines, dialkylarylamines, alkyldiarylamines, triarylamines, and the like. An “amino” group is a substituent of the form —NH₂, —NHR, —NR₂, —NR₃⁺, wherein each R is independently selected, and protonated forms of each. The term “amine” also includes ammonium ions as used herein.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and —NRC(O)R groups, respectively. Amide groups therefore include but are not limited to carbamoyl groups (—C(O)NH₂) and formamide groups (—NHC(O)H).

The term “urethane” (or “carbamyl”) includes N- and O-urethane groups, i.e., —NRC(O)OR and —OC(O)NR₂ groups, respectively.

The term “sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein.

A “hydrate” is a compound that exists in a composition with water molecules. The composition can include water in stoichiometric quantities, such as a monohydrate or a dihydrate, or can include water in random amounts.

A “solvate” is a similar composition except that a solvent other that water replaces the water. For example, methanol or ethanol can form an “alcoholate”, which can again be stoichiometric or non-stoichiometric.

“Tautomers” are two forms of a substance differing only by the position of a hydrogen atom in the molecular structures.

A “prodrug” as is well known in the art is a substance that can be administered to a patient where the substance is converted in vivo by the action of biochemicals within the patient's body, such as enzymes, to the active pharmaceutical ingredient. Examples of prodrugs include esters of carboxylic acid groups, which can be hydrolyzed by endogenous esterases as are found in the bloodstream of humans and other mammals.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

Provisos may apply to any of the disclosed categories or embodiments wherein any one or more of the other above disclosed embodiments or species may be excluded from such categories or embodiments.

In various embodiments, the compound or set of compounds, such as are used in the inventive methods, can be any one of any of the combinations and/or sub-combinations of the above-listed embodiments.

Various embodiments of the invention provide a compound of formula (I):

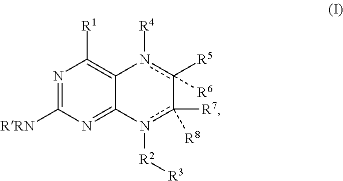

wherein

a dotted line indicates a bond that can be present or absent;

R and R′ are independently at each occurrence H, or (C₁-C₈)alkyl wherein any alkyl is optionally mono- or independently pluri-substituted with OR, halo, cyano, OC(O)R, OC(O)OR, CO₂R, NRR′, C(O)NRR′, N(R)C(O)R′, oxo, OC(O)NRR′, SR, S(O)R, S(O)₂R, S(O)₂OR, or SO₂NRR′ alkyl, acyl, alkoxy, cycloalkyl, heterocyclyl, aryl, or heteroaryl, wherein any alkyl, acyl, alkoxy, cycloalkyl, heterocyclyl, aryl, or heteroaryl is substituted with 0-4 Z; wherein any carbon atom of the alkyl, acyl, alkoxy, cycloalkyl, heterocyclyl, aryl, or heteroaryl, can be replaced by a heteroatom selected from the group consisting of NR, O, S, S(O), and S(O)₂; or, R and R′ can together with a nitrogen atom to which they are bonded form a heterocyclic or heteroaryl ring, wherein the heterocyclic or heteroaryl ring can further comprise 1-3 O, N, S, S(O), or S(O)₂ heteroatoms therewithin and is substituted with 0-4 Z groups wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R¹ comprises H, halo, NRR′, or OR; or comprises (C₁-C₆)alkyl, haloalkyl, cycloalkyl, or heterocyclyl, any of which can be optionally substituted by oxo, CO₂R, heterocylyl, aryl, OR, NRR′, or SR;

R² comprises (C(R″)₂)ₙ wherein n is 0-3, wherein R″ is independently at each occurrence H, alkyl, halo, haloalkyl, OR, or NRR′, or two R″ together with a carbon atom to which they are bonded comprise a carbonyl;

R³ comprises alkyl, cycloalkyl, heterocyclyl, aryl, or heteroaryl, wherein any alkyl, cycloalkyl, heterocyclyl, aryl, or heteroaryl is substituted with 0-5 Z groups,

R⁴ comprises H, alkyl, aryl, or heteroaryl, wherein any alkyl, aryl, heteroaryl, is substituted with 0-4 Z; provided that R⁴ is absent when a double bond is present between the C-6 carbon atom bearing R⁵ or R⁶ and the adjacent N-5 nitrogen atom; and

wherein R⁵ and R⁶ together with the carbon atom to which they are bonded can form a carbonyl group or R⁷ and R⁸ together with the carbon atom to which they are bonded can form a carbonyl group, or both, or neither R⁵ and R⁶ and the carbon atom to which they are bonded nor R⁷ and R⁸ and the carbon atom to which they are bonded forms a carbonyl group; wherein R⁵, R⁶, R⁷, or R⁸, when present, each independently comprises hydrogen or (C₁-C₆)alkyl wherein any alkyl group is substituted with 0-4 Z; wherein a carbon atom that is not within a carbonyl group can bear a single substituent and be double bonded to the adjacent nitrogen atom or can bear two substituents and be single bonded to the adjacent nitrogen atom.

More specifically, R¹ can be halo, such as chloro, or can be trifluoromethyl, methyl, or methoxyl.

More specifically, R⁵ and R⁶ can together form a carbonyl group, or R⁷ and R⁸ can together form a carbonyl group. Or, both R⁵ and R⁶ can together form a carbonyl group, and R⁷ and R⁸ can together form a carbonyl group.

More specifically, R³ can comprise a substituted pyridyl moiety, such as a 3,5-dimethyl-4-methoxypyrid-2-ylmethyl moiety. Or, R³ can comprise a substituted phenyl group, such as a methylenedioxyphenyl group optionally further comprising a halo substituent, such as a chloro substituent. R² can be a methylene group.

More specifically, R⁴ can comprise an alkyl group, such as an isopropyl, isobutyl, or 4-methylpentyl group.

In various embodiments, R is H. In other embodiments, there is a double bond present between N-5 and C-6, for example when R⁷ and R⁸ together form a carbonyl group, to provide a compound of the formula:

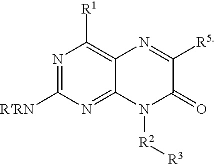

In various embodiments, the compound of formula (I) can be any of the following:

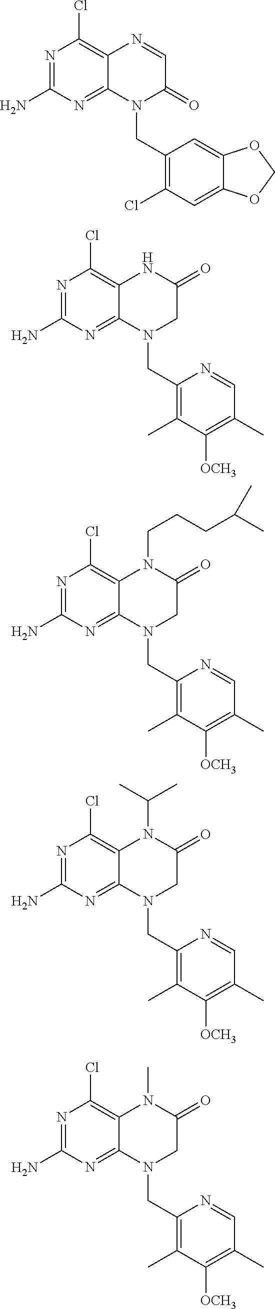

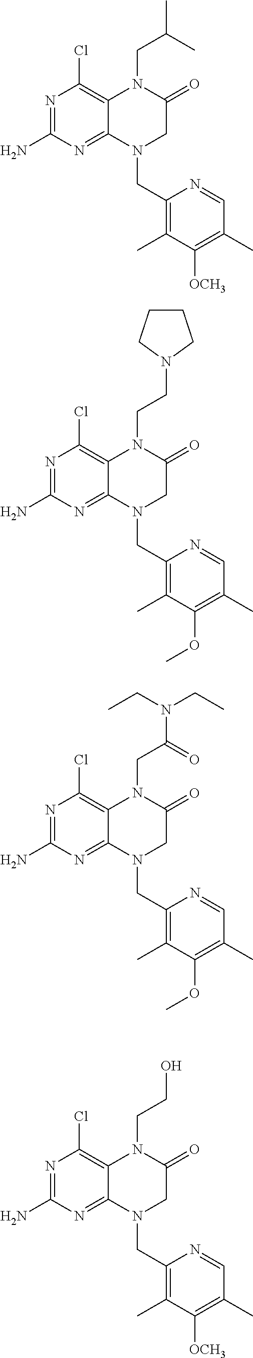

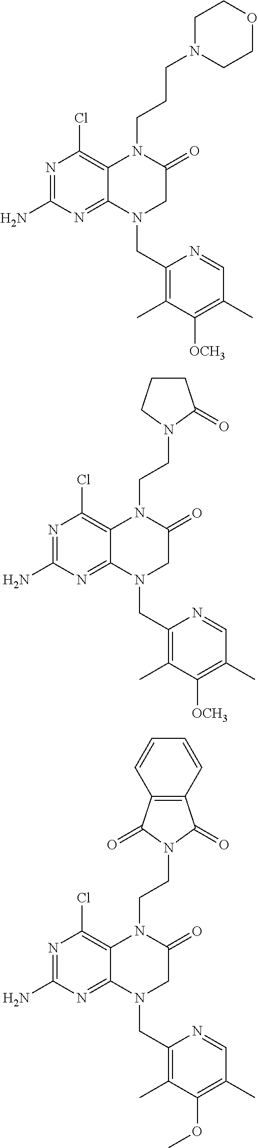

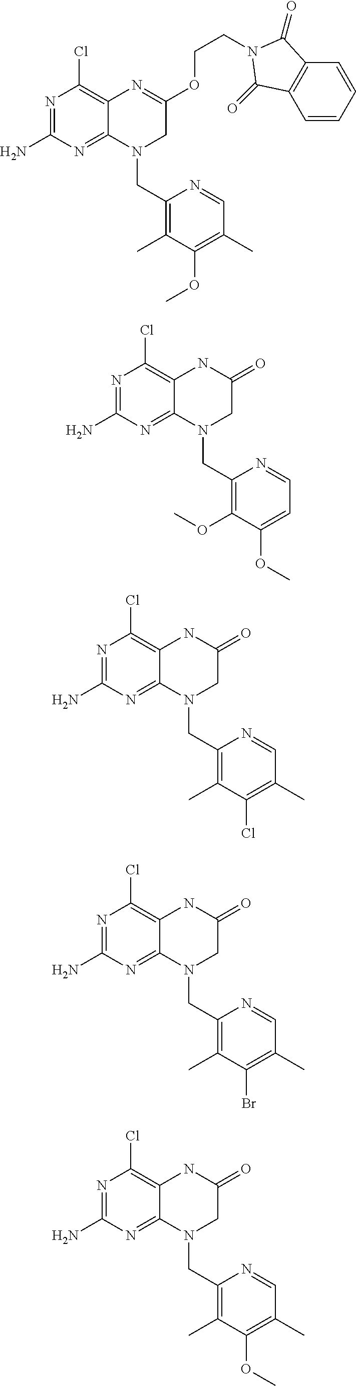

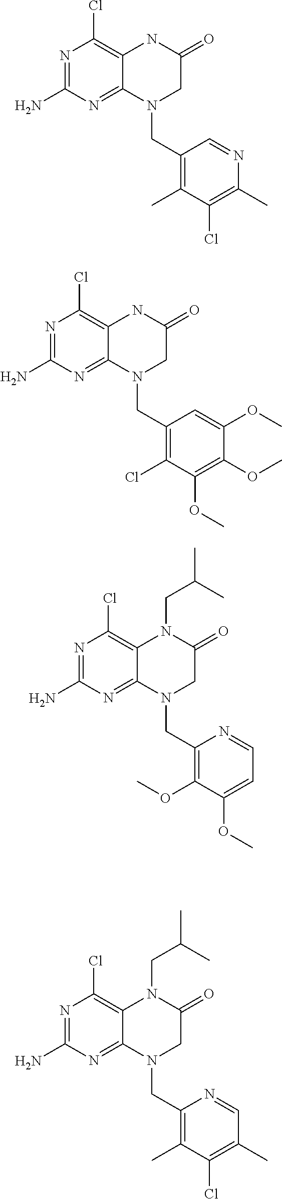

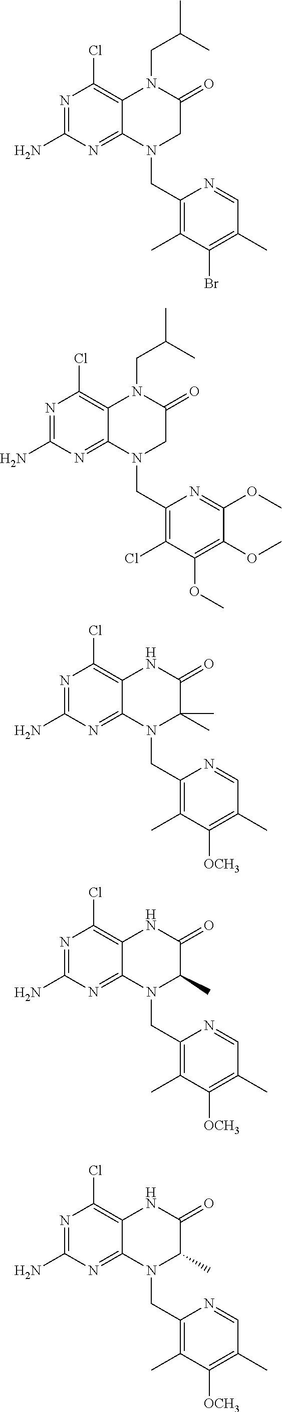

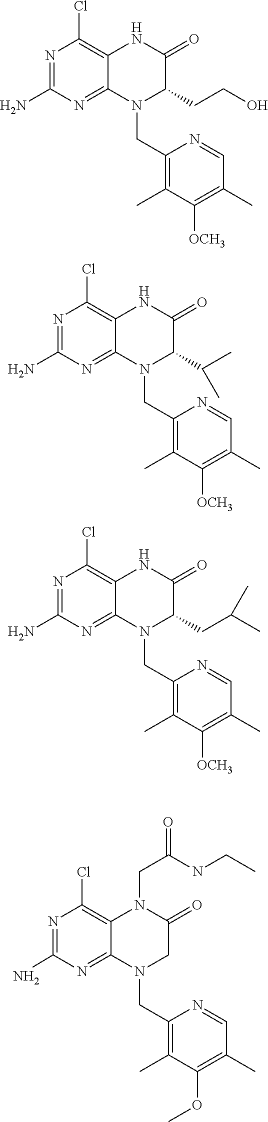

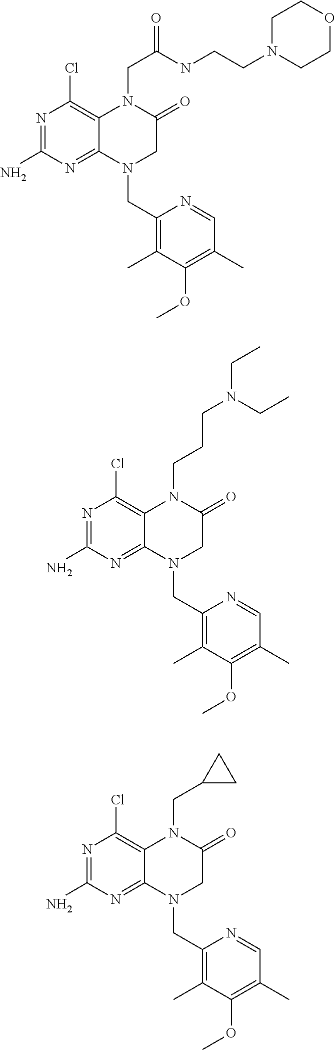

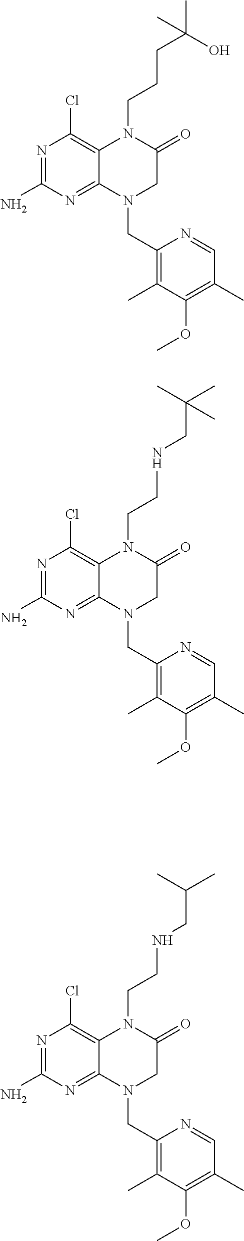

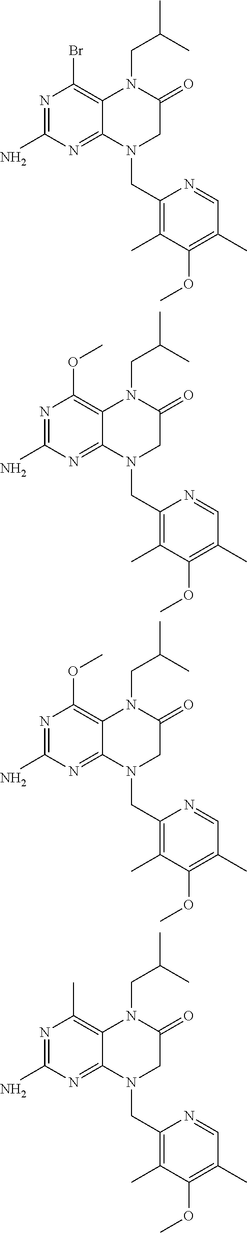

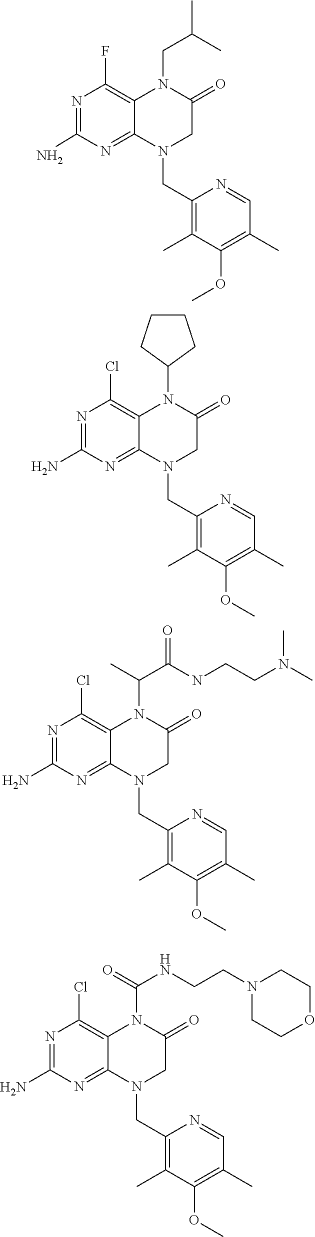

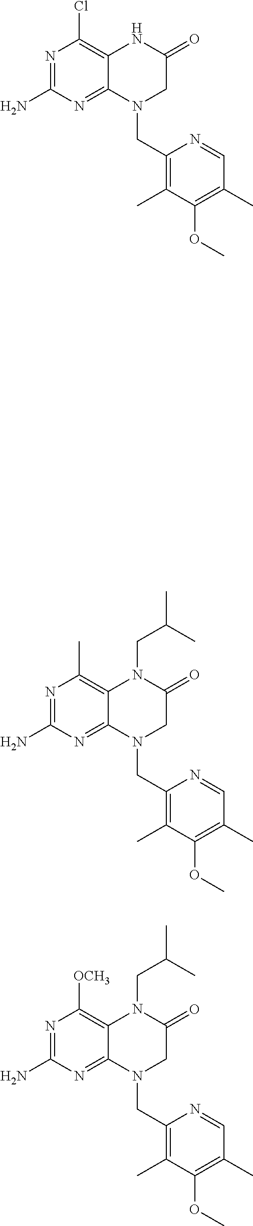

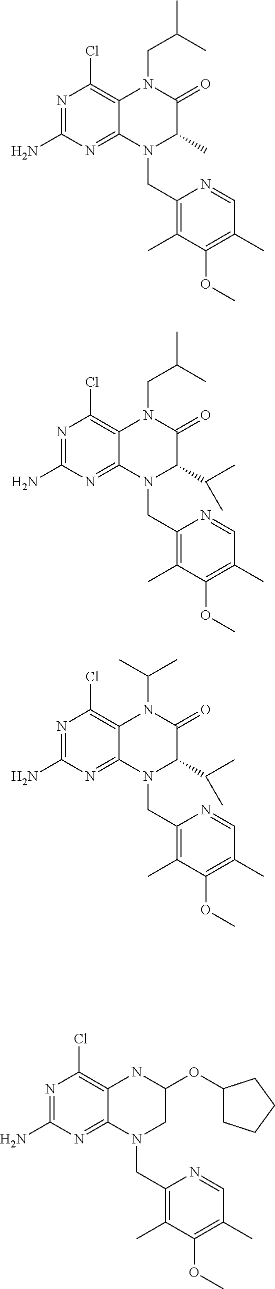

or any salt, solvate, hydrate, tautomer, stereoisomer, or prodrug thereof.

In various embodiments the invention provides a compound of formula (II):

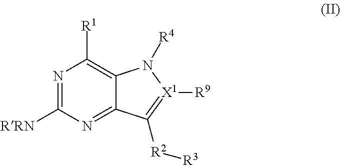

wherein

R and R′ are independently at each occurrence H, or (C₁-C₈)alkyl wherein any alkyl is optionally mono- or independently pluri-substituted with OR, halo, cyano, OC(O)R, OC(O)OR, CO₂R, NRR′, C(O)NRR′, N(R)C(O)R′, oxo, OC(O)NRR′, SR, S(O)R, S(O)₂R, S(O)₂OR, or SO₂NRR′ alkyl, acyl, alkoxy, cycloalkyl, heterocyclyl, aryl, or heteroaryl, wherein any alkyl, acyl, alkoxy, cycloalkyl, heterocyclyl, aryl, or heteroaryl is substituted with 0-4 Z; wherein any carbon atom of the alkyl, acyl, alkoxy, cycloalkyl, heterocyclyl, aryl, or heteroaryl, can be replaced by a heteroatom selected from the group consisting of NR, O, S, S(O), and S(O)₂; or, R and R′ can together with a nitrogen atom to which they are bonded form a heterocyclic or heteroaryl ring, wherein the heterocyclic or heteroaryl ring can further comprise 1-3 O, N, S, S(O), or S(O)₂ heteroatoms therewithin and is substituted with 0-4 Z groups wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R¹ comprises H, halo, NRR′, or hydroxyl; or comprises (C₁-C₆)alkyl, haloalkyl, cycloalkyl, or heterocyclyl, any of which can be optionally substituted by oxo, CO₂R, heterocylyl, aryl, OR, NRR′, or SR;

R² comprises (C(R″)₂)ₙ wherein n is 0-3, wherein R″ is independently at each occurrence H, alkyl, halo, haloalkyl, OR, or NRR′, or two R″ together with a carbon atom to which they are bonded comprise a carbonyl;

R³ comprises alkyl, cycloalkyl, heterocyclyl, aryl, or heteroaryl, wherein any alkyl, cycloalkyl, heterocyclyl, aryl, or heteroaryl is substituted with 0-5 Z groups,

R⁴ comprises H, alkyl, aryl, or heteroaryl, wherein any alkyl, aryl, heteroaryl, is substituted with 0-4 Z; provided that R⁴ is absent when a double bond is present between the carbon atom bearing R⁵ or R⁶ and the adjacent nitrogen atom;

X¹ comprises C or N, provided that when X′ is N, R⁹ is absent; and

R⁹ comprises H, aryl, or alkyl, wherein any aryl or alkyl is substituted with 0-4 Z, wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′.

More specifically, R³ can comprise a substituted pyridyl moiety, such as a 3,5-dimethyl-4-methoxypyrid-2-ylmethyl moiety. R² can be a methylene group.

More specifically, R′ can be halo, such as chloro, or can be trifluoromethyl, methyl, or methoxyl.

More specifically, X′ can be N and R⁹ consequently be absent.

In various embodiments, the compound of formula (II) can be any of the following:

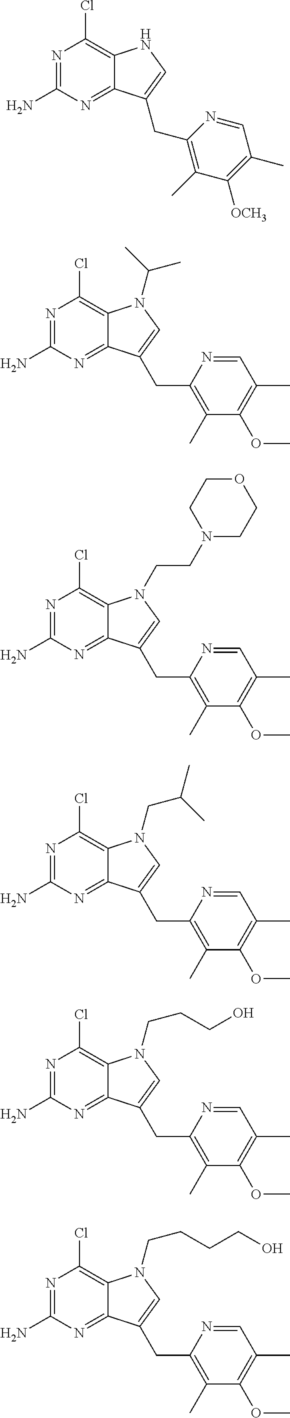

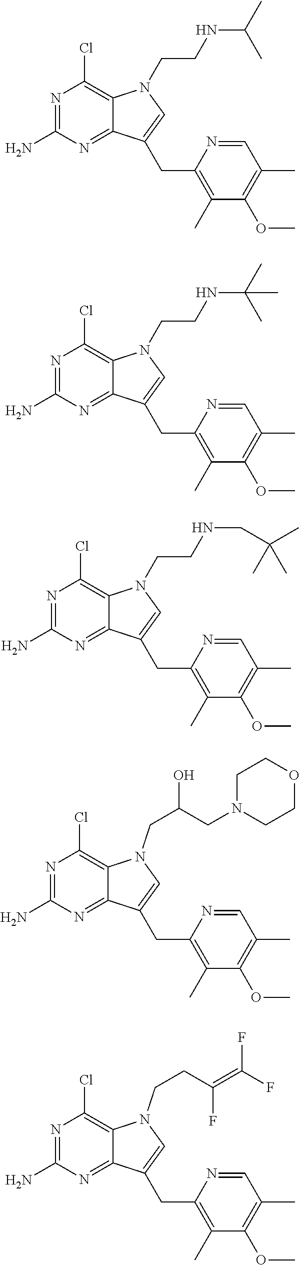

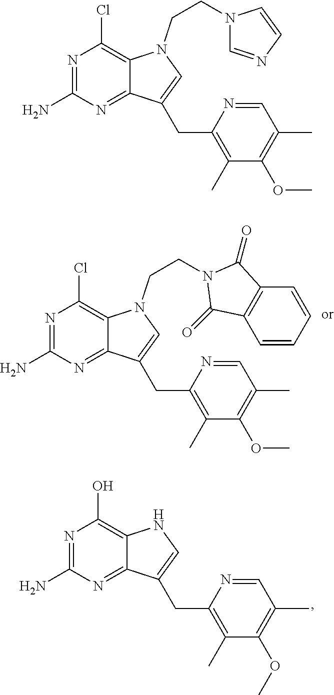

or any salt, solvate, hydrate, tautomer, stereoisomer, or prodrug thereof.

Various embodiments of the invention provide a compound of formula (III):

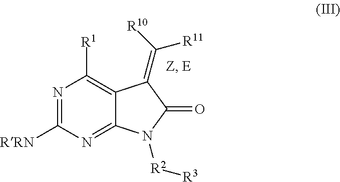

wherein

Y¹ is O or S;

R and R′ are independently at each occurrence H, or (C₁-C₈)alkyl wherein any alkyl is optionally mono- or independently pluri-substituted with OR, halo, cyano, OC(O)R, OC(O)OR, CO₂R, NRR′, C(O)NRR′, N(R)C(O)R′, oxo, OC(O)NRR′, SR, S(O)R, S(O)₂R, S(O)₂OR, or SO₂NRR′ alkyl, acyl, alkoxy, cycloalkyl, heterocyclyl, aryl, or heteroaryl, wherein any alkyl, acyl, alkoxy, cycloalkyl, heterocyclyl, aryl, or heteroaryl is substituted with 0-4 Z; wherein any carbon atom of the alkyl, acyl, alkoxy, cycloalkyl, heterocyclyl, aryl, or heteroaryl, can be replaced by a heteroatom selected from the group consisting of NR, O, S, S(O), and S(O)₂; or, R and R′ can together with a nitrogen atom to which they are bonded form a heterocyclic or heteroaryl ring, wherein the heterocyclic or heteroaryl ring can further comprise 1-3 O, N, S, S(O), or S(O)₂ heteroatoms therewithin and is substituted with 0-4 Z groups wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R¹ comprises H, halo, NRR′, or hydroxyl; or comprises (C₁-C₆)alkyl, haloalkyl, cycloalkyl, or heterocyclyl, any of which can be optionally substituted by oxo, CO₂R, heterocylyl, aryl, OR, NRR′, or SR;

R² comprises (C(R″)₂)ₙ wherein n is 0-3, wherein R″ is independently at each occurrence H, alkyl, halo, haloalkyl, OR, or NRR′, or two R″ together with a carbon atom to which they are bonded comprise a carbonyl;

R³ comprises alkyl, cycloalkyl, heterocyclyl, aryl, or heteroaryl, wherein any alkyl, cycloalkyl, heterocyclyl, aryl, or heteroaryl is substituted with 0-5 Z groups;

R¹⁰ and R¹¹ are each independently H, alkyl, aryl, cycloalkyl, heterocyclyl, or heteroaryl, wherein any alkyl, aryl, cycloalkyl, heterocyclyl, or heteroaryl is substituted with 0-4 Z; wherein any aryl, cycloalkyl, heterocyclyl, or heteroaryl can be fused to a five to seven-membered carbocyclic or heterocyclic ring which is substituted with 0-4 Z; or, R¹⁰ and R¹¹ together with a carbon atom to which they are bonded form an optionally substituted carbocyclic or heterocyclic ring which is substituted with 0-4 Z; wherein a double bond marked Z, E can be in either a Z or an E configuration, or a mixture thereof.

More specifically, R³ can comprise a substituted pyridyl moiety, such as a 3,5-dimethyl-4-methoxypyrid-2-ylmethyl moiety. R² can be methylene.

More specifically, R¹ can be halo, such as chloro, or can be trifluoromethyl, methyl, or methoxyl.

More specifically, R¹⁰ can be H and R¹¹ can be a substituted or unsubstituted heteroaryl group, for example R¹¹ can comprise a pyrrole or imidazole group, optionally substituted with methyl, methoxycarbonyl, or trifluoroacetyl groups.

In various embodiments, the compound of formula (III) can be any of the following:

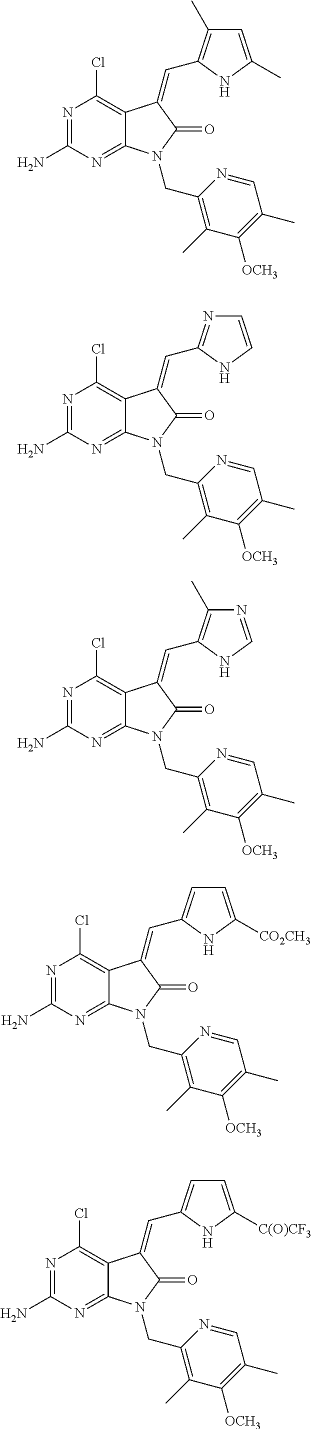

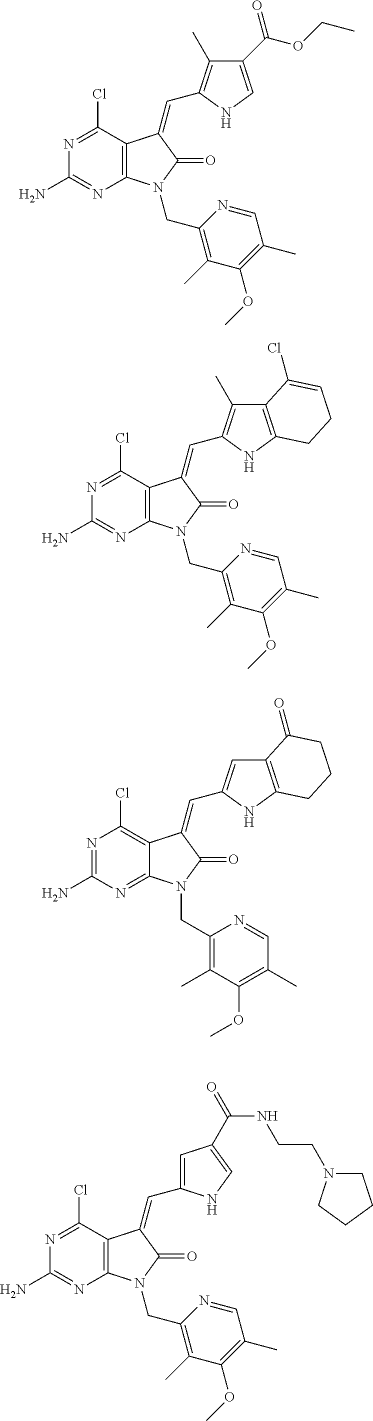

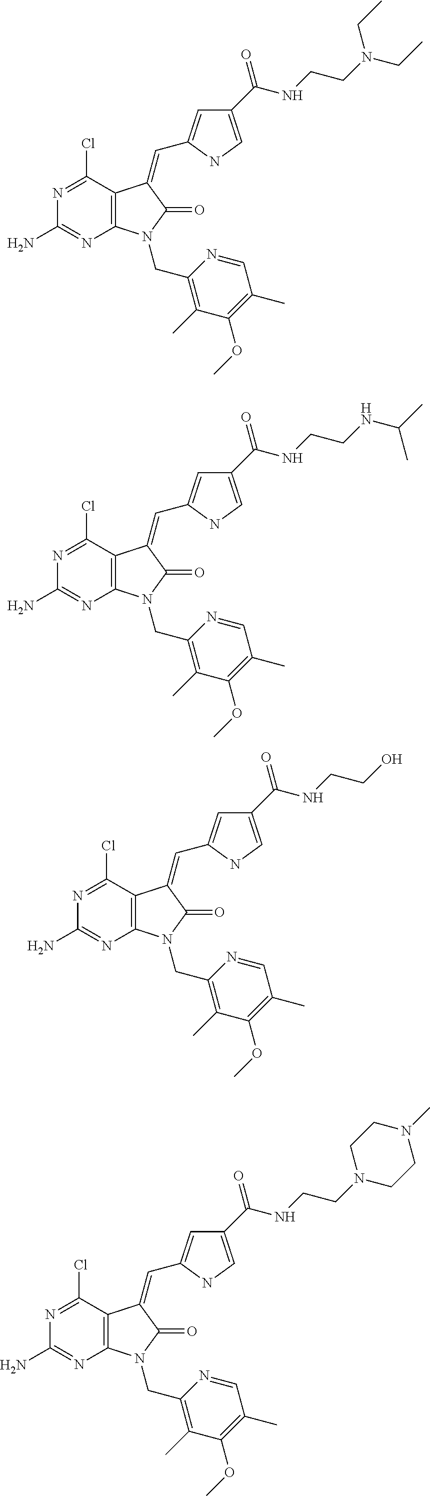

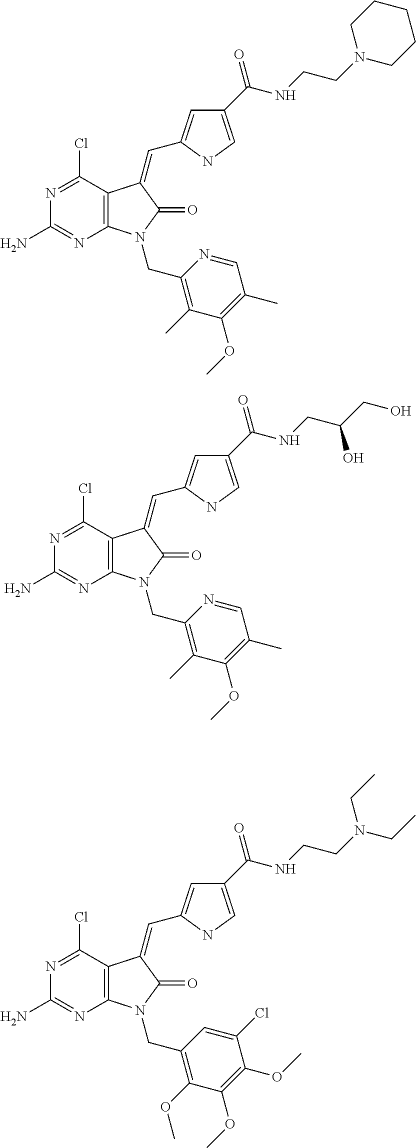

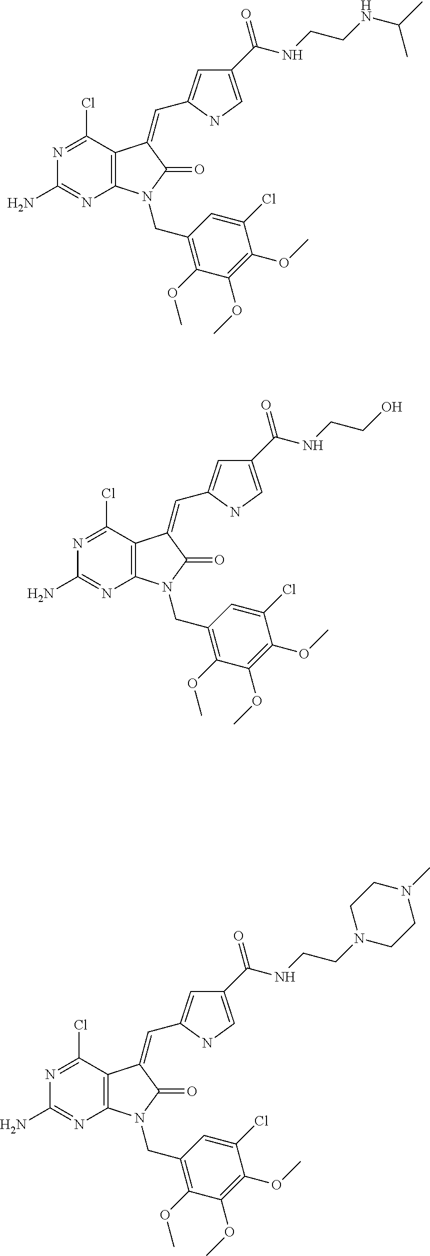

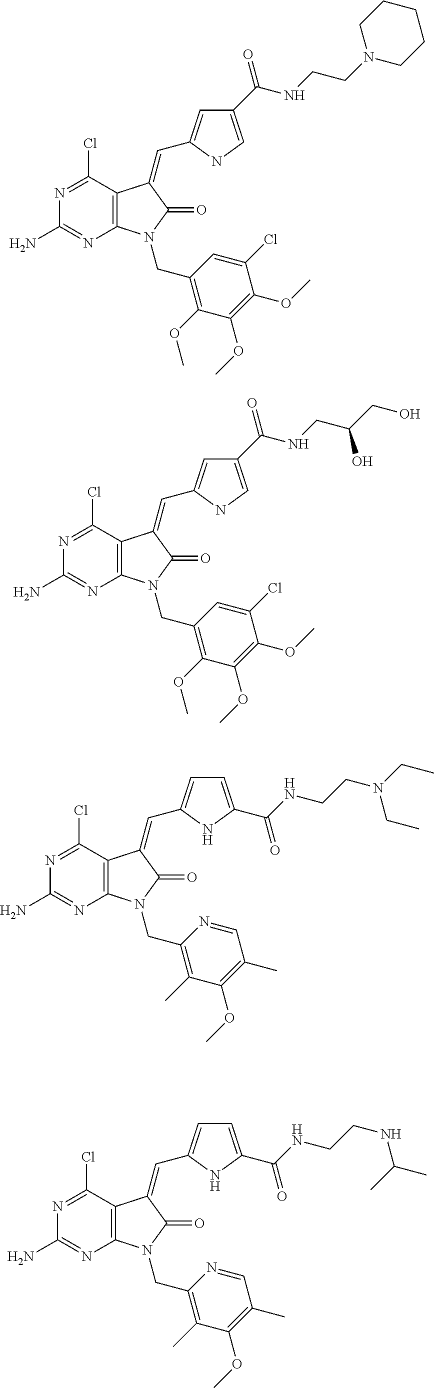

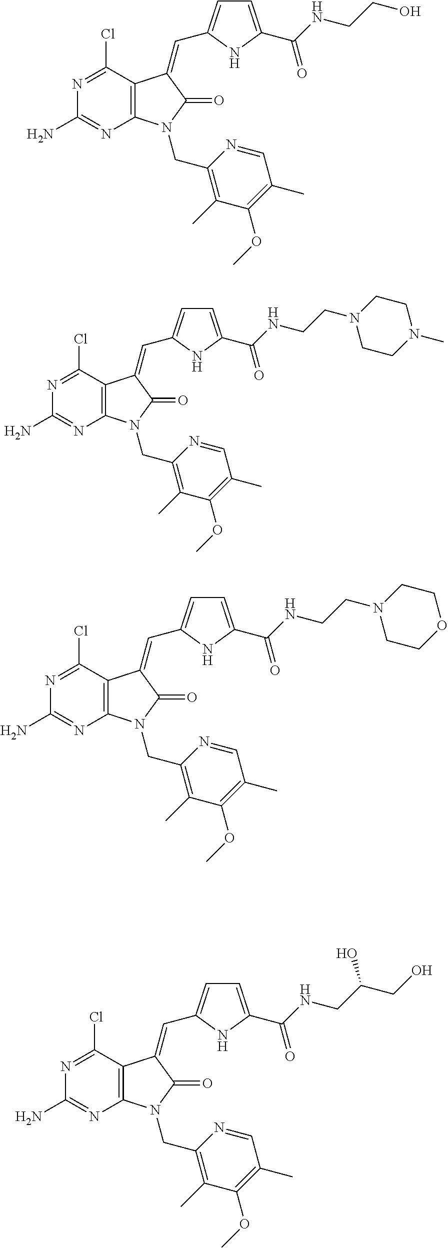

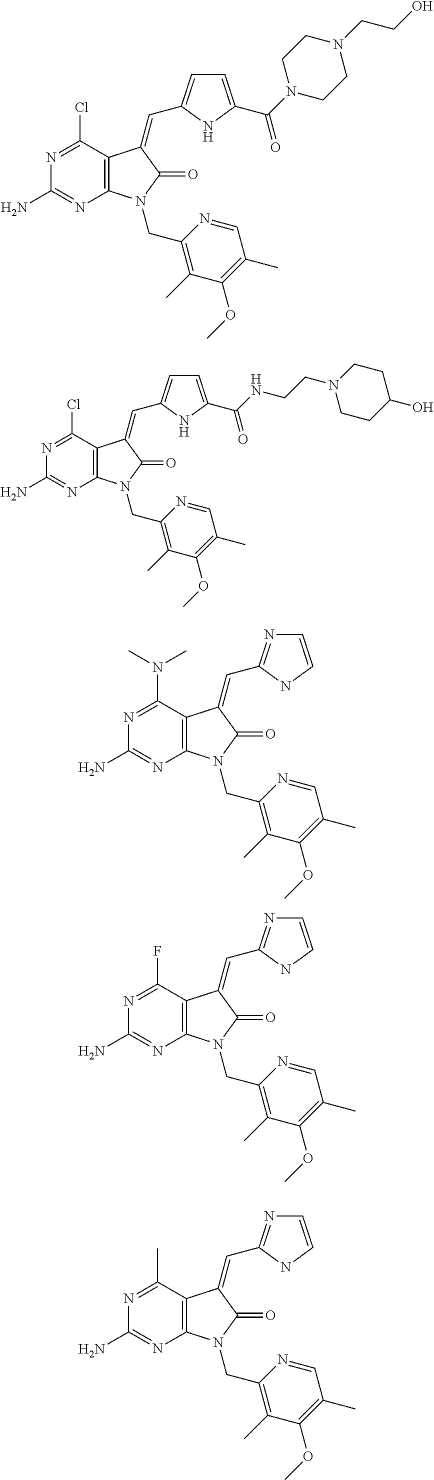

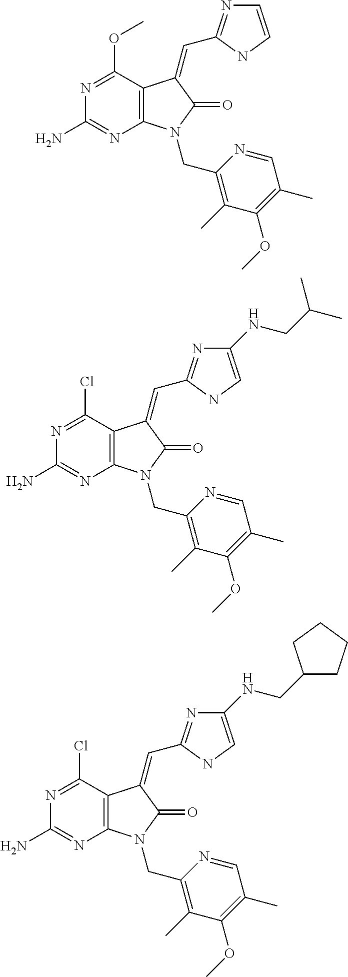

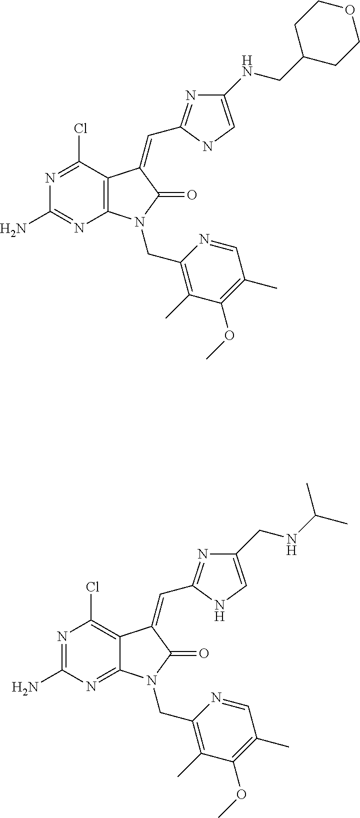

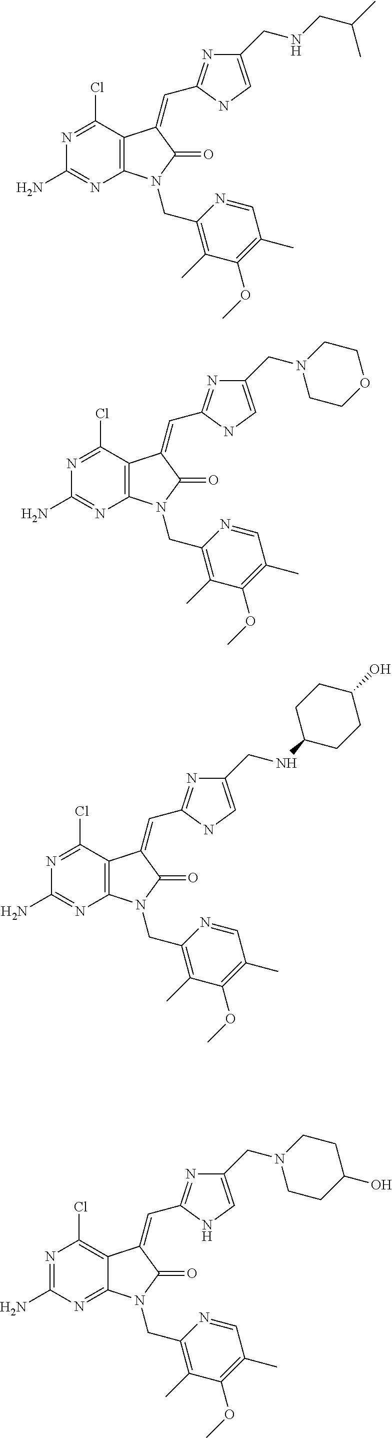

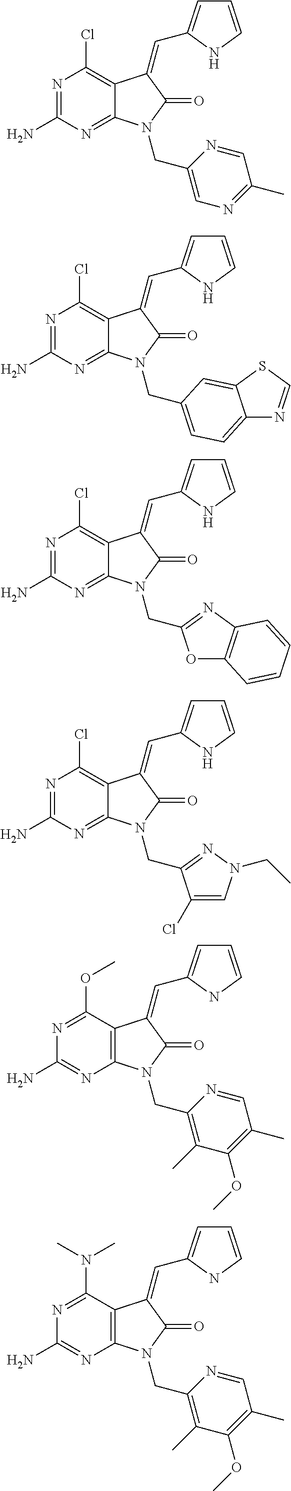

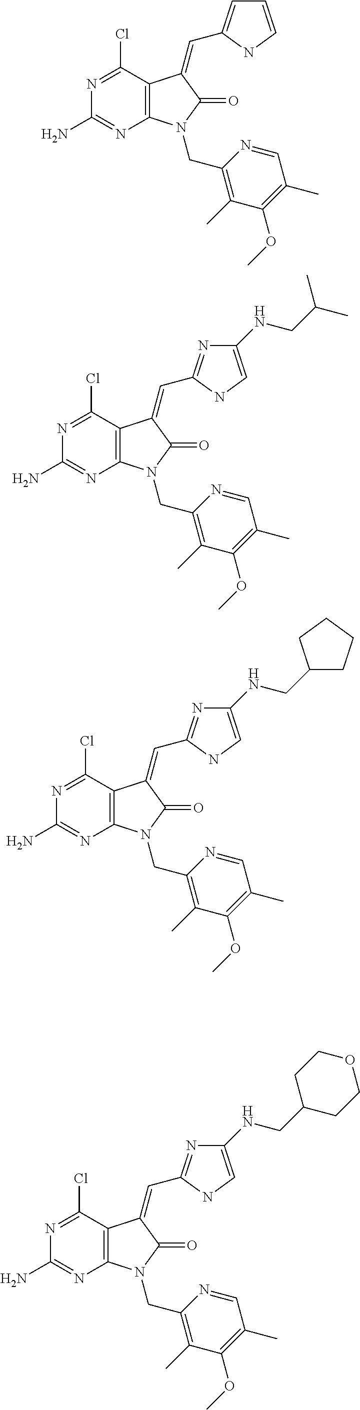

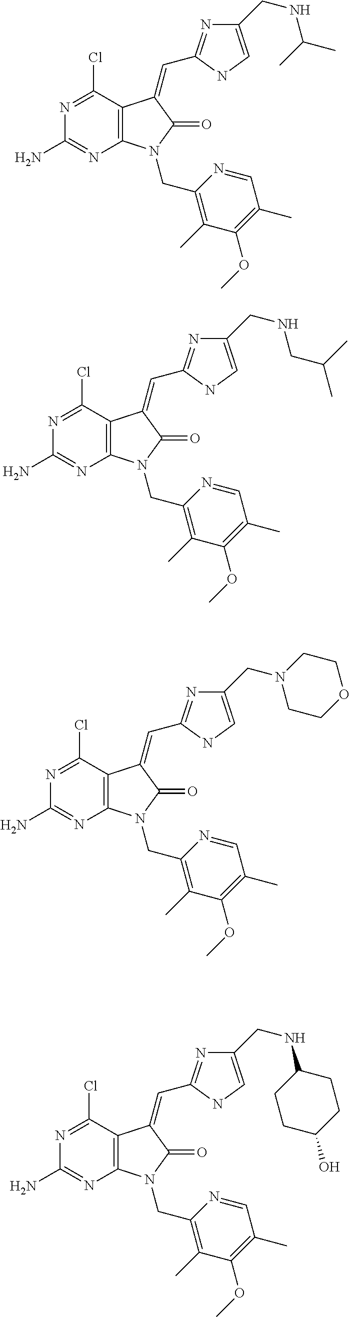

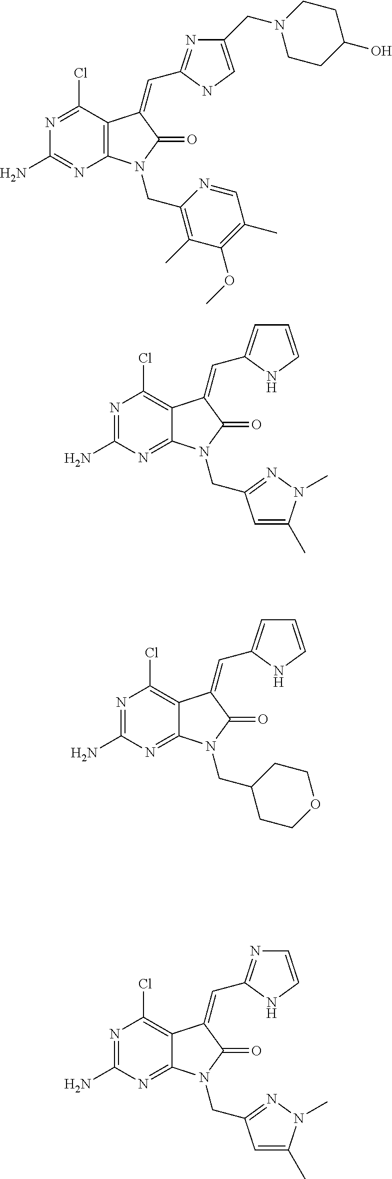

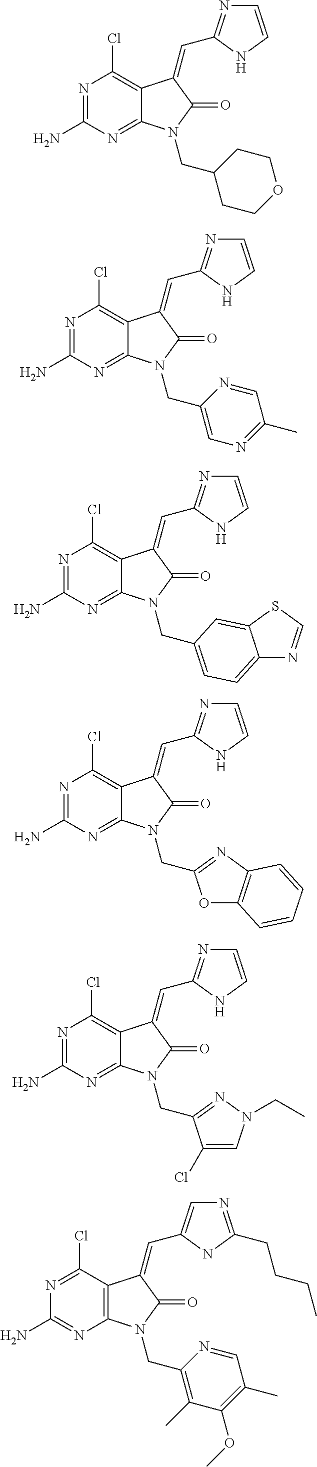

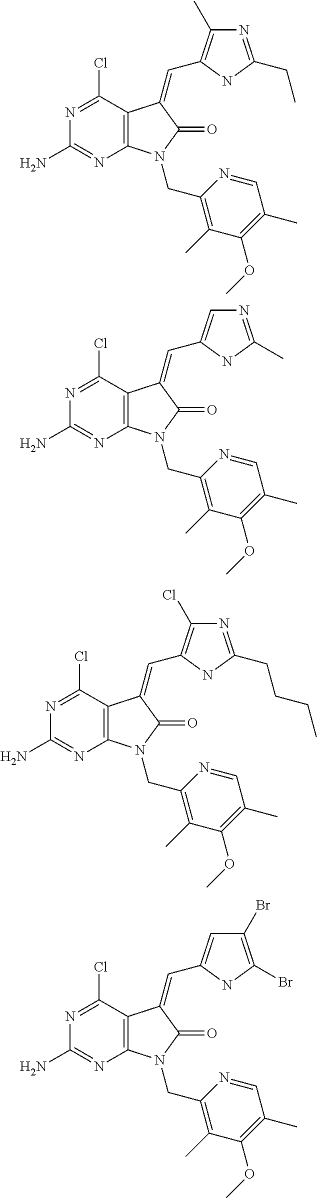

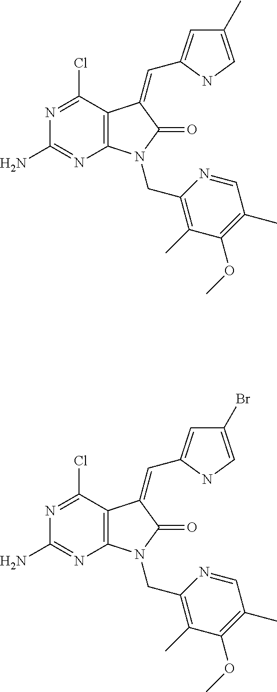

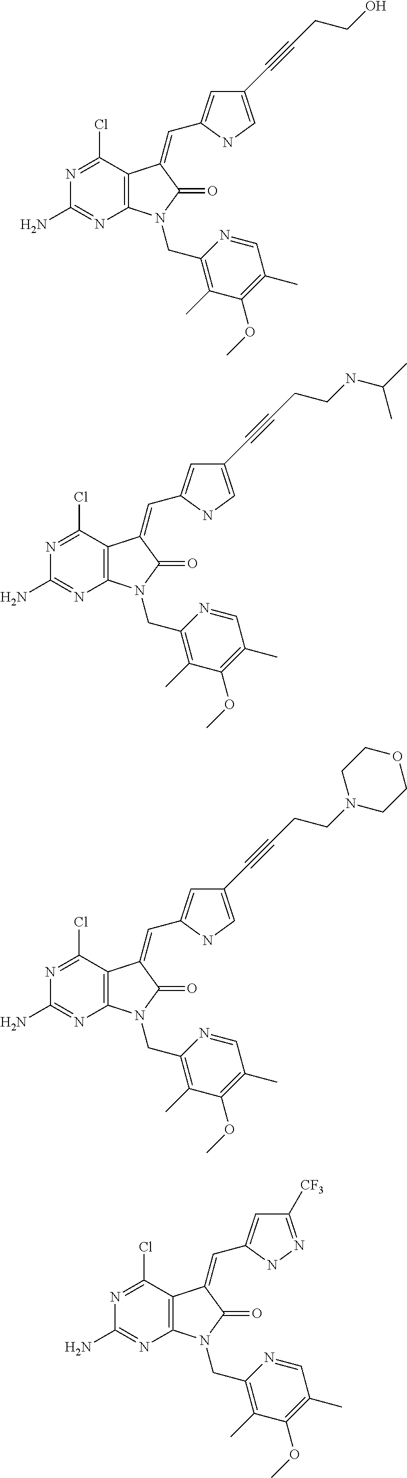

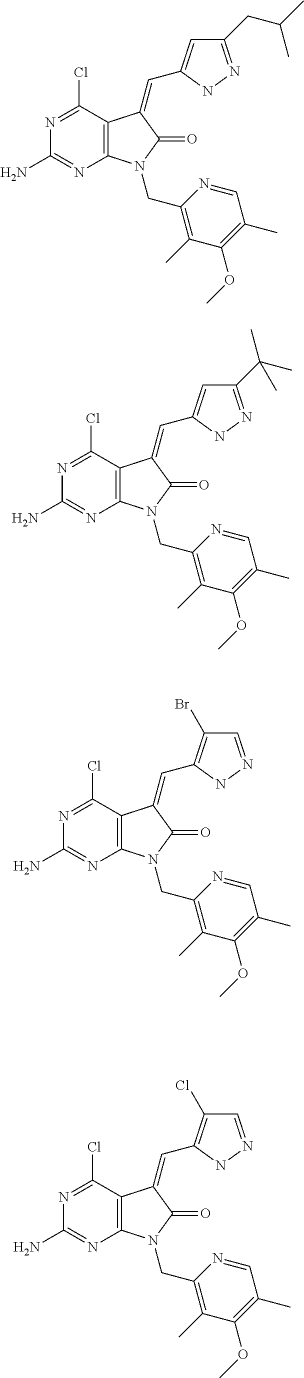

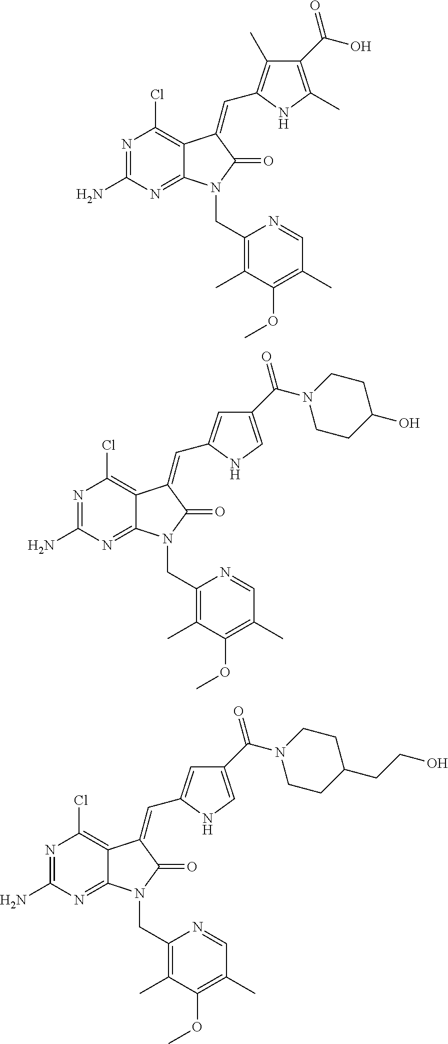

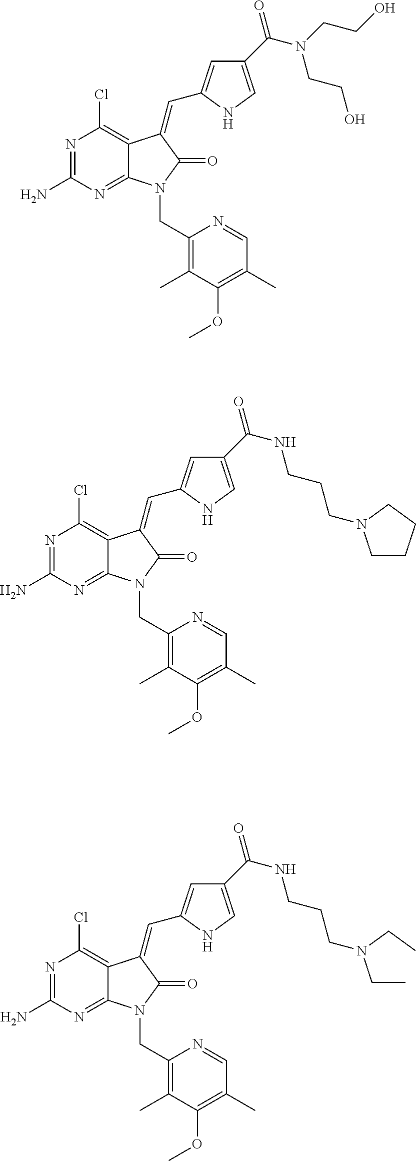

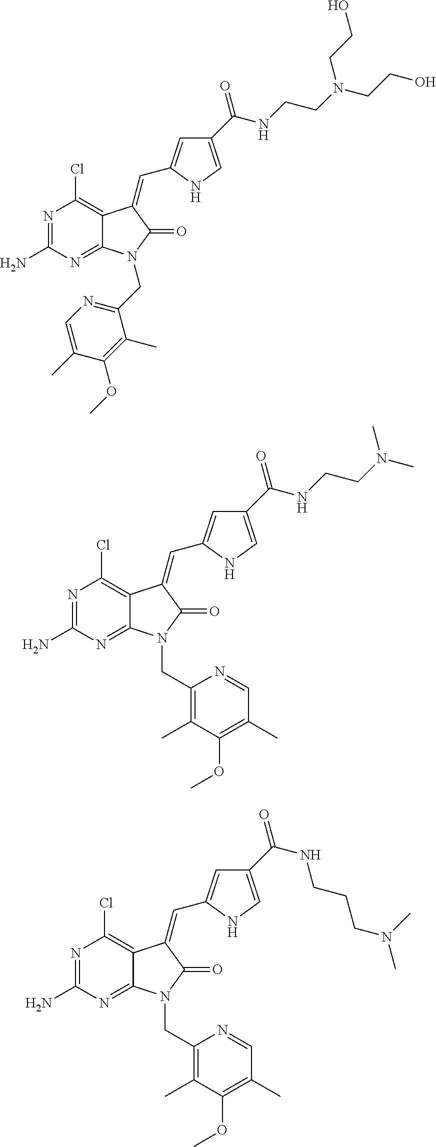

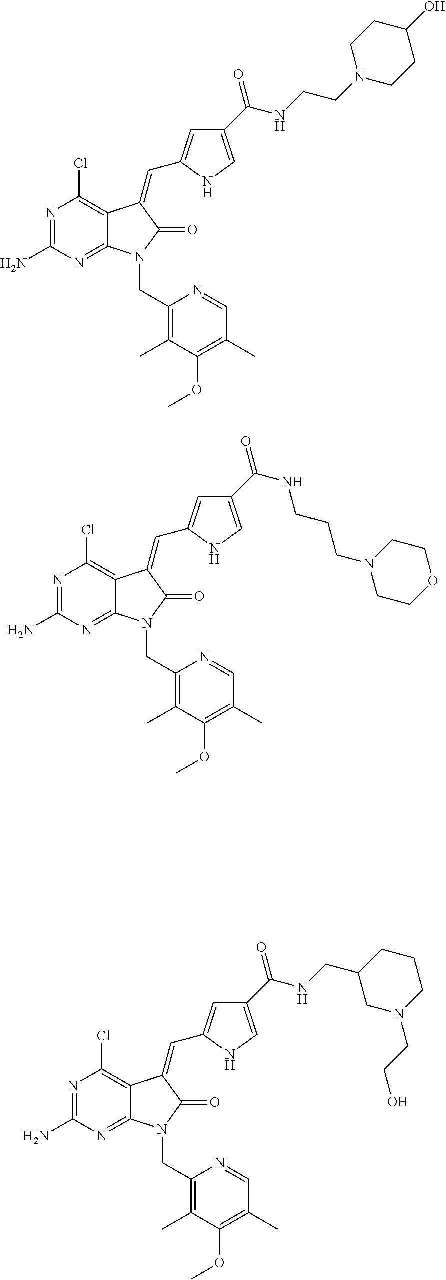

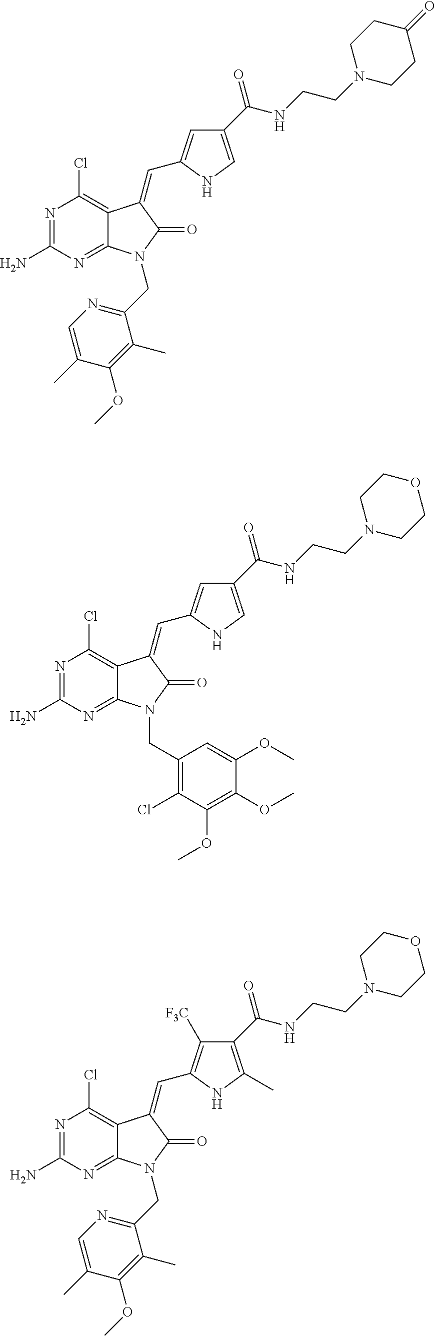

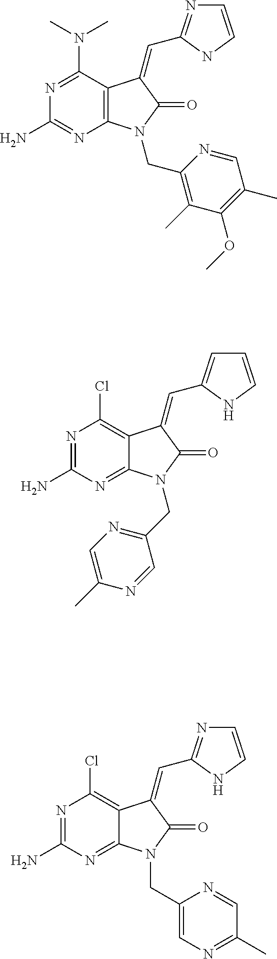

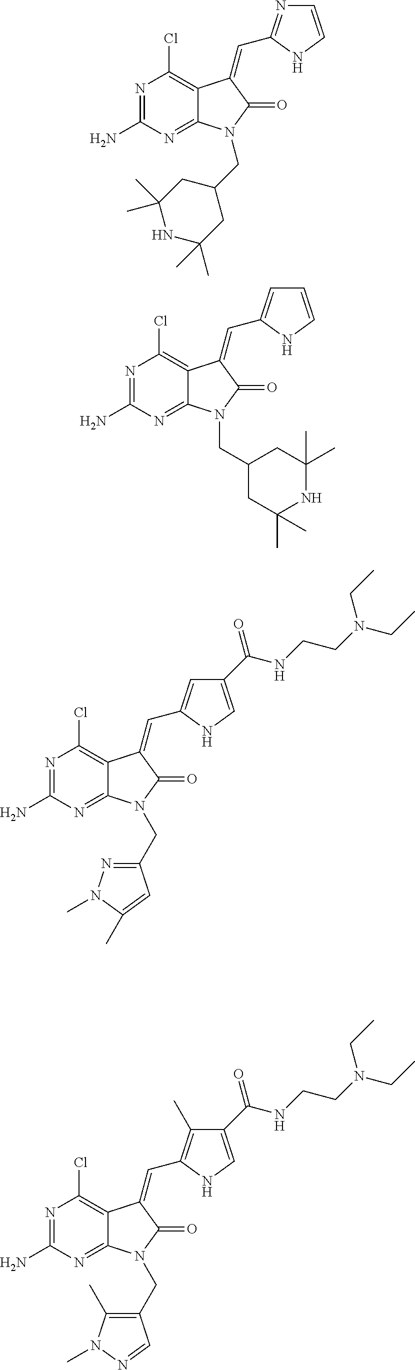

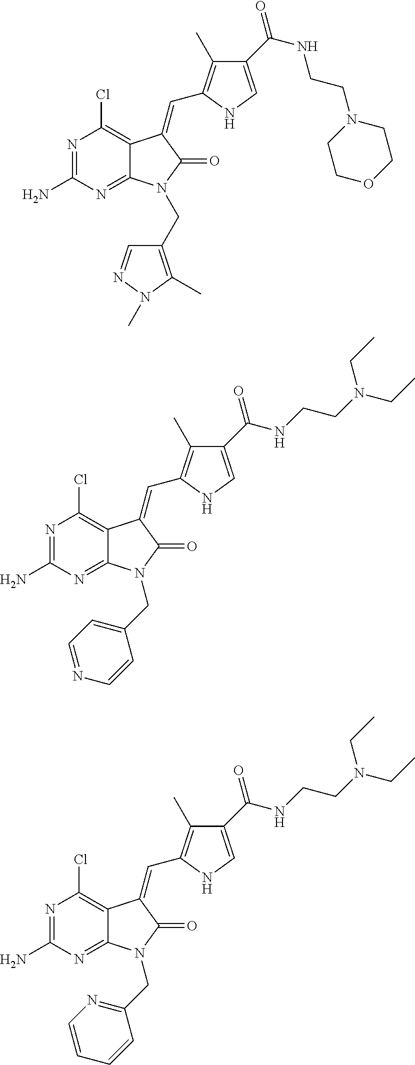

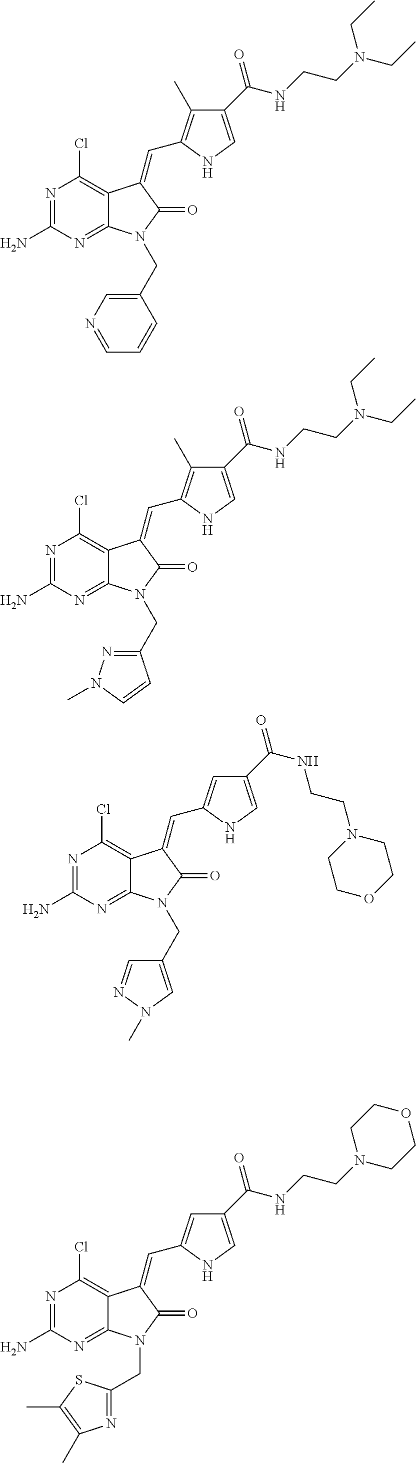

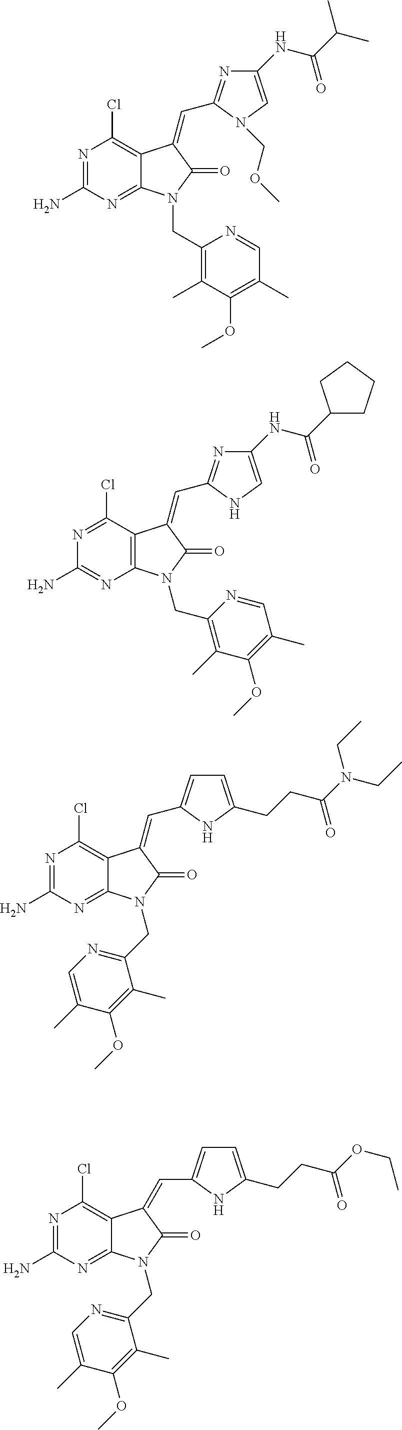

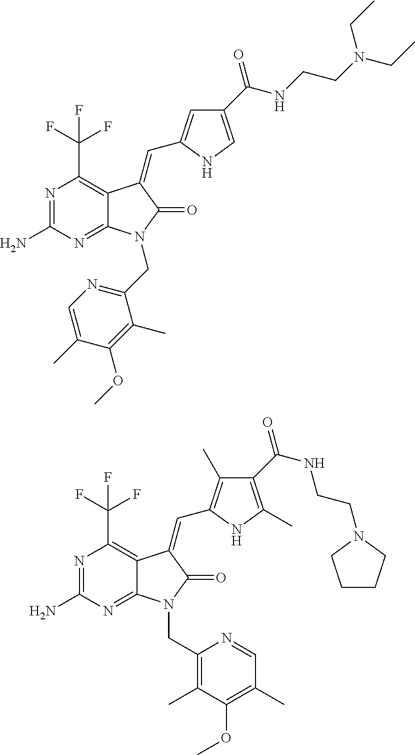

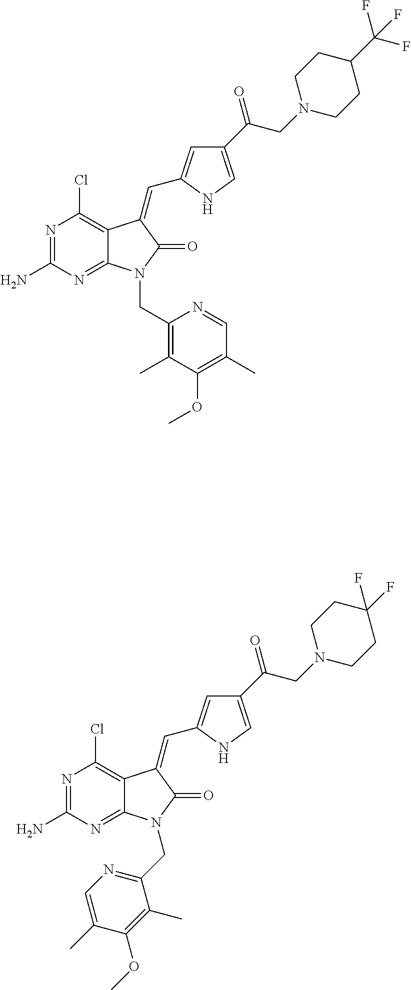

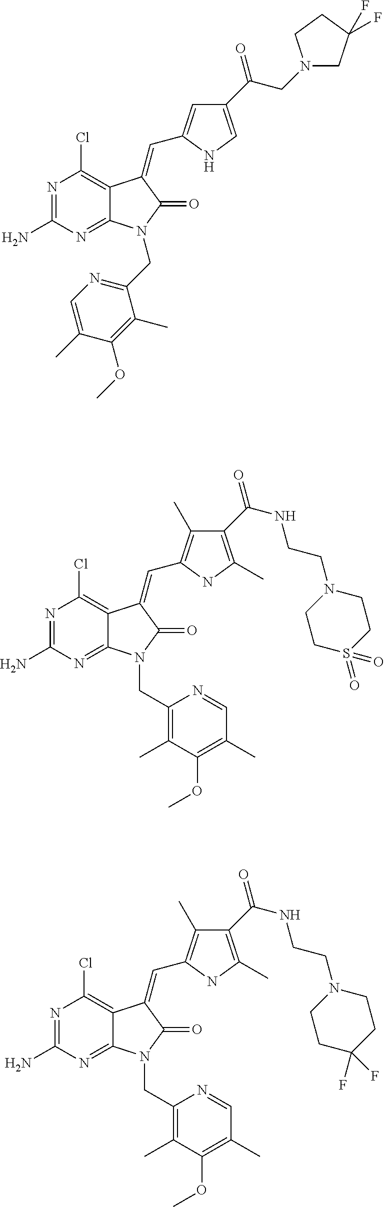

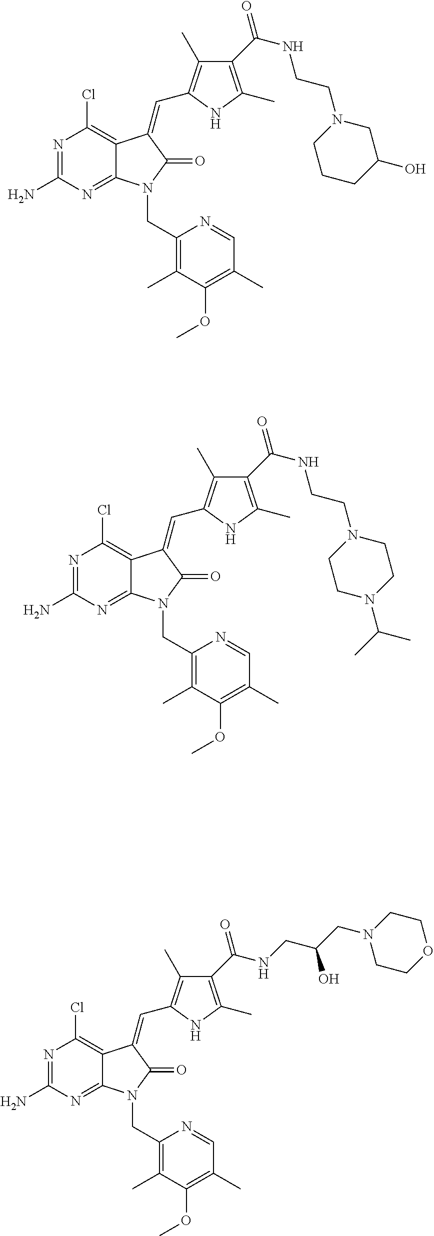

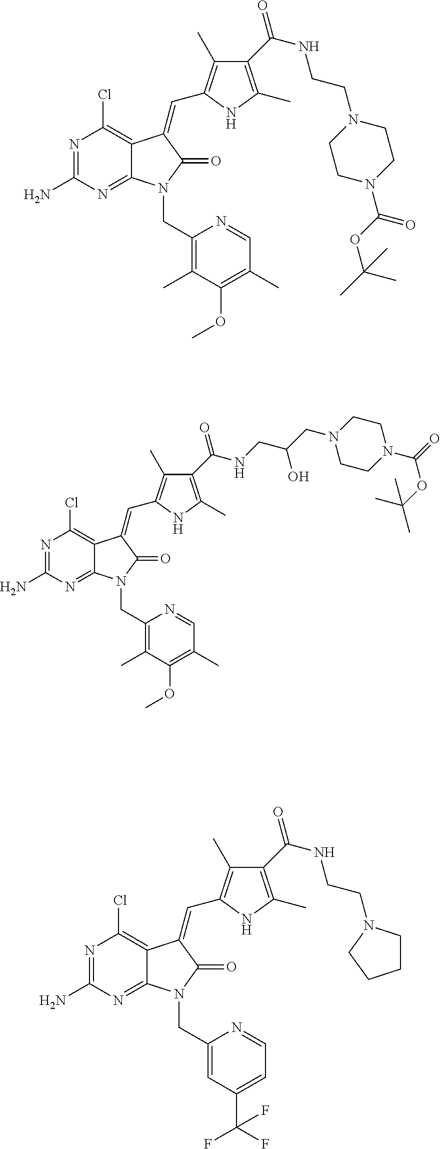

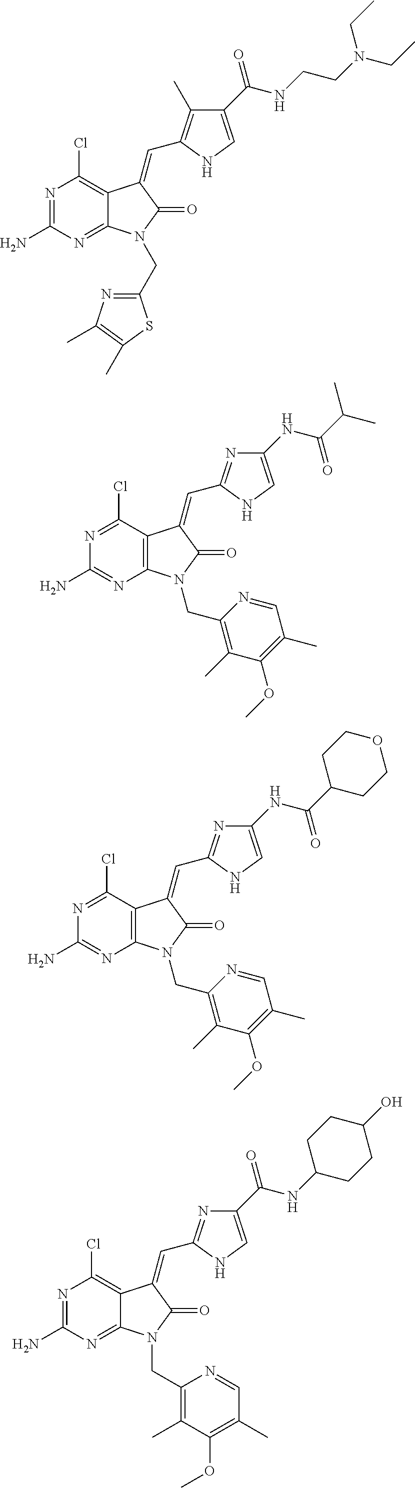

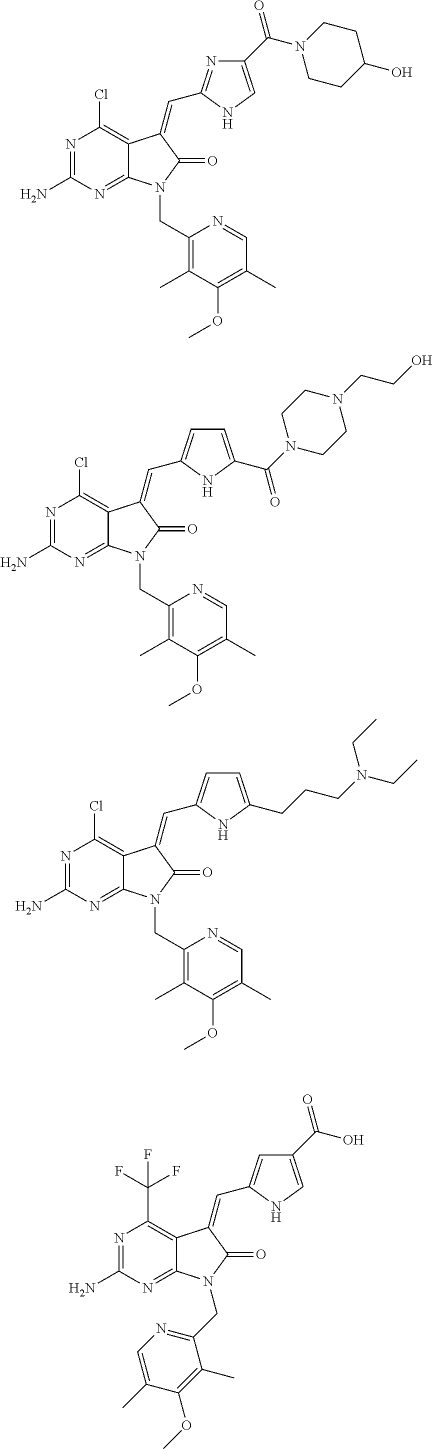

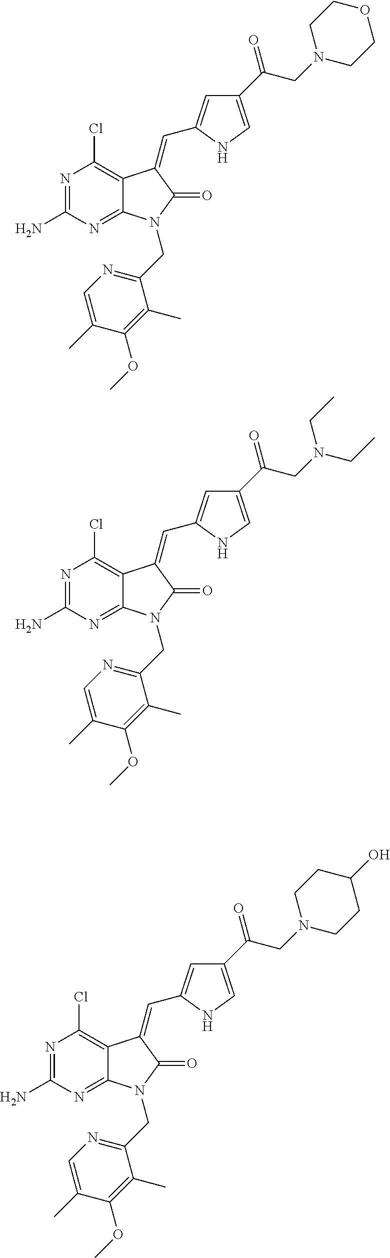

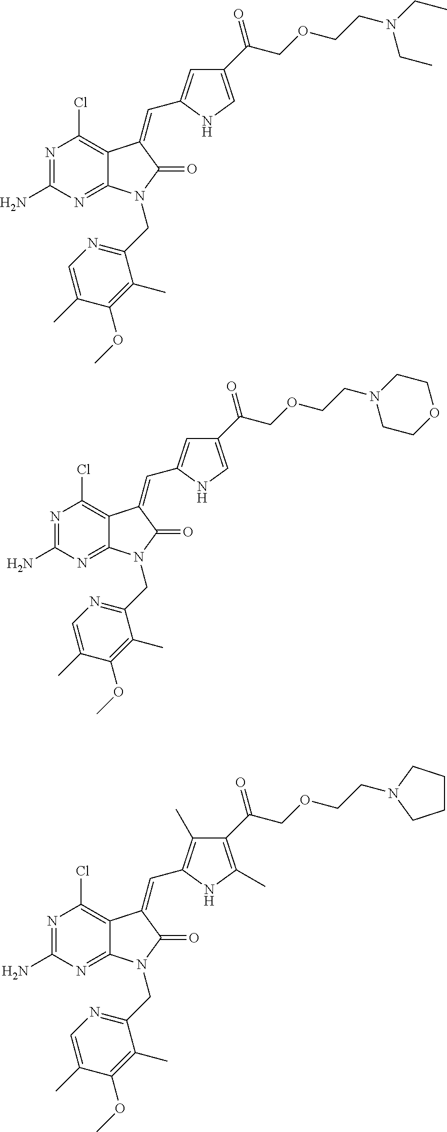

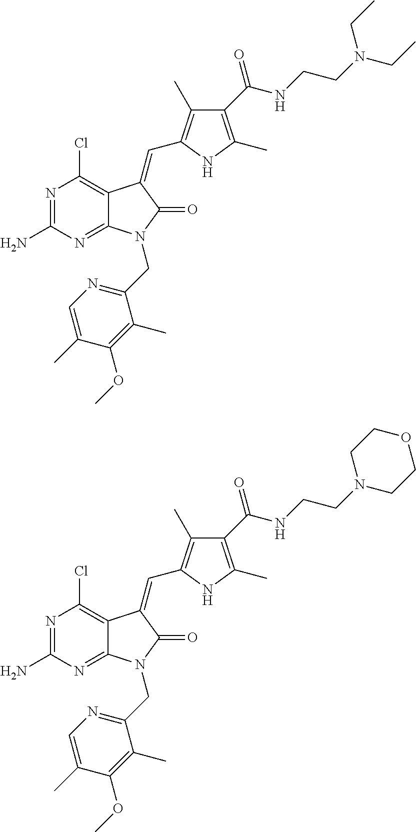

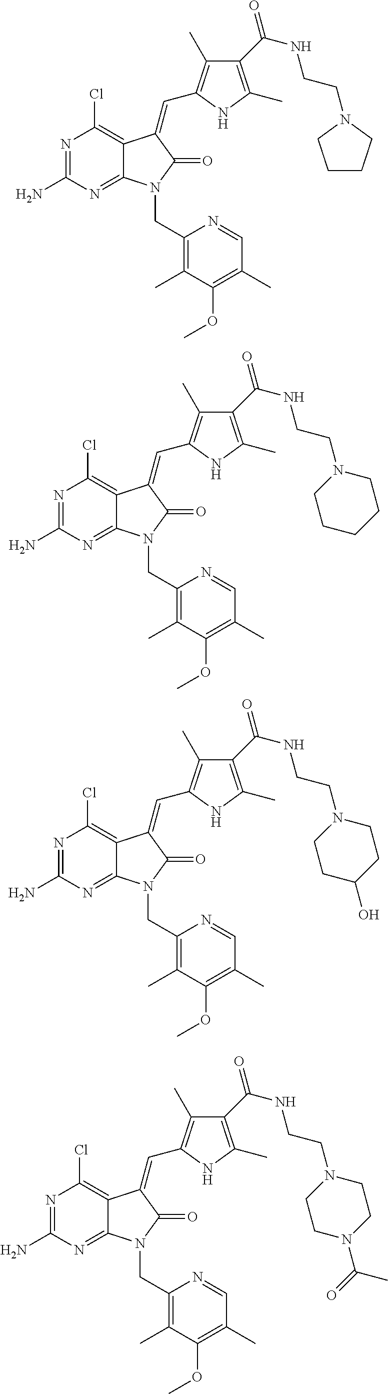

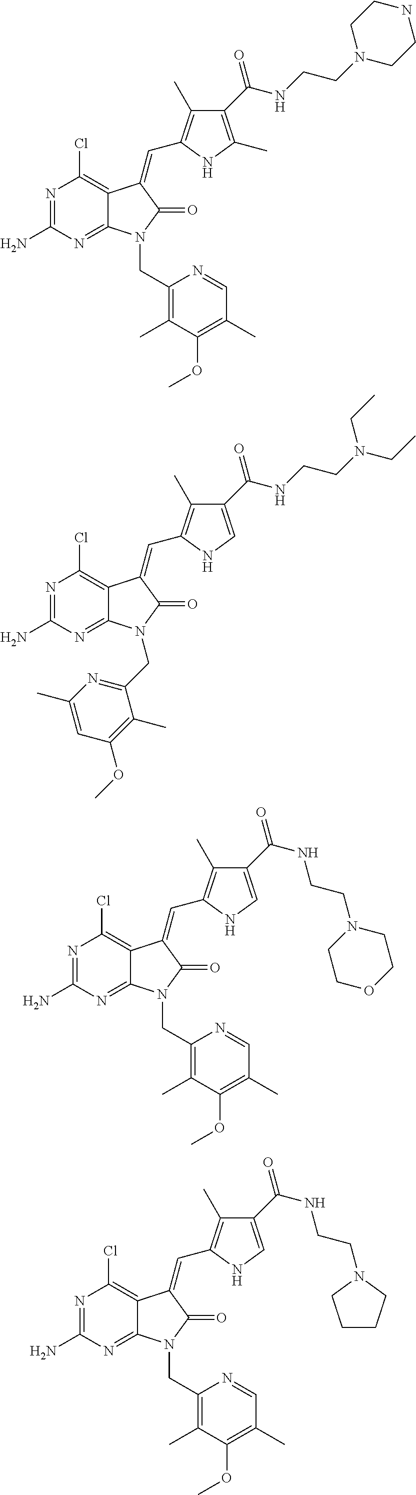

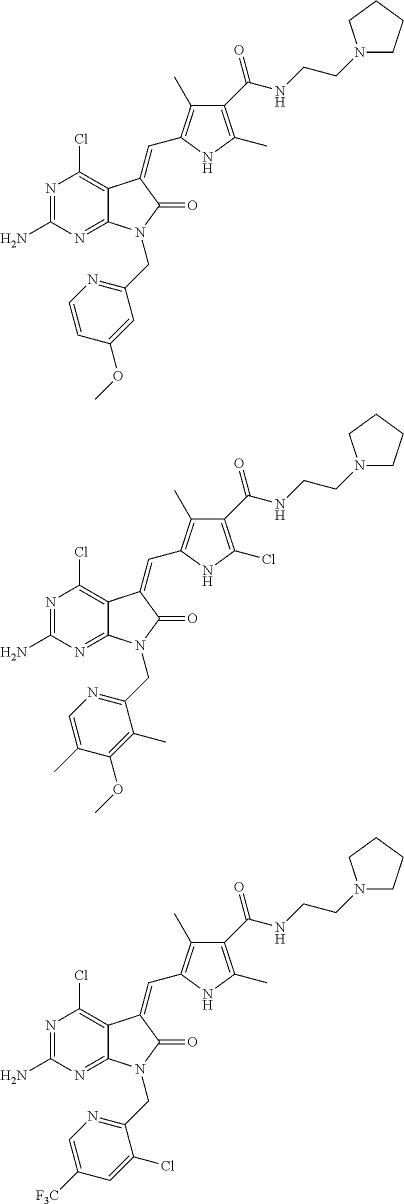

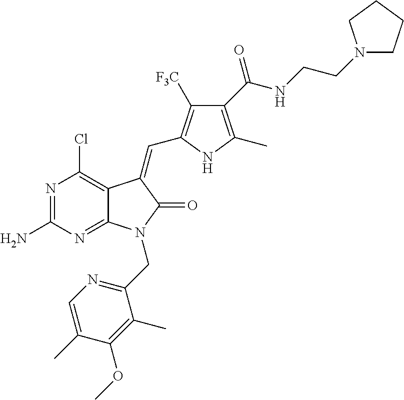

or any salt, solvate, hydrate, tautomer, stereoisomer, or prodrug thereof.

Various embodiments of the invention provide a compound of formula (IV):

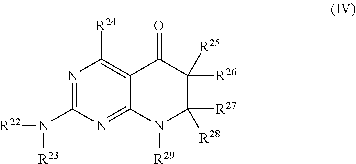

wherein

R²² and R²³ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z, wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²² and R²³ can together with the nitrogen atom to which they are bonded form a heterocyclic ring substituted with 0-4 Z; wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R²⁴ is H, halo, alkyl, haloalkyl, OR, NRR′, nitro, or cyano;

R²⁵ and R²⁶ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁵ and R²⁶ can together with a carbon atom to which they are bonded form a cycloalkyl ring substituted with 0-4 Z;

R²⁷ and R²⁸ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁷ and R²⁸ can together with a carbon atom to which they are bonded form a cycloalkyl ring substituted with 0-4 Z;

R²⁹ is H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁹ comprises a group of the formula R²-R³.

In various embodiments, R²⁹ can comprise a substituted pyridyl moiety, such as a (3,5-dimethyl-4-methoxypyridin-2-yl)methyl moiety or a (4,5-dimethoxypyridin-2-yl)methyl moiety.

In various embodiments, R²⁴ is halo, such as chloro.

In various embodiments, R²⁵ is H and R²⁶ comprises an alkyl group. For example R²⁶ can be isobutyl, cyclopentylmethyl, cyclopropylmethyl or (tetrahydro-2H-pyran-4-yl)methyl.

In various embodiments, R²², R²³, R²⁷ and R²⁸ are H.

In various embodiments, the compound of formula (IV) is any of the following:

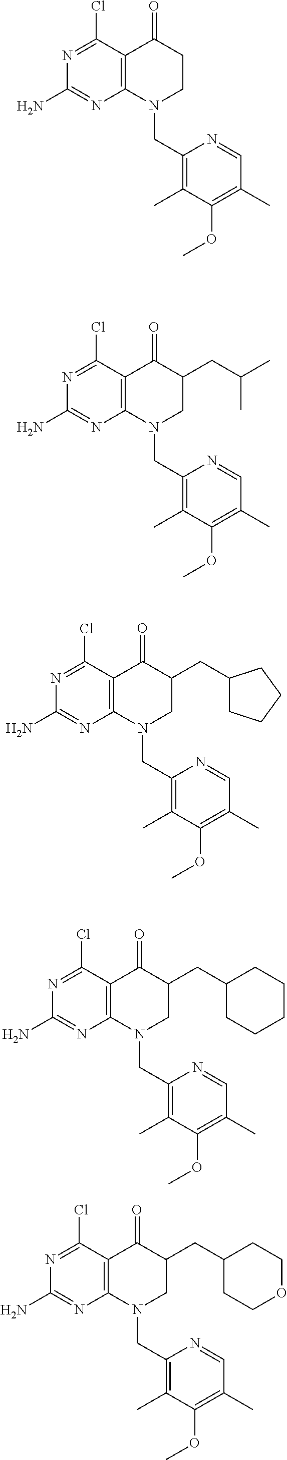

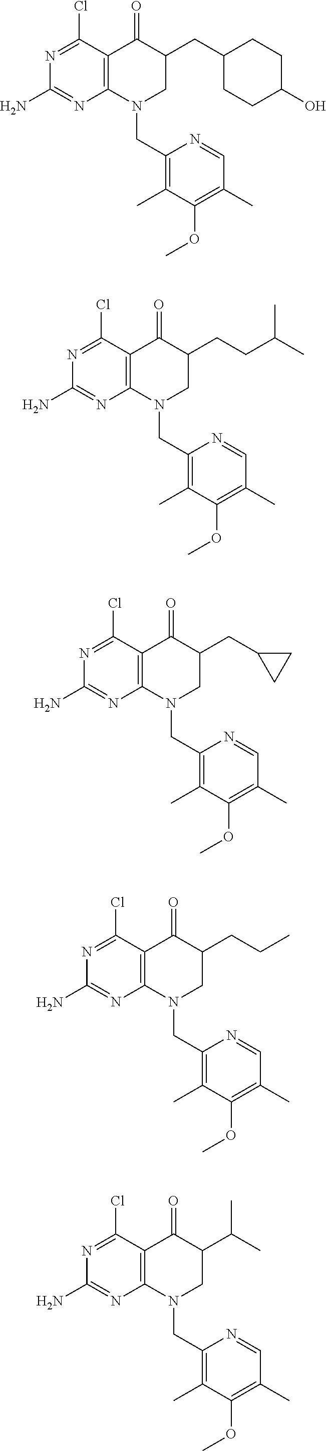

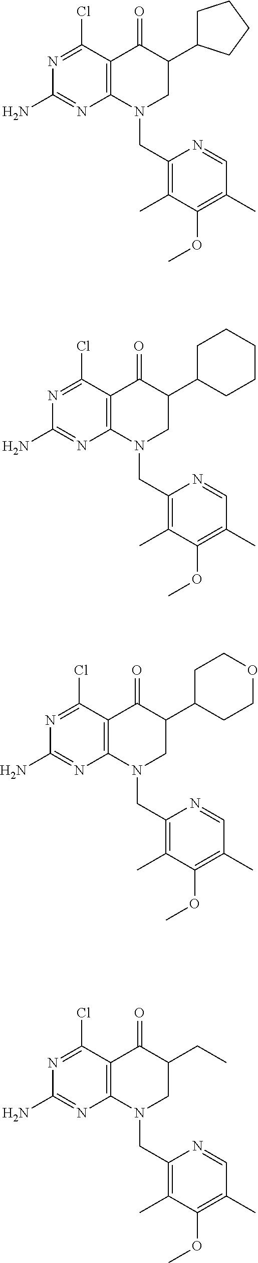

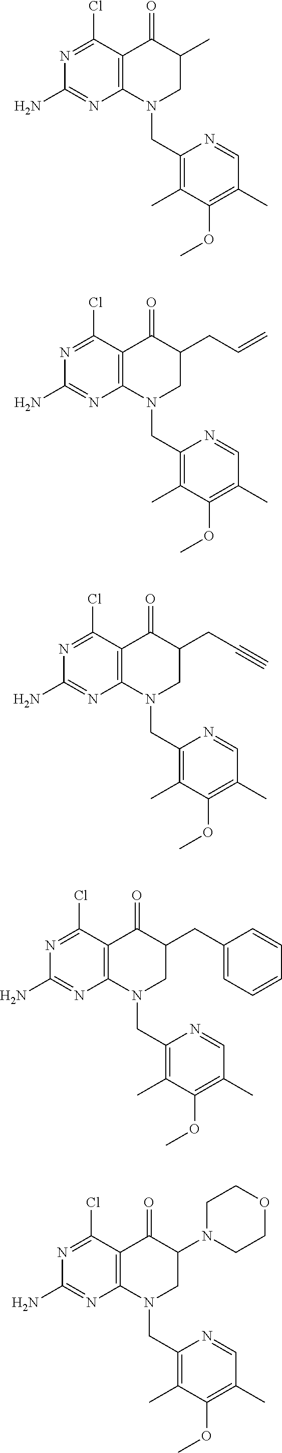

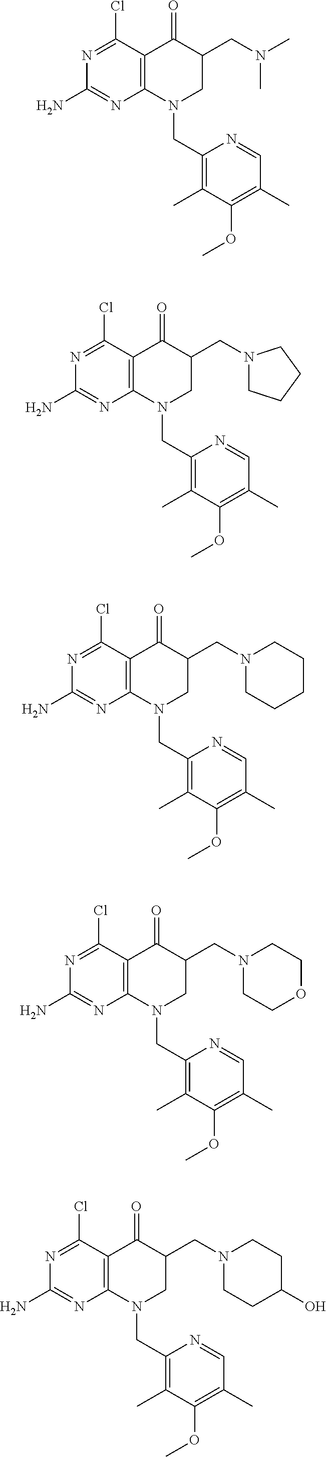

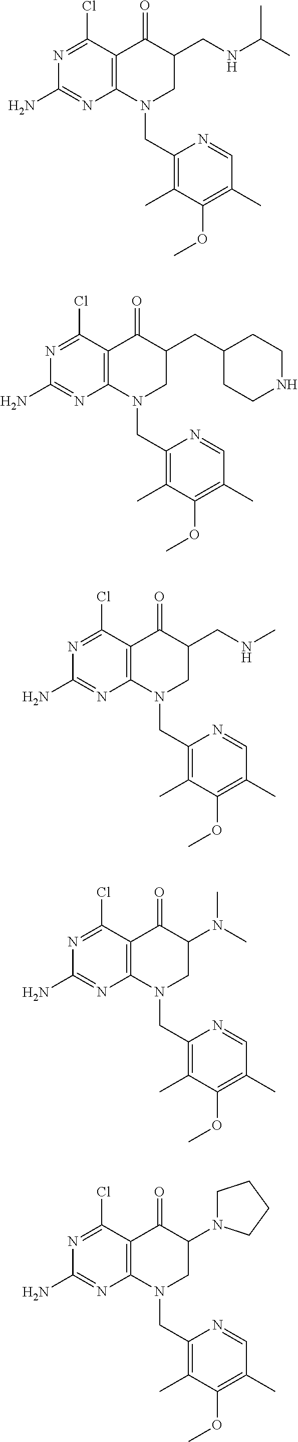

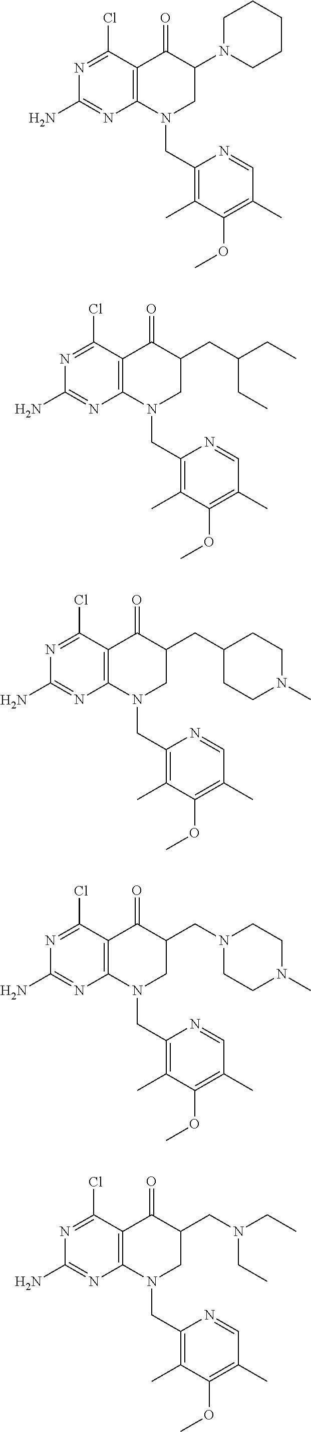

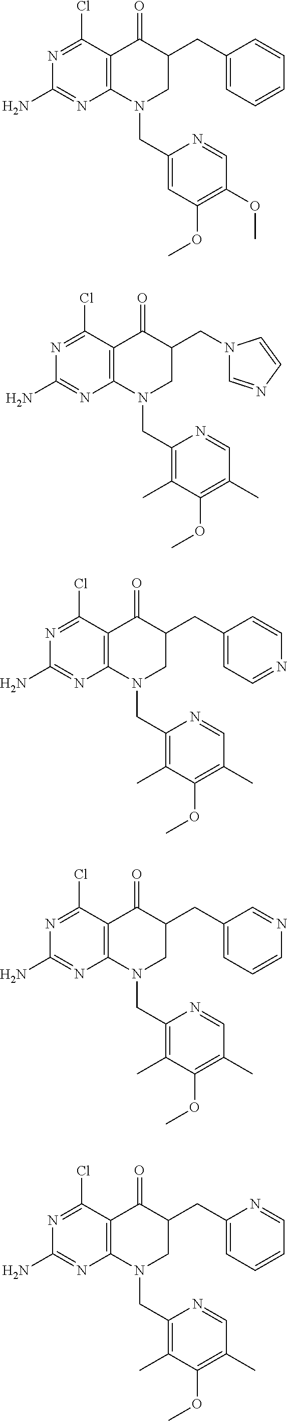

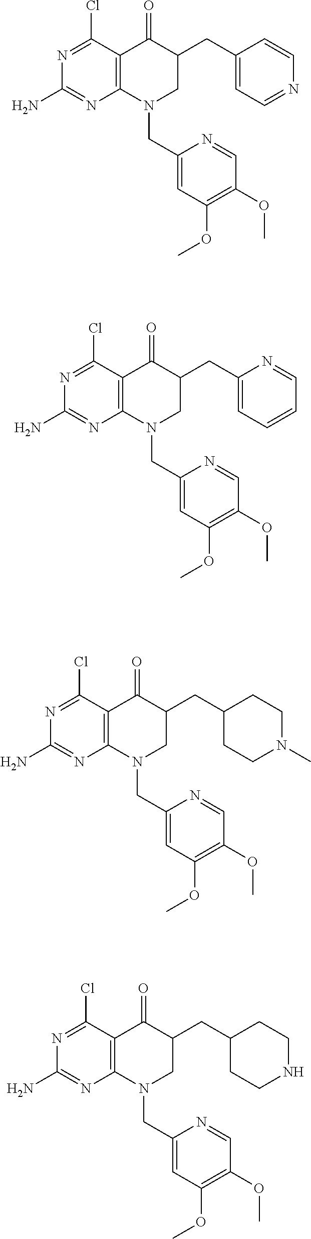

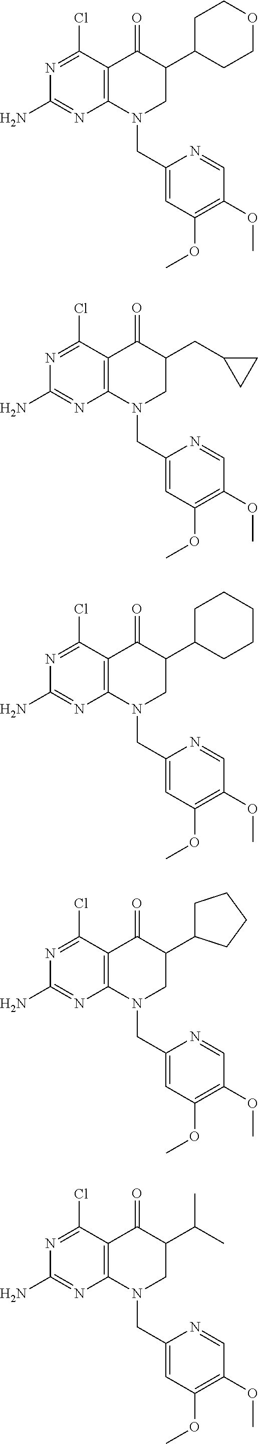

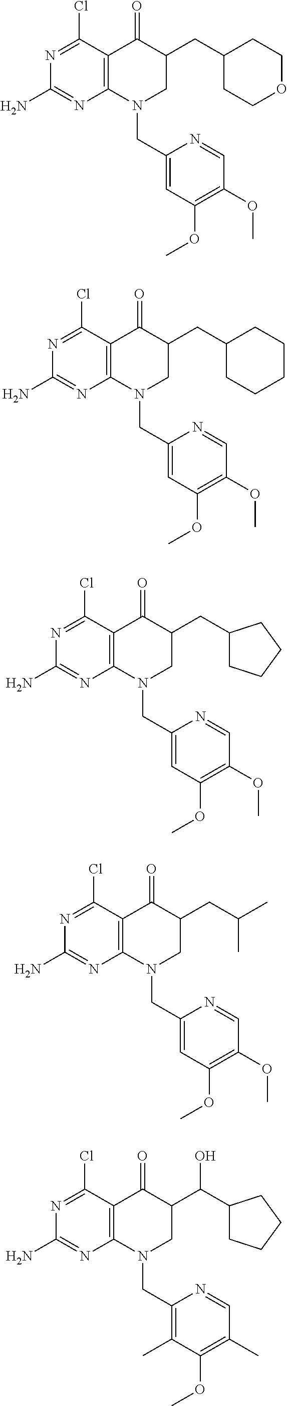

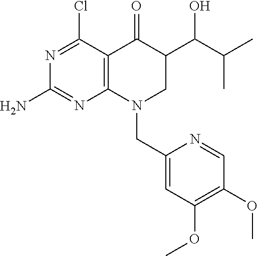

or any salt, solvate, hydrate, tautomer, stereoisomer, or prodrug thereof.

In various embodiments the invention provides a compound of formula (V)

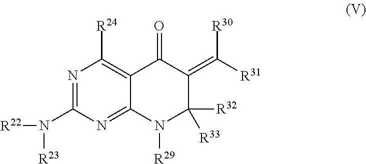

wherein

R²² and R²³ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z, wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²² and R²³ can together with the nitrogen atom to which they are bonded form a heterocyclic ring substituted with 0-4 Z; wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R²⁴ is H, halo, alkyl, haloalkyl, OR, NRR′, nitro, or cyano;

R²⁹ is H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁹ comprises a group of the formula R²-R³;

R³⁰ and R³¹ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R³⁰ and R³¹ can together with a carbon atom to which they are bonded form a cycloalkyl ring substituted with 0-4 Z;

R³² and R³³ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R³² and R³³ can together with a carbon atom to which they are bonded form a cycloalkyl ring substituted with 0-4 Z.

In various embodiments, R²⁹ can comprise a substituted pyridyl moiety, such as a (3,5-dimethyl-4-methoxypyridin-2-yl)methyl moiety or a (4,5-dimethoxypyridin-2-yl)methyl moiety.

In various embodiments, R²⁴ can be halo, such as chloro.

In various embodiments, R³⁰ is H and R³¹ comprises an alkyl group, such as an isobutyl, cyclopentylmethyl, cyclopropylmethyl or (tetrahydro-2H-pyran-4-yl)methyl group.

In various embodiments, R²², R²³, R³² and R³³ are H.

In various embodiments, a compound of formula (V) can be any of the following:

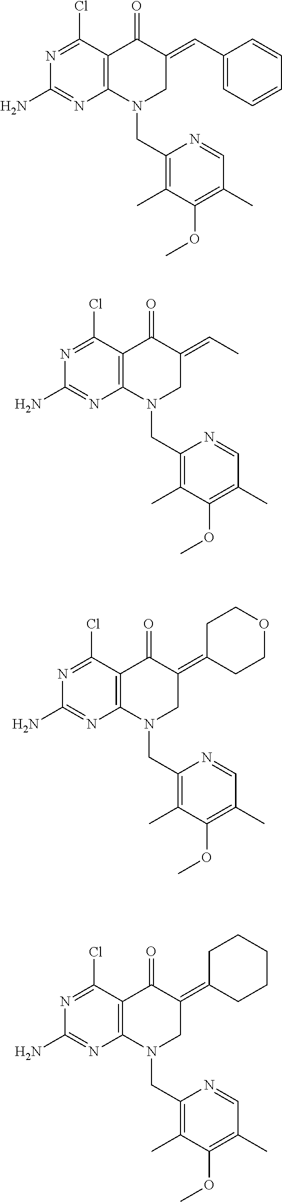

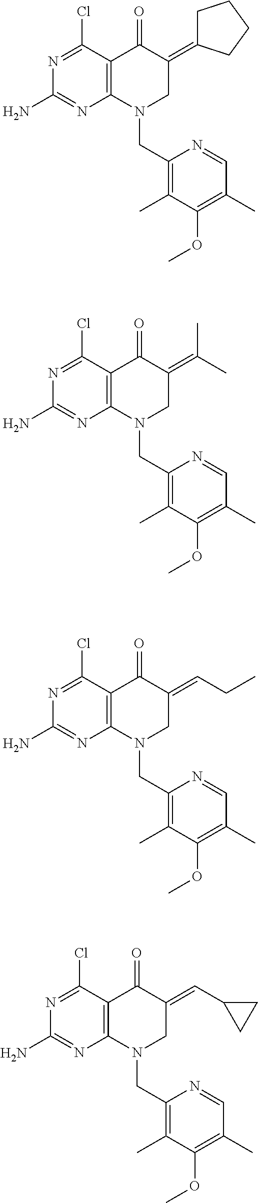

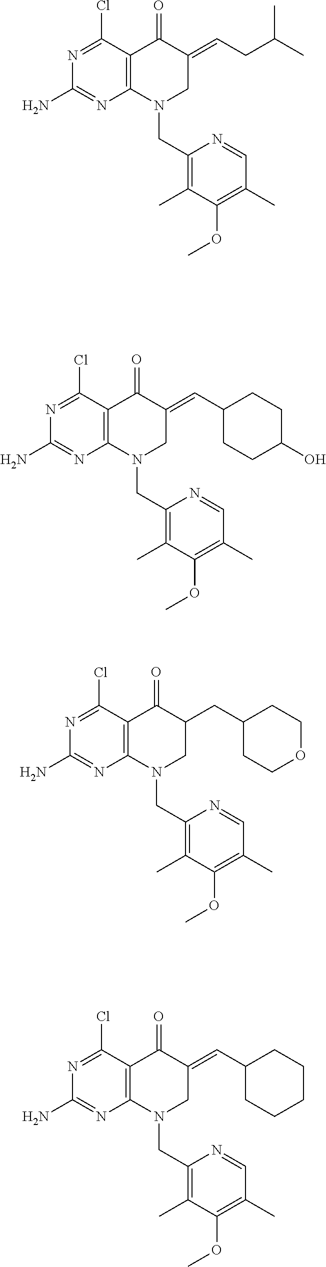

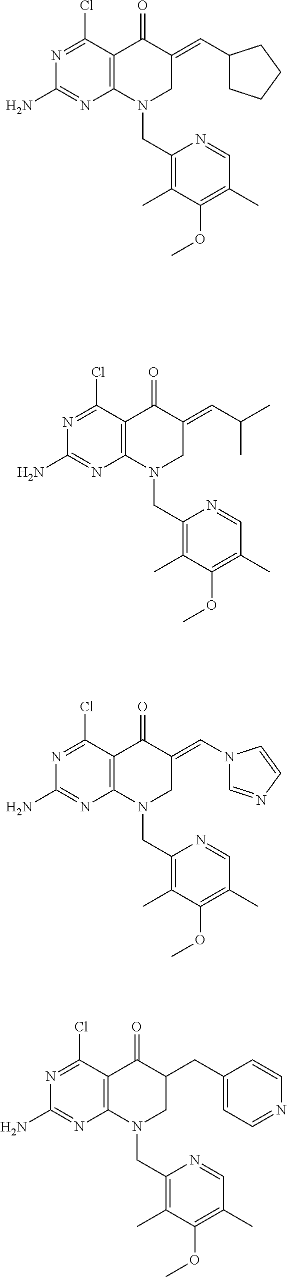

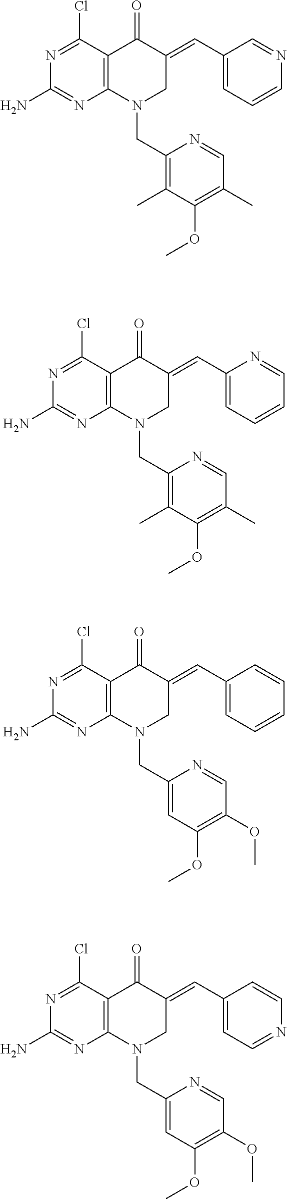

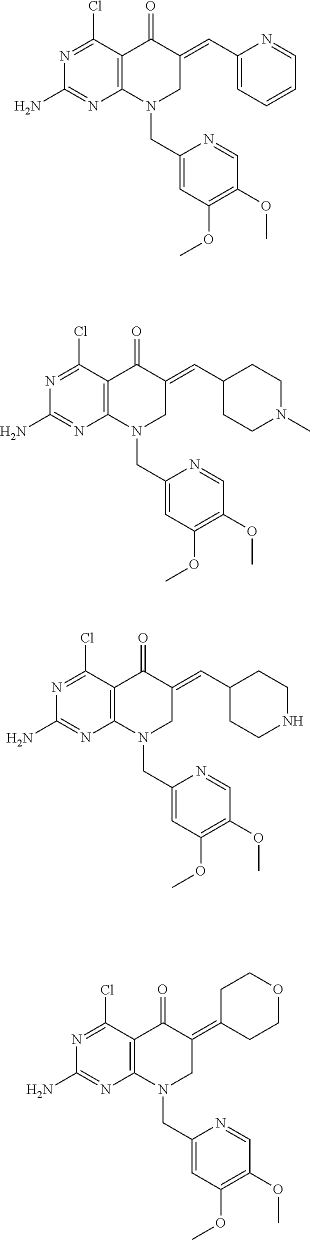

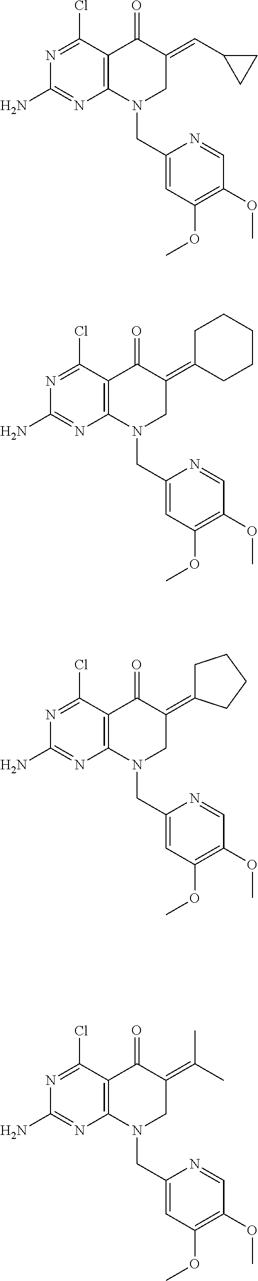

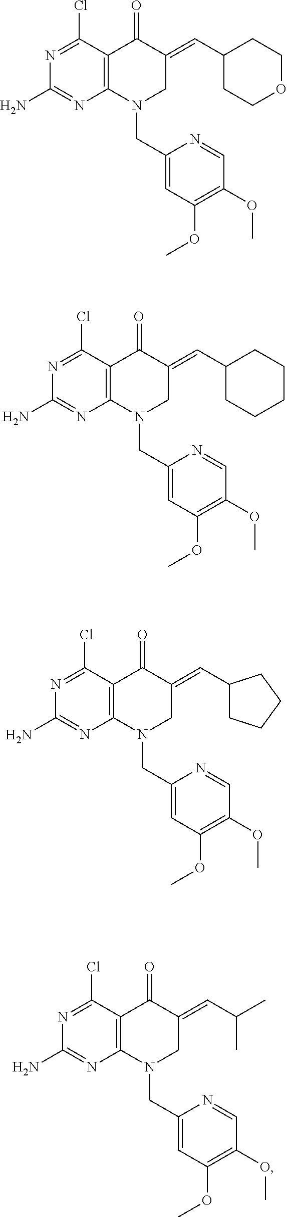

or any salt, solvate, hydrate, tautomer, stereoisomer, or prodrug thereof.

In various embodiments the invention provides a compound of formula (VI)

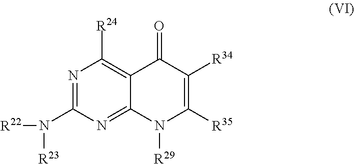

wherein

R²² and R²³ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z, wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²² and R²³ can together with the nitrogen atom to which they are bonded form a heterocyclic ring substituted with 0-4 Z; wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R²⁴ is H, halo, alkyl, haloalkyl, OR, NRR′, nitro, or cyano;

R²⁹ is H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁹ comprises a group of the formula R²-R³;

R³⁴ and R³⁵ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R³⁴ and R³⁵ can together with a carbon atom to which they are bonded form a cycloalkyl ring substituted with 0-4 Z.

In various embodiments, R²⁹ comprises a substituted pyridyl moiety, such as a (3,5-dimethyl-4-methoxypyridin-2-yl)methyl moiety or a (4,5-dimethoxypyridin-2-yl)methyl moiety.

In various embodiments, R²⁴ is halo, such as chloro.

In various embodiments, R³⁴ comprises an alkyl group, such as an isobutyl, cyclopentylmethyl, cyclopropylmethyl or (tetrahydro-2H-pyran-4-yl)methyl group.

In various embodiments, R²², R²³ and R³⁵ are H.

In various embodiments, a compound of formula (VI) can be any of the following:

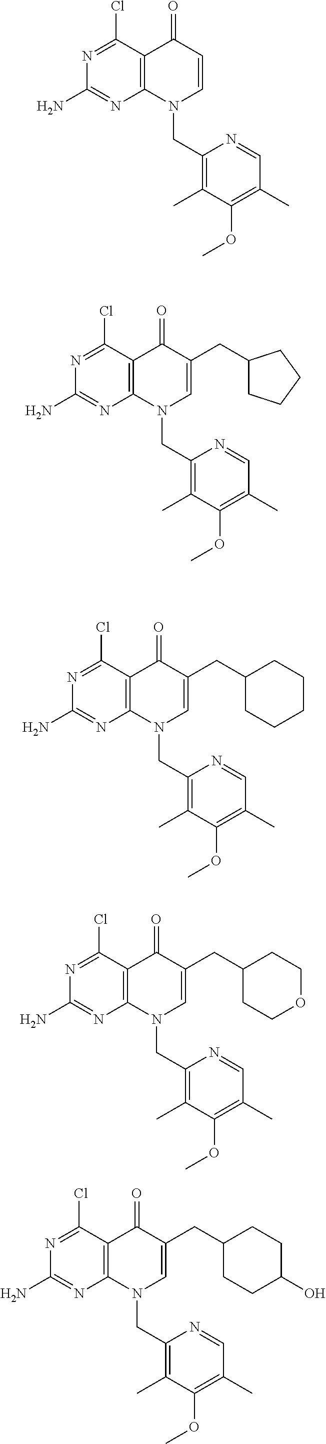

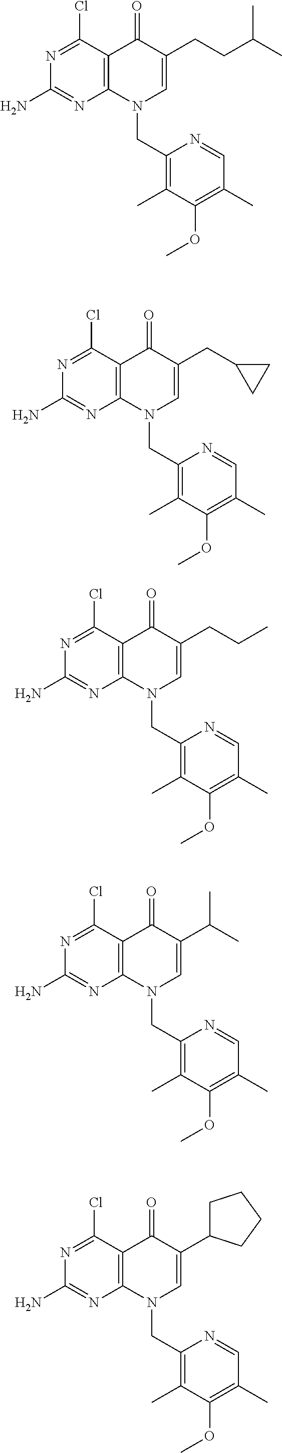

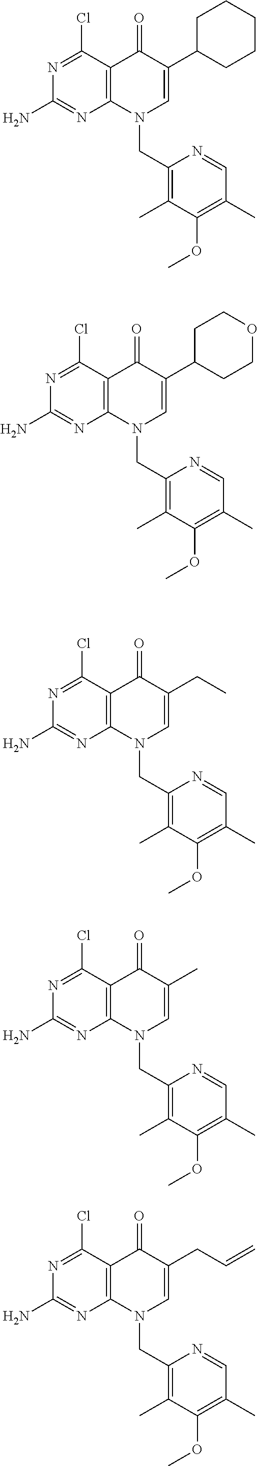

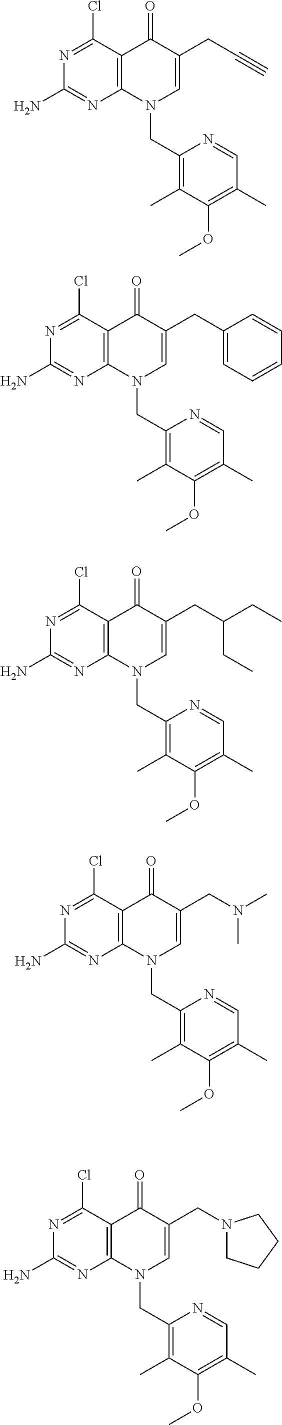

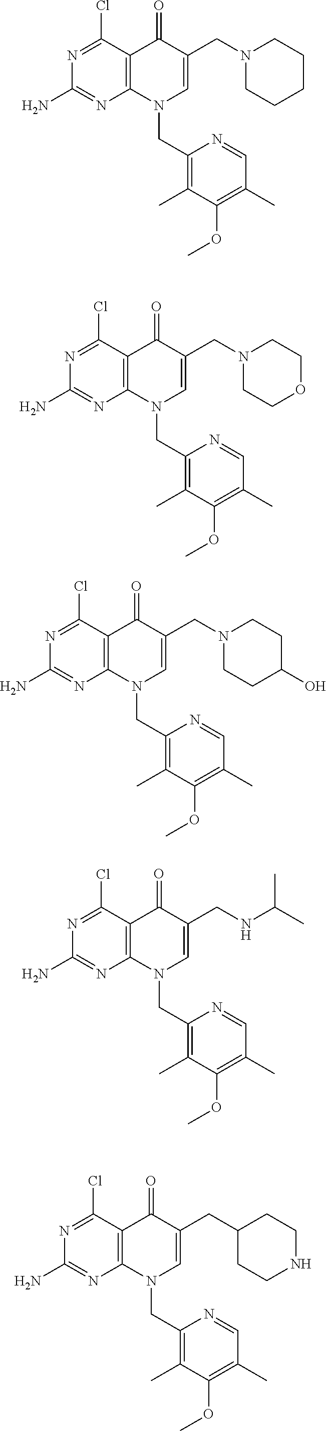

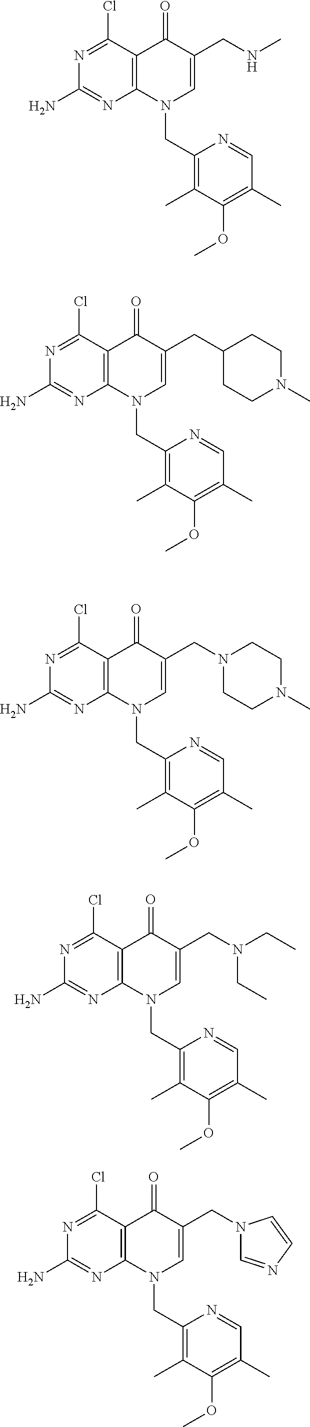

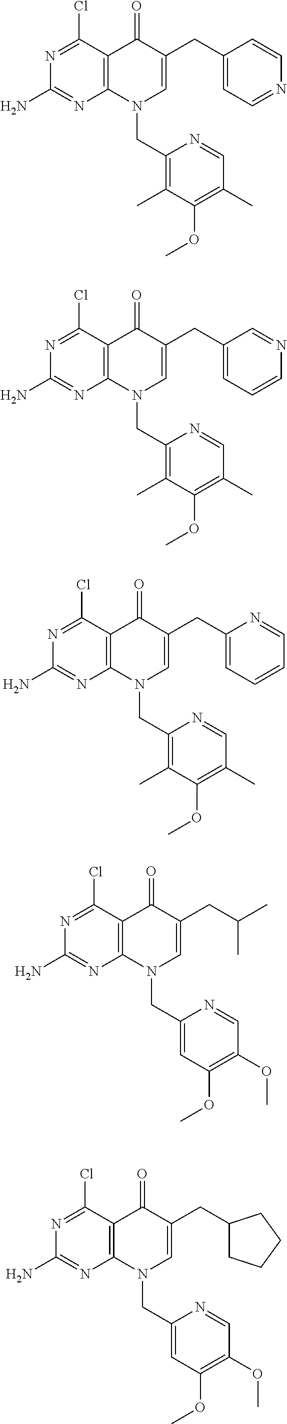

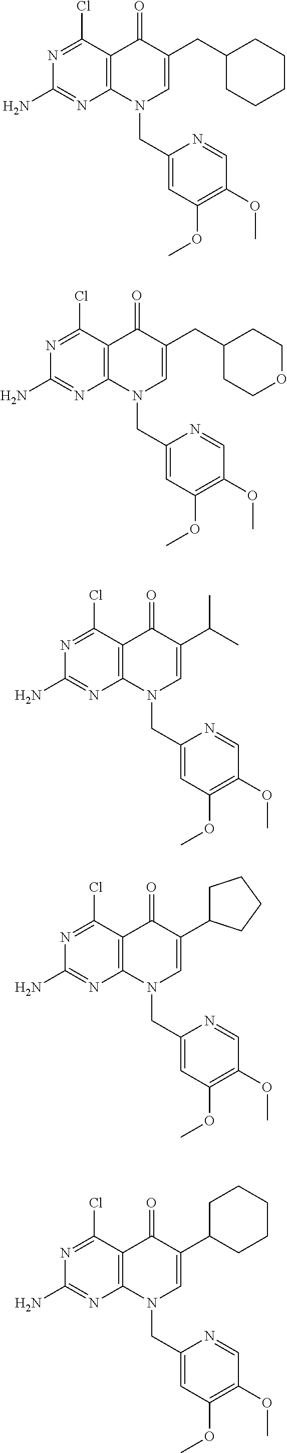

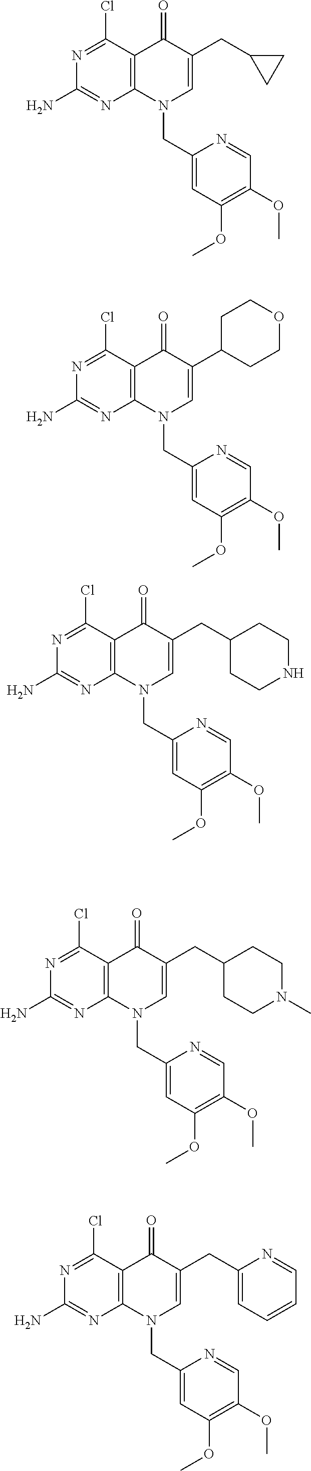

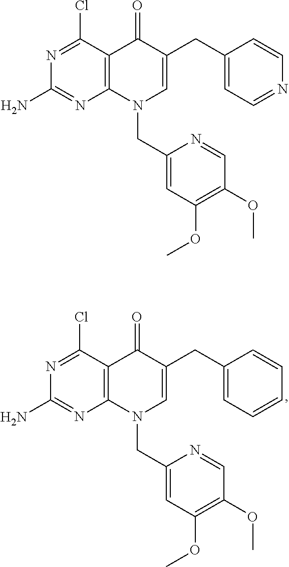

or any salt, solvate, hydrate, tautomer, stereoisomer, or prodrug thereof.

In various embodiments the invention provides a compound of formula (VII)

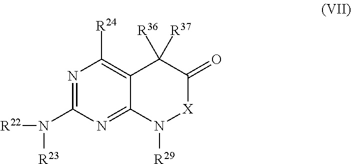

wherein

R²² and R²³ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z, wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²² and R²³ can together with the nitrogen atom to which they are bonded form a heterocyclic ring substituted with 0-4 Z; wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R²⁴ is H, halo, alkyl, haloalkyl, OR, NRR′, nitro, or cyano;

R²⁹ is H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R²⁹ comprises a group of the formula R²-R³;

R³⁶ and R³⁷ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R³⁶ and R³⁷ can together with a carbon atom to which they are bonded form a cycloalkyl ring substituted with 0-4 Z;

X is CR³⁸R³⁹ or NR⁴⁰ wherein R³⁸ and R³⁹ are independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂; or, R³⁸ and R³⁹ can together with a carbon atom to which they are bonded form a cycloalkyl ring substituted with 0-4 Z; and

R⁴⁰ is H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂.

In various embodiments, R²⁹ comprises a substituted pyridyl moiety, such as a (3,5-dimethyl-4-methoxypyridin-2-yl)methyl moiety or a (4,5-dimethoxypyridin-2-yl)methyl moiety.

In various embodiments, R²⁴ is halo, such as chloro.

In various embodiments, X is CH₂ or NH.

In various embodiments, R³⁶ and R³⁷ comprises an alkyl group.

In various embodiments, R³⁶ and R³⁷ are independently selected from H, methyl, ethyl, isopropyl, isobutyl, or R³⁶ and R³⁷ together to form a propylene, butylene or pentylene group.

In various embodiments, R²² and R²³ are H.

In various embodiments, the compound of formula (VII) can be any of the following:

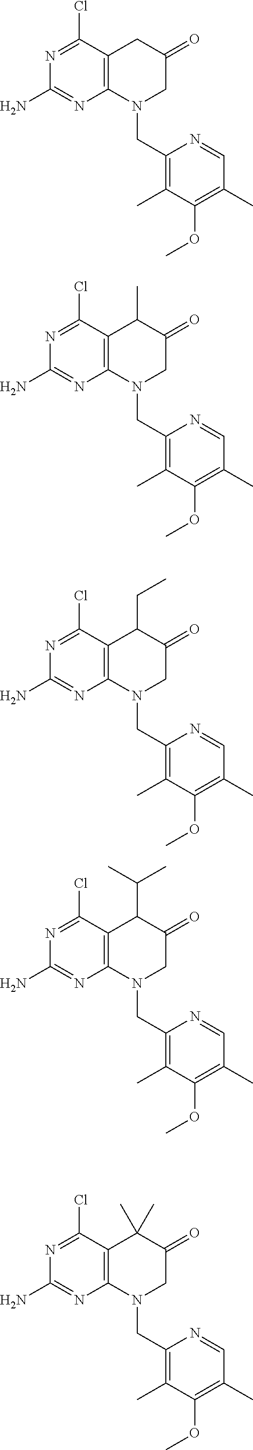

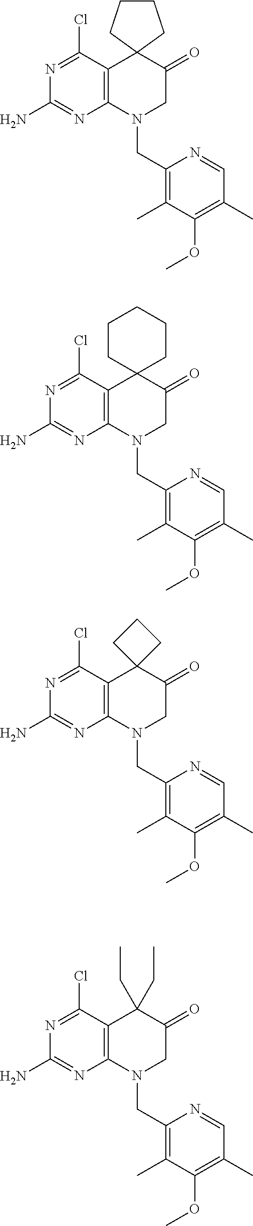

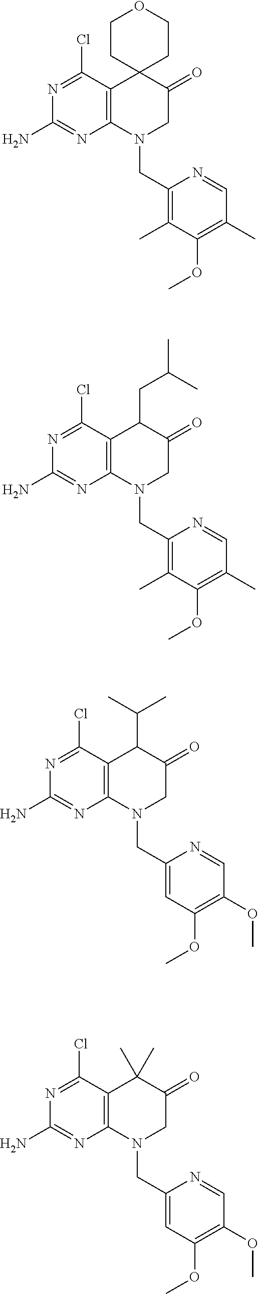

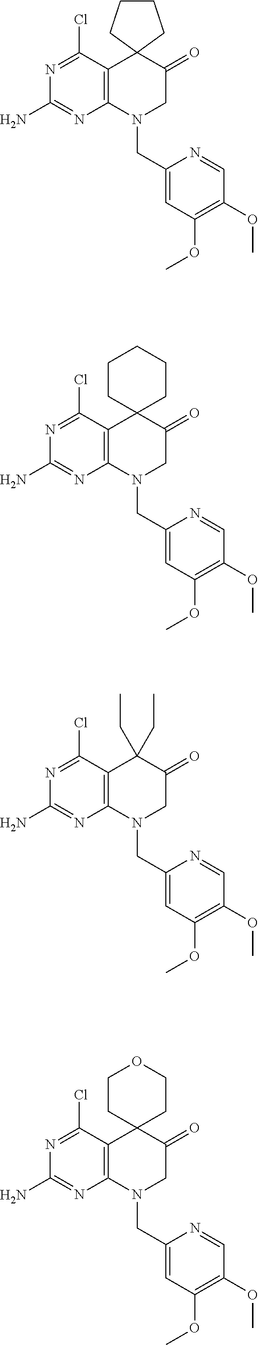

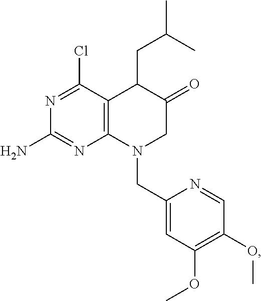

or any salt, solvate, hydrate, tautomer, stereoisomer, or prodrug thereof.

In various embodiments, the invention provides a compound of formula (VIII)

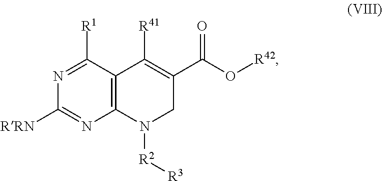

R and R′ are independently at each occurrence H, or (C₁-C₈)alkyl wherein any alkyl is optionally mono- or independently pluri-substituted with OR, halo, cyano, OC(O)R, OC(O)OR, CO₂R, NRR′, C(O)NRR′, N(R)C(O)R′, oxo, OC(O)NRR′, SR, S(O)R, S(O)₂R, S(O)₂OR, or SO₂NRR′ alkyl, acyl, alkoxy, cycloalkyl, heterocyclyl, aryl, or heteroaryl, wherein any alkyl, acyl, alkoxy, cycloalkyl, heterocyclyl, aryl, or heteroaryl is substituted with 0-4 Z; wherein any carbon atom of the alkyl, acyl, alkoxy, cycloalkyl, heterocyclyl, aryl, or heteroaryl, can be replaced by a heteroatom selected from the group consisting of NR, O, S, S(O), and S(O)₂; or, R and R′ can together with a nitrogen atom to which they are bonded form a heterocyclic or heteroaryl ring, wherein the heterocyclic or heteroaryl ring can further comprise 1-3 O, N, S, S(O), or S(O)₂ heteroatoms therewithin and is substituted with 0-4 Z groups wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;

R¹ comprises H, halo, NRR′, or hydroxyl; or comprises (C₁-C₆)alkyl, cycloalkyl, or heterocyclyl, any of which can be optionally substituted by oxo, CO₂R, heterocylyl, aryl, OR, NRR′, or SR;

R² comprises (C(R″)₂)ₙ wherein n is 0-3, wherein R″ is independently at each occurrence H, alkyl, halo, haloalkyl, OR, or NRR′, or two R″ together with a carbon atom to which they are bonded comprise a carbonyl;

R³ comprises alkyl, cycloalkyl, heterocyclyl, aryl, or heteroaryl, wherein any alkyl, cycloalkyl, heterocyclyl, aryl, or heteroaryl is substituted with 0-5 Z groups;

R⁴¹ and R⁴² are each independently at each occurrence H, (C₁-C₆)alkyl, (C₁-C₆)alkenyl, (C₁-C₆)alkynyl, (C₃-C₉)cycloalkyl, (C₆-C₁₀)aryl, or 5-10 membered heteroaryl, wherein any group is substituted with 0-4 Z; wherein any carbon atom of the alkyl, cycloalkyl, heterocyclyl, aryl, or heteroaryl, is optionally replaced by a heteroatom selected from the group consisting of NR, O, S, S(O), and S(O)₂.

In various embodiments, R and R′ can both be H.

In various embodiments, R¹ can be halo, such as chloro, or can be trifluoromethyl, methyl, or methoxyl.

In various embodiments, R⁴¹ can be H.

In various embodiments, R⁴² can be methyl.

In various embodiments, the compound of formula (VIII) is

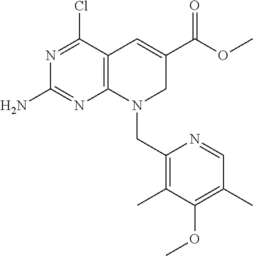

In various embodiments the invention provides a salt, solvate, hydrate, tautomer, stereoisomer, or prodrug of any of the compounds of any one of the formulas (I), (II), (III), (IV), (V), (VI), or (VII).

Another aspect of an embodiment of the invention provides compositions of the compounds of the invention, alone or in combination with another medicament. As set forth herein, compounds of the invention include stereoisomers, tautomers, hydrates, solvates, prodrugs, salts including pharmaceutically acceptable salts, and mixtures thereof. Compositions containing a compound of the invention can be prepared by conventional techniques, e.g. as described in Remington: The Science and Practice of Pharmacy, 19th Ed., 1995, incorporated by reference herein. The compositions can appear in conventional forms, for example capsules, tablets, aerosols, solutions, suspensions or topical applications.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation can be tabletted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that can be prepared by conventional tabletting techniques can contain:

| Core: | | |
|---|---|---|
| Active compound (as free compound or salt thereof) | 250 | mg |
| Colloidal silicon dioxide (Aerosil) ® | 1.5 | mg |
| Cellulose, microcryst. (Avicel) ® | 70 | mg |
| Modified cellulose gum (Ac-Di-Sol) ® | 7.5 | mg |
| Magnesium stearate | Ad. | |
| Coating: | | |
| HPMC approx. | 9 | mg |
| *Mywacett 9-40 T approx. | 0.9 | mg |

*Acylated monoglyceride used as plasticizer for film coating.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

The compounds of the invention can be administered to a mammal, especially a human in need of such treatment, prevention, elimination, alleviation or amelioration of a malcondition. Such mammals include also animals, both domestic animals, e.g. household pets, farm animals, and non-domestic animals such as wildlife.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge.

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily, such as every other day, or weekly, if found to be advisable by a prescribing physician.

As described in detail in the Examples, various embodiments of the invention provide methods of synthesis of the inventive compounds herein. For example, in various embodiments, the invention provides a method of synthesis of a compound of formula (I) comprising a compound of formula (Ia):

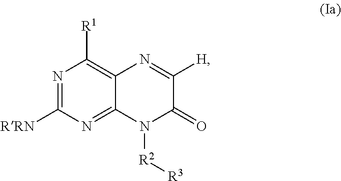

wherein R, R′, R¹, R², and R³ are as defined in claim 1,
the method comprising contacting a compound of formula (Ib):

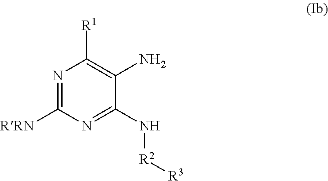

and a glyoxalate ester under conditions suitable to bring about the formation of the compound of formula (Ia).

In various embodiments, the invention provides a method of synthesis of a compound of formula (I) comprising a compound of formula (Ic):

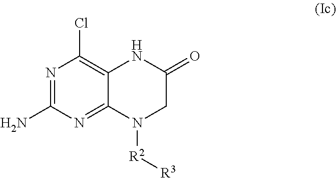

comprising contacting 4,6-dichloropyrimidine-2,5-diamine and a compound of formula R³-R²—NH—CH₂—CO₂E, wherein E is lower alkyl and R² and R³ are as defined in claim 1, under conditions suitable to bring about the formation of the compound of formula (Ic).

In various embodiments, the invention provide a method of synthesis of a compound of formula (III):

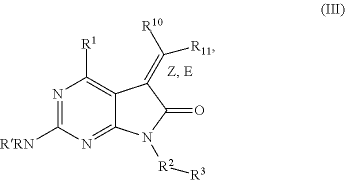

wherein R, R¹, R², R³, R¹⁰ and R¹¹ are as defined in claim 1, and Y¹ is O, the method comprising contacting a compound of formula (IIIa):

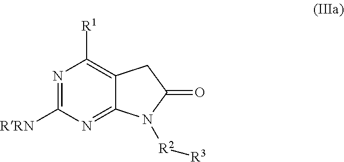

and R¹⁰—C(O)—R¹¹ under conditions suitable to bring about the formation of the compound of formula (III), wherein a double bond marked Z, E can be in either a Z or an E configuration, or a mixture thereof.

### EXAMPLES

The following abbreviations are used throughout.
¹H NMR proton nuclear magnetic resonance (given as δ (multiplicity, coupling constant if determined, relative number of resonating protons))
¹³C NMR ¹³C nuclear magnetic resonance (given as δ)
AcOH acetic acid
BINAP 2,2′-bis(diphenylphosphino)-1,1′-binaphthyl
DCM dichloromethane
DMF N,N-dimethylformamide
DMSO dimethylsulfoxide
Et₃N triethylamine
EtOAc ethyl acetate
EtOH ethanol
h hours
HPLC high performance liquid chromatography
L liters
LCMS liquid chromatography-mass spectrometry
MeOH methanol
min minutes
mL milliliters
MS mass spectrum (given as m/z), mass spectrometry
n-BuOH n-butanol
Ph phenyl
RT room temperature
t-BuOH tert-butanol
t-BuOK potassium tert-butoxide
TEA triethylamine
THF tetrahydrofuran
TLC thin layer chromatography
TsOH p-toluenesulfonic acid
Xantphos 4,5-bis(diphenylphosphino)-9,9-dimethylxanthene

### Exemplary Compounds of Formula (I)

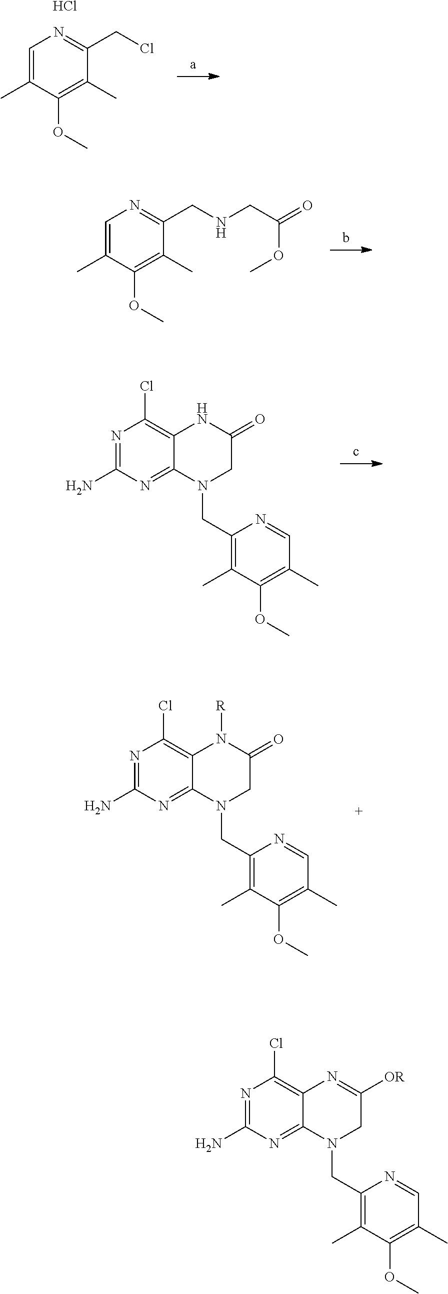

Scheme 1. Synthesis of 5-substituted-2-amino-4-chloro-8-((4-methoxy-3,5-dimethylpyridin-2-yl)methyl)-7,8-dihydropteridin-6(5H)-ones: (a) methyl 2-aminoacetate hydrochloride, K₂CO₃, EtOH, reflux, 1 day; 43% yield (b) Et₃N, BuOH, 4,6-dichloropyrimidine-2,5-diamine, reflux, 18 h; 50% yield (c) NaH, DMF, RX., room temperature, overnight.

### Example I-1

### 2-Amino-4-chloro-8-(6-chloro-benzo[1,3]dioxol-5-ylmethyl)-8H-pteridin-7-one

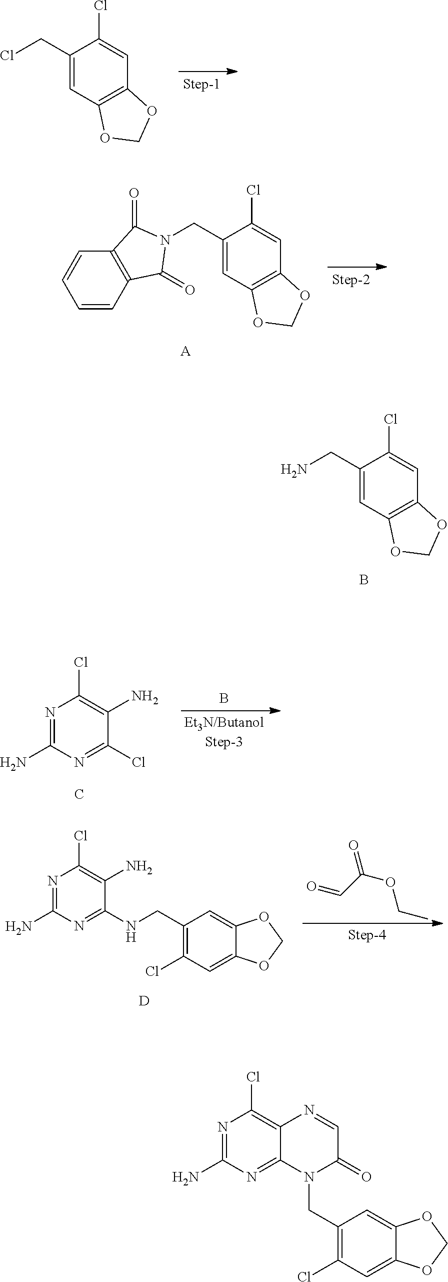

### Step-1: 2-((6-Chlorobenzo[d][1,3]dioxol-5-yl)methyl)isoindoline-1,3-dione (A)

To the solution of potassium phthalimide (3.26 g, 17.6 mmol) and 5-chloro-6-(chloromethyl)benzo[d][1,3]dioxole (3.28 g, 16 mmol) in DMF (4 mL) was added tetrabutylammonium bromide (520 mg, 1.6 mmol). The reaction mixture was stirred at 50° C. for 4 h, diluted with brine, filtered, concentrated, and dried to give the title compound as a white solid (4.00 g, 79%). ¹H NMR (400 MHz, DMSO-d₆) δ: 4.73 (s, 2H), 6.04 (s, 2H), 6.92 (s, 1H), 7.11 (s, 1H), 7.85-7.92 (m, 4H).

### Step-2: (6-Chlorobenzo[ ][d1,3]dioxol-5-yl)methanamine (B)

The reaction mixture of 2-((6-chlorobenzo[d][1,3]dioxol-5-yl)methyl)isoindoline-1,3-dione (1.40 g, 4.4 mmol) and hydrazine hydrate (10 mL) was stirred at 50° C. for 4 h, quenched with ice water, and extracted with dichloromethane (DCM, 3×20 mL). The organic layers were combined, washed with brine (2×20 mL), dried over anhydrous Na₂SO₄, and concentrated. The residue was purified by silica gel chromatography eluted with DCM-MeOH (10:1) to give the title compound as a yellow liquid (550 mg, 67%). ¹H NMR (400 MHz, CDCl₃) δ: 3.84 (s, 2H), 5.98 (s, 2H), 6.86 (s, 1H), 6.88 (s, 1H),

### Step-3: 6-Chloro-N4-((6-chlorobenzo[d][1,3]-dioxol-5-yl)methyl)pyrimidine-2,4,5-triamine (D)

The reaction solution of 4,6-dichloropyrimidine-2,5-diamine (338 mg, 1.89 mmol), 246-chlorobenzo[d][1,3]dioxol-5-yl)methyl)isoindoline-1,3-dione (700 mg, 3.77 mmol) and Et₃N (1.3 mL, 51.33 mmol) in n-BuOH (13 mL) was heated at reflux overnight, cooled to room temperature, and concentrated. The residue was purified by silica gel chromatography eluted with petroleum ether-ethyl acetate (1:1) to give the title compound as a yellow solid (600 mg, 97%). ¹H NMR (400 MHz, DMSO-d₆) δ: 3.96 (s, 2H), 4.48 (d, J=6.4 Hz, 2H), 5.68 (s, 2H), 6.03 (s, 2H), 6.94 (s, 2H), 7.07 (s, 1H).

### Step-4: 2-Amino-4-chloro-8-(6-chloro-benzo[1,3]dioxol-5-ylmethyl)-8H-pteridin-7-one

To the reaction solution of 6-chloro-N4-((6-chlorobenzo[d][1,3]dioxol-5-yl)methyl)pyrimidine-2,4,5-triamine (300 mg, 0.91 mmol) in acetic acid (0.29 mL) and ethanol (5.4 mL) was added a solution of ethyl glyoxylate in toluene (ca. 50%) (210 mg, 1.0 mmol). The reaction mixture was stirred at reflux for 4 h and concentrated. The residue was triturated with NH₃.H₂O and filtered. The filter cake was washed with water and dried to afford the title compound as a yellow solid (200 mg, 60%). ¹H NMR (400 MHz, DMSO-d₆) δ: 5.20 (s, 2H), 6.00 (s, 2H), 6.64 (s, 1H), 7.13 (s, 1H), 7.24-7.83 (d, 2H), 7.93 (s, 1H). MS m/z: 354 [M−1]^{+·}.

### Example I-2

### 2-Amino-4-chloro-8-((4-methoxy-3,5-dimethyl-pyridin-2-ylmethyl)-7,8-dihydro-5H-pteridin-6-one

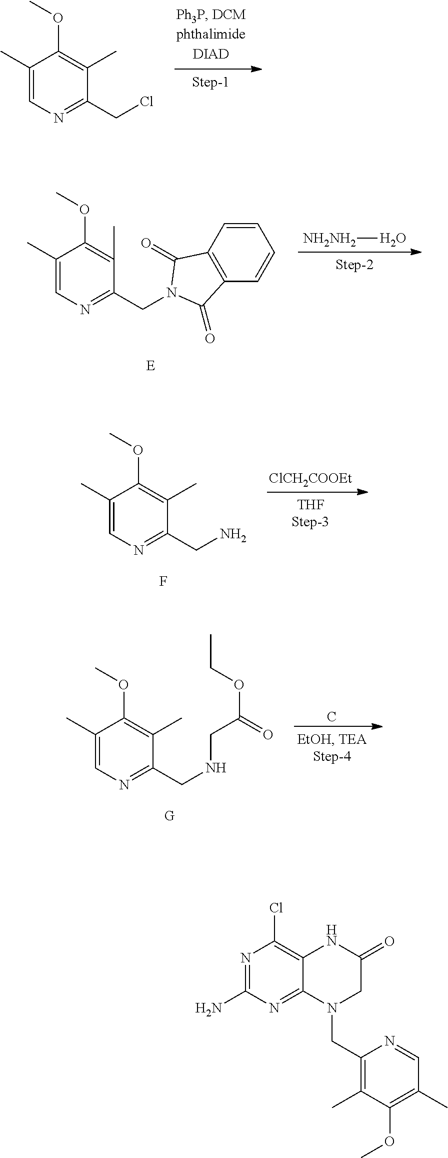

### Step-1: 2-((4-Methoxy-3,5-dimethylpyridin-2-yl)methyl)isoindoline-1,3-dione (E)

To a 3.0 L three neck flask were charged with (4-methoxy-3,5-dimethylpyridin-2-yl)methanol (50.0 g, 0.30 mol), phthalimide (88.0 g, 0.60 mol) and Ph₃P (156.9 g, 0.60 mol), and DCM (2.0 L). The reaction mixture was cooled to 0° C., purged by nitrogen, and added DIAD (90 mL, 0.45 mol) over a period of 30 min with stirring. The final reaction mixture was then warmed to room temperature, stirred overnight, and concentrated. The residue was dissolved in 4 N HCl (1 L) and filtered. The filtrate was washed with diethyl ether (2×500 mL), neutralized with sodium bicarbonate to pH 9, and extracted with EtOAc (3×500 mL). The combined organic layers were dried over anhydrous Na₂SO₄ and concentrated to give crude product (75.0 g) which was further crystallized from MeOH to give the title compound as a white solid (55 g, 62%).

### Step-2: (4-Methoxy-3,5-dimethylpyridin-2-yl)methanamine (F)

The reaction mixture of 2-((4-methoxy-3,5-dimethylpyridin-2-yl)methyl)isoindoline-1,3-dione (30.0 g, 101 mmol) and hydrazine hydrate (120 mL) was stirred at 50° C. for 1 h, quenched with ice water, and extracted with ethyl acetate (3×10 mL). The combined organic layers were washed with brine (10 mL), dried over anhydrous sodium sulphate, and concentrated. The residue was purified by silica gel chromatography eluted with DCM-MeOH (10:1) to give the title compound as a colorless oil (12.0 g, 71%).

### Step-3: Ethyl 2-((4-methoxy-3,5-dimethylpyridin-2-yl)methylamino)acetate (G)

To the reaction mixture of (4-methoxy-3,5-dimethylpyridin-2-yl)methanamine (2.42 g, 14.55 mmol), K₂CO₃ (1.00 g, 7.26 mmol) and THF (12 mL) at 50° C. under argon was added a solution of ethyl chloroacetate (1.1 mL, 13.10 mmol) in THF (24 mL) dropwise. The reaction mixture was further stirred at 50° C. for 3.5 h, cooled to room temperature, and filtered. The filtrate was concentrated and the residue was purified by silica gel chromatography eluted with petroleum ether-ethyl acetate (1:1.5) to give the title compound as a light yellow liquid (1.83 g, 50%). ¹H NMR (400 MHz, CDCl₃) δ: 1.27 (t, 3H, J=7.2 Hz), 2.22 (s, 6H), 3.50 (s, 2H), 3.74 (s, 3H), 3.86 (s, 2H), 4.20 (q, J=7.2 Hz 2H), 8.18 (s, 1H).

### Step-4: 2-Amino-4-chloro-8-((4-methoxy-3,5-dimethyl-pyridin-2-ylmethyl)-7,8-dihydro-5H-pteridin-6-one

The reaction mixture of ethyl 2-((4-methoxy-3,5-dimethylpyridin-2-yl)methylamino)acetate (600 mg, 2.38 mmol), 4,6-dichloropyrimidine-2,5-diamine (282 mg, 1.58 mmol), NEt₃ (3 mL) and n-BuOH (16 mL) was heated to reflux for 24 h and filtered The filter cake was washed with MeOH (2 mL) and dried to give the title compound as a light pink solid (224 mg, 41%). ¹H NMR (400 MHz, CDCl₃) δ: 2.17 (s, 6H,), 3.71 (s, 3H), 4.04 (s, 2H), 4.71 (s, 2H), 6.20 (s, 2H), 8.14 (s, 1H). MS m/z: 347 [M−1]^{+·}.

### Example I-3

### 2-Amino-4-chloro-8-((4-methoxy-3,5-dimethyl-pyridin-2-ylmethyl)-5-(4-methyl-pentyl)-7,8-dihydro-5H-pteridin-6-one

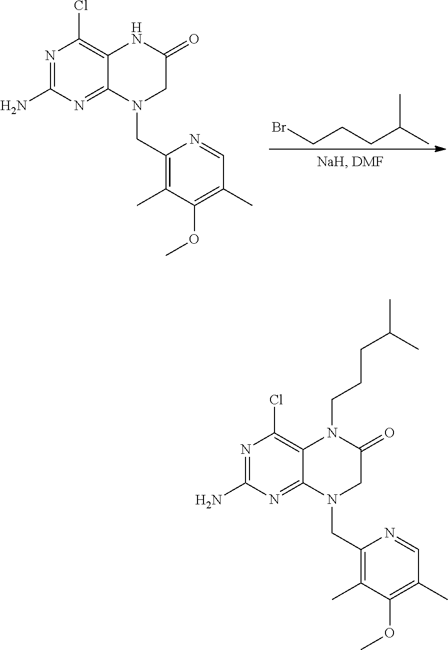

### Example 2

To a suspension of sodium hydride (51 mg, 2.13 mmol) in DMF (10 mL) was added-2-amino-4-chloro-8-((4-methoxy-3,5-dimethyl-pyridin-2-ylmethyl)-7,8-dihydro-5H-pteridin-6-one (620 mg, 1.78 mmol). The mixture was stirred at 0° C. for 0.5 h and added 1-bromo-4-methyl-pentane (440 mg, 2.67 mmol). The reaction mixture was stirred at room temperature overnight, quenched with water (30 mL), and extracted with ethyl acetate (3×20 mL). The combined organic layer was washed with brine (3×30 mL), dried over anhydrous Na₂SO₄ and concentrated. The residue was purified by silica gel chromatography eluted with petroleum ether-ethyl acetate (1:1) to give the title compound as a light yellow solid (216 mg, 28%). ¹H NMR (400 MHz, CDCl₃) δ: 0.75 (d, 6H), 0.95 (m, 2H), 1.41 (m, 3H), 2.18 (d, 6H), 3.72 (s, 3H), 3.97 (d, 4H,), 4.76 (s, 2H), 6.53 (s, 2H), 8.13 (s, 1H). MS m/z: 432 [M+1]^{+·}.

The following compounds were prepared using the procedure described for the Example I-3.

### Example I-4

### 2-Amino-4-chloro-5-isopropyl-8-((4-methoxy-3,5-dimethyl-pyridin-2-ylmethyl)-7,8-dihydro-5H-pteridin-6-one

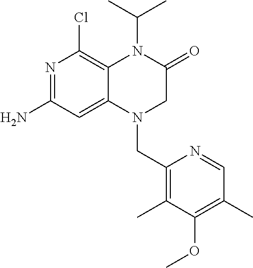

The title compound was prepared in 28% yield. ¹H NMR (400 MHz, DMSO-d₆) δ: 1.27 (d, 6H,), 2.18 (s, 6H), 3.42 (s, 3H), 4.13 (s, 2H), 4.75 (s, 2H), 5.17-5.20 (m, 1H), 6.29 (s, 2H), 8.16 (s, 1H). MS m/z: 391 [M+1]^{+·}.

### Example I-5

### 2-Amino-4-chloro-8-((4-methoxy-3,5-dimethyl-pyridin-2-ylmethyl)-5-methyl-7,8-dihydro-5H-pteridin-6-one

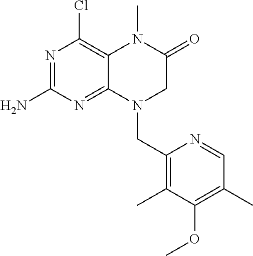

The title compound was obtained in 58% yield. ¹H NMR (400 MHz, CDCl₃) δ: 2.23 (s, 3H), 2.25 (s, 3H), 3.42 (s, 3H), 3.78 (s, 3H), 4.02 (s, 2H), 4.74 (s, 2H), 4.79 (s, 2H), 6.29 (s, 2H), 8.16 (s, 1H). MS m/z: 363 [M+1]^{+·}.

### Example I-6

### 2-Amino-4-chloro-5-isobutyl-8-((4-methoxy-3,5-dimethyl-pyridin-2-ylmethyl)-7,8-dihydro-5H-pteridin-6-one

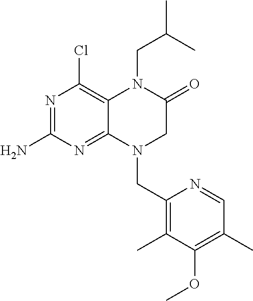

The title compound was obtained in 31%. NMR (400 MHz, CDCl₃) δ: 0.78 (s, 3H), 0.79 (s, 3H), 1.75-1.78 (m, 1H), 2.22 (s, 3H), 2.27 (s, 3H), 3.78 (s, 3H), 3.96 (s, 2H), 4.06-4.08 (d, 2H), 4.79 (s, 2H), 4.80 (s, 2H), 8.16 (s, 1H). MS m/z: 405 [M+1]^{+·}.

### Example I-7

### 2-Amino-4-chloro-8-((4-methoxy-3,5-dimethyl-pyridin-2-ylmethyl)-5-(2-pyrrolidin-1-yl-ethyl)-7,8-dihydro-5H-pteridin-6-one

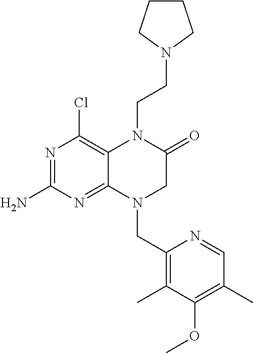

The title compound was obtained in 12% yield. ¹H NMR (400 MHz, DMSO-d₆) δ: 1.65-1.67 (m, 4H), 2.18 (s, 6H), 2.47-2.49 (m, 4H), 2.67-2.73 (t, 2H), 3.73 (s, 3H), 4.77 (s, 2H), 6.30 (s, 2H), 8.16 (s, 1H). MS m/z: 445 [M+1]+·.

### Example I-8

### 2-[2-Amino-4-chloro-8-((4-methoxy-3,5-dimethyl-pyridin-2-ylmethyl)-6-oxo-7,8-dihydro-6H-pteridin-5-yl]-N,N-diethyl-acetamide

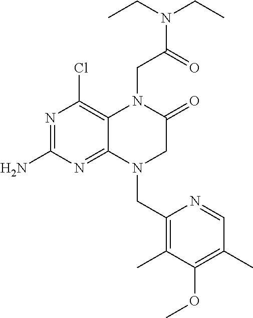

The title compound was obtained in 7.6% yield. ¹H NMR (400 MHz, CDCl₃) δ: 1.09-1.13 (t, 3H), 1.26-1.30 (t, 3H), 2.26 (s, 3H), 2.28 (s, 3H), 2.27 (s, 3H), 3.34-3.38 (m, 4H), 3.78 (s, 3H), 4.11 (s, 2H), 4.70 (s, 2H), 4.85 (s, 2H), 4.91 (s, 2H), 8.16 (s, 1H). MS m/z: 484 [M+Na]^{+·}.

### Example I-9

### 2-Amino-4-chloro-5-(2-hydroxy-ethyl)-8-((4-methoxy-3,5-dimethyl-pyridin-2-ylmethyl)-7,8-dihydro-5H-pteridin-6-one

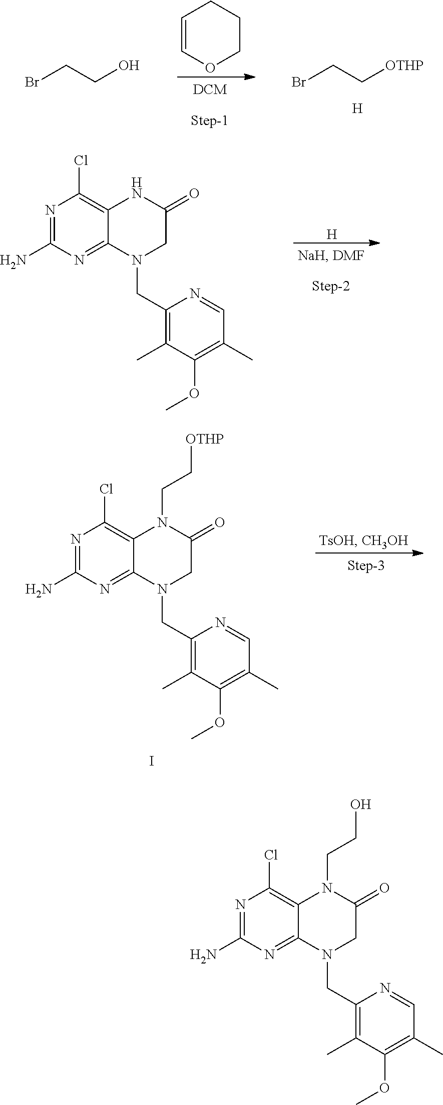

### Step-1: 2-(2-Bromoethoxy)tetrahydro-2H-pyran

To the solution of 2-bromoethanol (5.0 g, 40 mmol) and 3,4-dihydro-2H-pyran (8.4 g, 100 mmol) in DCM (50 mL) was added TsOH (76 mg, 0.4 mmol). The mixture was stirred at room temperature overnight, quenched with brine (2×50 mL), and extracted with ethyl acetate. The combined organic layer was dried over anhydrous Na₂SO₄ and concentrated. The residue was purified via distillation at 60° C. under 10 mmHg to give the title compound as yellow oil (3 g).

### Step-2: 2-Amino-4-chloro-8-((4-methoxy-3,5-dimethylpyridin-2-yl)methyl)-7,8-dihydropteridin-6(5H)-one

The title compound was obtained (320 mg) from Example I-2 (349 mg, 1.0 mmol) using the procedure described in Example I-3.

### Step-3: 2-Amino-4-chloro-5-(2-hydroxyethyl)-8-((4-methoxy-3,5-dimethylpyridin-2-yl)methyl)-7,8-dihydropteridin-6(5H)-one

To the solution of 2-amino-4-chloro-8-((4-methoxy-3,5-dimethyl-pyridin-2-ylmethyl)-5-[2-(tetrahydro-pyran-2-yloxy)-ethyl]-7,8-dihydro-5H-pteridin-6-one (320 mg, 6.7 mmol) in MeOH (5 mL) was added TsOH (120 mg, 7 mmol). The reaction mixture was stirred for 1 h and concentrated. The residue was purified by silica gel chromatography to the title compound (125 mg, 32%). ¹H NMR (400 MHz, DMSO-d₆) δ: 1.27 (s, 3H,), 1.29 (s, 3H), 3.41-3.45 (m, 2H), 3.72 (s, 3H), 3.97 (s, 2H), 4.02-4.05 (t, 2H), 4.65-4.68 (t, 1H),

### Example I-10

### 2-Amino-4-chloro-8-((4-methoxy-3,5-dimethyl-pyridin-2-ylmethyl)-5-(3-morpholin-4-yl-propyl)-7,8-dihydro-5H-pteridin-6-one

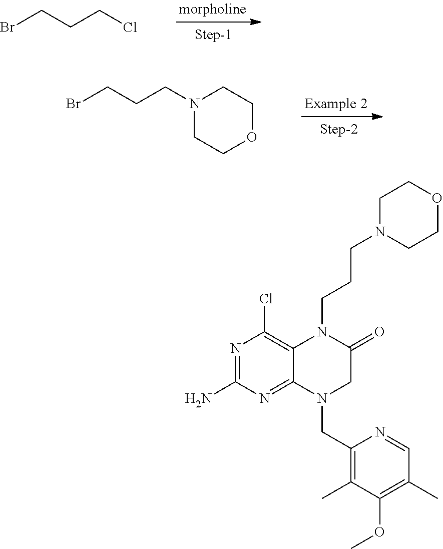

### Step-1: 4-(3-Bromopropyl)morpholine

The reaction solution of morpholine (52.2 mL, 593 mmol) and 1-bromo-3-chloropropane (30 mL, 303 mmol) in toluene (180 mL) was heated to 70° C. for 3 h and concentrated. The residue was purified by distillation to give the title compound as colorless oil (39 g, 80%).

### Step-2: 2-Amino-4-chloro-8-((4-methoxy-3,5-dimethyl-pyridin-2-ylmethyl)-5-(3-morpholin-4-yl-propyl)-7,8-dihydro-5H-pteridin-6-one

The title compound was prepared in 6.3% yield using the procedure described for Example I-3. ¹H NMR (400 MHz, CDCl₃) δ: 1.76-1.80 (m, 2H), 2.23-2.36 (m, 12H), 3.68-3.70 (t, 4H) 3.78 (s, 3H), 3.98 (s, 2H), 4.12-4.16 (t, 2H), 4.76 (s, 2H), 4.79 (s, 2H), 8.16 (s, 1H). MS m/z: 498 [M+Na]^{+·}.

### Example I-11

### 2-Amino-4-chloro-8-((4-methoxy-3,5-dimethyl-pyridin-2-ylmethyl)-5-[2-(2-oxo-pyrrolidin-1-yl)-ethyl]-7,8-dihydro-5H-pteridin-6-one

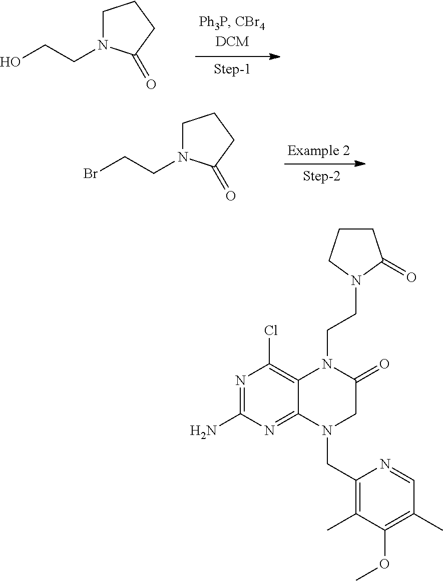

### Step-1:1-(2-Bromoethyl)pyrrolidin-2-one

To a reaction mixture of 1-(2-hydroxyethyl)pyrrolidin-2-one (516 mg, 2 mmol) and Ph₃P (1.36 g, 5.2 mmol) in DCM (10 mL) at −10° C. was added CBr₄ (1.59 g, 4.8 mmol), stirred at 0° C. for 1 h and room temperature for 2 h, diluted with light petroleum (50 mL) and filtered. The filtrate was concentrated to give the title compound as a colorless liquid (500 mg, 65%).

### Step-2: 2-Amino-4-chloro-8-((4-methoxy-3,5-dimethyl-pyridin-2-ylmethyl)-5-[2-(2-oxo-pyrrolidin-1-yl)-ethyl]-7,8-dihydro-5H-pteridin-6-one

The title compound was obtained in 12% yield using the procedure described in Example I-2. ¹H NMR (400 MHz, CDCl₃) δ: 1.89-1.93 (m, 2H), 2.20-2.27 (m, 8H), 3.34-3.37 (t, 2H), 3.46-3.49 (t, 2H), 3.78 (s, 3H), 4.04 (s, 2H), 4.33-4.36 (t, 2H), 4.78 (s, 2H), 8.15 (s, 1H). MS m/z: 460 [M+1]^{+·}.

### Example I-12

### 2-{2-[2-Amino-4-chloro-8-((4-methoxy-3,5-dimethyl-pyridin-2-ylmethyl)-6-oxo-7,8-dihydro-6H-pteridin-5-yl]-ethyl}-isoindole-1,3-dione

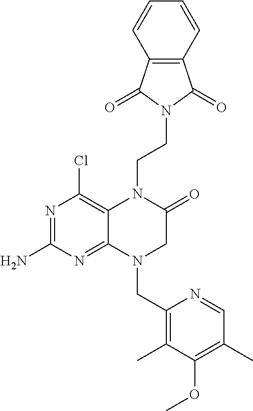

The title compound was obtained in 10% yield. ¹H NMR (400 MHz, CDCl₃) δ: 2.23 (s, 3H), 2.25 (s, 3H), 3.76 (s, 3H), 3.95-3.98 (m, 4H), 4.49-4.52 (t, 2H), 4.62 (s, 2H), 4.73 (s, 2H), 7.27-7.70 (m, 2H), 7.79-7.81 (m, 2H), 8.08 (s, 1H). MS m/z: 522 [M+1]^{+·}.

### Example I-13

### 2-{2-[2-Amino-4-chloro-8-((4-methoxy-3,5-dimethyl-pyridin-2-ylmethyl)-7,8-dihydro-pteridin-6-yloxy]-ethyl}-isoindole-1,3-dione

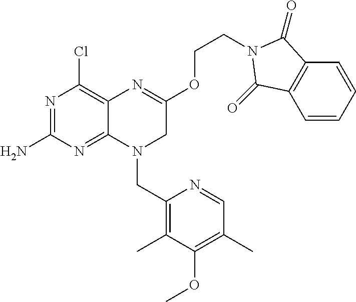

The title compound was obtained 10% yield. NMR (400 MHz, CDCl₃) δ: 2.24 (s, 3H), 2.25 (s, 3H), 3.76 (s, 3H), 4.05-4.07 (m, 4H), 4.56 (t, 2H), 4.65 (s, 2H), 4.81 (s, 2H), 7.72 (dd, 2H), 7.84 (dd, 2H), 8.17 (s, 1H). MS m/z: 522 [M+1]^{+·}.

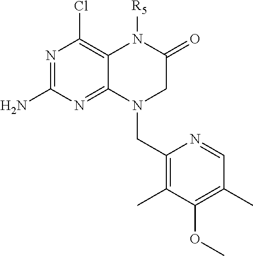

| Series | R |
|---|---|
| I-14 | cyclopentyl |
| I-15 | (CH₃)₂N(CH₂)₂NHCOCH(CH₃)— |
| I-16 | cyclopentyl enol ether |
| I-17 | (morpholin-1-yl)(CH₂)₂NHCO— |

Additional exemplary compounds of formula (I) are shown above.

Exemplary compounds of formula (I) can also be prepared as shown in Schemes 2-4 below.

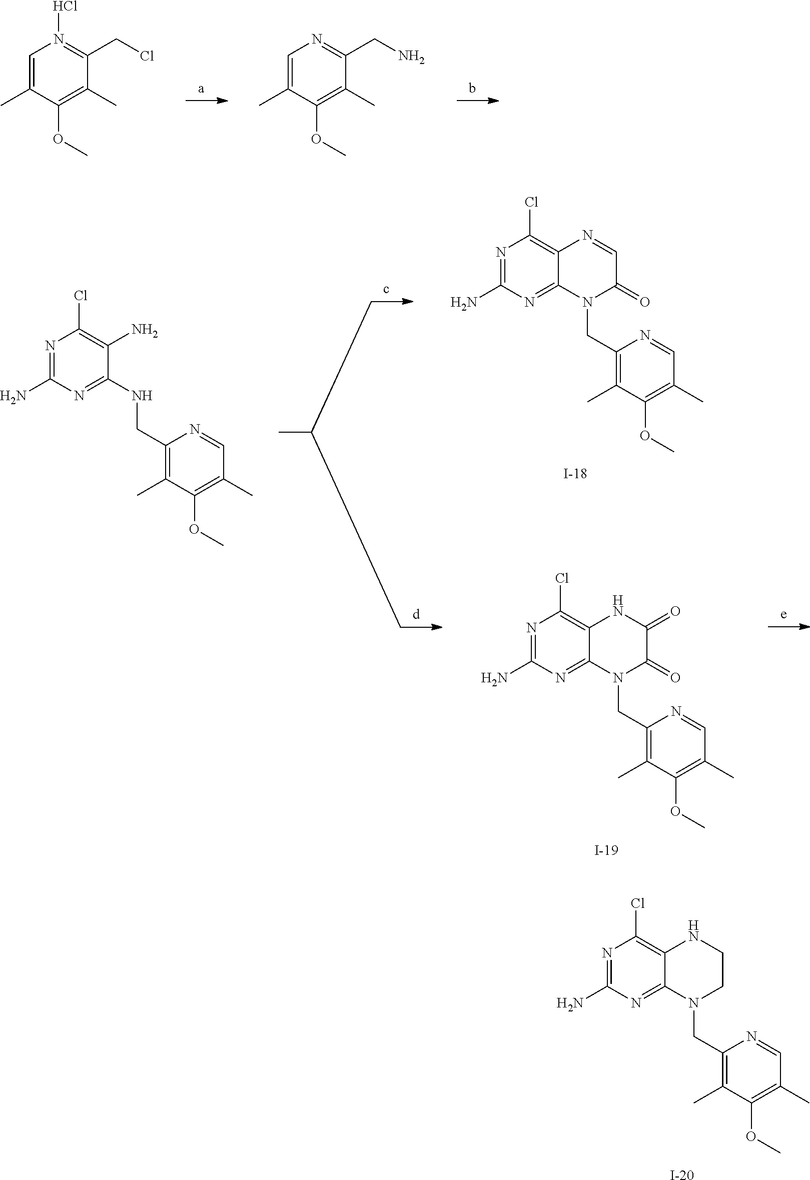

Scheme 2. Diversification of the pteridinone core: (a) i) K₂CO₃, DMF, isoindole-1,3-dione, room temperature, 24 h; ii) NH₂NH₂, 50° C., 1 h; 48% yield (b) Et₃N, BuOH, 4,6-dichloropyrimidine-2,5-diamine, reflux, 3 h; 50% yield (c) ethyl 2-oxoacetate, acetic acid, toluene, reflux, 4 h; 54% yield (d) i) ethyl oxalyl chloride, K₂CO₃, acetone, room temperature, overnight; ii) Et₃N, EtOH, reflux, 4 h; 90% yield e) LAH, THF, room temperature for 60 min; 90% yield.

Modification at the 4-position was accomplished under the conditions shown below in Scheme 3.

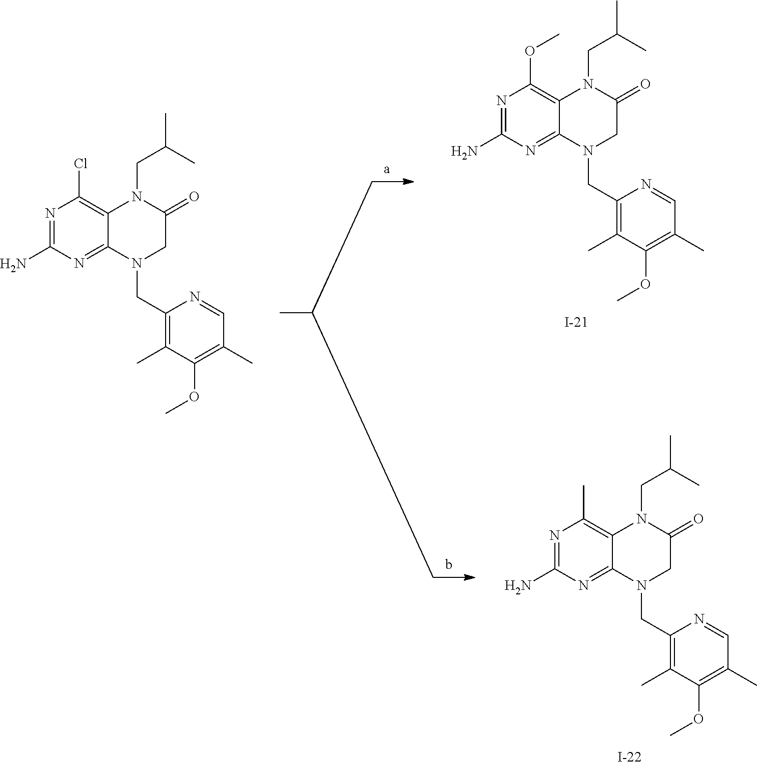

Scheme 3. Synthesis of 4-substituted-2-amino-5-isobutyl-8-((4-methoxy-3,5-dimethylpyridin-2-yl)methyl)-7,8-dihydropteridin-6(5H)-ones: (a) NaOH, CH₃OH—H₂O (6:1), reflux, 48 h; 70% yield (b) Pd(PPh₃)₄, AlMe₃, THF, reflux, overnight; 50% yield.

Adding various substituents at the 7-position was also accomplished as shown in Scheme 4 below.

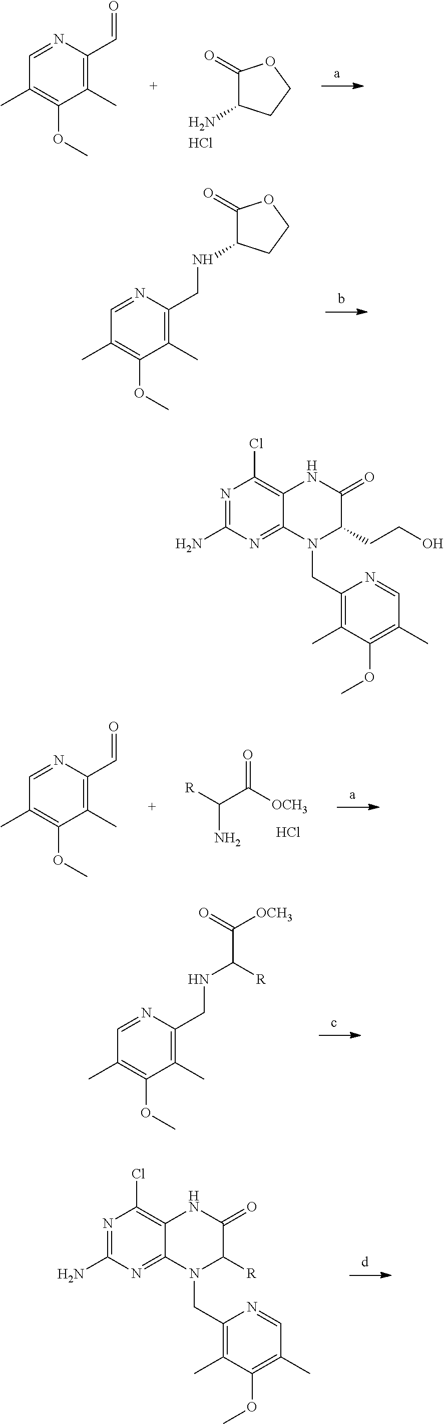

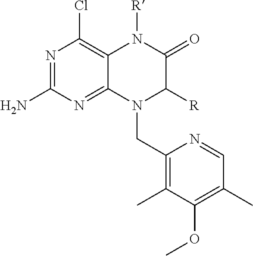

Scheme 4. Synthesis of 7-substituted-2-amino-4-chloro-8-((4-methoxy-3,5-dimethylpyridin-2-yl)methyl)-7,8-dihydropteridin-6(5H)-one: (a) NaBH(OAc)₃, TEA, CH₂Cl₂, room temperature, 2 hr; 57-76% yield (b) 4,6-dichloropyrimidine-2,5-diamine, H₂SO₄, MeOH, reflux, overnight; 2.1% yield c) DIEA, nBuOH, 150° C. in sealed tube, 48 h; 5-10% yield (d) NaH, DMF, RX., room temperature, overnight.

Following compounds were prepared according to Scheme 4:

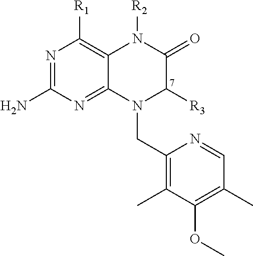

| ID | R₁ | R₂ | R₃ |
|---|---|---|---|
| I-23 | OCH₃ | (CH₃)₂CHCH₂— | H |
| I-24 | CH₃ | (CH₃)₂CHCH₂— | H |
| I-25 | Cl | H | (S)-CH₂CH₂OH |
| I-26 | Cl | H | (R)-CH₃— |
| I-27 | Cl | H | (S)-CH₃— |
| I-28 | Cl | H | (S)-(CH₃)₂CH— |
| I-29 | Cl | H | (S)-(CH₃)₂CHCH₂— |
| I-30 | Cl | (CH₃)₂CHCH₂— | (S)-CH₃— |
| I-31 | Cl | (CH₃)₂CHCH₂— | (S)-(CH₃)₂CH— |
| I-32 | Cl | (CH₃)₂CH— | (S)-(CH₃)₂CH— |

Following compounds were prepared according to Scheme-1

| I-36 | | | |
|---|---|---|---|
| 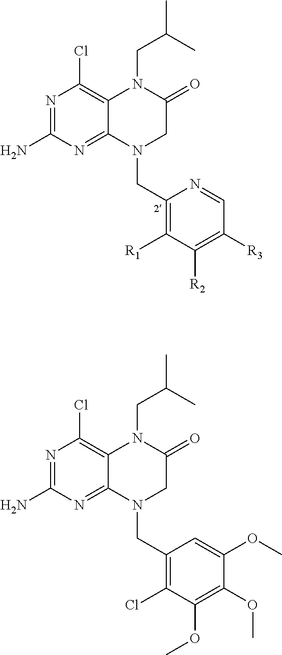 | | | |
| ID | R1 | R2 | R3 |
| I-33 | CH₃ | Cl | CH₃ |
| I-34 | CH₃ | Br | CH₃ |
| I-35 | OCH₃ | OCH₃ | H |
| I-36 | — | — | — |

Exemplary Compounds of Formula (II)

### Example II-1

### 2-Amino-7-((4-methoxy-3,5-dimethylpyridin-2-yl)methyl)-5H-pyrrolo[3,2-d]pyrimidin-4-ol

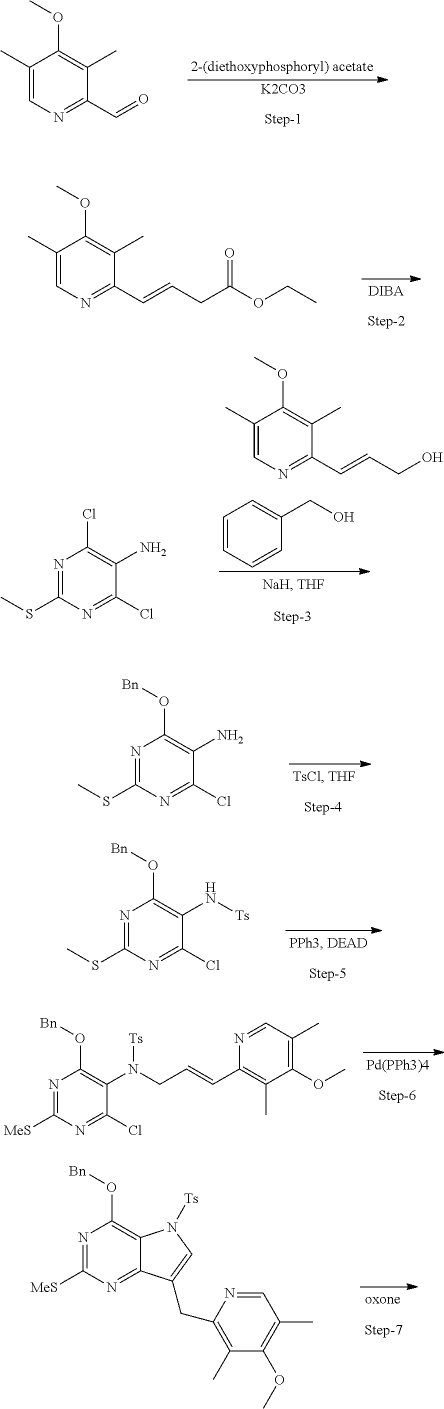

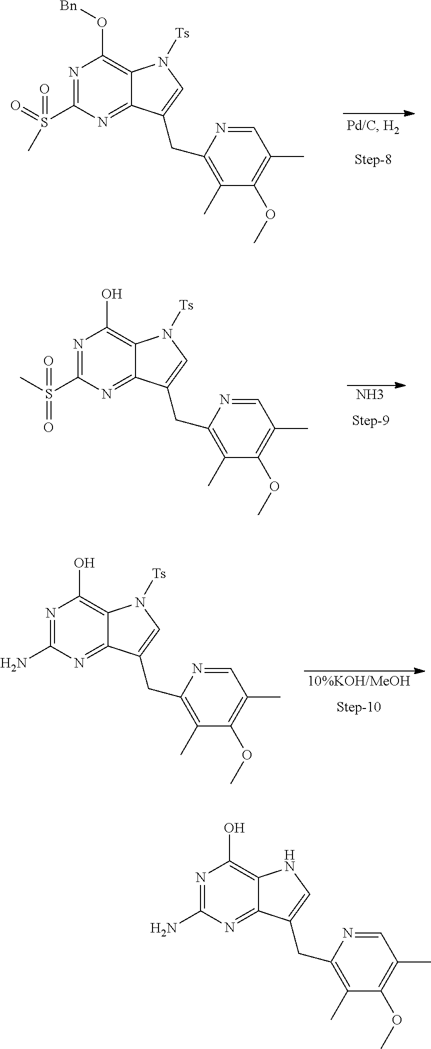

### Step-1: (E)-Ethyl 3-(4-methoxy-3,5-dimethylpyridin-2-yl)acrylate

To a solution of 4-methoxy-3,5-dimethylpicolinaldehyde (2.7 g, 16.36 mmol) in EtOH (50 mL) was added ethyl 2-(diethoxyphosphoryl)acetate (7.3 g, 32.59 mmol) and K₂CO₃ (6.8 g, 49.28 mmol). The resulting mixture was stirred for 4 hours at 70° C. A filtration was performed. The filtrate was concentrated under vacuum, treated with 40 ml of water, and then extracted with 3×40 mL of EtOAc. The organic layers were combined and washed with 20 mL of brine. The mixture was dried over Na₂SO₄ and concentrated under vacuum, resulted in 4 g of (E)-ethyl 3-(4-methoxy-3,5-dimethylpyridin-2-yl)acrylate as colorless oil.

### Step-2: (E)-3-(4-Methoxy-3,5-dimethylpyridin-2-yl) prop-2-en-1-ol

To (E)-ethyl 4-(4-methoxy-3,5-dimethylpyridin-2-yl) but-3-enoate (20 g, 80.32 mmol) in THF (300 mL) was added DMA (220 mL) dropwise with stirring at −7° C. over a time period of an hour. The resulting solution was stirred for an hour at −78° C. It was then quenched by the adding 50 mL of H₂O/ice. A filtration was performed and the filtrate was concentrated under vacuum. The residue was washed with 100 mL of CH₃OH and purified through a silica gel column with CH₂Cl₂/MeOH (100:1), resulted in 14 g (89%) of (E)-3-(4-methoxy-3,5-dimethylpyridin-2-yl) prop-2-en-1-ol as a yellow solid.

### Step-3: 4-(Benzyloxy)-6-chloro-2-(methylthio) pyrimidin-5-amine

To a solution of phenylmethanol (1.8 g, 16.67 mmol) in THF (40 ml) under N₂ was added NaH (680 mg, 17.00 mmol, 60%) at 0° C. and then a solution of 4,6-dichloro-2-(methylthio) pyrimidin-5-amine (3 g, 14.35 mmol) in THF (40 mL). The resulting solution was stirred for overnight at room temperature. The reaction mixture was then quenched by the adding 100 mL of H₂O/ice. The resulting solution was extracted with 3×200 mL of EtOAc and the organic layers were combined. The resulting mixture was washed with 2×200 mL of H₂O and with 100 mL of saturated NaCl. The mixture was dried over Na₂SO₄ and concentrated by evaporation under vacuum. The residue was purified through a silica gel column with EtOAc/PE (1:20), resulted in 3.5 g (94%) of 4-(benzyloxy)-6-chloro-2-(methylthio) pyrimidin-5-amine as a yellow solid. ¹H NMR (300 MHz, CDCl₃) δ 2.52 (3H, s), 3.97 (2H, s), 5.45 (2H, s), 7.35-7.45 (5H, m).

### Step-4: N-(4-(Benzyloxy)-6-chloro-2-(methylthio) pyrimidin-5-yl)-4-methylbenzenesulfonamide

To a solution of 4-(benzyloxy)-6-chloro-2-(methylthio)pyrimidin-5-amine (5 g, 17.62 mmol) in THF (50 mL) was added n-BuLi (21 mL, 2.5N) dropwise with stirring at −78° C. To the mixture was added TsCl (6.7 g, 35.26 mmol). The resulting solution was stirred at −78° C. for 3 hours. The reaction was then quenched by the adding 20 mL of H₂O. The resulting solution was extracted with 3×50 mL of EtOAc. The organic layers were combined, dried, and concentrated by evaporation under vacuum. The residue was purified by eluting through a silica gel column with PE:EA (5:1) solvent system, resulted in 5.0 g (64%) of N-(4-(benzyloxy)-6-chloro-2-(methylthio) pyrimidin-5-yl)-4-methylbenzenesulfonamide as a yellow solid. ¹H NMR (300 MHz, CDCl₃) δ 2.38 (3H, s), 2.52 (3H, s), 5.22 (2H, s), 5.94 (1H, s), 7.14-7.17 (2H, d), 7.23 (2H, m), 7.32 (1H, m), 7.64-7.67 (2H, d).

### Step-5: (E)-N-(4-(Benzyloxy)-6-chloro-2-(methylthio) pyrimidin-5-yl)-N-(3-(4-methoxy-3,5-dimethylpyridin-2-yl) allyl)-4-methylbenzenesulfonamide

To a mixture of N-(4-(benzyloxy)-6-chloro-2-(methylthio) pyrimidin-5-yl)-4-methylbenzenesulfonamide (50 mg, 0.11 mmol) in THF (20 mL) and (E)-3-(4-methoxy-3,5-dimethylpyridin-2-yl) prop-2-en-1-ol (22 mg, 0.11 mmol) was added PPh₃ (45 mg, 0.17 mmol) and a solution of DEAD (34 mg, 0.17 mmol) in THF (5 mL) at 0° C. The resulting solution was stirred for 2 hours at room temperature. The reaction progress was monitored by TLC (CH₂Cl₂/MeOH=10:1). The reaction was then quenched by the adding 30 ml of H₂O and extracted with 4×50 mL of EtOAc. The organic layers were combined, dried over Na₂SO₄, and concentrated under vacuum. The residue was purified through a silica gel column with EtOAc/PE (1:5), resulted in 0.05 g (71%) of (E)-N-(4-(benzyloxy)-6-chloro-2-(methylthio) pyrimidin-5-yl)-N-(3-(4-methoxy-3,5-dimethylpyridin-2-yl)allyl)-4-methylbenzenesulfonamide as a yellow solid.

### Step-6: 4-(Benzyloxy)-7-((4-methoxy-3,5-dimethylpyridin-2-yl)methyl)-2-(methylthio)-5-tosyl-5H-pyrrolo[3,2-d]pyrimidine

To (E)-N-(4-(benzyloxy)-6-chloro-2-(methylthio) pyrimidin-5-yl)-N-(3-(4-methoxy-3,5-dimethylpyridin-2-yl)allyl)-4-methylbenzenesulfonamide (1 g, 1.64 mmol) was added Pd(pph₃)₄ (600 mg, 0.52 mmol), KOAc (2.4 g, 24.49 mmol), and toluene (6 mL). The reaction mixture was irradiated with microwave radiation for 20 min at 180° C. The solids were filtered out and the residue was purified onto a silica gel column with ethyl acetate/PE (5:1), resulted in 200 mg (21%) of 4-(benzyloxy)-7-((4-methoxy-3,5-dimethylpyridin-2-yl)methyl)-2-(methylthio)-5-tosyl-5H-pyrrolo[3,2-d]pyrimidine as a yellow solid.

### Step-7: 4-(Benzyloxy)-7-((4-methoxy-3,5-dimethylpyridin-2-yl)methyl)-2-(methylsulfonyl)-5-tosyl-5H-pyrrolo[3,2-d]pyrimidine

To 4-(benzyloxy)-7-((4-methoxy-3,5-dimethylpyridin-2-yl)methyl)-2-(methylthio)-5-tosyl-5H-pyrrolo[3,2-d]pyrimidine (750 mg, 1.31 mmol) was added CH₂Cl₂ (20 mL) and KHSO₅ (8.2 g). The resulting mixture was stirred for 36 hours at room temperature. The reaction progress was monitored by TLC (CH₂Cl₂/MeOH=10:1). A filtration was performed and the filtrate was washed with 10 mL of NaHCO₃ saturated solution. The resulting mixture was washed with 10 mL of brine and dried over Na₂SO₄. The residue was purified through a silica gel column with EtOAc/PE (2:1), resulted in 0.68 g (86%) of 4-(benzyloxy)-7-((4-methoxy-3,5-dimethylpyridin-2-yl)methyl)-2-(methylsulfonyl)-5-tosyl-5H-pyrrolo[3,2-d]pyrimidine as a white solid.

### Step-8: 7-((4-Methoxy-3,5-dimethylpyridin-2-yl)methyl)-2-(methylsulfonyl)-5-tosyl-5H-pyrrolo[3,2-d]pyrimidin-4-ol

To a mixture of 4-(benzyloxy)-7-((4-methoxy-3,5-dimethylpyridin-2-yl)methyl)-2-(methylsulfonyl)-5-tosyl-5H-pyrrolo[3,2-d]pyrimidine (6.5 g, 10.71 mmol) in CH₃OH (400 mL) was added Pd/C (0.65 g). The resulting mixture was stirred for 12 hours at room temperature. The reaction progress was monitored by TLC (CH₂Cl₂/MeOH=10:1). A filtration was performed and the filtrate was concentrated under vacuum using a rotary evaporator, resulted in 5.6 g (96%) of 7-((4-methoxy-3,5-dimethylpyridin-2-yl)methyl)-2-(methylsulfonyl)-5-tosyl-5H-pyrrolo[3,2-d]pyrimidin-4-ol as a white solid.

### Step-9: 2-Amino-7-((4-methoxy-3,5-dimethylpyridin-2-yl)methyl)-5-tosyl-5H-pyrrolo[3,2-d]pyrimidin-4-ol

A pressure tank reactor (1 L) was purged, flushed and maintained with NH₃ (10 atm). To it was added 7-((4-methoxy-3,5-dimethylpyridin-2-yl)methyl)-2-(methylsulfonyl)-5-tosyl-5H-pyrrolo[3,2-d]pyrimidin-4-ol (5.6 g, 10.29 mmol) and 1,4-dioxane (500 ml). The resulting solution was stirred for 12 minutes at 160° C. The mixture was concentrated under vacuum using a rotary evaporator. The residue was purified through a silica gel column with CH₂Cl₂/MeOH (20:1), resulted in 3 g (64%) of 2-amino-7-((4-methoxy-3,5-dimethylpyridin-2-yl)methyl)-5-tosyl-5H-pyrrolo[3,2-d]pyrimidin-4-ol as a yellow solid.

### Step-10: 2-Amino-7-((4-methoxy-3,5-dimethylpyridin-2-yl)methyl)-5H-pyrrolo[3,2-d]pyrimidin-4-ol

To 2-amino-7-((4-methoxy-3,5-dimethylpyridin-2-yl)methyl)-5-tosyl-5H-pyrrolo[3,2-d]pyrimidin-4-ol (200 mg, 0.42 mmol) was added 10 mL of KOH/CH₃OH (10% solution). The resulting solution was heated to reflux for 2 min. The reaction progress was monitored by TLC (CH₂Cl₂/MeOH=5:1). The pH of the mixture was adjusted to 7-8 with HCl (1 M). The resulting mixture was concentrated under vacuum using a rotary evaporator. The residue was washed with 2×50 mL of CH₂Cl₂/CH₃OH. The solids was filtered, resulted in 0.12 g (91%) of 2-amino-7-(4-methoxy-3,5-dimethylpyridin-2-yl)methyl)-5H-pyrrolo[3,2-d]pyrimidin-4-ol as a white solid. ¹H NMR (300 MHz, DMSO-d₆) δ 2.17 (6H, s), 3.7 (3H, s), 3.89 (2H, s), 5.92 (2H, s), 6.62 (1H, s), 8.13 (1H, s), 11.21 (1H, s). MS m/z: 300 [M+1]^{+·}.

### Example II-2

### 4-Chloro-7-((4-methoxy-3,5-dimethylpyridin-2-yl)methyl)-5H-pyrrolo[3,2-d]pyrimidin-2-amine

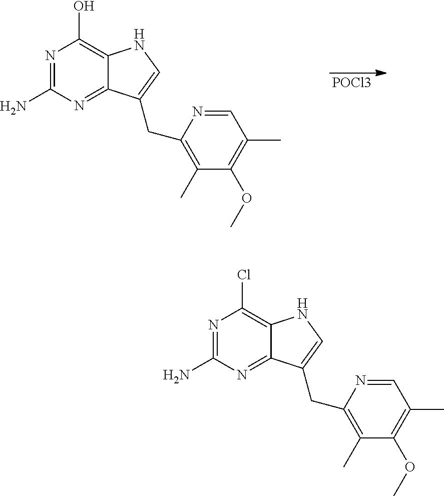

To a solution of 2-amino-7-((4-methoxy-3,5-dimethylpyridin-2-yl)methyl)-5H-pyrrolo[3,2-d]pyrimidin-4-ol (1 g, 3.34 mmol) in CH₃CN (20 mL) was added diethylaniline (1 g, 6.71 mmol) and POCl₃ (20 mL). The resulting solution was refluxed for 4 hours. The reaction was then quenched with 30 g of H₂O/ice. Adjustment of the pH to 8 was accomplished by the addition of K₂CO₃ (20% solution). The resulting solution was extracted with 4×120 mL of CH₂Cl₂. The organic layers were combined, dried over Na₂SO₄, and concentrated under vacuum using a rotary evaporator. The residue was purified by eluting through a silica column with CH₂Cl₂/MeOH (50:1-15:1), resulted in 104 mg (28%) of the title compound as a white solid. ¹H NMR (300 MHz, CD₃COCD₃) δ 2.19 (3H, s), 2.27 (3H, s), 3.75 (3H, s), 4.08 (2H, s), 5.63 (2H, s), 7.22 (1H, s), 8.15 (1H, s), 10.45 (1H, s). MS m/z: 318 [M+1]^{+·}.

### Example-3

### 4-Chloro-7-((4-methoxy-3,5-dimethylpyridin-2-yl)methyl)-5-methyl-5H-pyrrolo[3,2-d]pyrimidin-2-amine

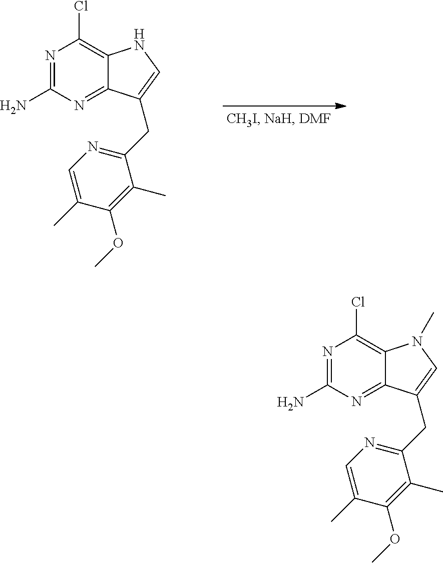

To a solution of 4-chloro-7-((4-methoxy-3,5-dimethylpyridin-2-yl)methyl)-5H-pyrrolo[3,2-d]pyrimidin-2-amine (32 mg, 0.10 mmol) in DMF (5 mL) was added NaH (4 mg, 0.17 mmol) at 0° C. and iodomethane (100 mg, 0.70 mmol). The resulting mixture was stirred for 12 hr at room temperature. The reaction was then quenched by the addition of 10 mL of water and extracted with 3×20 mL of dichloromethane. The organic layers were combined and washed with 3×20 mL of brine. The organic solution was dried over anhydrous sodium sulfate and concentrated to dryness. The residue was purified onto a silica gel column with dichloromethane/methanol (15:1), resulted in 14 mg (42%) of the title compound as a white solid. ¹H NMR (300 MHz, CD₃COCD₃) δ 2.22 (3H, s), 2.26 (3H, s), 3.76 (3H, s), 3.94 (3H, s), 4.05 (2H, s), 5.61 (2H, s), 7.08 (1H, s), 8.15 (1H, s). MS m/z: 332 [M+1]^{+·}.

### Example II-4

### 2-(2-Amino-4-chloro-7-((4-methoxy-3,5-dimethylpyridin-2-yl)methyl)-5H-pyrrolo[3,2-d]pyrimidin-5-yl)ethanol

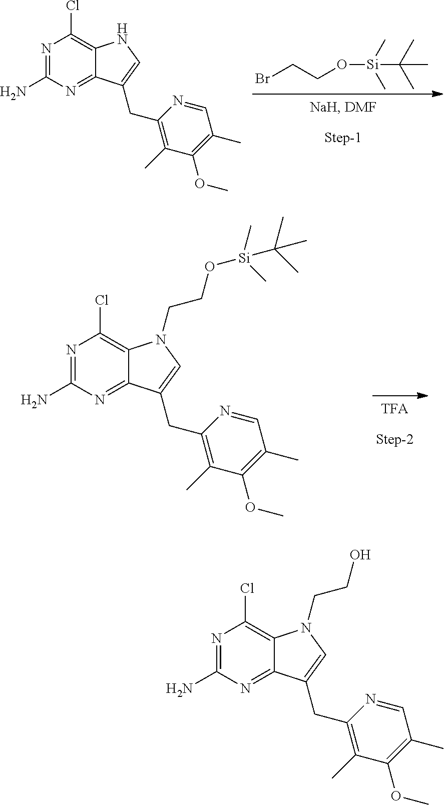

### Step-1: 5-(2-(tert-Butyldimethylsilyloxy)ethyl)-4-chloro-7-((4-methoxy-3,5-dimethylpyridin-2-yl)methyl)-5H-pyrrolo[3,2-d]pyrimidin-2-amine

To 4-chloro-7-((4-methoxy-3,5-dimethylpyridin-2-yl)methyl)-5H-pyrrolo[3,2-d]pyrimidin-2-amine (30 mg, 0.09 mmol) under N₂ was added DMF (5 mL), NaH (3 mg, 0.12 mmol), and (2-bromoethoxy)(tert-butyl) dimethylsilane (27 mg, 0.11 mmol) at 0° C. in duration of 2 hrs. The resulting mixture was stirred for 12 hrs at room temperature. The reaction was then quenched by the addition of 10 mL of water. The resulting solution was extracted with 3×20 mL of dichloromethane. The organic layers were combined, washed with 10 mL of brine, dried over anhydrous sodium sulfate, and concentrated to dryness. The residue was purified onto a silica gel column with dichloromethane/methanol (15:1), resulted in 22 mg (49%) of 5-(2-(tert-butyldimethylsilyloxy)ethyl)-4-chloro-7-((4-methoxy-3,5-dimethylpyridin-2-yl)methyl)-5H-pyrrolo[3,2-d]pyrimidin-2-amine as a yellow solid.

### Step-2: 2-(2-Amino-4-chloro-7-((4-methoxy-3,5-dimethylpyridin-2-yl)methyl)-5H-pyrrolo[3,2-d]pyrimidin-5-yl)ethanol

To 5-(2-(tert-butyldimethylsilyloxy)ethyl)-4-chloro-7-((4-methoxy-3,5-dimethylpyridin-2-yl)methyl)-5H-pyrrolo[3,2-d]pyrimidin-2-amine (20 mg, 0.04 mmol) was added 5 mL of a solution (THF:H₂O:CF₃COOH=1:1:3). The resulting mixture was stirred for 2 hrs at room temperature and then concentrated under vacuum. The crude product was re-crystallized from CH₂Cl₂/ether (2:3), resulted in 8 mg (51%) of the title compound as a white solid. ¹H NMR (400 MHz, CD₃COCD₃) δ 2.21 (3H, s), 2.23 (3H, s), 3.83 (3H, s), 3.85 (2H, m), 3.96 (1H, s), 4.05 (2H, s), 4.37 (2H, m), 5.59 (2H, s), 7.13 (1H, s), 8.14 (1H, s). MS m/z: 362 [M+1]^{+·}.

### Example II-5

### 7-(3,4-Dimethoxybenzyl)-4-chloro-5-(4-methylpentyl)-5H-pyrrolo[3,2-d]pyrimidin-2-amine

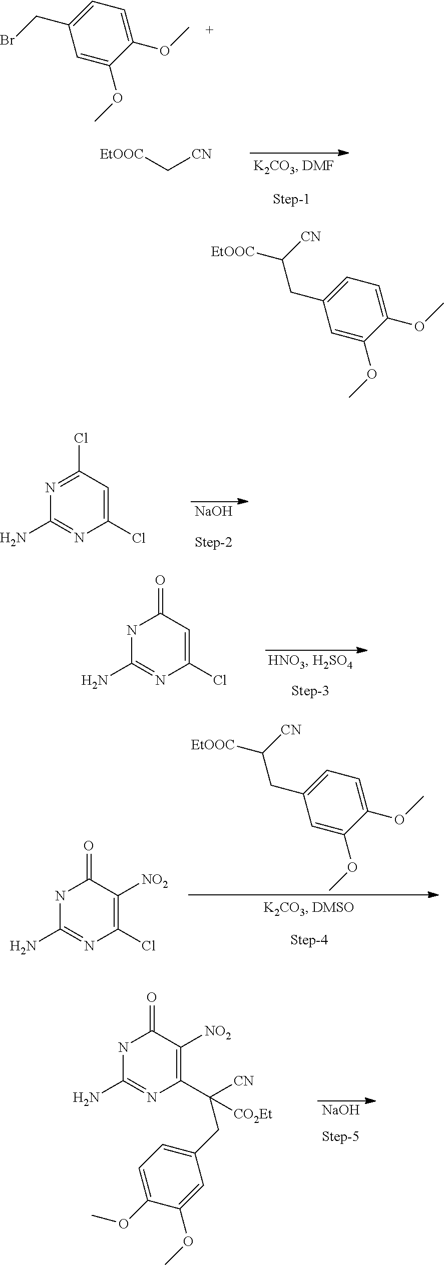

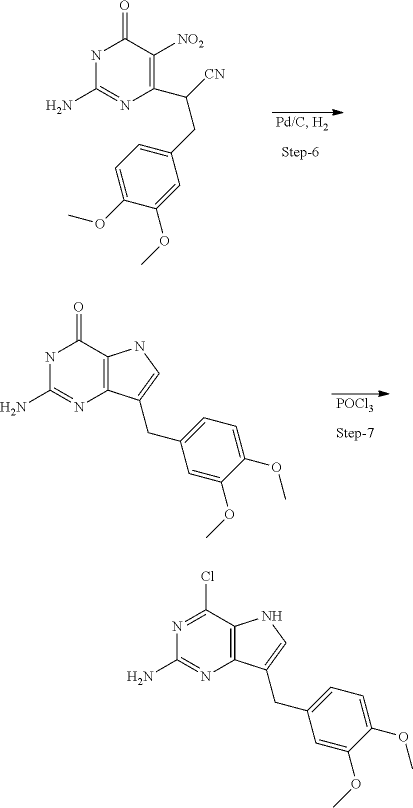

### Step-1: Ethyl 2-cyano-3-(3,4-dimethoxyphenyl)propanoate

To a mixture of K₂CO₃ (1.8 g, 13.04 mmol) in DMF (10 mL) at 0° C. was added ethyl 2-cyanoacetate (4.9 g, 43.36 mmol) and a solution of 4-(bromomethyl)-1,2-dimethoxybenzene (1 g, 4.35 mmol) in DMF (5 mL) over a period of 5 min. The resulting solution was stirred for 3 hours at room temperature and then quenched by the adding 20 mL of H₂O. The mixture was extracted with 3×100 mL of EtOAc. The organic layers were combined, dried over Na₂SO₄, and concentrated under vacuum using a rotary evaporator. The residue was purified by eluting through a silica gel column with PE:EA (20:1) solvent system, resulted in 0.7 g (21%) of ethyl 2-cyano-3-(3,4-dimethoxyphenyl)propanoate as a colorless oil.

### Step-2: 2-Amino-6-chloropyrimidin-4(3H)-one

To a solution of 4,6-dichloropyrimidin-2-amine (21.2 g, 129.27 mmol) in 200 mL of 1N NaOH was added NaOH (4 g, 100.00 mmol) in several batches. The resulting solution was refluxed for 5 hours. The reaction mixture was cooled in a bath of H₂O/ice. Adjustment of the pH to 5-6 was accomplished by the addition of CH₃COOH. A filtration was performed and the filter cake was collected. The solid was washed with water and dried under an oven with reduced pressure. This resulted in 17 g (86%) of 2-amino-6-chloropyrimidin-4(3H)-one as a white solid.

### Step-3: 2-Amino-6-chloro-5-nitropyrimidin-4(3H)-one

To a solution of 2-amino-6-chloropyrimidin-4(3H)-one (10 g, 68.73 mmol) in H₂SO₄ (46 mL, 98%) at 40° C. was added HNO₃ (9 g, 65%) dropwise with stirring. The resulting solution was stirred for an hour at room temperature. The solution was poured into 100 g of ice. A filtration was performed. The filter cake was collected and dried under an oven with reduced pressure, resulted in 12 g (87%) of 2-amino-6-chloro-5-nitropyrimidin-4(3H)-one as a yellow solid. ¹H NMR (300 MHz, DMSO-d₆) δ 7.13 (1H, s), 8.59 (1H, s), 12.16 (1H, s).

### Step-4: Ethyl 2-(2-amino-5-nitro-6-oxo-1,6-dihydropyrimidin-4-yl)-2-cyano-3-(3,4-dimethoxyphenyl)propanoate

To a solution of ethyl 2-cyano-3-(3,4-dimethoxyphenyl)propanoate (1 g, 3.80 mmol) in DMSO (10 mL) under N₂ was added K₂CO₃ (1.57 g, 11.38 mmol) and 2-amino-6-chloro-5-nitropyrimidin-4(3H)-one (720 mg, 3.79 mmol). The resulting mixture was stirred for 24 hours at 65-70° C. in a bath of oil. The reaction was quenched by the adding 20 mL of H₂O. The resulting solution was extracted with 3×50 mL of EtOAc. The organic layers were combined, dried over Na₂SO₄, and concentrated under vacuum using a rotary evaporator. The residue was purified by eluting through a silica column with CH₂Cl₂/MeOH (50:1). This resulted in 0.1 g (6%) of ethyl 2-(2-amino-5-nitro-6-oxo-1,6-dihydropyrimidin-4-yl)-2-cyano-3-(3,4-dimethoxyphenyl)propanoate as a yellow solid.

### Step-5: 2-(2-Amino-5-nitro-6-oxo-1,6-dihydropyrimidin-4-yl)-3-(3,4-dimethoxyphenyl)propanenitrile

To ethyl 2-(2-amino-5-nitro-6-oxo-1,6-dihydropyrimidin-4-yl)-2-cyano-3-(3,4-dimethoxyphenyl)propanoate (350 mg, 0.84 mmol) was added 20 mL of 1 N NaOH. The resulting solution was stirred for an hour at room temperature. The pH of reaction mixture was adjusted by the addition of HCl (4 N). A filtration was performed and the filter cake was collected and dried in an oven under reduced pressure. This resulted in 0.23 g (79.4%) of 2-(2-amino-5-nitro-6-oxo-1,6-dihydropyrimidin-4-yl)-3-(3,4-dimethoxyphenyl)propanenitrile as a red solid.

### Step-6: 7(3,4-Dimethoxybenzyl)-2-amino-3H-pyrrolo[3,2-d]pyrimidin-4(5H)-one

To 2-(2-amino-5-nitro-6-oxo-1,6-dihydropyrimidin-4-yl)-3-(3,4-dimethoxyphenyl)propanenitrile (1.5 g, 4.35 mmol) in EA/HOAc (20/10 mL) was added Pd/C (0.75 g, 10%). The resulting solution was stirred under H₂ for overnight. A filtration was performed and the filtrate was concentrated under vacuum using a rotary evaporator. The resulting mixture was treated with 10 mL of saturated solution of Na₂CO₃ and was extracted with 3×20 mL of EtOAc. The organic layers were combined, dried over Na₂CO₃, and concentrated under vacuum using a rotary evaporator. The residue was purified by eluting through a silica column with CH₂Cl₂/MeOH (30:1). This resulted in 1 g (25%) of 7-(3,4-dimethoxybenzyl)-2-amino-3H-pyrrolo[3,2-d]pyrimidin-4(5H)-one as a white solid. ¹H NMR (300 MHz, CD₃COCD₃) δ 3.77 (6H, s), 3.85 (2H, s), 6.73-6.88 (3H, m), 6.93 (1H, s).

### Step-7: 7-(3,4-Dimethoxybenzyl)-4-chloro-5H-pyrrolo[3,2-d]pyrimidin-2-amine

To a solution of 7-(3,4-dimethoxybenzyl)-2-amino-3H-pyrrolo[3,2-d]pyrimidin-4(5H)-one (600 g, 2.00 mol) in CH₃CN (50 mL) was added N,N-dimethylbenzenamine (120 mg, 0.99 mmol), N,N,N-trimethylbenzenammonium chloride (500 mg, 2.19 mmol), and POCl₃ (3.1 g, 20.20 mmol). The resulting solution was refluxed for 4 hours. The reaction progress was monitored by TLC (CH₂Cl₂/MeOH=10:1). The mixture was concentrated under vacuum and treated with 50 mL of CH₂Cl₂. The resulting mixture was washed with 3×40 mL of Na₂CO₃ (saturated solution). The resulting solution was extracted with 3×40 mL of CH₂Cl₂. The organic layers were combined, dried over Na₂SO₄, and concentrated under vacuum using a rotary evaporator. The residue was purified by eluting through a silica column with CH₂Cl₂/MeOH (25:1) solvent system, resulted in 120 mg (19%) of 7-(3,4-dimethoxybenzyl)-4-chloro-5H-pyrrolo[3,2-d]pyrimidin-2-amine as a yellow solid. ¹H NMR (300 MHz, CD₃COCD₃) δ 4.0 (6H, s), 4.2 (2H, s), 7.02-7.12 (2H, m), 7.19 (1H, s), 7.54 (1H, s). MS m/z: 319 [M+1]^{+·}.

### Example II-6

### 7-(3,4-Dimethoxybenzyl)-4-chloro-5-(4-methylpentyl)-5H-pyrrolo[3,2-d]pyrimidin-2-amine

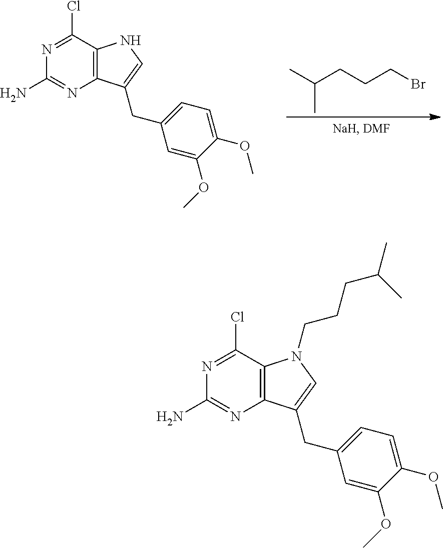

To a solution of 7-(3,4-dimethoxybenzyl)-4-chloro-5H-pyrrolo[3,2-d]pyrimidin-2-amine (227 mg, 0.71 mmol, 1.00 equiv) in DMF (50 mL) was added 1-bromo-4-methylpentane (141 mg, 0.85 mmol) and then NaH (34 mg, 1.42 mmol). The resulting solution was stirred for 4 hours at room temperature. The reaction was then quenched by the adding 50 mL of H₂O/ice. The resulting solution was extracted with 3×50 mL of EtOAc. The organic layers were combined, dried over Na₂SO₄, and concentrated under vacuum using a rotary evaporator. The residue was purified by recrystallization from EA:hexane (1:5), resulted in 39 mg (14%) of 7-(3,4-dimethoxybenzyl)-4-chloro-5-(4-methylpentyl)-5H-pyrrolo[3,2-d]pyrimidin-2-amine as a yellow solid. ¹H NMR (300 MHz, CDCl₃) δ 0.86 (6H, s), 1.15 (2H, m), 1.61 (1H, m), 1.75 (2H, m), 3.85 (6H, s), 3.95 (2H, s), 4.19 (2H, s), 4.87 (2H, s), 6.80-6.89 (4H, m). MS m/z: 403 [M+1]^{+·}.

Additional exemplary compounds of formula (II) are shown above.

### Exemplary Compounds of Formula (III)

### Example III-1

### 2-Amino-4-chloro-7-(4-methoxy-3,5-dimethyl-pyridin-2-ylmethyl)-5-[1-(1H-pyrrol-2-yl)-meth-(Z)-ylidene]-5,7-dihydro-pyrrolo[2,3-d]pyrimidin-6-one

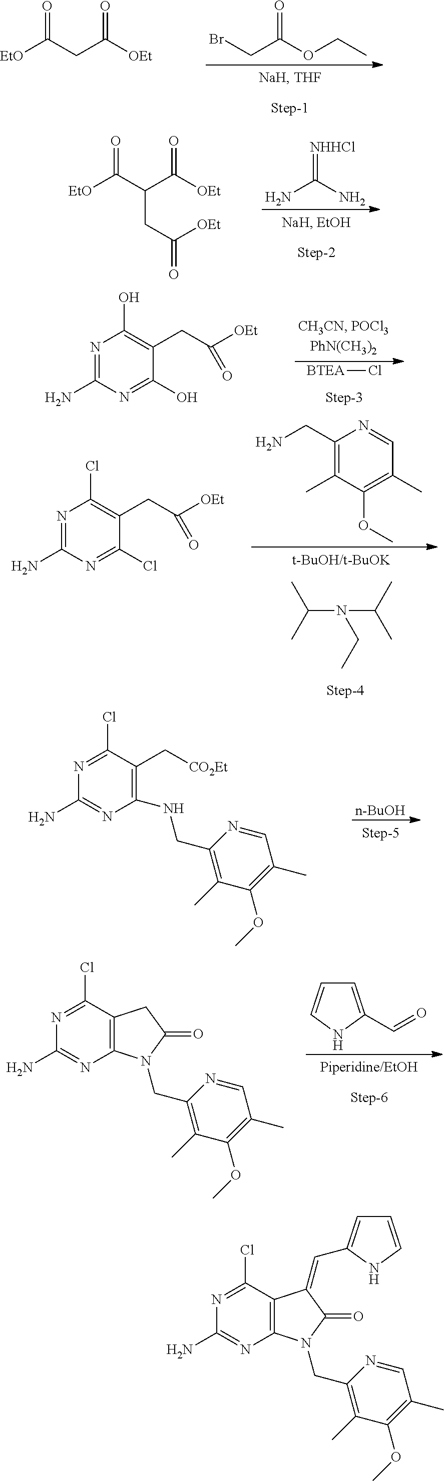

### Step-1: Triethyl ethane-1,1,2-tricarboxylate

To a suspension of NaH (24 g, 600 mmol) in THF (200 ml) at −10 to 0° C. was added diethyl malonate (100 g, 624.34 mmol) dropwise with stirring followed by addition of ethyl 2-bromoacetate (114 g, 682.63 mmol) dropwise over 80 minutes. The reaction mixture was warmed to room temperature, stirred for 1 h, and filtered. The filter cake was washed with THF and the filtrate was concentrated to afford the crude title compound (63.3 g) as a white liquid which was used without further purification. MS (m/z):247 [M+H]^{+·}.

### Step-2: Ethyl 2-(2-amino-4,6-dihydropyrimidin-5-yl)acetate

To a solution of NaH (10.25 g, 427.08 mmol) in EtOH (200 ml) at 0° C. was added guanidine hydrochloride (23.3 g, 243.88 mmol). The reaction mixture was warmed to room temperature, stirred for 1 h and filtered. The filter cake was washed 3 times with EtOH. To the filtrate was cooled to −10-0° C. and added triethyl ethane-1,1,2-tricarboxylate (63.3 g, 257.32 mmol). The reaction mixture was warmed to room temperature, refluxed overnight, and filtered. The solid was dried in a vacuum oven to give the title compound (35 g, 36%) as a white solid. MS (m/z): 214 [M+H]^{+·}.

### Step-3: Ethyl 2-(2-amino-4,6-dichloropyrimidin-5-yl)acetate

To a solution of ethyl 2-(2-amino-4,6-dihydroxypyrimidin-5-yl)acetate (23.3 g, 109.39 mmol), BTEA-Cl (127 g, 559.47 mmol) and N,N-dimethylbenzenamine (26.46 g, 218.68 mmol) in acetonitrile (500 ml) was added POCl₃ (161.9 g, 1.06 mol). The reaction mixture was refluxed for 2 h, stirred at 80° C. overnight, concentrated, cooled with ice bath, and diluted with water (10 mL). To the above ice-cooled solution was added sodium carbonate to adjust pH. The resulting mixture was extracted three times with EtOAc (100 ml). The combined organic layers were dried over anhydrous MgSO₄. The residue was purified by silica gel chromatography eluting with EtOAc-petroleum ether (1:15) to give the title compound (23 g, 84%) as a white solid.

### Step-4: Ethyl 2-(2-amino-4-chloro-6-((4-methoxy-3,5-dimethylpyridin-2-yl)methylamino)pyrimidin-5-yl)acetate

To a solution of (4-methoxy-3,5-dimethylpyridin-2-yl)methanamine dihydrochloride (10.75 g, 44.98 mmol) in t-BuOH (300 ml) was added t-BuOK (10 g, 89.29 mmol, 2.00 equiv). Then the mixture was stirred for 20 minutes. Addition of N-ethyl-N-isopropylpropan-2-amine (17.4 g, 134.63 mmol) was next. To the mixture was added ethyl 2-(2-amino-4,6-dichloropyrimidin-5-yl)acetate (11.2 g, 44.80 mmol). The resulting solution was allowed to react, with stirring, for 20 hours while the temperature was maintained at reflux. The reaction progress was monitored by TLC (EtOAc-petroleum=1:1). The mixture was concentrated by evaporation under vacuum using a rotary evaporator. The residue was dissolved in 300 ml of DCM. The resulting mixture was washed three times with 100 ml of water. The mixture was dried over MgSO₄ and concentrated by evaporation under vacuum using a rotary evaporator. The residue was purified by eluting through a column with a 1:5-1:1 EtOAc/PE solvent system. This resulted in 13 g (crude) of ethyl 2-(2-amino-4-chloro-6-((4-methoxy-3,5-dimethylpyridin-2-yl)methylamino) pyrimidin-5-yl)acetate as a yellow solid.

### Step-5: 2-amino-4-chloro-7-((4-methoxy-3,5-dimethylpyridin-2-yl)methyl)-5H-pyrrolo[2,3-d]pyrimidin-6(7H)-one

The reaction solution of ethyl 2-(2-amino-4-chloro-6-((4-methoxy-3,5-dimethylpyridin-2-yl)methylamino)pyrimidin-5-yl)acetate (1.65 g, 4.35 mmol) in n-BuOH (100 mL) was refluxed overnight and concentrated. The residue was purified by silica gel column chromatography eluted with ethyl acetate-petroleum ether (1:10˜1:1) to afford the title compound as a light yellow solid (1.1 g, 76%). MS m/z: 334 [M+H]^{+·}.

### Step-6: 2-Amino-4-chloro-7-(4-methoxy-3,5-dimethyl-pyridin-2-ylmethyl)-5-[1-(1H-pyrrol-2-yl)-meth-(Z)-ylidene]-5,7-dihydro-pyrrolo[2,3-d]pyrimidin-6-one

The reaction mixture of 2-amino-4-chloro-7-((4-methoxy-3,5-dimethylpyridin-2-yl)methyl)-5H-pyrrolo[2,3-d]pyrimidin-6(7H)-one (400 mg, 1.20 mmol), 1H-pyrrole-2-carbaldehyde (114.1 mg, 1.20 mmol), and 1 drop piperidine in EtOH (70 mL) was stirred at room temperature for 20 h and 40° C. for 18 h and filtered. The filter cake was washed four times with 5 mL of EtOH and dried to afford the title compound as a yellow solid (245 mg, 50%). ¹H NMR (300 MHz, CDCl₃,) δ 12.95 (1H, s), 8.25 (1H, s), 7.95 (1H, s), 7.13 (1H, s), 6.82 (1H, t, J=1.2 Hz), 6.40 (1H, m), 5.31 (2H, s), 5.156 (2H, s), 3.83 (3H, s), 2.39 (3H, s), 2.25 (3H, s). MS m/z: 411 [M+H]^{+·}.

The following compounds were prepared using the same protocols for the synthesis of Example III-1 with different aldehydes replacing 1H-pyrrole-2-carboxaldehyde.

### Example III-2

### 2-Amino-4-chloro-5-[1-(3,5-dimethyl-1H-pyrrol-2-yl)-meth-(Z)-ylidene]-7-(4-methoxy-3,5-dimethyl-pyridin-2-ylmethyl)-5,7-dihydro-pyrrolo[2,3-d]pyrimidin-6-one

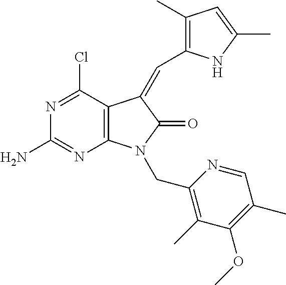

The title compound was obtained in 46% yield. ¹H NMR (300 MHz, CDCl₃,) δ 12.67 (1H, s), 8.10 (1H, s), 7.95 (1H, s), 5.98 (1H, s), 5.11 (2H, s), 4.95 (2H, s), 3.77 (3H, s), 2.32 (m, 9H), 2.19 (3H, s). MS m/z: 439 [M+1]^{+·}.

### Example III-3

### 2-Amino-4-chloro-5-[1-(1H-imidazol-2-yl)-meth-(Z)-ylidene]-7-(4-methoxy-3,5-dimethyl-pyridin-2-ylmethyl)-5,7-dihydro-pyrrolo[2,3-d]pyrimidin-6-one

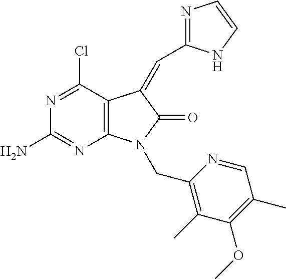

The title compound was prepared in 52% yield as a yellow solid. ¹H NMR (300 MHz, CDCl₃,) δ 2.20 (3H, s), 2.36 (3H, s), 3.78 (3H, s), 5.10 (2H, s), 5.21 (2H, s), 8.08 (1H, s), 8.16 (1H, s), 13.73 (1H, s). MS m/z: 412 [M+1]^{+·}.

### Example III-4

### 2-Amino-4-chloro-7-(4-methoxy-3,5-dimethyl-pyridin-2-ylmethyl)-5-[1-(5-methyl-3H-imidazol-4-yl)-meth-(Z)-ylidene]-5,7-dihydro-pyrrolo[2,3-d]pyrimidin-6-one

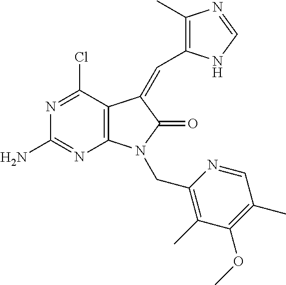

The title compound was prepared in 39% yield as a yellow powder. ¹H NMR (300 MHz, CDCl₃) δ 2.20 (3H, s), 2.35 (3H, s), 2.50 (3H, s), 3.78 (3H, s), 5.09 (4H, s), 7.69 (1H, s), 7.99 (1H, s), 8.07 (1H, s), 13.42 (1H, s). MS m/z: 426 [M+1]^{+·}.

### Example III-5

### 5-[2-Amino-4-chloro-7-(4-methoxy-3,5-dimethyl-pyridin-2-ylmethyl)-6-oxo-6,7-dihydro-pyrrolo[2,3-d]pyrimidin-(5Z)-ylidenemethyl]-1H-pyrrole-2-carboxylic acid methyl ester

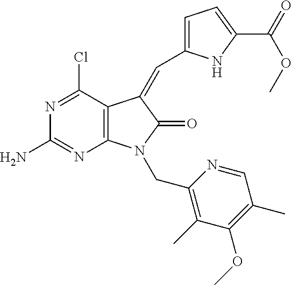

The title compound was prepared in 91% yield as a yellow solid. ¹H NMR (300 MHz, CDCl₃) δ 2.22 (3H, s), 2.37 (3H, s), 3.81 (3H, s), 3.89 (3H, s), 5.13 (2H, s), 5.28 (2H, s), 6.71 (1H, s), 6.97 (1H, s), 7.87 (1H, s), 8.15 (1H, s). MS m/z: 469 [M+1]^{+·}.

### Example III-6

### 5-[2-Amino-4-chloro-7-(4-methoxy-3,5-dimethyl-pyridin-2-ylmethyl)-6-oxo-6,7-dihydro-pyrrolo[2,3-d]pyrimidin-(5Z)-ylidenemethyl]-4-methyl-1H-pyrrole-3-carboxylic acid ethyl ester

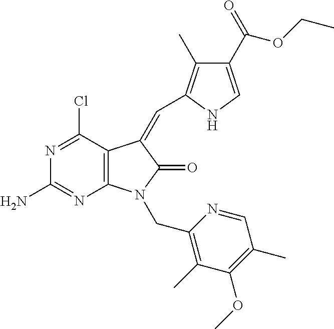

To the ice-cold solution of DMF (600 mg, 8.22 mmol) and POCl₃ (1.0 g, 6.54 mmol) in dichloromethane (DCM, 5 mL) was added a solution of ethyl 4-methyl-1H-pyrrole-3-carboxylate (500 mg, 3.27 mmol) in DCM (10 mL). The reaction solution was stirred at 50° C. for 30 minutes and cooled with ice bath. To this solution was slowly added a solution of NaHCO₃ (sat.) to adjust pH to 7 before extracting two times with 50 mL of EtOAc. The organic layers were combined, washed twice with brine (100 mL), dried over anhydrous MgSO₄, and concentrated. The residue was purified by silica gel column chromatography eluted with ethyl acetate-petroleum ether (1:20) to afford 5-formyl-4-methyl-1H-pyrrole-3-carboxylate as a white solid (0.5 g, 85%). MS m/z: 182 [M+1]^{+·}.

The title compound was prepared in 80% yield. ¹H NMR (300 MHz, CDCl₃,) δ 1.36 (5H, t), 2.24 (3H, s), 2.35 (3H, d), 2.548 (3H, s), 3.82 (3H, s), 4.31 (2H, q), 5.14 (3H, d), 7.59 (1H, d), 8.04 (1H, s), 8.23 (1H, s). MS m/z: 497 [M+1]^{+·}.

### Example III-7

### 2-Amino-4-chloro-5-[1-(4-chloro-6,7-dihydro-1H-indol-2-yl)-meth-(Z)-ylidene]-7-(4-methoxy-3,5-dimethyl-pyridin-2-ylmethyl)-5,7-dihydro-pyrrolo[2,3-d]pyrimidin-6-one

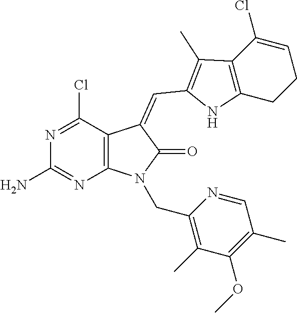

To the ice-cooled solution of DMF (700 mg, 9.59 mmol) and POCl₃ (1.3 g, 8.48 mmol) in DCM (5 mL) was added a solution of 6,7-dihydro-1H-indol-4(5H)-one (1 g, 7.40 mmol) in DCM (10 mL). The reaction mixture was stirred at 0° C. for 15 minutes, refluxed for 50 minutes, cooled with ice bath, and diluted with water (30 mL) and ethyl acetate (30 mL). The pH of the above mixture was adjusted with potassium carbonate (10%) to 7 before extraction with EtOAc (100 mL). The combined organic layers were washed twice with brine (50 mL), dried over anhydrous MgSO₄, and concentrated. The residue was purified by column chromatography eluted with CH₂Cl₂-MeOH (200:1 to 100:1) to afford 4-chloro-6,7-dihydro-1H-indole-2-carbaldehyde (1.1 g) as a red solid.

The title compound was obtained as an orange solid in 30% yield. ¹H NMR (300 MHz, CDCl₃) δ 2.19 (3H, s), 2.3 (3H, s), 2.55 (2H, m), 2.85 (2H, m), 3.77 (3H, s), 5.10 (2H, s), 5.30 (2H, s), 5.70 (1H, m), 6.74 (1H, s), 7.86 (1H, s), 8.11 (1H, s). MS m/z: 497 [M+1]^{+·}.

### Example III-8

### 2-Amino-4-chloro-7-(4-methoxy-3,5-dimethyl-pyridin-2-ylmethyl)-5-[1-[5-(2,2,2-trifluoro-acetyl)-1H-pyrrol-2-yl]-meth-(Z)-ylidene]-5,7-dihydro-pyrrolo[2,3-d]pyrimidin-6-one

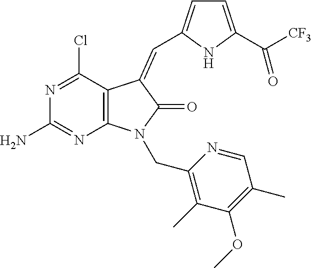

The title compound was obtained as a yellow solid in 79% yield using 5-(2,2,2-trifluoroacetyl)-1H-pyrrole-2-carbaldehyde which was synthesized as a white solid using the same procedure for the preparation of 5-formyl-4-methyl-1H-pyrrole-3-carboxylate. ¹H NMR (300 MHz, CDCl₃) δ 2.14 (3H, s), 2.28 (3H, s), 3.75 (3H, s), 4.99 (2H, s), 7.24 (2H, s), 7.91 (1H, s), 7.99 (1H, s), 8.32 (1H, s), 8.49 (1H, s), 13.21 (1H, s). MS m/z: 507 [M+1]^{+·}.

### Example III-9

### 2-Amino-4-chloro-7-(4-methoxy-3,5-dimethyl-pyridin-2-ylmethyl)-5-[1-(4-oxo-4,5,6,7-tetrahydro-1H-indol-2-yl)-meth-(Z)-ylidene]-5,7-dihydro-pyrrolo[2,3-d]pyrimidin-6-one

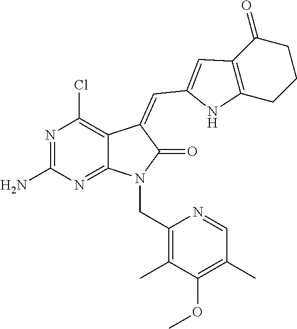

To an ice-cooled round bottom flask containing 4-chloro-6,7-dihydro-1H-indole-2-carbaldehyde (50 mg, 0.28 mmol) was added sulfuric acid (1 ml, 98%). The reaction mixture was stirred at 0° C. for 30 minutes, added ice water, and extracted with ethyl acetate. The combined organic layer was dried over anhydrous sodium sulfate and concentrated. The residue was purified by silica gel column chromatography eluted with ethyl acetate-petroleum ether (1:1˜2:3) to afforded 4-oxo-4,5,6,7-tetrahydro-1H-indole-2-carbaldehyde (40 mg, 89%) as a yellow solid. MS m/z: 164 [M+1]+·.

The title compound was obtained as an orange solid in 57% yield with 4-oxo-4,5,6,7-tetrahydro-1H-indole-2-carbaldehyde prepared above. ¹H NMR (300 MHz, DMSO-d₆) δ 2.14 (3H, s), 2.28 (4H, m), 2.39 (3H, s), 2.90 (2H, m), 3.74 (3H, s), 5.03 (2H, s), 7.26 (3H, m), 7.80 (1H, s), 8.0 (1H, s), 12.75 (1H, s). MS m/z: 479 [M+1]^{+·}.

### Example III-10

### (Z)-2-Amino-5-((4-bromo-1H-pyrazol-5-yl)methylene)-4-chloro-7-((4-methoxy-3,5-dimethylpyridin-2-yl)methyl)-5H-pyrrolo[2,3-d]pyrimidin-6(7H)-one

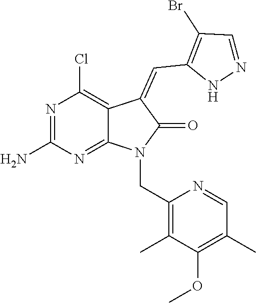

The title compounds were prepared in 89% yield using the same protocols for the synthesis of example-1 with the corresponding aldehydes replacing 1H-pyrrole-2-carbaldehyde. ¹H NMR (400 MHz, DMSO-d₆) δ 2.14 (3H, s), 2.28 (3H, s), 3.73 (3H, s), 5.02 (2H, s), 7.60 (2H, b), 7.63 (1H, s), 7.80 (1H, s), 8.00 (1H, s), 13.83 (1H, s).

### Example III-11

### (Z)-2-Amino-5-((4-bromo-1H-pyrrol-2-yl)methylene)-4-chloro-7-((4-methoxy-3,5-dimethylpyridin-2-yl)methyl)-5H-pyrrolo[2,3-d]pyrimidin-6(7H)-one

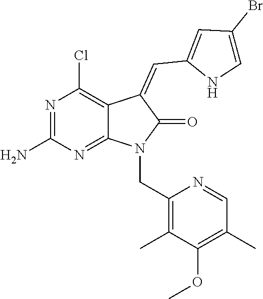

The title compounds were prepared in 94% yield using the same protocols for the synthesis of example-1 with the corresponding aldehydes replacing 1H-pyrrole-2-carbaldehyde. ¹H NMR (400 MHz, DMSO-d₆) δ 2.14 (3H, s), 2.28 (4H, m), 2.74 (3H, s), 5.01 (2H, s), 7.14 (1H, t), 7.22 (1H, s), 7.34 (1H, m), 7.72 (1H, s), 7.99 (1H, s), 12.71 (1H, s).

### Example III-12

### (Z)-2-Amino-5-((2-butyl-1H-imidazol-5-yl)methylene)-4-chloro-7-((4-methoxy-3,5-dimethylpyridin-2-yl)methyl)-5H-pyrrolo[2,3-d]pyrimidin-6(7H)-one

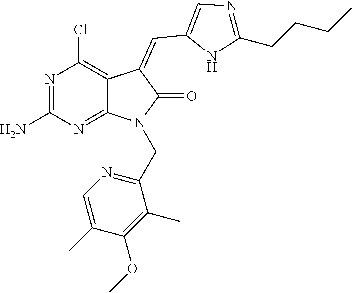

The title compounds were prepared in 86% yield using the same protocols for the synthesis of example-1 with the corresponding aldehydes replacing 1H-pyrrole-2-carbaldehyde. ¹H NMR (300 MHz, DMSO-d₆) δ 0.88 (3H, q), 1.29 (2H, m), 1.64 (2H, m), 2.14 (3H, s), 2.28 (3H, s), 2.74 (1H, t), 3.74 (3H, s), 5.01 (2H, s), 7.32 (2H, s), 7.77 (2H, s), 7.88 (1H, s), 7.99 (1H, b), 8.62 (1H, s), 12.82 (1H, s).

### Example III-13

### (Z)-2-Amino-5-((2-butyl-4-chloro-1H-imidazol-5-yl)methylene)-4-chloro-7-((4-methoxy-3,5-dimethylpyridin-2-yl)methyl)-5H-pyrrolo[2,3-d]pyrimidin-6(7H)-one

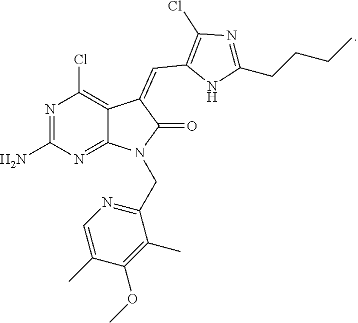

The title compounds were prepared in 56% yield using the same protocols for the synthesis of example-1 with the corresponding aldehydes replacing 1H-pyrrole-2-carbaldehyde. ¹H NMR (300 MHz, DMSO-d₆) δ 0.88 (3H, q), 1.29 (2H, m), 1.64 (2H, m), 2.14 (3H, s), 2.28 (3H, s), 2.75 (1H, t), 3.74 (3H, s), 5.02 (2H, s), 7.64 (1H, s), 8.00 (1H, s), 12.95 (1H, s).

### Example III-14

### (Z)-2-Amino-4-chloro-5-((3-isobutyl-1H-pyrazol-5-yl)methylene)-7-((4-methoxy-3,5-dimethylpyridin-2-yl)methyl)-5H-pyrrolo[2,3-d]pyrimidin-6(7H)-one

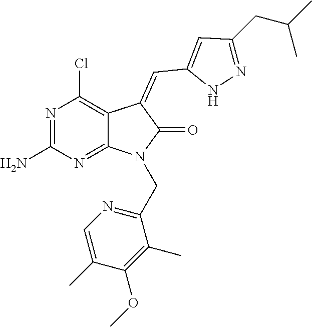

The title compounds were prepared in 92% yield using the same protocols for the synthesis of example-1 with the corresponding aldehydes replacing 1H-pyrrole-2-carbaldehyde. ¹H NMR (400 MHz, DMSO-d₆) δ 0.88 (6H, d), 1.87 (1H, m), 2.14 (3H, s), 2.28 (3H, s), 2.44 (3H, m), 3.74 (3H, s), 5.00 (2H, s), 7.25 (2H, s), 7.45 (1H, s), 7.88 (1H, s), 8.00 (1H, s), 12.13 (1H, s).

### Example III-15

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2,4-dimethyl-1H-pyrrole-3-carboxylic acid

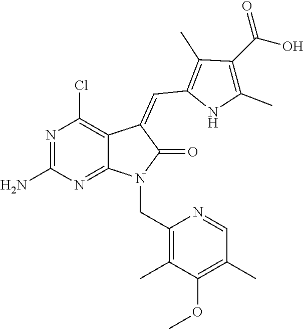

The title compounds were prepared in 100% yield using the same protocols for the synthesis of example-1 with the corresponding aldehydes replacing 1H-pyrrole-2-carbaldehyde. ¹H NMR (400 MHz, DMSO-d₆) δ 2.14 (3H, s), 2.28 (3H, s), 2.39 (3H, s), 2.50 (3H, s), 3.74 (3H, s), 5.02 (2H, s), 7.13 (2H, s), 7.79 (1H, s), 7.98 (1H, s), 12.00 (1H, b), 12.92 (1H, s). MS m/z: 483 [M+1]^{+·}.

### Example III-16

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-morpholinoethyl)-1H-pyrrole-3-carboxamide

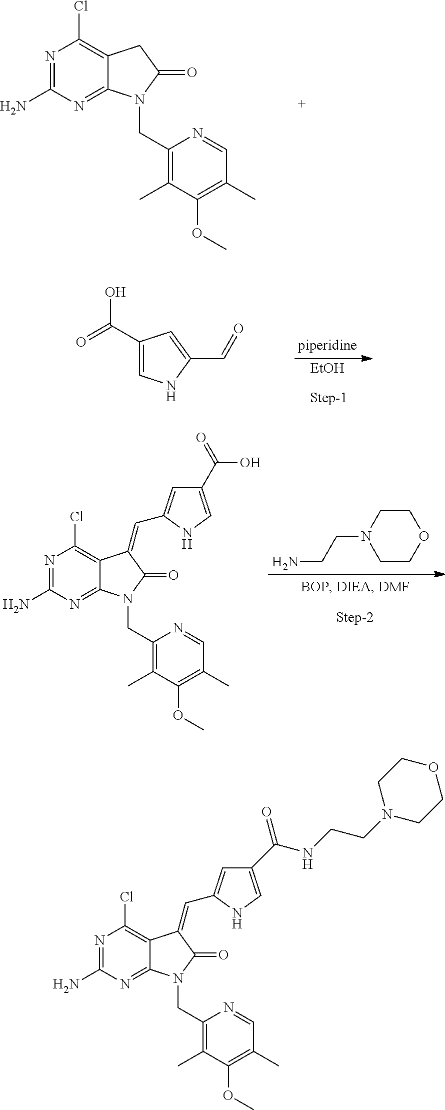

### Step 1: (Z)-5-((2-amino-4-chloro-7-((4-methoxy-3,5-dimethylpyridin-2-yl)methyl)-6-oxo-6,7-dihydropyrrolo[2,3-d]pyrimidin-5-ylidene)methyl)-1H-pyrrole-3-carboxylic acid

To the 2-amino-4-chloro-7-((4-methoxy-3,5-dimethylpyridin-2-yl)methyl)-5H-pyrrolo[2,3-d]pyrimidin-6(7H)-one (240 mg, 0.72 mmol) was added 5-formyl-1H-pyrrole-3-carboxylic acid (100 mg, 0.72 mmol), a drop of piperidine, and EtOH (60 mL). The resulting solution was refluxed for 12 hrs. The reaction mixture was cooled and solids were filtered, resulted in 260 mg (72%) of (Z)-5-((2-amino-4-chloro-7-((4-methoxy-3,5-dimethylpyridin-2-yl)methyl)-6-oxo-6,7-dihydropyrrolo[2,3-d]pyrimidin-5-ylidene)methyl)-1H-pyrrole-3-carboxylic acid as a yellow solid.

### Step 2: (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-morpholinoethyl)-1H-pyrrole-3-carboxamide

To a solution of (Z)-5-((2-amino-4-chloro-7-((4-methoxy-3,5-dimethylpyridin-2-yl)methyl)-6-oxo-6,7-dihydropyrrolo[2,3-d]pyrimidin-5-ylidene)methyl)-1H-pyrrole-3-carboxylic acid (100 mg, 0.22 mmol) in DMF (30 mL) at 0° C. was added 2-morpholinoethanamine (34.4 mg, 0.26 mmol), BOP (116.83 mg, 0.26 mmol), and DIEA (34.1 mg, 0.26 mmol). The resulting solution was stirred for an hour at 0° C. The reaction mixture was treated with 100 mL of H₂O. The solids were filtered and washed 3×30 mL of EtOAc, resulted in 50 mg (13%) of the title compound as a yellow solid. ¹H NMR (300 MHz, DMSO-d₆) δ 2.16 (3H, s), 2.30 (3H, s), 2.42 (4H, m), 3.34 (4H, m), 3.58 (4H, m), 3.76 (3H, s), 5.04 (2H, s), 7.24 (2H, s), 7.34 (1H, s), 7.72 (1H, s), 7.76 (1H, s), 8.02 (2H, m), 12.71 (1H, s) MS m/z: 567 [M+1]^{+·}.

### Example III-17

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4-methylpiperazin-1-yl)ethyl)-1H-pyrrole-3-carboxamide

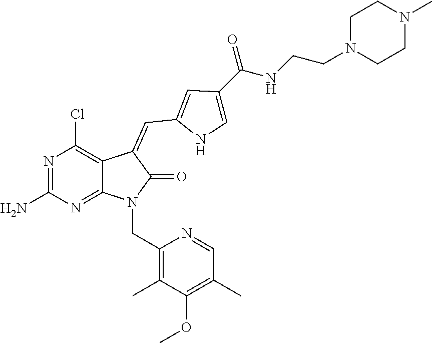

The title compound was prepared in 25% yield using the same protocols for the synthesis of example-16 with the corresponding amine replacing 2-morpholinoethanamine. ¹H NMR (300 MHz, DMSO-d₆) δ 2.15 (3H, s), 2.21 (3H, s), 2.30 (3H, s), 2.43 (10H, m), 3.38 (2H, m), 3.76 (3H, s), 5.04 (2H, s), 7.24 (2H, s), 7.33 (1H, s), 7.72 (1H, s), 7.76 (1H, s), 7.99 (1H, m), 8.01 (1H, s), 12.71 (1H, s). MS m/z: 580 [M+1]^{+·}.

### Example III-18

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-hydroxyethyl)-1H-pyrrole-3-carboxamide

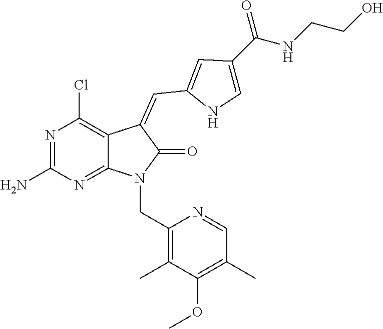

The title compound was prepared in 13% yield using the same protocols for the synthesis of example-16 with the corresponding amine replacing 2-morpholinoethanamine. ¹H NMR (300 MHz, DMSO-d₆) δ 2.13 (3H, s), 2.28 (3H, s), 3.26 (2H, m), 3.46 (2H, m), 3.73 (3H, s), 4.69 (1H, s, br), 5.01 (2H, s), 7.22 (2H, m), 7.32 (1H, s), 7.72 (2H, s), 8.00 (2H, m), 12.69 (1H, s). MS m/z: 498 [M+1]+·.

### Example III-19

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(diethylamino)ethyl)-1H-pyrrole-3-carboxamide

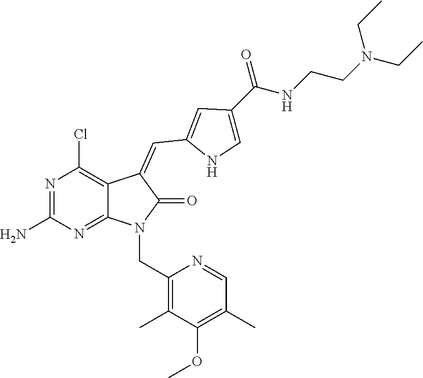

The title compound was prepared in 13% yield using the same protocols for the synthesis of example-16 with the corresponding amine replacing 2-morpholinoethanamine. ¹H NMR (300 MHz, CD₃COCD₃) δ 1.45 (6H, t), 2.20 (3H, s), 2.39 (3H, s), 3.50-3.65 (6H, m), 3.84 (3H, s), 3.88 (2H, m), 5.12 (2H, s), 6.53 (2H, s), 7.27 (1H, s), 7.88 (2H, s), 7.99 (1H, s), 8.43 (1H, s), 13.18 (1H, s). MS m/z: 553 [M+1]^{+·}.

### Example III-20

### (Z)-5-((2-Amino-4-chloro-7-(2-chloro-3,4,5-trimethoxybenzyl)-6-oxo-6,7-dihydro-5H-pyrrolo[2,3-d]pyrimidin-5-ylidene)methyl)-N-(2-hydroxyethyl)-1H-pyrrole-3-carboxamide

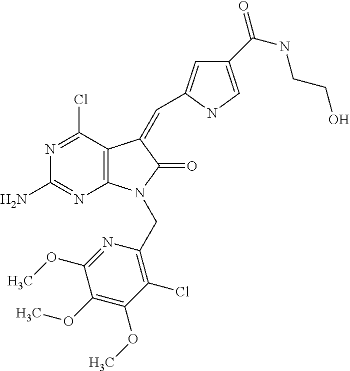

The title compound was prepared in 16% yield using the same protocols for the synthesis of example-16 with the corresponding amine replacing 2-morpholinoethanamine. ¹H NMR (400 MHz, DMSO-d₆) δ 2.14 (3H, s), 2.29 (3H, s), 3.15 (2H, m), 3.58 (1H, m), 3.74 (3H, s), 4.56 (1H, m), 4.80 (1H, m), 5.03 (2H, s), 7.23 (2H, s), 7.35 (1H, s), 7.74 (2H, s), 8.00 (2H, m), 12.71 (1H, s). MS m/z: 528 [M+1]^{+·}.

### Example III-21

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(isopropylamino)ethyl)-1H-pyrrole-3-carboxamide

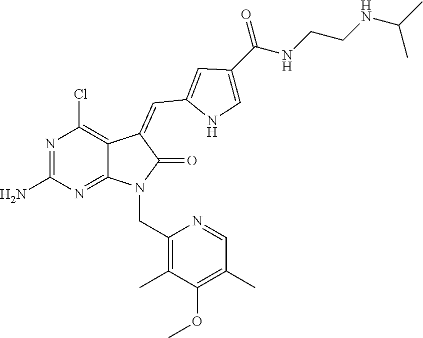

The title compound was prepared in 13% yield using the same protocols for the synthesis of example-16 with the corresponding amine replacing 2-morpholinoethanamine. ¹H NMR (400 MHz, DMSO-d₆) δ 1.21 (6H, d), 2.13 (3H, s), 2.28 (3H, s), 3.04 (2H, m), 3.32 (1H, m), 3.47 (2H, m), 3.73 (3H, s), 5.02 (2H, s), 7.23 (2H, s), 7.34 (1H, s), 7.73 (1H, s), 7.75 (1H, s) 7.99 (1H, s), 8.26 (2H, m), 12.72 (1H, s). MS m/z: 539 [M+1]^{+·}.

### Example III-22

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2,3-dihydroxypropyl)-1H-pyrrole-3-carboxamide

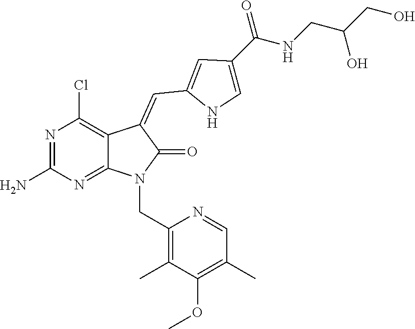

The title compound was prepared in 16% yield using the same protocols for the synthesis of example-16 with the corresponding amine replacing 2-morpholinoethanamine. ¹H NMR (400 MHz, DMSO-d₆) δ 2.14 (3H, s), 2.29 (3H, s), 3.15 (2H, m), 3.58 (1H, m), 3.74 (3H, s), 4.56 (1H, m), 4.80 (1H, m), 5.03 (2H, s), 7.23 (2H, s), 7.35 (1H, s), 7.74 (2H, s), 8.00 (2H, m), 12.71 (1H, s). MS m/z: 528 [M+1]^{+·}.

### Example III-23

### (Z)-2-Amino-4-chloro-5-((4-(4-hydroxypiperidine-1-carbonyl)-1H-pyrrol-2-yl)methylene)-7-((4-methoxy-3,5-dimethylpyridin-2-yl)methyl)-5H-pyrrolo[2,3-d]pyrimidin-6(7H)-one

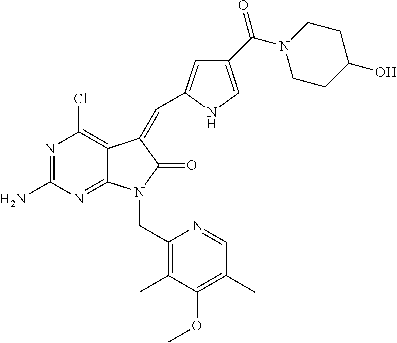

The title compound was prepared in 77% yield using the same protocols for the synthesis of example-16 with the corresponding amine replacing 2-morpholinoethanamine. NMR (300 MHz, DMSO-d₆) δ, 1.36 (2H, m), 1.78 (2H, m) 2.15 (3H, s), 2.30 (3H, s), 3.24 (2H, m), 3.76 (3H, s), 3.98 (2H, d, J=8.0 Hz), 4.77 (1H, d, J=4.2 Hz), 5.04 (2H, s), 7.20 (3H, m), 7.54 (1H, s), 7.82 (1H, s), 8.01 (1H, s), 12.76 (1H, s). MS m/z: 538 [M+1]^{+·}.

### Example III-24

### (Z)-2-Amino-4-chloro-5-((4-(4-(2-hydroxyethyl)piperazine-1-carbonyl)-1H-pyrrol-2-yl)methylene)-7-((4-methoxy-3,5-dimethylpyridin-2-yl)methyl)-5H-pyrrolo[2,3-d]pyrimidin-6(7H)-one

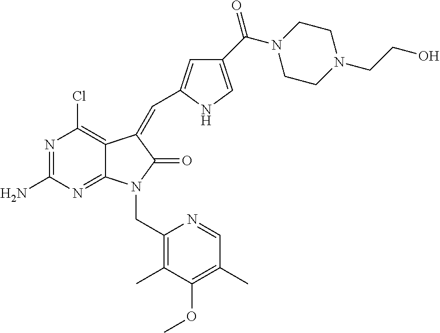

The title compound was prepared in 46% yield using the same protocols for the synthesis of example-16 with the corresponding amine replacing 2-morpholinoethanamine. ¹H NMR (300 MHz, DMSO-d₆) δ 2.14 (3H, s), 2.29 (3H, s), 2.51 (2H, m), 3.21 (6H, m), 3.48 (2H, m), 3.75 (3H, s), 4.38 (2H, m), 5.03 (2H, s), 5.38 (1H, m), 7.25 (3H, m), 7.61 (1H, s), 7.80 (1H, s), 8.00 (1H, s), 12.81 (1H, s). MS m/z: 567 [M+1]^{+·}.

### Example III-25

### (Z)-2-Amino-4-chloro-5-((4-(4-(2-hydroxyethyl)piperidine-1-carbonyl)-1H-pyrrol-2-yl)methylene)-7-((4-methoxy-3,5-dimethylpyridin-2-yl)methyl)-5H-pyrrolo[2,3-d]pyrimidin-6(7H)-one

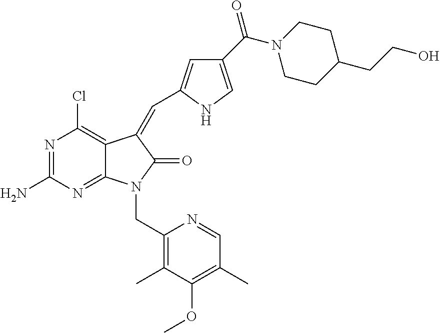

The title compound was prepared in 54% yield using the same protocols for the synthesis of example-16 with the corresponding amine replacing 2-morpholinoethanamine. ¹H NMR (300 MHz, DMSO-d₆) δ, 1.08 (2H, m), 1.38 (2H, m), 1.70 (3H, m), 2.14 (3H, s), 2.29 (3H, s), 2.73-2.95 (2H, m), 3.46 (2H, m), 3.74 (2H, s), 4.27 (2H, m), 4.38 (1H, t), 5.03 (2H, s), 7.16 (1H, s), 7.22 (2H, s), 7.52 (1H, s), 7.80 (1H, s), 8.00 (1H, s), 12.75 (1H, s). MS m/z: 566 [M+1]^{+·}.

### Example III-26

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(pyrrolidin-1-yl)ethyl)-1H-pyrrole-3-carboxamide

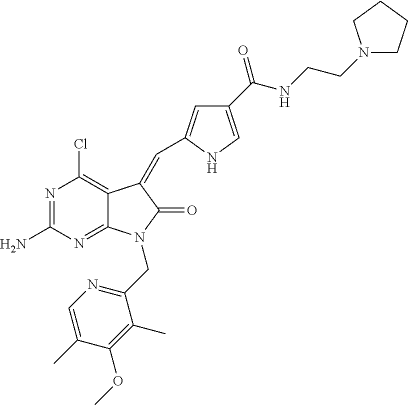

The title compound was prepared in 64% yield using the same protocols for the synthesis of example-16 with the corresponding amine replacing 2-morpholinoethanamine. NMR (300 MHz, DMSO-d₆) δ 1.84 (4H, m), 2.14 (3H, s), 2.29 (3H, s), 3.01 (6H, m), 3.44 (2H, m), 3.74 (3H, s), 5.03 (2H, s), 7.24 (2H, s), 7.34 (1H, s), 7.74 (2H, m), 8.00 (1H, s), 8.20 (1H, t), 12.72 (1H, s). MS m/z: 551 [M+1]^{+·}.

### Example III-27

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N,N-bis(2-hydroxyethyl)-1H-pyrrole-3-carboxamide

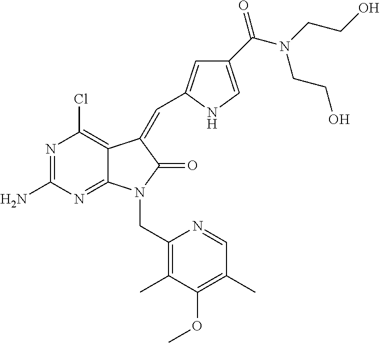

The title compound was prepared in 43% yield using the same protocols for the synthesis of Example-16 with the corresponding amine replacing 2-morpholinoethanamine. ¹H NMR (300 MHz, DMSO-d₆) δ 2.14 (3H, s), 2.29 (3H, s), 3.42-3.65 (8H, m), 3.75 (3H, s), 4.80 (2H, m), 5.03 (2H, s), 7.19 (2H, s), 7.21 (1H, s), 7.59 (1H, s), 7.78 (1H, s), 8.00 (1H, s), 12.75 (1H, s). MS m/z: 542 [M+1]^{+·}.

### Example III-28

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(3-(pyrrolidin-1-yl)propyl)-1H-pyrrole-3-carboxamide

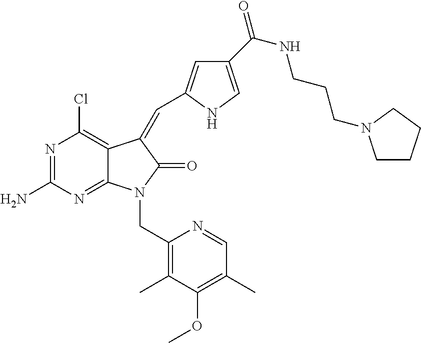

The title compound was prepared in 51% yield using the same protocols for the synthesis of example-16 with the corresponding amine replacing 2-morpholinoethanamine. ¹H NMR (300 MHz, DMSO-d₆) δ 1.71 (6H, m), 2.14 (3H, s), 2.29 (3H, s), 2.48 (6H, m), 3.24 (2H, m), 3.74 (3H, s), 5.03 (2H, s), 7.23 (2H, s), 7.31 (1H, s), 7.70 (1H, s), 7.74 (1H, s), 8.00 (1H, s), 8.10 (1H, t), 12.69 (1H, s). MS m/z: 565 [M+1]^{+·}.

### Example III-29

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(3-(diethylamino)propyl)-1H-pyrrole-3-carboxamide

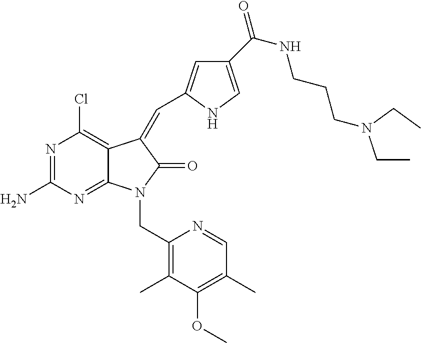

The title compound was prepared in 54% yield using the same protocols for the synthesis of Example-16 with the corresponding amine replacing 2-morpholinoethanamine. NMR (300 MHz, DMSO-d₆) δ 0.98 (6H, t), 1.64 (2H, m), 2.14 (3H, s), 2.29 (3H, s), 2.55 (6H, m), 3.22 (2H, m), 3.74 (3H, s), 5.03 (2H, s), 7.20 (2H, s), 7.31 (1H, s), 7.70 (1H, s), 7.74 (1H, s), 8.00 (1H, s), 8.06 (1H, t), 12.69 (1H, s). MS m/z: 568 [M+1]^{+·}.

### Example III-30

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(bis(2-hydroxyethyl)amino)ethyl)-1H-pyrrole-3-carboxamide

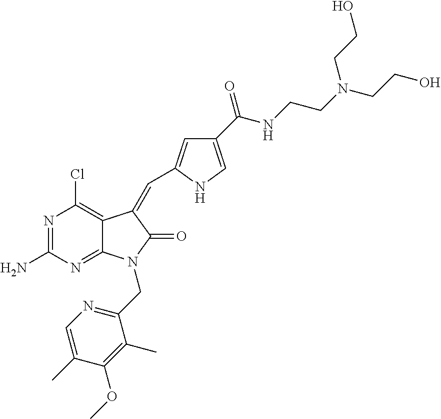

The title compound was prepared using the same protocols for the synthesis of Example-16 with the corresponding amine replacing 2-morpholinoethanamine. ¹H NMR (300 MHz, DMSO-d₆) δ 2.14 (3H, s), 2.29 (3H, s), 2.88 (4H, m), 3.34 (2H, m), 3.56 (4H, m), 3.74 (3H, s), 4.81 (2H, m), 5.03 (2H, s), 7.24 (2H, s), 7.31 (1H, s), 7.71 (1H, s), 7.75 (1H, s), 8.00 (1H, s), 8.11 (1H, t), 12.72 (1H, s). MS m/z: 585 [M+1]^{+·}.

### Example III-31

### (Z)-5-((2-amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(dimethylamino)ethyl)-1H-pyrrole-3-carboxamide

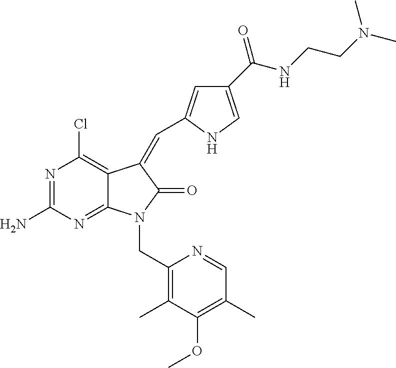

The title compound was prepared in 33% yield using the same protocols for the synthesis of example-16 with the corresponding amine replacing 2-morpholinoethanamine. ¹H NMR (300 MHz, DMSO-d₆) δ 2.14 (3H, s), 2.19 (6H, s), 2.29 (3H, s), 2.40 (2H, m), 3.29 (2H, t), 3.74 (3H, s), 5.03 (2H, s), 7.20 (2H, s), 7.32 (1H, s), 7.70 (1H, s), 7.74 (1H, s), 7.93 (1H, t), 8.00 (1H, s), 12.69 (1H, s). MS m/z: 525 [M+1]^{+·}.

### Example III-32

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(3-(dimethylamino)propyl)-1H-pyrrole-3-carboxamide

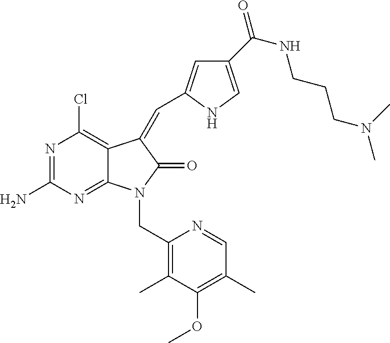

The title compound was prepared in 28% yield using the same protocols for the synthesis of Example-16 with the corresponding amine replacing 2-morpholinoethanamine. ¹H NMR (300 MHz, DMSO-d₆) 5, 1.61 (2H, m), 2.13 (6H, s), 2.14 (3H, s), 2.24 (2H, m), 2.29 (3H, s), 3.20 (2H, m), 3.74 (3H, s), 5.03 (2H, s), 7.20 (2H, s), 7.31 (1H, s), 7.69 (1H, s), 7.74 (1H, s), 8.00 (1H, s), 8.04 (1H, t), 12.69 (1H, s). MS m/z: 539 [M+1]^{+·}.

### Example III-33 (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4-hydroxypiperidin-1-yl)ethyl)-1H-pyrrole-3-carboxamide

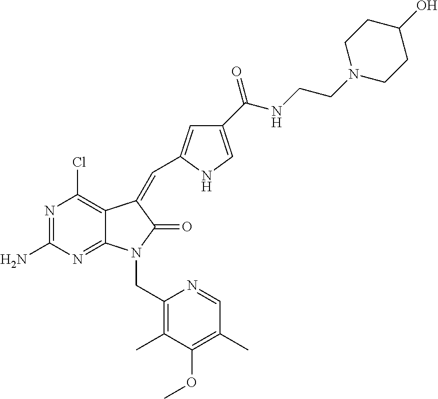

The title compound was prepared in 33% yield using the same protocols for the synthesis of example-16 with the corresponding amine replacing 2-morpholinoethanamine. NMR (300 MHz, DMSO-d₆) δ 1.39 (2H, m), 1.71 (2H, m), 2.08 (2H, m), 2.15 (3H, s), 2.30 (3H, s), 2.43 (2H, m), 2.74 (2H, m), 3.18 (2H, m), 3.44 (1H, m), 3.76 (3H, s), 4.55 (1H, s), 5.04 (2H, s), 7.24 (2H, s), 7.33 (1H, s), 7.72 (1H, s), 7.76 (1H, s), 7.93-8.08 (2H, m), 12.71 (1H, s). MS m/z: 581 [M+1]^{+·}.

### Example III-34

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(3-morpholinopropyl)-1H-pyrrole-3-carboxamide

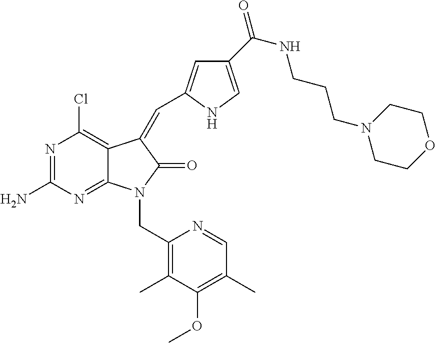

The title compound was prepared in 38% yield using the same protocols for the synthesis of example-16 with the corresponding amine replacing 2-morpholinoethanamine. ¹H NMR (300 MHz, DMSO-d₆) δ, 1.64 (2H, m), 2.14 (3H, s), 2.23-2.37 (9H, m), 3.22 (2H, m), 3.56 (4H, m), 3.74 (3H, s), 5.03 (2H, s), 7.20 (2H, s), 7.31 (1H, s), 7.70 (1H, s), 7.74 (1H, s), 8.01 (2H, m), 12.69 (1H, s). MS m/z: 581 [M+1]^{+·}.

### Example III-35

### (Z)-5-((2-Amino-4-chloro-7-((4-methoxy-3,5-dimethylpyridin-2-yl)methyl)-6-oxo-6,7-dihydro-51/-pyrrolo[2,3-d]pyrimidin-5-ylidene)methyl)-N-((1-(2-hydroxyethyl)piperidin-3-yl)methyl)-1H-pyrrole-3-carboxamide

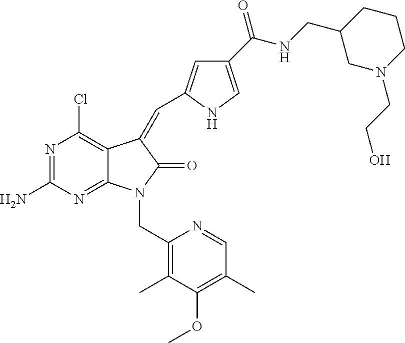

The title compound was prepared in 50% yield using the same protocols for the synthesis of example-16 with the corresponding amine replacing 2-morpholinoethanamine. ¹H NMR (300 MHz, DMSO-d₆) δ 1.12 (2H, m), 1.76 (3H, m), 2.01 (2H, m), 2.16 (3H, s), 2.30 (3H, s), 2.56 (1H, m), 2.75 (1H, m), 3.06 (2H, m), 3.15 (2H, m), 3.70 (2H, m), 3.76 (3H, s), 5.04 (2H, s), 5.15 (1H, s, br), 7.25 (2H, s), 7.36 (1H, s), 7.74 (1H, s), 7.76 (1H, m), 8.02 (1H, s), 8.20 (1H, t), 12.72 (1H, s). MS m/z: 595 [M+1]^{+·}.

### Example III-36

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4-oxopiperidin-1-yl)ethyl)-1H-pyrrole-3-carboxamide

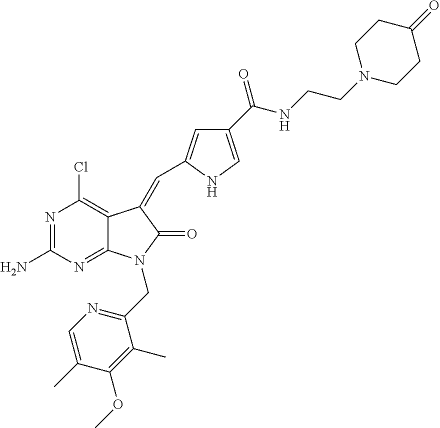

The title compound was prepared in 79% yield using the same protocols for the synthesis of example-16 with the corresponding amine replacing 2-morpholinoethanamine. ¹H NMR (400 MHz, CDCl₃) δ 2.22 (3H, s), 2.38 (3H, s), 2.54 (4H, m), 2.76 (2H, m), 2.88 (4H, m), 3.61 (2H, m), 3.80 (3H, s), 5.11 (2H, s), 5.14 (2H, s), 6.45 (1H, s), 6.96 (1H, s), 7.58 (1H, t), 7.90 (1H, s), 8.09 (1H, s), 13.21 (1H, s). MS m/z: 579 [M+1]^{+·}.

### Example III-37

### (Z)-5-((2-Amino-4-chloro-7-(2-chloro-3,4,5-trimethoxybenzyl)-6-oxo-6,7-dihydro-5H-pyrrolo[2,3-d]pyrimidin-5-ylidene)methyl)-N-(2-(diethylamino)ethyl)-1H-pyrrole-3-carboxamide

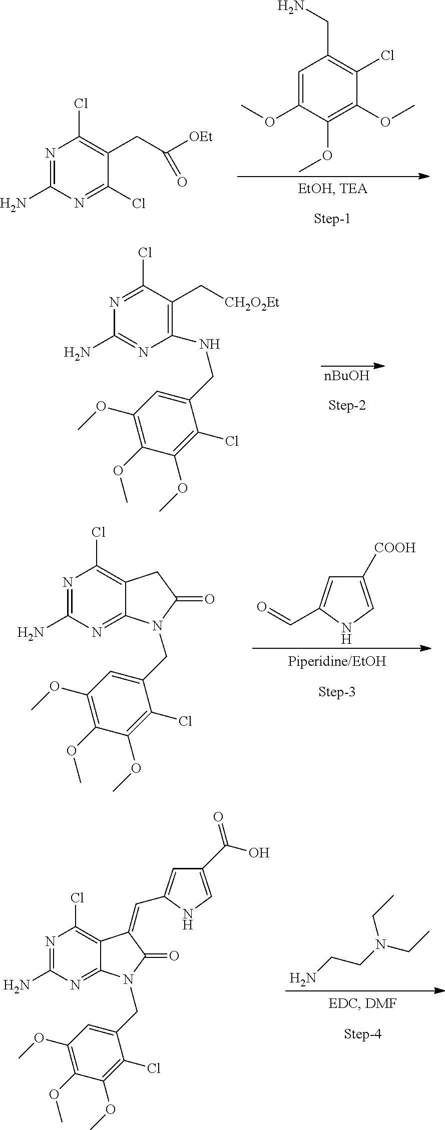

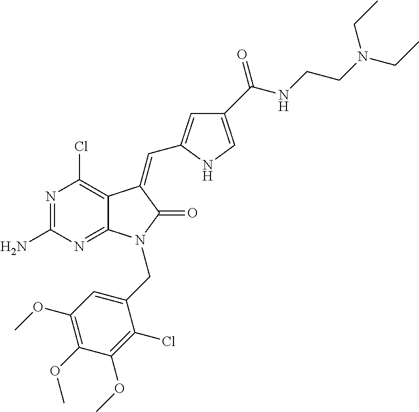

### Step-1: 6-Chloro-N4-(2-chloro-3,4,5-trimethoxybenzyl)-5-(2-(ethylperoxy)ethyl)pyrimidine-2,4-diamine

To a solution of ethyl 2-(2-amino-4,6-dichloropyrimidin-5-yl)acetate (200 mg, 0.76 mmol) in EtOH (30 mL) was added (2-chloro-3,4,5-trimethoxyphenyl)methanamine (200 mg, 0.82 mmol) and triethylamine (300 mg, 2.94 mmol). The resulting solution was refluxed for overnight. The mixture was concentrated and the residue was purified by eluting through a column with EtOAc/PE (1:5) solvent system to give 0.2 g (58%) of ethyl 2-(4-(2-chloro-3,4,5-trimethoxybenzylamino)-2-amino-6-chloropyrimidin-5-yl)acetate as a pale yellow solid.

### Step-2: 2-amino-4-chloro-7-(2-chloro-3,4,5-trimethoxybenzyl)-5H-pyrrolo[2,3-d]pyrimidin-6(7H)-one

A solution of above product (500 mg, 1.11 mmol) in n-BuOH (100 mL) was heated to reflux for overnight and the resulted mixture was concentrated. The residue was purified by eluting through a column with a 1:1 EtOAc/PE solvent system, resulted in 0.4 g (90%) of 7-(2-chloro-3,4,5-trimethoxybenzyl)-2-amino-4-chloro-5H-pyrrolo[2,3-d]pyrimidin-6(7H)-one as a white solid.

### Step-3: (Z)-5-((2-Amino-4-chloro-7-(2-chloro-3,4,5-trimethoxybenzyl)-6-oxo-6,7-dihydro-5H-pyrrolo[2,3-d]pyrimidin-5-ylidene)methyl)-1H-pyrrole-3-carboxylic acid

To a solution of 7-(2-chloro-3,4,5-trimethoxybenzyl)-2-amino-4-chloro-5H-pyrrolo[2,3-d]pyrimidin-6(7H)-one (430 mg, 1.08 mmol) in EtOH (150 mL) was added 5-formyl-1H-pyrrole-3-carboxylic acid (150 mg, 1.07 mmol), and piperidine (0.015 mL). The resulting solution was stirred for overnight at 25° C. The reaction progress was monitored by TLC (EtOAc/PE=1:1) and the reaction mixture was concentrated, resulted in 0.4 g (68%) of 5-((7-(2-chloro-3,4,5-trimethoxybenzyl)-2-amino-4-chloro-6-oxo-6,7-dihydropyrrolo[2,3-d]pyrimidin-5-ylidene)methyl)-1H-pyrrole-3-carboxylic acid as a brown solid.

### Step-4: (Z)-5-((2-Amino-4-chloro-7-(2-chloro-3,4,5-trimethoxybenzyl)-6-oxo-6,7-dihydro-5H-pyrrolo[2,3-d]pyrimidin-5-ylidene)methyl)-N-(2-(diethylamino)ethyl)-1H-pyrrole-3-carboxamide

To a solution of 5-((7-(2-chloro-3,4,5-trimethoxybenzyl)-2-amino-4-chloro-6-oxo-6,7-dihydropyrrolo[2,3-d]pyrimidin-5-ylidene)methyl)-1H-pyrrole-3-carboxylic acid (100 mg, 0.19 mmol) in DMF (50 mL) was added N1,N1-diethylethane-1,2-diamine (33 mg, 0.28 mmol), and EDC (44 mg, 0.23 mmol). The resulting mixture was stirred at 25° C. and was monitored by TLC (MeOH:DCM=1:5). The mixture was concentrated, the solid was filtered and washed with 5 mL of acetone, followed by 10 mL of EtOEt to afford 54 mg (45%) of the title compound as a yellow solid. ¹H NMR (300 MHz, DMSO-d₆) δ 0.95 (6H, t), 3.23 (4H, m), 3.32 (4H, m), 3.63 (3H, s), 3.74 (3H, s), 3.81 (3H, s), 4.96 (2H, s), 6.54 (1H, s), 7.32 (3H, m), 7.72 (1H, s), 7.74 (1H, s), 7.94 (1H, s), 12.62 (1H, s) MS m/z: 618 [M+1]^{+·}.

### Example III-38

### (Z)-5-((2-Amino-4-chloro-7-(2-chloro-3,4,5-trimethoxybenzyl)-6-oxo-6,7-dihydro-5H-pyrrolo[2,3-d]pyrimidin-5-ylidene)methyl)-N-(2-(4-methylpiperazin-1-yl)ethyl)-1H-pyrrole-3-carboxamide

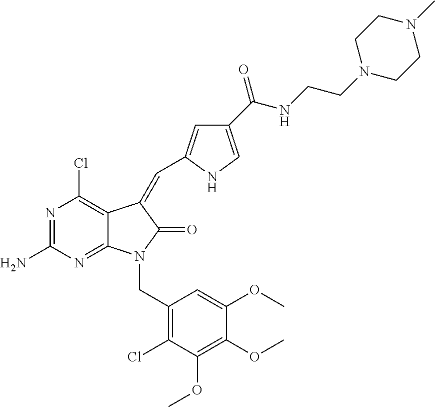

The title compound was prepared in 34% yield using the same protocols for the synthesis of Example-37 with the corresponding amine replacing N1,N1-diethylethane-1,2-diamine. ¹H NMR (400 MHz, DMSO-d₆) δ 2.16 (3H, s), 2.30-2.48 (10H, m), 3.31 (2H, m), 3.65 (3H, s), 3.75 (3H, s), 3.83 (3H, s), 4.97 (2H, s), 6.55 (1H, s), 7.31 (2H, s), 7.34 (1H, s), 7.73 (1H, s), 7.76 (1H, s), 7.96 (1H, s), 12.62 (1H, s). MS m/z: 645 [M+1]^{+·}.

### Example III-39

### (Z)-5-((2-Amino-4-chloro-7-(2-chloro-3,4,5-trimethoxybenzyl)-6-oxo-6,7-dihydro-5H-pyrrolo[2,3-d]pyrimidin-5-ylidene)methyl)-N-(2-morpholinoethyl)-1H-pyrrole-3-carboxamide

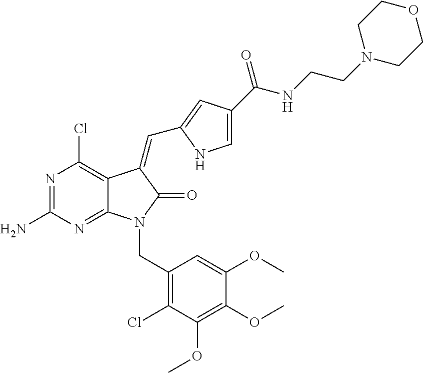

The title compound was prepared in 34% yield using the same protocols for the synthesis of example-37 with the corresponding amine replacing N1,N1-diethylethane-1,2-diamine. NMR (400 MHz, DMSO-d₆) δ 2.42 (6H, m), 3.34 (2H, m), 3.56 (4H, m), 3.65 (3H, s), 3.75 (3H, s), 3.82 (3H, s), 4.97 (2H, s), 6.55 (1H, s), 7.31 (2H, s), 7.34 (1H, s), 7.73 (1H, s), 7.76 (1H, s), 7.98 (1H, s), 12.63 (1H, s). MS m/z: 632 [M+1]^{+·}.

### Example III-40

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2-methyl-N-(2-morpholinoethyl)-4-(trifluoromethyl)-1H-pyrrole-3-carboxamide

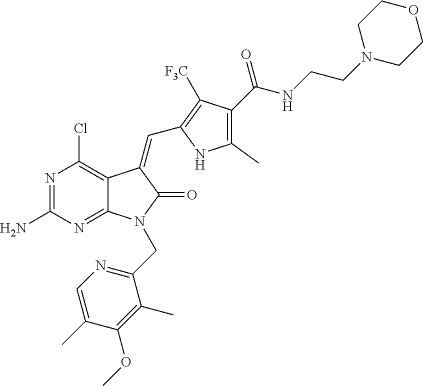

The title compound was prepared using the same protocols for the synthesis of example-123 with the corresponding aldehyde. ¹H NMR (400 MHz, DMSO-d₆) δ 2.14 (3H, s), 2.23 (3H, s), 2.32 (3H, s), 2.35 (6H, m) 3.29 (2H, m), 3.55 (4H, m), 3.74 (3H, s), 5.05 (2H, s), 7.40 (2H, s), 7.96 (1H, s), 7.99 (1H, s), 8.10 (1H, t), 13.19 (1H, s). MS m/z: 649 [M+1]^{+·}.

### Example III-41

### (Z)-5-((2-Amino-4-chloro-7-(2-chloro-3,4,5-trimethoxybenzyl)-6-oxo-6,7-dihydro-5H-pyrrolo[2,3-d]pyrimidin-5-ylidene)methyl)-N-(2-(isopropylamino)ethyl)-1H-pyrrole-3-carboxamide

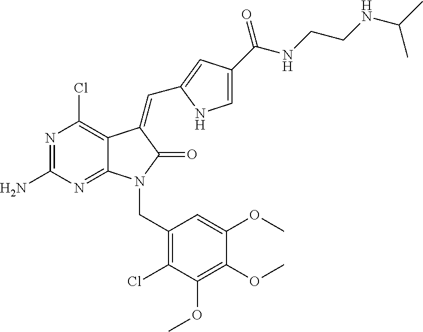

The title compound was prepared in 33% yield using the same protocols for the synthesis of example-37 with the corresponding amine replacing N1,N1-diethylethane-1,2-diamine. ¹H NMR (400 MHz, CD₃OD) δ 1.33 (6H, d), 3.16 (2H, t), 3.32 (1H, m), 3.65 (2H, t), 3.73 (3H, s), 3.87 (3H, s), 3.93 (3H, s), 5.15 (2H, s), 6.59 (1H, s), 7.23 (1H, s), 7.77 (1H, s), 7.90 (1H, s), 13.12 (1H, s) MS m/z: 604 [M+1]^{+·}.

### Example III-42

### (S,Z)-5-((2-Amino-4-chloro-7-(2-chloro-3,4,5-trimethoxybenzyl)-6-oxo-6,7-dihydro-5H-pyrrolo[2,3-d]pyrimidin-5-ylidene)methyl)-N-(2,3-dihydroxypropyl)-1H-pyrrole-3-carboxamide

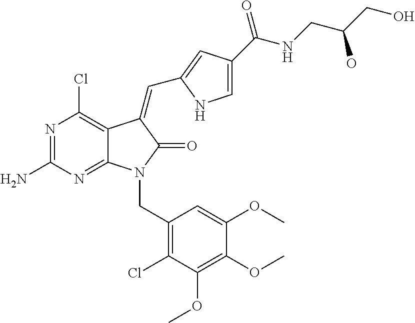

The title compound was prepared in 62% yield using the same protocols for the synthesis of example-37 with the corresponding amine replacing N1,N1-diethylethane-1,2-diamine. ¹H NMR (400 MHz, DMSO-d₆) δ 3.13 (1H, m), 3.33 (1H, m), 3.57 (1H, m), 3.69 (3H, s), 3.74 (3H, s), 3.82 (3H, s), 4.55 (1H, m), 4.80 (1H, m), 4.96 (2H, s), 6.54 (1H, s), 7.32 (2H, s), 7.37 (1H, s), 7.75 (1H, s), 7.77 (1H, s), 8.01 (1H, t), 12.63 (1H, s). MS m/z: 593 [M+1]^{+·}.

### Example III-43

### (Z)-5-((2-Amino-4-chloro-7-(2-chloro-3,4,5-trimethoxybenzyl)-6-oxo-6,7-dihydro-5H-pyrrolo[2,3-d]pyrimidin-5-ylidene)methyl)-N-(2,3-dihydroxypropyl)-1H-pyrrole-3-carboxamide

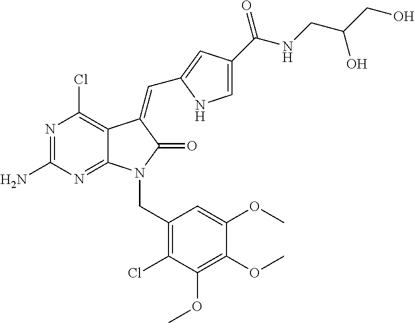

The title compound was prepared in 45% yield using the same protocols for the synthesis of example-37 with the corresponding amine replacing N1,N1-diethylethane-1,2-diamine. ¹H NMR (400 MHz, DMSO-d₆) δ 3.15 (1H, m), 3.35 (1H, m), 3.58 (1H, m), 3.65 (3H, s), 3.76 (3H, s), 3.83 (3H, s), 4.54 (1H, m), 4.78 (1H, m), 4.97 (2H, s), 6.55 (1H, s), 7.31 (2H, s), 7.38 (1H, s), 7.77 (2H, m), 8.00 (1H, t), 12.64 (1H, s). MS m/z: 593 [M+1]+·.

### Example III-44

### (Z)-5-((1H-Imidazol-2-yl)methylene)-2-amino-4-chloro-7-((4-methoxy-3,5-dimethylpyridin-2-yl)methyl)-5H-pyrrolo[2,3-d]pyrimidin-6(7H)-one

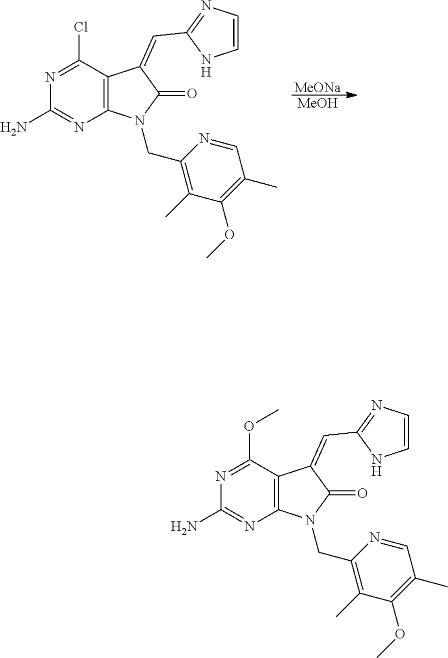

To a solution of (Z)-5-((1H-imidazol-2-yl)methylene)-2-amino-4-chloro-7-((5-methoxy-4,6-dimethylpyridin-2-yl)methyl)-5H-pyrrolo[2,3-d]pyrimidin-6(7H)-one (150 mg, 0.36 mmol) in MeOH (50 mL) was added sodium methanolate (98.5 mg, 1.81 mmol. The resulting solution was heated to reflux for 30 minutes. The mixture was concentrated and the residue was purified by eluting through a column with a 1:1 EtOAc/PE solvent system, resulted in 99 mg (66%) of the title compound as a yellow solid.

### Example III-45

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4-methylpiperazin-1-yl)ethyl)-1H-pyrrole-3-carboxamide

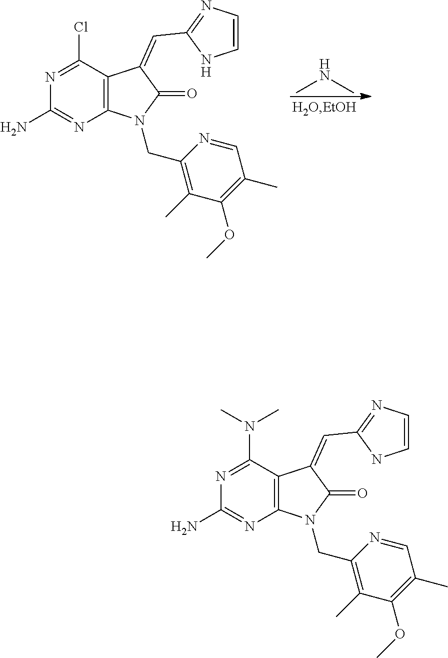

A solution of (Z)-5-((1H-imidazol-2-yl)methylene)-2-amino-4-chloro-7-((5-methoxy-4,6-dimethylpyridin-2-yl)methyl)-5H-pyrrolo[2,3-d]pyrimidin-6(7H)-one (100 mg, 0.24 mmol) in EtOH (50 mL) and dimethylamine 30% in water (600 mg) was heated in a sealed tube at 80° C. for 15 hrs. The mixture was concentrated and the residue was purified with a silica gel column (ethyl acetate/PE 1:1), resulted in 35 mg (33%) of the title compound as a yellow solid. ¹H NMR (300 MHz, DMSO-d₆) δ 2.14 (3H, s), 2.28 (3H, s), 2.96 (6H, s), 3.74 (3H, s), 4.99 (2H, s), 6.71 (2H, s), 6.92 (1H, s), 7.21 (1H, s), 7.36 (1H, s), 8.01 (1H, s), 13.29 (1H, s). MS m/z: 421 [M+1]^{+·}.

### Example III-46

### (Z)-5-(1H-Pyrrol-2-yl)methylene)-2-amino-4-chloro-7-((5-methylpyrazin-2-yl)methyl)-5H-pyrrolo[2,3-d]pyrimidin-6(7H)-one

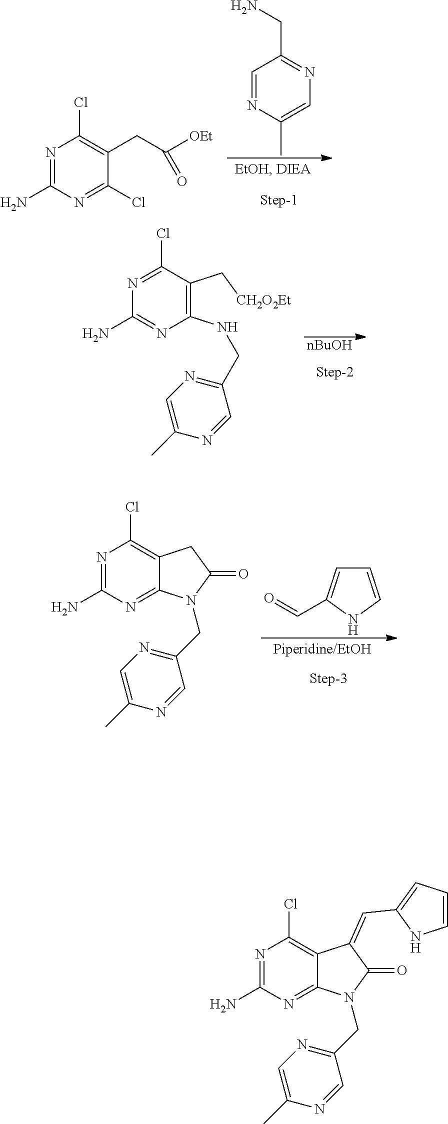

### Step-1: Ethyl 2-(2-amino-4-chloro-6-((5-methylpyrazin-2-yl)methylamino) pyrimidin-5-yl)acetate

To ethyl 2-(2-amino-4,6-dichloropyrimidin-5-yl)acetate (600 mg, 2.41 mmol) was added (5-methylpyrazin-2-yl)methanamine (330 mg, 2.68 mmol), DIEA (928 mg, 7.19 mmol, 3.00 equiv), and t-BuOH (20 mL). The resulting solution was stirred at 78° C. for 12 hr. The reaction mixture was cooled with a water bath and the solids were collected by filtration. This resulted in 0.65 g (80%) of ethyl 2-(2-amino-4-chloro-6-((5-methylpyrazin-2-yl)methylamino) pyrimidin-5-yl)acetate as a yellow solid

### Step-2: 2-Amino-4-chloro-7-((5-methylpyrazin-2-yl)methyl)-5H-pyrrolo[2,3-d]pyrimidin-6(7H)-one

The mixture of ethyl 2-(2-amino-4-chloro-6-((5-methylpyrazin-2-yl)methylamino) pyrimidin-5-yl)acetate (650 mg, 1.93 mmol) in n-BuOH (10 mL) was stirred at 110° C. for 12 hrs under N₂. The reaction mixture was cooled with a water bath and the solids were collected by filtration. This resulted in 0.6 g (crude) of 2-amino-4-chloro-7-((5-methylpyrazin-2-yl)methyl)-5H-pyrrolo[2,3-d]pyrimidin-6(7H)-one as a yellow solid.

### Step 3: (Z)-5-((1H-Pyrrol-2-yl)methylene)-2-amino-4-chloro-7-((5-methylpyrazin-2-yl)methyl)-5H-pyrrolo[2,3-d]pyrimidin-6(7H)-one

To 2-amino-4-chloro-7-((5-methylpyrazin-2-yl)methyl)-5H-pyrrolo[2,3-d]pyrimidin-6(7H)-one (100 mg, 0.34 mmol) under nitrogen was added 1H-pyrrole-2-carbaldehyde (36.65 mg, 0.39 mmol), piperidine (a drop), and EtOH (30 mL). The resulting mixture was stirred at 70° C. for 12 hrs. The reaction mixture was cooled with a water bath and then treated by the addition of Et₂O. The solids were collected by filtration, resulted in 16 mg (13%) of the title compound as a yellow solid.

### Example III-47

### (Z)-5-(1H-Imidazol-2-yl)methylene)-2-amino-4-chloro-7-((5-methylpyrazin-2-yl)methyl)-5H-pyrrolo[2,3-d]pyrimidin-6(7H)-one

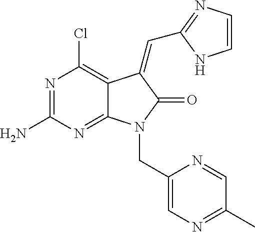

The title compound was prepared in 53% yield using the same protocols for the synthesis of example-46 with 1H-imidazole-2-carbaldehyde replacing 1H-pyrrole-2-carbaldehyde. NMR (300 MHz, DMSO-d₆) δ 2.47 (3H, s), 5.14 (2H, s), 7.33 (1H, s), 7.50 (2H, s), 7.79 (1H, s), 8.44 (1H, s), 8.65 (1H, s), 13.24 (1H, s). MS m/z: 369 [M+1]^{+·}.

### Example III-48

### (Z)-5-(1H-Imidazol-2-yl)methylene)-2-amino-4-chloro-7-((2,2,6,6-tetramethylpiperidin-4-yl)methyl)-5H-pyrrolo[2,3-d]pyrimidin-6(7H)-one

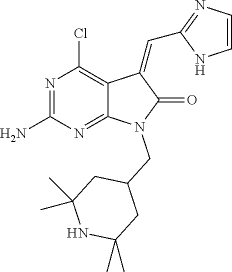

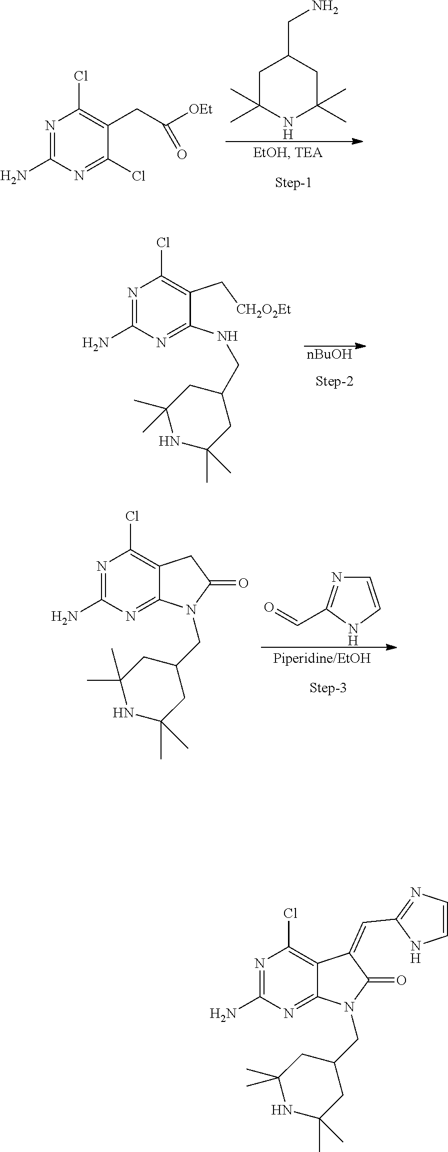

### Step-1: Ethyl 2-(2-amino-4-chloro-6-((2,2,6,6-tetramethylpiperidin-4-yl)methylamino)pyrimidin-5-yl)acetate

To a solution of ethyl 2-(2-amino-4,6-dichloropyrimidin-5-yl)acetate (1.3 g, 4.96 mmol) in EtOH (40 mL) was added (2,2,6,6-tetramethylpiperidin-4-yl)methanamine (1.3 g, 7.26 mmol) and TEA (1.6 g, 15.68 mmol). The resulting solution was heated to reflux with stirring for overnight. The resulting mixture was concentrated under vacuum and the residue was purified with a silica gel column (ethyl acetate/petroleum ether 1:6), resulted in 1.7 g (89%) of ethyl 2-(2-amino-4-chloro-6-((2,2,6,6-tetramethylpiperidin-4-yl)methylamino)pyrimidin-5-yl)acetate as brown oil.

### Step-2: 2-Amino-4-chloro-7-((2,2,6,6-tetramethylpiperidin-4-yl)methyl)-5H-pyrrolo[2,3-d]pyrimidin-6(7H)-one

A solution of ethyl 2-(2-amino-4-chloro-6-((2,2,6,6-tetramethylpiperidin-4-yl)methylamino)pyrimidin-5-yl)acetate (1.7 g, 4.21 mmol) in n-BuOH (100 mL) was refluxed for overnight. The resulting mixture was concentrated under vacuum to give 1.6 g (crude) of 2-amino-4-chloro-7-((2,2,6,6-tetramethylpiperidin-4-yl)methyl)-5H-pyrrolo[2,3-d]pyrimidin-6(7H)-one as brown oil.

### Step-3: (Z)-5-(1H-imidazol-2-yl)methylene)-2-amino-4-chloro-7-((2,2,6,6-tetramethylpiperidin-4-yl)methyl)-5H-pyrrolo[2,3-d]pyrimidin-6(7H)-one

The above product (880 mg, 2.58 mmol) in EtOH (80 mL) was added 1H-imidazole-2-carbaldehyde (260 mg, 2.74 mmol) and piperidine (0.03 mL). The mixture was stirred for overnight at 25° C. The resulting solids were collected by filtration and washed with 2×20 mL of EtOH, gave 315 mg (29%) of the title compound as a yellow solid. ¹H NMR (300 MHz, DMSO-d₆) δ 0.84 (2H, m), 1.06 (12H, m), 1.46 (2H, m), 2.42 (2H, m), 3.63 (2H, d), 7.32 (1H, s), 7.51 (3H, m), 7.75 (1H, s), 13.34 (1H, s). MS m/z: 416 [M+1]^{+·}.

### Example III-49

### (Z)-5-(1H-Pyrrol-2-yl)methylene)-2-amino-4-chloro-7-((2,2,6,6-tetramethylpiperidin-4-yl)methyl)-5H-pyrrolo[2,3-d]pyrimidin-6(7H)-one

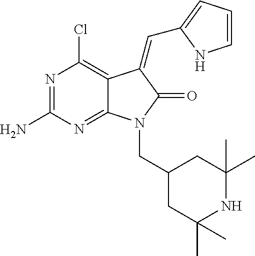

The title compound was prepared in 39% yield using the same protocols for the synthesis of example-48 with 1H-pyrrole-2-carbaldehyde replacing 1H-imidazole-2-carbaldehyde. ¹H NMR (300 MHz, DMSO-d₆) δ 0.82 (2H, m), 0.99 (6H, s), 1.04 (6H, s), 1.42 (2H, d), 2.40 (2H, m), 3.62 (2H, d), 6.35 (1H, t), 6.96 (1H, s), 7.20 (2H, s), 7.35 (1H, s), 7.77 (1H, s), 12.71 (1H, s). MS m/z: 415 [M+1]^{+·}.

### Example III-50

### (Z)-5-(1H-Pyrrol-2-yl)methylene)-2-amino-7-(benzo[d]thiazol-6-ylmethyl)-4-chloro-5H-pyrrolo[2,3-d]pyrimidin-6(7H)-one

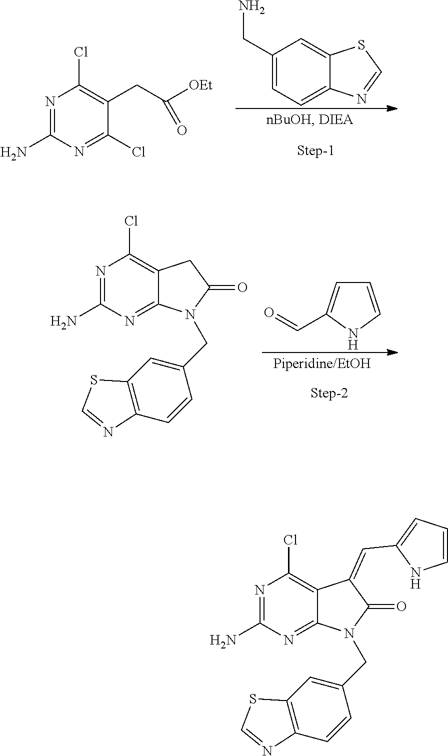

### Step-1: 2-Amino-7-(benzo[d]thiazol-5-ylmethyl)-4-chloro-5H-pyrrolo[2,3-d]pyrimidin-6(7H)-one

To ethyl 2-(2-amino-4,6-dichloropyrimidin-5-yl)acetate (350 mg, 1.41 mmol) under nitrogen was added benzo[d]thiazol-6-ylmethanamine (255 mg), DIEA (545.67 mg) was next, and n-BuOH (25 mL). The resulting solution was stirred for 12 hr at 120° C. The reaction mixture was cooled with a water/ice bath and the solids were collected by filtration, resulted in 100 mg (21%) of 2-amino-7-(benzo[d]thiazol-5-ylmethyl)-4-chloro-5H-pyrrolo[2,3-d]pyrimidin-6(7H)-one as a red solid.

### Step-2: (Z)-5-((1H-pyrrol-2-yl)methylene)-2-amino-7-(benzo[d]thiazol-6-ylmethyl)-4-chloro-5H-pyrrolo[2,3-d]pyrimidin-6(7H)-one

A mixture of 2-amino-7-(benzo[d]thiazol-5-ylmethyl)-4-chloro-5H-pyrrolo[2,3-d]pyrimidin-6(7H)-one (180 mg, 0.54 mmol), 1H-pyrrole-2-carbaldehyde (54.1 mg, 0.57 mmol), piperidine (a drop), and EtOH (20 mL) was stirred at 70° C. for 12 hr. The reaction mixture was cooled and the solids were collected by filtration, washed with 30 mL of EA: Et₂O, resulted in 163 mg (74%) of the title compound as a yellow solid. ¹H NMR (300 MHz, DMSO-d₆) δ 5.15 (2H, s), 6.37 (1H, s), 7.01 (1H, s), 7.26 (2H, s), 7.35 (1H, s), 7.54 (1H, d), 7.82 (1H, s), 8.08 (2H, m), 9.37 (1H, s), 12.66 (1H, s). MS m/z: 409 [M+1]^{+·}.

### Example III-51

### (Z)-5-(1H-Imidazol-2-yl)methylene)-2-amino-7-(benzo[d]thiazol-6-ylmethyl)-4-chloro-5H-pyrrolo[2,3-d]pyrimidin-6(7H)-one

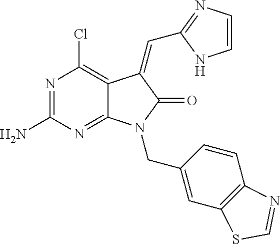

The title compound was prepared in 56% yield using the same protocols for the synthesis of Example-46 with 1H-imidazole-2-carbaldehyde replacing 1H-pyrrole-2-carbaldehyde. ¹H NMR (300 MHz, DMSO-d₆) δ 5.14 (2H, s), 7.32 (1H, s), 7.53 (4H, m), 7.78 (1H, s), 8.06 (1H, d), 8.13 (1H, s), 9.36 (1H, s), 13.29 (1H, s). MS m/z: 410 [M+1]^{+·}.

### Example III-52

### (Z)-2-Amino-5-((3-tert-butyl-1H-pyrazol-5-yl)methylene)-4-chloro-7-((4-methoxy-3,5-dimethylpyridin-2-yl)methyl)-5H-pyrrolo[2,3-d]pyrimidin-6(7H)-one

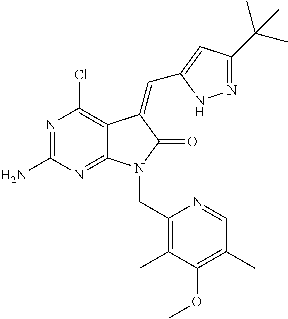

The title compound was prepared in 81% yield using the same protocols for the synthesis of example-1 with the corresponding aldehyde replacing 1H-pyrrole-2-carbaldehyde. ¹H NMR (400 MHz, DMSO-d₆) δ 2.17 (9H, s), 2.74 (3H, s), 2.14 (3H, s), 3.74 (3H, s), 4.97 (2H, s), 7.3 (3H, m), 7.88 (2H, m), 12.71 (1H, s). MS m/z: [M+1]^{+·}.

### Example III-53

### (Z)-5-(1H-Pyrrol-2-yl)methylene)-2-amino-4-chloro-7-((4-chloro-1-ethyl-1H-pyrazol-3-yl)methyl)-5H-pyrrolo[2,3-d]pyrimidin-6(7H)-one

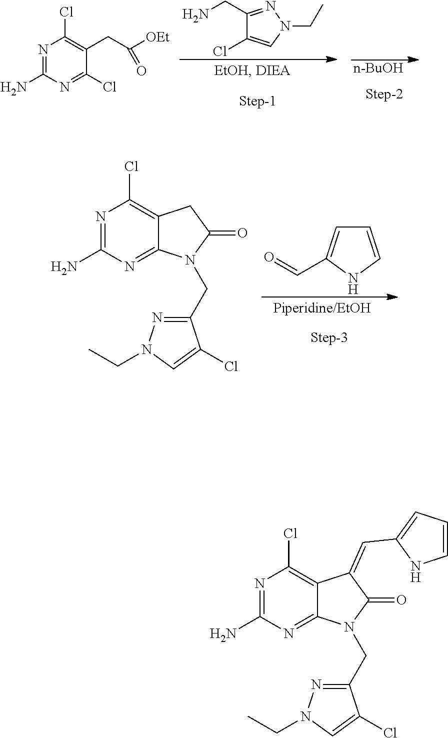

### Step-1: Ethyl 2-(2-amino-4-chloro-6-((4-chloro-1-ethyl-1H-pyrazol-3-yl)methylamino)pyrimidin-5-yl)acetate

To a solution of ethyl 2-(2-amino-4,6-dichloropyrimidin-5-yl)acetate (447 mg, 1.71 mmol) in EtOH (25 mL) was added (4-chloro-1-ethyl-1H-pyrazol-3-yl)methanamine (300 mg, 1.79 mmol) and TEA (544 mg, 5.39 mmol). The resulting solution was heated to reflux with stirring for overnight. The resulting mixture was concentrated under vacuum and the residue was purified on a silica gel column with ethyl acetate/petroleum ether (1:4), resulted in 0.4 g (60%) of ethyl 2-(2-amino-4-chloro-6-((4-chloro-1-ethyl-1H-pyrazol-3-yl)methylamino)pyrimidin-5-yl)acetate as a white solid.

### Step-2: 2-Amino-4-chloro-7-((4-chloro-1-ethyl-1H-pyrazol-3-yl)methyl)-5H-pyrrolo[2,3-d]pyrimidin-6(7H)-one

A solution of ethyl 2-(2-amino-4-chloro-6-((4-chloro-1-ethyl-1H-pyrazol-3-yl)methylamino)pyrimidin-5-yl)acetate (400 mg, 1.05 mmol) in n-BuOH (25 mL) was refluxed for overnight. The resulting mixture was concentrated under vacuum. The residue was purified on a silica gel column with ethyl acetate/petroleum ether (1:1), resulted in 0.2 g (58%) of 2-amino-4-chloro-7-((4-chloro-1-ethyl-1H-pyrazol-3-yl)methyl)-5H-pyrrolo[2,3-d]pyrimidin-6(7H)-one as a yellow solid.

### Step-3: (Z)-5-((1H-pyrrol-2-yl)methylene)-2-amino-4-chloro-7-((4-chloro-1-ethyl-1H-pyrazol-3-yl)methyl)-5H-pyrrolo[2,3-d]pyrimidin-6(7H)-one

To 2-amino-4-chloro-7-((4-chloro-1-ethyl-1H-pyrazol-3-yl)methyl)-5H-pyrrolo[2,3-d]pyrimidin-6(7H)-one (200 mg, 0.58 mmol) in EtOH (20 mL) was added 1H-pyrrole-2-carbaldehyde (58 mg, 0.60 mmol) and piperidine (0.03 mL). The resulting solution was stirred at 40° C. for overnight. The solids were collected by filtration and washed with 10 mL of EtOH, resulted in 83 mg (34%) of the title compound as a yellow solid. ¹H NMR (400 MHz, DMSO-d₆) δ 1.26 (3H, t), 3.99 (2H, m), 4.92 (2H, s), 6.35 (1H, m), 6.97 (1H, m), 7.27 (2H, s), 7.49 (1H, s), 7.78 (1H, s), 7.94 (1H, s). MS m/z: 404 [M+1]^{+·}.

### Example III-54

### (Z)-5-(1H-Imidazol-2-yl)methylene)-2-amino-4-chloro-7-((4-chloro-1-methyl-1H-pyrazol-3-yl)methyl)-5H-pyrrolo[2,3-d]pyrimidin-6(7H)-one

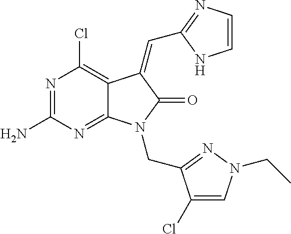

The title compound was prepared in 100% yield using the same protocols for the synthesis of example-46 with 1H-imidazole-2-carbaldehyde replacing 1H-pyrrole-2-carbaldehyde. ¹H NMR (400 MHz, DMSO-d₆) δ 1.27 (3H, t), 4.00 (2H, m), 4.94 (2H, s), 7.33 (1H, s), 7.52 (3H, m), 7.77 (1H, s), 7.96 (1H, s), 13.24 (1H, s). MS m/z: 405 [M+1]^{+·}.

### Example III-55

### (Z)-5-((1H-Imidazol-2-yl)methylene)-2-amino-4-chloro-7-(morpholinomethyl)-5H-pyrrolo[2,3-d]pyrimidin-6(7H)-one

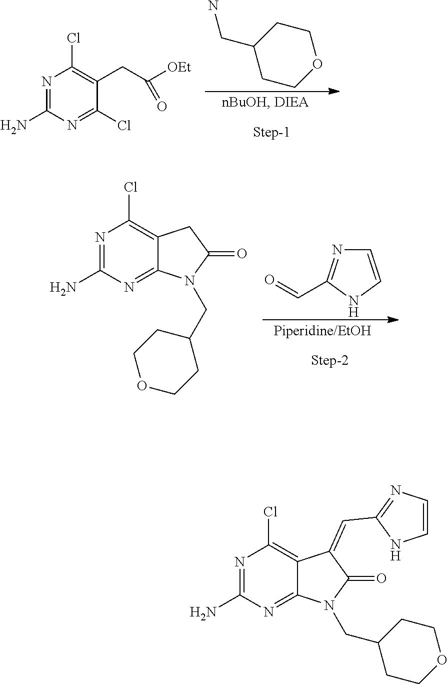

### Step-1: 2-Amino-4-chloro-7-((tetrahydro-2H-pyran-4-yl)methyl)-5H-pyrrolo[2,3-d]pyrimidin-6(7H)-one

To a solution of ethyl 2-(2-amino-4-chloro-6-((tetrahydro-2H-pyran-4-yl)methylamino)pyrimidin-5-yl)acetate (1 g, 3.05 mmol) in n-BuOH (100 mL) was added t-BuOK (30 mg, 0.27 mmol,). The solution was heated to reflux with stirring for 5 min. The resulting mixture was concentrated under vacuum. The residue was purified on a silica gel column with ethyl acetate/petroleum ether (1:10), resulted in 640 mg (74%) of 2-amino-4-chloro-7-((tetrahydro-2H-pyran-4-yl)methyl)-5H-pyrrolo[2,3-d]pyrimidin-6(7H)-one as a yellow solid.

### Step-2: (Z)-5-((1H-Imidazol-2-yl)methylene)-2-amino-4-chloro-7-(morpholinomethyl)-5H-pyrrolo[2,3-d]pyrimidin-6(7H)-one

To a solution of 2-amino-4-chloro-7-((tetrahydro-2H-pyran-4-yl)methyl)-5H-pyrrolo[2,3-d]pyrimidin-6(7H)-one (350 mg, 1.24 mmol) in EtOH (60 mL) was added 1H-imidazole-2-carbaldehyde (119 mg, 1.24 mmol) and piperidine (105 mg, 1.24 mmol). The solution was stirred at 40° C. for overnight. The resulting mixture was concentrated under vacuum. The solids were collected by filtration and washed with 2×10 mL of EtOH and 2×100 mL of ethoxyethane, resulted in 120 mg (27%) of the title compound as a yellow solid. ¹H NMR (400 MHz, DMSO-d₆) δ 1.28 (2H, m), 1.53 (2H, m), 2.08 (1H, m), 3.23 (2H, m), 3.67 (2H, d), 3.83 (2H, d), 7.32 (1H, s), 7.50 (3H, m), 7.75 (1H, s), 13.40 (1H, s). MS m/z: 361 [M+1]^{+·}.

### Example III-56

### (Z)-5-(1H-Pyrrol-2-yl)methylene)-2-amino-4-chloro-7-((tetrahydro-2H-pyran-4-yl)methyl)-5H-pyrrolo[2,3-d]pyrimidin-6(7H)-one

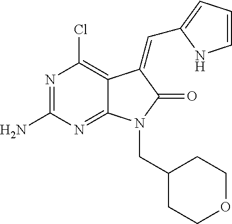

The title compound was prepared in 43% yield using the same protocols for the synthesis of example-48 with 1H-pyrrole-2-carbaldehyde replacing 1H-imidazole-2-carbaldehyde. ¹H NMR (300 MHz, DMSO-d₆) δ 1.26 (2H, m), 1.50 (2H, m), 2.09 (1H, m), 3.23 (2H, m), 3.67 (2H, d), 3.83 (2H, m), 6.36 (1H, d), 6.96 (1H, s), 7.20 (2H, s), 7.35 (1H, s), 7.77 (1H, s), 12.72 (1H, s). MS m/z: 360 [M+l]

### Example III-57

### (Z)-541H-Pyrrol-2-yl)methylene)-2-amino-4-chloro-7-((1-methyl-1H-pyrazol-3-yl)methyl)-5H-pyrrolo[2,3-d]pyrimidin-6(7H)-one

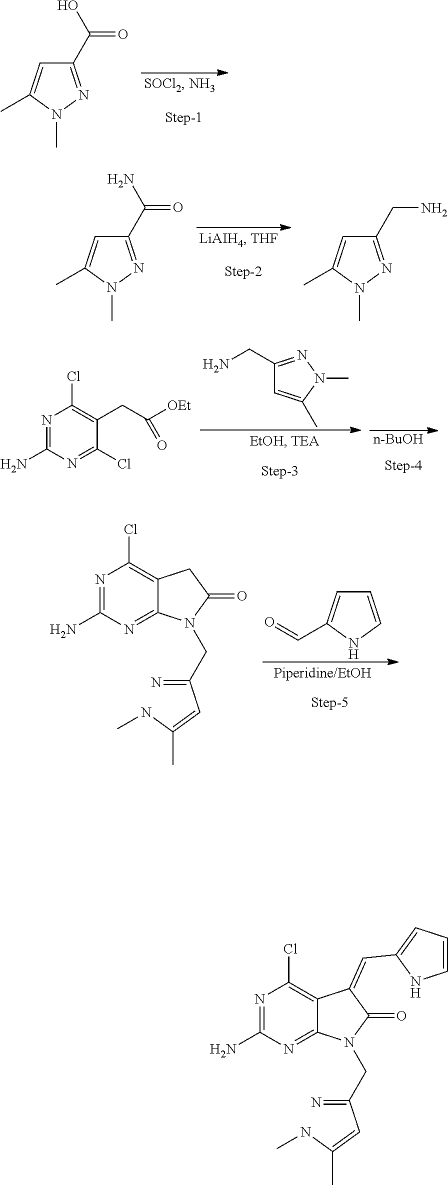

### Step-1: 1,5-Dimethyl-1H-pyrazole-3-carboxamide

A mixture of 1,5-dimethyl-1H-pyrazole-3-carboxylic acid (10 g, 71.43 mmol) and SOCl₂ (25.5 g, 214.29 mmol) was refluxed for 2 hr. The mixture was concentrated under vacuum. The residue was added with 200 mL of NH₃.H₂O dropwise with stirring at 0° C. The solids were collected by filtration, resulted in 8 g (81%) of 1,5-dimethyl-1H-pyrazole-3-carboxamide as a white solid.

### Step-2: 1,5-Dimethyl-1H-pyrazol-3-yl)methanamine

To the above product (5 g, 35.97 mmol) in THF (70 mL) was added LiAlH₄ (4.1 g, 107.89 mmol) in several batches at 0° C. The resulting solution was refluxed for 1 hr. The solids were filtered out and the filtrate was concentrated, resulted in 3 g (67%) of 1,5-dimethyl-1H-pyrazol-3-yl)methanamine as yellow oil.

### Step-3: Ethyl 2-(2-amino-4-chloro-6-1(1,5-dimethyl-1H-pyrazol-3-yl)methylamino)pyrimidin-5-yl)acetate

To a solution of ethyl 2-(2-amino-4,6-dichloropyrimidin-5-yl)acetate (200 mg, 0.80 mmol) in EtOH (30 mL) was added (1,5-dimethyl-1H-pyrazol-3-yl)methanamine (300 mg, 2.40 mmol) and Et₃N (400 mg, 3.96 mmol). The resulting solution was refluxed for 3 hours. The reaction mixture was concentrated under vacuum. The residue was purified on a silica gel column with DCM:MeOH (50:1), resulted in 0.25 g (31%) of ethyl 2-(2-amino-4-chloro-6-((1,5-dimethyl-1H-pyrazol-3-yl)methylamino)pyrimidin-5-yl)acetate as a yellow solid

### Step-4: 2-Amino-4-chloro-7((1,3-dimethyl-1H-pyrazol-5-yl)methyl)-5H-pyrrolo[2,3-d]pyrimidin-6(7H)-one

To a solution of ethyl 2-(2-amino-4-chloro-6-((1,3-dimethyl-1H-pyrazol-5-yl)methylamino)pyrimidin-5-yl)acetate (1 g, 2.96 mmol) in t-BuOH (50 mL) was added Et₃N (1.2 g, 11.88 mmol). The mixture was refluxed for 3 hours and concentrated under vacuum, resulted in 0.7 g (81%) of 2-amino-4-chloro-7-((1,3-dimethyl-1H-pyrazol-5-yl)methyl)-5H-pyrrolo[2,3-d]pyrimidin-6(7H)-one as a yellow solid.

### Step-5: (Z)-5-((1H-Pyrrol-2-yl)methylene)-2-amino-4-chloro-7-((1-methyl-1H-pyrazol-3-yl)methyl)-5H-pyrrolo[2,3-d]pyrimidin-6(7H)-one

To a solution of 2-amino-4-chloro-7-((1,3-dimethyl-1H-pyrazol-5-yl)methyl)-5H-pyrrolo[2,3-d]pyrimidin-6(7H)-one (300 mg, 1.03 mmol) in 20 mL of EtOH was added 1H-pyrrole-2-carbaldehyde (98.2 mg, 1.03 mmol) and two drops of piperidine. The resulting mixture was stirred for overnight at room temperature. The solids were collected by filtration, resulted in 228 mg (60%) of the title compound as a yellow solid. ¹H NMR (400 MHz, DMSO-d₆) δ 2.16 (3H, s), 3.61 (3H, s), 4.84 (2H, s), 5.90 (1H, s), 6.35 (1H, s), 6.97 (1H, s), 7.22 (2H, s), 7.34 (1H, s), 7.78 (1H, s), 12.69 (1H, s). MS m/z: 370 [M+1]^{+·}.

### Example III-58

### (Z)-5-(1H-Imidazol-2-yl)methylene)-2-amino-4-chloro-7-((1-methyl-1H-pyrazol-3-yl)methyl)-5H-pyrrolo[2,3-d]pyrimidin-6(7H)-one

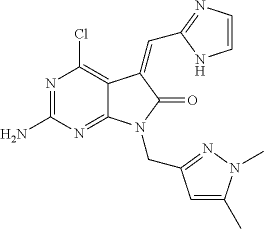

The title compound was prepared in 61% yield using the same protocols for the synthesis of example-57 with 1H-imidazole-2-carbaldehyde replacing 1H-pyrrole-2-carbaldehyde. NMR (300 MHz, DMSO-d₆) δ 2.16 (3H, s), 3.61 (3H, s), 4.85 (2H, s), 5.95 (1H, s), 7.32 (1H, s), 7.51 (1H, s), 7.53 (2H, s), 7.75 (1H, s), 13.33 (1H, s). MS m/z: 371 [M+1]^{+·}.

### Example III-59

### (Z)-5-((2-Amino-4-chloro-7-((1,5-dimethyl-1H-pyrazol-3-yl)methyl)-6-oxo-6,7-dihydro-5H-pyrrolo[2,3-d]pyrimidin-5-ylidene)methyl)-N-(2-(diethylamino)ethyl)-1H-pyrrole-3-carboxamide

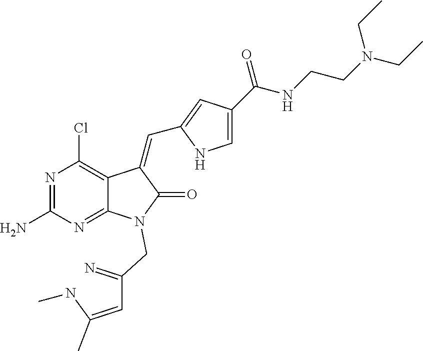

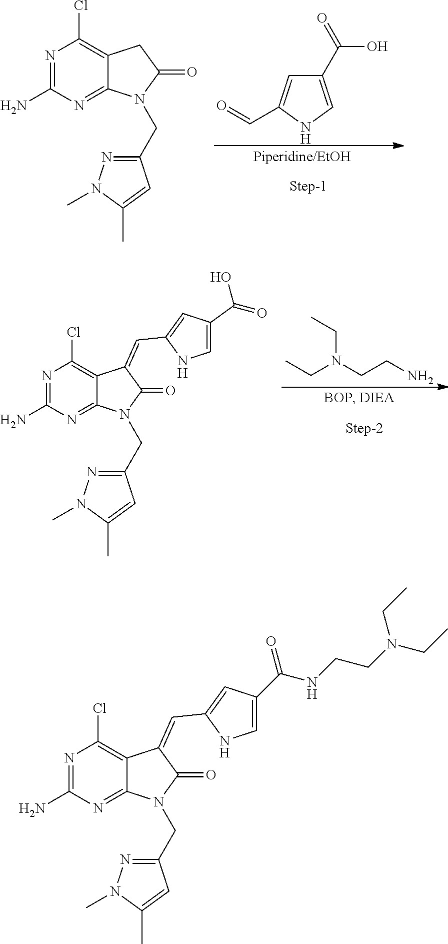

### Step-1: (Z)-5-((2-Amino-4-chloro-7-((1,5-dimethyl-1H-pyrazol-3-yl)methyl)-6-oxo-6,7-dihydropyrrolo[2,3-d]pyrimidin-5-ylidene)methyl)-1H-pyrrole-3-carboxylic acid

To a solution of 2-amino-4-chloro-7-((1,5-dimethyl-1H-pyrazol-3-yl)methyl)-5H-pyrrolo[2,3-d]pyrimidin-6(7H)-one (280 mg, 0.96 mmol, 1.00 equiv) in 30 mL of EtOH under nitrogen was added 5-formyl-1H-pyrrole-3-carboxylic acid (140 mg, 1.01 mmol) and piperidine (a drop). The resulting solution was stirred at 40° C. for overnight. The reaction mixture was cooled to room temperature and solids were collected by filtration, resulted in 0.28 g (71%) of (Z)-5-((2-amino-4-chloro-7-((1,5-dimethyl-1H-pyrazol-4-yl)methyl)-6-oxo-6,7-dihydropyrrolo[2,3-d]pyrimidin-5-ylidene) methyl)-1H-pyrrole-3-carboxylic acid as a yellow solid

### Step-2: (Z)-5-((2-Amino-4-chloro-7-((1,5-dimethyl-1H-pyrazol-3-yl)methyl)-6-oxo-6,7-dihydro-5H-pyrrolo[2,3-d]pyrimidin-5-ylidene)m ethyl)-N-(2-(diethylamino)ethyl)-1H-pyrrole-3-carboxamide

To the above product (300 mg, 0.73 mmol) in 20 mL of THF under nitrogen was added N1,N1-diethylethane-1,2-diamine (116 mg, 1.00 mmol), BOP (368 mg, 0.83 mmol), and DIEA (112.5 mg, 0.87 mmol). The resulting solution was stirred at R.T. for 12 hr. The reaction mixture was added with water and solid was collected by filtration, resulted in 243 mg (65%) of title compound as a yellow solid. ¹H NMR (300 MHz, DMSO-d₆) δ 0.97 (6H, t), 2.16 (3H, s), 2.54 (6H, m), 3.25 (2H, m), 3.61 (3H, s), 4.84 (2H, s), 5.92 (1H, s), 7.31 (1H, s), 7.33 (2H, s), 7.71 (1H, s), 7.74 (1H, s), 7.96 (1H, m). MS m/z: 512 [M+1]^{+·}.

### Example III-60

### (Z)-5-((2-Amino-4-chloro-7-((1,5-dimethyl-1H-pyrazol-4-yl)methyl)-6-oxo-6,7-dihydro-5H-pyrrolo[2,3-d]pyrimidin-5-ylidene)methyl)-N-(2-(diethylamino)ethyl)-4-methyl-1H-pyrrole-3-carboxamide

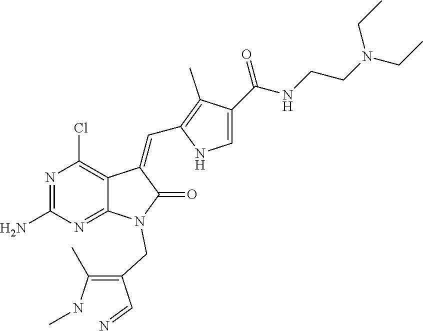

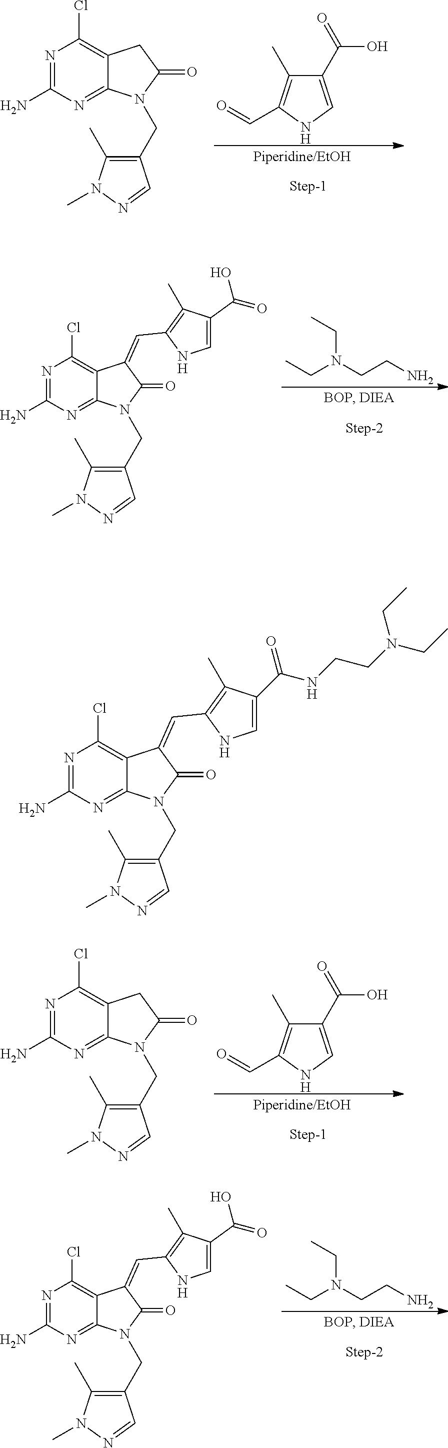

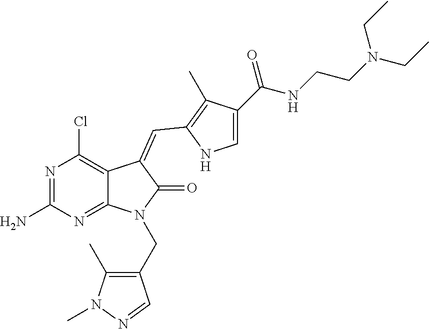

### Step-1: (Z)-5-((2-Amino-4-chloro-7-((1,5-dimethyl-1H-pyrazol-4-yl)methyl)-6-oxo-6,7-dihydropyrrolo[2,3-d]pyrimidin-5-ylidene)methyl)-4-methyl-1H-pyrrole-3-carboxylic acid

To a solution of 2-amino-4-chloro-7-((1,5-dimethyl-1H-pyrazol-4-yl)methyl)-5H-pyrrolo[2,3-d]pyrimidin-6(7H)-one (400 mg, 1.37 mmol) in 20 ml of EtOH (20 mL) under the nitrogen was added 5-formyl-4-methyl-1H-pyrrole-3-carboxylic acid (230 mg, 1.50 mmol) and piperidine (a drop). The resulting solution was stirred for 30 min at room temperature. The solids were collected by filtration, resulted in 400 mg (69%) of (Z)-5-((2-amino-4-chloro-7-((1,5-dimethyl-1H-pyrazol-3-yl)methyl)-6-oxo-6,7-dihydropyrrolo[2,3-d]pyrimidin-5-ylidene)methyl)-4-methyl-1H-pyrrole-3-carboxylic acid as a yellow solid.

### Step-2: (Z)-5-((2-Amino-4-chloro-7-((1,5-dimethyl-1H-pyrazol-4-yl)methyl)-6-oxo-6,7-dihydro-5H-pyrrolo[2,3-d]pyrimidin-5-ylidene)methyl)-N-(2-(diethylamino)ethyl)-4-methyl-1H-pyrrole-3-carboxamide

To a solution of (Z)-5-((2-amino-4-chloro-7-((1,5-dimethyl-1H-pyrazol-4-yl)methyl)-6-oxo-6,7-dihydropyrrolo[2,3-d]pyrimidin-5-ylidene)methyl)-4-methyl-1H-pyrrole-3-carboxylic acid (400 mg, 0.94 mmol) in 20 ml of DMF was added BOP (495.04 mg, 1.12 mmol), DIEA (376 mg, 2.91 mmol), and N1,N1-diethylethane-1,2-diamine (132.24 mg, 1.14 mmol). The resulting solution was stirred for an hour at room temperature and quenched by the addition of 20 mL of water. The solids were collected by filtration, resulted in 157 mg (32%) of the title compound as a yellow solid. ¹H NMR (400 MHz, DMSO-d₆) δ 0.97 (6H, t), 2.16 (3H, s), 2.43 (3H, s), 2.48 (6H, m), 3.24 (2H, m), 3.61 (3H, s), 4.84 (2H, s), 5.91 (1H, s), 7.28 (2H, s), 7.75 (1H, d), 7.78 (1H, s), 7.84 (1H, t), 12.80 (1H, s). MS m/z: 526 [M+1]^{+·}.

### Example III-61

### (Z)-5-((2-Amino-4-chloro-7-((1,5-dimethyl-1H-pyrazol-4-yl)methyl)-6-oxo-6,7-dihydro-5H-pyrrolo[2,3-d]pyrimidin-5-ylidene)methyl)-4-methyl-N-(2-morpholinoethyl)-1H-pyrrole-3-carboxamide

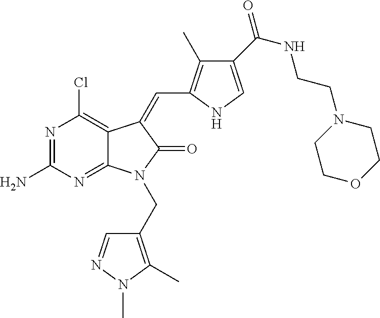

The title compound was prepared in 63% yield using the same protocols for the synthesis of example-60 with the 2-morpholinoethanamine replacing N1,N1-diethylethane-1,2-diamine. ¹H NMR (400 MHz, DMSO-d₆) 2.16 (3H, s), 2.41 (6H, m), 2.43 (3H, s), 3.30 (2H, m), 3.58 (4H, t), 3.61 (3H, s), 4.84 (2H, s), 5.91 (1H, s), 7.27 (2H, s), 7.76 (1H, d), 7.79 (1H, s), 7.87 (1H, t), 12.81 (1H, s). MS m/z: 540 [M+1]^{+·}.

### Example III-62

### (Z)-5-(1H-Pyrrol-2-yl)methylene)-2-amino-7-(benzo[d]oxazol-2-ylmethyl)-4-chloro-5H-pyrrolo[2,3-d]pyrimidin-6(7H)-one

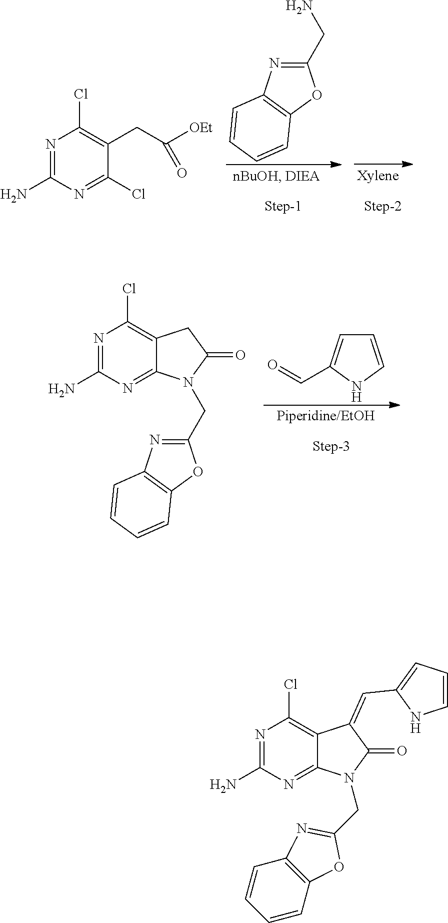

### Step-1: Ethyl 2-(2-amino-4-(benzo[d]oxazol-2-ylmethylamino)-6-chloropyrimidin-5-yl)acetate

To a solution of ethyl 2-(2-amino-4,6-dichloropyrimidin-5-yl)acetate (80 mg, 0.32 mmol) in 20 mL of n-BuOH under nitrogen was added benzo[d]oxazol-2-ylmethanamine (95 mg, 0.64 mmol) and DIEA (124 mg, 0.96 mmol). The resulting solution was refluxed for overnight. The reaction mixture was cooled to 20° C. and the solids were collected by filtration, resulted in 50 mg (39%) of ethyl 2-(2-amino-4-(benzo[d]oxazol-2-ylmethylamino)-6-chloropyrimidin-5-yl)acetate as a white solid.

### Step-2: 2-Amino-7-(benzo[d]oxazol-2-ylmethyl)-4-chloro-5H-pyrrolo[2,3-d]pyrimidin-6(7H)-one

A solution of above product (100 mg, 0.28 mmol) in mL of o-xylene was refluxed for 10 hrs and the mixture was cooled to 20° C. The solids were collected by filtration, resulted in 50 mg (57%) of 2-amino-7-(benzo[d]oxazol-2-ylmethyl)-4-chloro-5H-pyrrolo[2,3-d]pyrimidin-6(7H)-one as a white solid.

### Step-3: (Z)-5-((1H-Pyrrol-2-yl)methylene)-2-amino-7-(benzo[d]oxazol-2-ylmethyl)-4-chloro-5H-pyrrolo[2,3-d]pyrimidin-6(7H)-one

A mixture of 2-amino-7-(benzo[d]oxazol-2-ylmethyl)-4-chloro-5H-pyrrolo[2,3-d]pyrimidin-6(7H)-one (160 mg, 0.81 mmol) in 30 mL of EtOH, 1H-pyrrole-2-carbaldehyde (48.2 mg, 0.51 mmol), and piperidine (a drop) was stirred at 50° C. for overnight and then cooled to 20° C. The solids were collected by filtration, resulted in 212 mg (67%) of the title compound as a yellow solid. ¹H NMR (300 MHz, DMSO-d₆) δ 5.32 (2H, s), 6.37 (1H, s), 7.05 (1H, s), 7.25 (2H, s), 7.38 (3H, m), 7.71 (2H, m), 7.85 (1H, s), 12.53 (1H, s). MS m/z: 393 [M+1]^{+·}.

### Example III-63

### (Z)-5-((1H-Imidazol-2-yl)methylene)-2-amino-7-(benzo[d]oxazol-2-ylmethyl)-4-chloro-5H-pyrrolo[2,3-d]pyrimidin-6(7H)-one

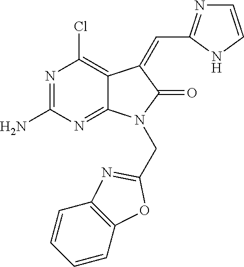

The title compound was prepared in 66% yield using the same protocols for the synthesis of Example-62 with 1H-imidazole-2-carbaldehyde replacing 1H-pyrrole-2-carbaldehyde. ¹H NMR (400 MHz, DMSO-d₆) δ 5.32 (2H, s), 7.34-7.42 (3H, m), 7.50-7.56 (2H, m), 7.72-7.74 (2H, m), 7.82 (1H, s), 13.17 (1H, s). MS m/z: 394 [M+1]^{+·}.

### Example III-64

### (Z)-5-((2-Amino-4-chloro-6-oxo-7-(pyridin-3-ylmethyl)-6,7-dihydro-5H-pyrrolo[2,3-d]pyrimidin-5-ylidene)methyl)-N-(2-(diethylamino)ethyl)-1H-pyrrole-3-carboxamide

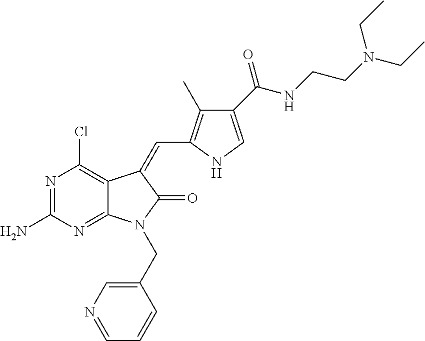

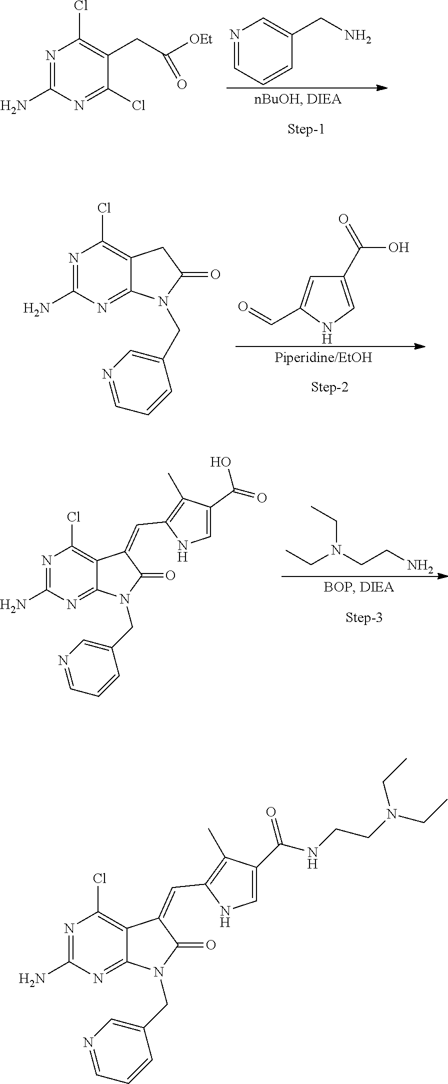

### Step-1: 2-Amino-4-chloro-7-(pyridin-3-ylmethyl)-5H-pyrrolo[2,3-d]pyrimidin-6(7H)-one

To a solution of ethyl 2-(2-amino-4,6-dichloropyrimidin-5-yl)acetate (500 mg, 2.01 mmol) in 20 mL of n-BuOH under nitrogen was added pyridin-3-ylmethanamine (228 mg, 1.85 mmol) and DIEA (778.87 mg, 6.037 mmol). The resulting solution was stirred for 12 hr at 120° C. The reaction mixture was cooled with a water bath and the solids were collected by filtration, resulted in 600 mg (109%) of 2-amino-4-chloro-7-(pyridin-3-ylmethyl)-5H-pyrrolo[2,3-d]pyrimidin-6(7H)-one as a yellow solid.

### Step-2: (Z)-5-((2-Amino-4-chloro-6-oxo-7-(pyridin-3-ylmethyl)-6,7-dihydropyrrolo[2,3-d]pyrimidin-5-ylidene)methyl)-1H-pyrrole-3-carboxylic acid

To above product (500 mg, 1.82 mmol) in 30 mL of EtOH was added 5-formyl-1H-pyrrole-3-carboxylic acid (250 mg, 1.80 mmol) and piperidine (a drop). The resulting solution was stirred for 12 hr at RT. The solids were collected by filtration, resulted in 600 mg (83%) of (Z)-5-((2-amino-4-chloro-6-oxo-7-(pyridin-3-ylmethyl)-6,7-dihydropyrrolo[2,3-d]pyrimidin-5-ylidene)methyl)-1H-pyrrole-3-carboxylic acid as a yellow solid.

### Step-3: (Z)-5-((2-Amino-4-chloro-6-oxo-7-(pyridin-3-ylmethyl)-6,7-dihydro-5H-pyrrolo[2,3-d]pyrimidin-5-ylidene)methyl)-N-(2-(diethylamino)ethyl)-1H-pyrrole-3-carboxamide

A mixture of (Z)-5-((2-amino-4-chloro-6-oxo-7-(pyridin-3-ylmethyl)-6,7-dihydropyrrolo[2,3-d]pyrimidin-5-ylidene)methyl)-1H-pyrrole-3-carboxylic acid (600 mg, 1.52 mmol) in 20 mL of THF, N1,N1-diethylethane-1,2-diamine (263.6 mg, 2.27 mmol), BOP (806.2 mg, 1.82 mmol), and DIEA (235.3 mg, 1.82 mmol) was stirred for 12 hr at room temperature. The resulting solids were collected by filtration, yielded in 404 mg (54%) of the title compound as a yellow solid. ¹H NMR (300 MHz, DMSO-d₆) δ 0.96 (6H, t), 2.47 (6H, m), 3.25 (2H, m), 5.00 (2H, s), 7.28-7.43 (4H, m), 7.73 (3H, m), 7.94 (1H, m), 8.50 (1H, d, J=4.2 Hz), 8.62 (1H, s). MS m/z: 495 [M+1]^{+·}.

### Example III-65

### (Z)-5-((2-Amino-4-chloro-6-oxo-7-(pyridin-2-ylmethyl)-6,7-dihydro-5H-pyrrolo[2,3]pyrimidin-5-ylidene)methyl)-N-(2-(diethylamino)ethyl)-1H-pyrrole-3-carboxamide

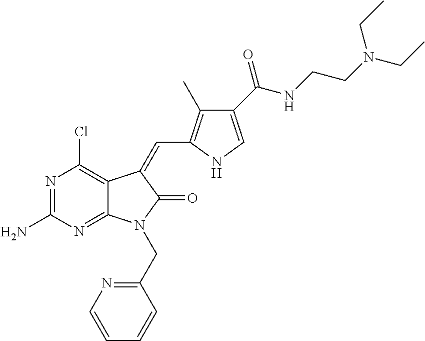

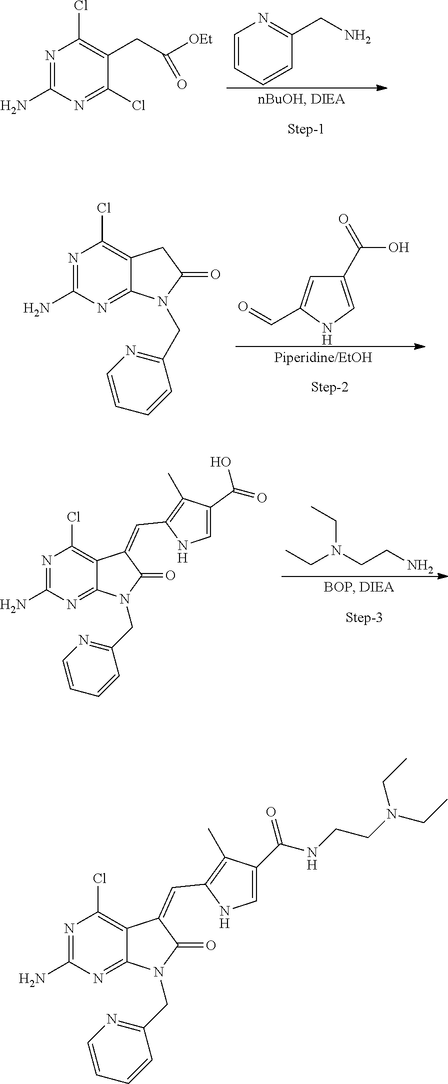

### Step-1: 2-Amino-4-chloro-7-(pyridin-2-ylmethyl)-5H-pyrrolo[2,3-d]pyrimidin-6(7H)-one

To a solution of ethyl 2-(2-amino-4,6-dichloropyrimidin-5-yl)acetate (500 mg, 2.01 mmol) in n-BuOH (20 mL) was added pyridin-2-ylmethanamine (228 mg, 1.85 mmol) and DIEA (778 mg, 6.03 mol). The resulting solution was stirred at 120° C. for 12 hr. The reaction mixture was cooled and the solids were collected by filtration, resulted in 300 mg (54%) of 2-amino-4-chloro-7-(pyridin-2-ylmethyl)-5H-pyrrolo[2,3-d]pyrimidin-6(7H)-one as a yellow solid.

### Step-2: (Z)-5-((2-Amino-4-chloro-6-oxo-7-(pyridin-2-ylmethyl)-6,7-dihydropyrrolo[2,3-d]pyrimidin-5-ylidene) methyl)-1H-pyrrole-3-carboxylic acid

A solution of 2-amino-4-chloro-7-(pyridin-2-ylmethyl)-5H-pyrrolo[2,3-d]pyrimidin-6(7H)-one (500 mg, 1.82 mmol) in EtOH (40 ml), 5-formyl-1H-pyrrole-3-carboxylic acid (300 mg, 2.16 mmol), and piperidine (1 d g, 11.76 mmol, 6.47 equiv) was stirred at 40-50° C. for overnight. The reaction mixture was cooled to 10° C. and the solids were collected by filtration, resulted in 550 mg (76%) of (Z)-5-((2-amino-4-chloro-6-oxo-7-(pyridin-2-ylmethyl)-6,7-dihydropyrrolo[2,3-d]pyrimidin-5-ylidene) methyl)-1H-pyrrole-3-carboxylic acid as a yellow solid.

### Step-3: (Z)-5-((2-Amino-4-chloro-6-oxo-7-(pyridin-2-ylmethyl)-6,7-dihydro-5H-pyrrolo[2,3-d]pyrimidin-5-ylidene)methyl)-N-(2-(diethylamino)ethyl)-1H-pyrrole-3-carboxamide

To a solution of (Z)-5-((2-amino-4-chloro-6-oxo-7-(pyridin-2-ylmethyl)-6,7-dihydropyrrolo[2,3-d]pyrimidin-5-ylidene) methyl)-1H-pyrrole-3-carboxylic acid (550 mg, 1.39 mmol) in 40 mL of THF was added N1,N1-diethylethane-1,2-diamine (242 mg, 2.09 mmol), DIEA (215 mg, 1.67 mmol), and BOP (558 mg, 1.26 mmol). The reaction was carried out with stirring at room temperature for overnight. The reaction was treated with 40 mL of water and the solids were collected by filtration, washed with 2×10 mL of EA and 3×30 mL of Et₂O, resulted in 212 mg (31%) of the title compound as a yellow solid. ¹H NMR (300 MHz, CDCl₃) δ 1.07 (6H, t), 2.50-2.72 (6H, m), 3.48 (2H, m), 5.18 (2H, s), 5.28 (2H, s), 6.79 (1H, s), 6.94 (1H, s), 7.20 (2H, m), 7.55˜7.70 (2H, m), 7.84 (1H, s), 8.56 (1H, s). MS m/z: 495 [M+1]^{+·}.

### Example III-66

### (Z)-5-((2-Amino-4-chloro-6-oxo-7-(pyridin-4-ylmethyl)-6,7-dihydro-5H-pyrrolo[2,3-d]pyrimidin-5-ylidene)methyl)-N-(2-(diethylamino)ethyl)-1H-pyrrole-3-carboxamide

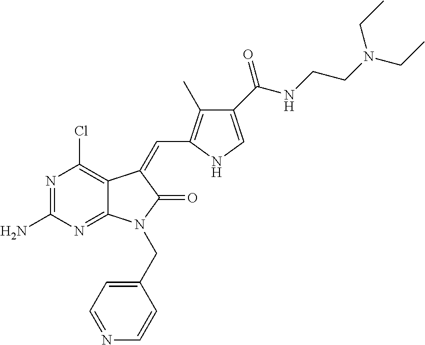

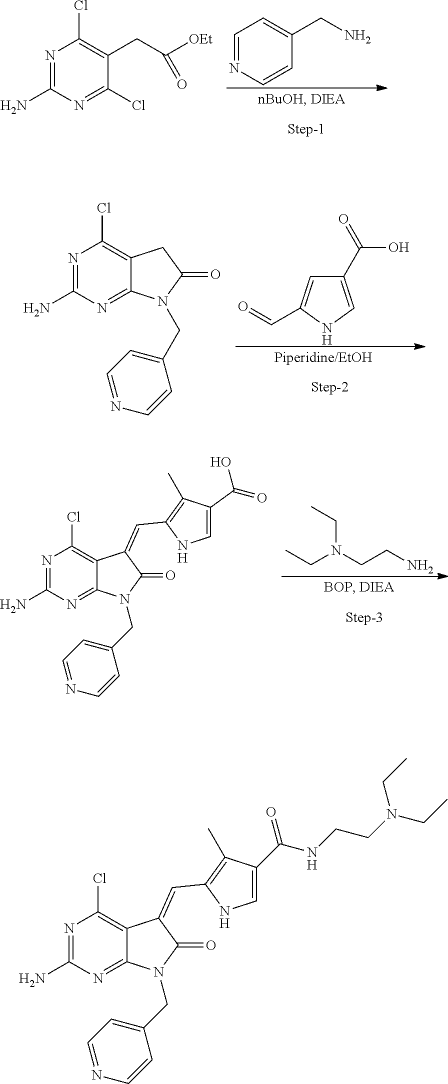

### Step-1: 2-Amino-4-chloro-7-(pyridin-4-ylmethyl)-5H-pyrrolo[2,3-d]pyrimidin-6(7H)-one

To a solution of ethyl 2-(2-amino-4,6-dichloropyrimidin-5-yl)acetate (700 mg, 2.81 mmol) in 20 mL of n-BuOH (20 mL) under nitrogen was added pyridin-4-ylmethanamine (338.5 mg, 2.75 mmol) and DIEA (1.079 mg, 8.36 mmol). The resulting solution was stirred at 120° C. for 12 hr. The reaction mixture was cooled and the solids were collected by filtration, resulted in 300 mg (39%) of 2-amino-4-chloro-7-(pyridin-4-ylmethyl)-5H-pyrrolo[2,3-d]pyrimidin-6(7H)-one as a yellow solid.

### Step-2: (Z)-5-((2-Amino-4-chloro-6-oxo-7-(pyridin-4-ylmethyl)-6,7-dihydropyrrolo[2,3-d]pyrimidin-5-ylidene)methyl)-1H-pyrrole-3-carboxylic acid

A mixture of 2-amino-4-chloro-7-(pyridin-4-ylmethyl)-5H-pyrrolo[2,3-d]pyrimidin-6(7H)-one (500 mg, 1.82 mmol, 1.00 equiv) in 30 mL of EtOH, 5-formyl-1H-pyrrole-3-carboxylic acid (265 mg, 1.91 mmol), and piperidine (a drop) under nitrogen was stirred at 60° C. overnight. The reaction mixture was cooled to room temperature and the solids were collected by filtration, the resulted in 300 mg (41%) of (Z)-5-((2-amino-4-chloro-6-oxo-7-(pyridin-4-ylmethyl)-6,7-dihydropyrrolo[2,3-d]pyrimidin-5-ylidene) methyl)-1H-pyrrole-3-carboxylic acid as a yellow solid.

### Step-3: (Z)-5-((2-Amino-4-chloro-6-oxo-7-(pyridin-4-ylmethyl)-6,7-dihydro-5H-pyrrolo[2,3-d]pyrimidin-5-ylidene)methyl)-N-(2-(diethylamino)ethyl)-1H-pyrrole-3-carboxamide

A mixture of (Z)-5-((2-amino-4-chloro-6-oxo-7-(pyridin-4-ylmethyl)-6,7-dihydropyrrolo[2,3-d]pyrimidin-5-ylidene) methyl)-1H-pyrrole-3-carboxylic acid (400 mg, 1.01 mmol) in THF (30 mL), BOP (616 mg, 1.39 mmol), DIEA (150 mg, 1.16 mmol), and N1,N1-diethylethane-1,2-diamine (201 mg, 1.73 mmol) was stirred at room temperature for overnight. The reaction was then added with 30 mL of water and the solids were collected by filtration, washed with 2×50 mL of Et₂O, resulted in 153 mg (30%) of the title compound as a yellow solid. ¹H NMR (300 MHz, DMSO-d₆) δ 1.15 (6H, t), 3.05 (6H, m), 3.47 (2H, m), 5.01 (2H, s), 7.20-7.39 (5H, m), 7.76 (2H, s), 8.25 (1H, s), 8.51 (2H, s), 12.65 (1H, s). MS m/z: 495 [M+1]^{+·}.

### Example III-67

### (Z)-5-((2-Amino-4-chloro-7-((1-methyl-1H-pyrazol-4-yl)methyl)-6-oxo-6,7-dihydro-5H-pyrrolo[2,3-d]pyrimidin-5-ylidene)methyl)-N-(2-(diethylamino)ethyl)-4-methyl-1H-pyrrole-3-carboxamide

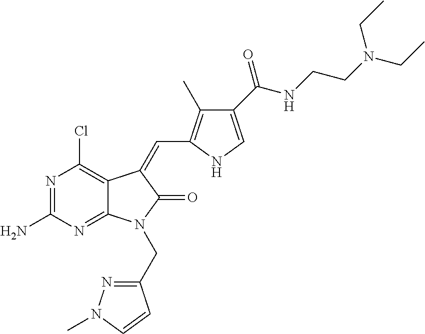

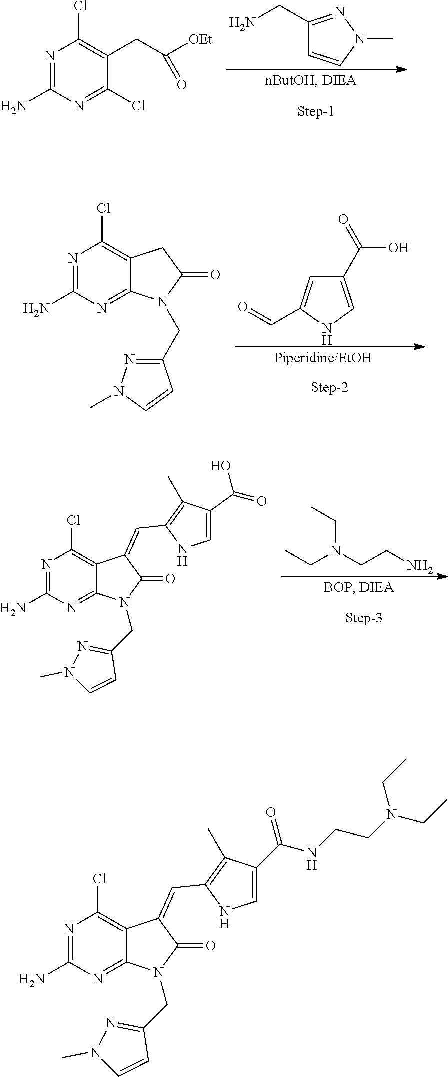

### Step-1: 2-Amino-4-chloro-7-((1-methyl-1H-pyrazol-3-yl)methyl)-5H-pyrrolo[2,3-d]pyrimidin-6(7H)-one

To a solution of ethyl 2-(2-amino-4-chloro-6-((1-methyl-1H-pyrazol-3-yl)methylamino)pyrimidin-5-yl)acetate (2.8 g, 8.64 mmol) in n-BuOH (20 mL) was added DIEA (2.5 g, 19.38 mmol). The resulting solution was stirred at 130° C. for 12 hr and the solids were collected by filtration, resulted in 2.0 g (83%) of 2-amino-4-chloro-7-((1-methyl-1H-pyrazol-3-yl)methyl)-5H-pyrrolo[2,3-d]pyrimidin-6(7H)-one as a white solid.

### Step-2: (Z)-5-((2-Amino-4-chloro-7-((1-methyl-1H-pyrazol-3-yl)methyl)-6-oxo-6,7-dihydropyrrolo[2,3-d]pyrimidin-5-ylidene)methyl)-1H-pyrrole-3-carboxylic acid

A solution of 2-amino-4-chloro-7-((1-methyl-1H-pyrazol-3-yl)methyl)-5H-pyrrolo[2,3-d]pyrimidin-6(7H)-one (460 mg, 1.65 mmol) in EtOH (10 mL), 5-formyl-1H-pyrrole-3-carboxylic acid (276 mg, 1.99 mmol) and piperidine under nitrogen was stirred for 12 hr at 70° C. The reaction mixture was cooled with a water/ice bath and the solids were collected by filtration, resulted in 550 mg (83%) of (Z)-5-((2-amino-4-chloro-7-((1-methyl-1H-pyrazol-3-yl)methyl)-6-oxo-6,7-dihydropyrrolo[2,3-d]pyrimidin-5-ylidene)methyl)-1H-pyrrole-3-carboxylic acid as a red solid.

### Step-3: (Z)-5-((2-amino-4-chloro-7-((1-methyl-1H-pyrazol-4-yl)methyl)-6-oxo-6,7-dihydro-5H-pyrrolo[2,3-d]pyrimidin-5-ylidene)methyl)-N-(2-(diethylamino)ethyl)-4-methyl-1H-pyrrole-3-carboxamide

To a solution of (Z)-5-((2-amino-4-chloro-7-((1-methyl-1H-pyrazol-3-yl)methyl)-6-oxo-6,7-dihydropyrrolo[2,3-d]pyrimidin-5-ylidene)methyl)-1H-pyrrole-3-carboxylic acid (450 mg, 1.13 mmol) in DMF (30 ml) was added BOP (581.7 mg, 1.32 mmol), DIEA (291.3 mg, 2.26 mmol), and N1,N1-diethylethane-1,2-diamine (200 mg, 1.72 mmol). The resulting mixture was stirred for 1 hr at room temperature and then treated with 20 ml of water. The solids were collected by filtration, resulted in 340 mg (61%) of the title compound as a red solid. ¹H NMR (400 MHz, DMSO-d₆) δ, 0.96 (6H, t), 2.48 (6H, m), 3.25 (2H, m), 3.74 (3H, s), 4.90 (2H, s), 6.14 (1H, s), 7.31 (1H, s), 7.33 (2H, s), 7.58 (1H, d), 7.71 (1H, s), 7.74 (1H, m), 7.97 (1H, t), 12.71 (1H, s). MS m/z: 498 [M+l]

### Example III-68

### (Z)-5-((2-Amino-4-chloro-7-((1-methyl-1H-pyrazol-4-yl)methyl)-6-oxo-6,7-dihydro-5H-pyrrolo[2,3-d]pyrimidin-5-ylidene)methyl)-N-(2-morpholinoethyl)-1H-pyrrole-3-carboxamide

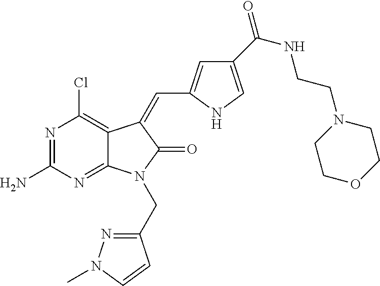

The title compound was prepared using the same protocols for the synthesis of example-67 with the 2-morpholinoethanamine replacing N1,N1-diethylethane-1,2-diamine. ¹H NMR (400 MHz, DMSO-d₆) δ 2.41 (6H, m), 3.30 (2H, m), 3.57 (4H, m), 3.74 (3H, s), 4.91 (2H, s), 6.15 (1H, d), 7.32 (1H, s), 7.33 (2H, s), 7.58 (1H, d), 7.72 (1H, s), 7.75 (1H, d), 8.00 (1H, t), 12.71 (1H, s). MS m/z: 512 [M+1]^{+·}.

### Example III-69

### (Z)-5-((2-Amino-4-chloro-7-((4,5-dimethylthiazol-2-yl)methyl)-6-oxo-6,7-dihydro-5H-pyrrolo[2,3-d]pyrimidin-5-ylidene)methyl)-N-(2-(diethylamino)ethyl)-1H-pyrrole-3-carboxamide

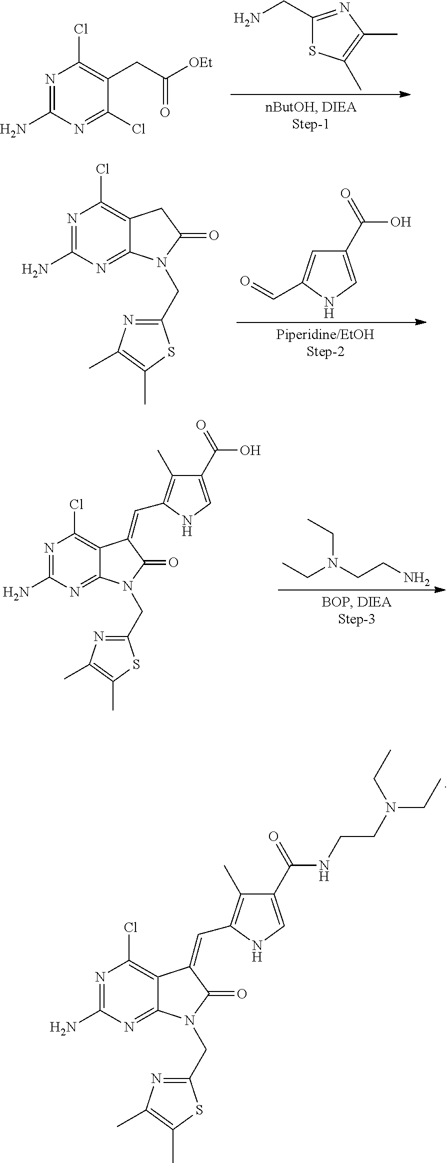

### Step-1: 2-Amino-4-chloro-7-((4,5-dimethylthiazol-2-yl)methyl)-5H-pyrrolo[2,3-d]pyrimidin-6(7H)-one

To a solution of ethyl 2-(2-amino-4-chloro-6-((1-methyl-1H-pyrazol-3-yl)methylamino)pyrimidin-5-yl)acetate (1.0 g, 4.00 mmol) in n-BuOH (20 mL) was added (4,5-dimethylthiazol-2-yl)methanamine (900 mg, 6.34 mmol) and DIEA (2.0 g, 15.50 mmol, 3.00 equiv). The resulting solution was heated to reflux with stirring for 20 hr. The reaction mixture was cooled to 25° C. and the solids were collected by filtration, resulted in 800 mg (65%) of 2-amino-4-chloro-7-((4,5-dimethylthiazol-2-yl)methyl)-5H-pyrrolo[2,3-d]pyrimidin-6(7H)-one as a yellow solid.

### Step-2: (Z)-5-((2-Amino-4-chloro-7-((4,5-dimethylthiazol-2-yl)methyl)-6-oxo-6,7-dihydropyrrolo[2,3-d]pyrimidin-5-ylidene)methyl)-1H-pyrrole-3-carboxylic acid

A mixture of 2-amino-4-chloro-7-((4,5-dimethylthiazol-2-yl)methyl)-5H-pyrrolo[2,3-d]pyrimidin-6(7H)-one (700 mg, 2.26 mmol) in EtOH (60 mL) and 5-formyl-1H-pyrrole-3-carboxylic acid (350 g, 2.52 mol), and piperidine (a catalytic amount) was refluxed with stirring for overnight. The reaction was cooled to 25° C. and the solids were collected by filtration, resulted in 900 mg (92%) of (Z)-5-((2-amino-4-chloro-7-((4,5-dimethylthiazol-2-yl)methyl)-6-oxo-6,7-dihydropyrrolo[2,3-d]pyrimidin-5-ylidene)methyl)-1H-pyrrole-3-carboxylic acid as a Yellow solid.

### Step-3: (Z)-5-((2-Amino-4-chloro-7-((4,5-dimethylthiazol-2-yl)methyl)-6-oxo-6,7-dihydro-5H-pyrrolo[2,3-d]pyrimidin-5-ylidene)methyl)-N-(2-(diethylamino)ethyl)-1H-pyrrole-3-carboxamide

To a solution of (Z)-5-((2-amino-4-chloro-7-((4,5-dimethylthiazol-2-yl)methyl)-6-oxo-6,7-dihydropyrrolo[2,3-d]pyrimidin-5-ylidene)methyl)-1H-pyrrole-3-carboxylic acid (200 mg, 0.47 mmol) in DMF (20 mL) at 0° C. was added BOP (247 mg, 0.56 mmol), DIEA (63 mg, 0.56 mmol), and N1,N1-diethylethane-1,2-diamine (65 mg, 0.56 mmol). The resulting solution was stirred for 1 hr at room temperature and treated with 20 mL of water/ice. The mixture was extracted with 3×20 mL of ethyl acetate. The organic layers were combined and dried over anhydrous sodium sulfate and concentrated under vacuum. The resulting solids were washed with 2×20 mL of EtOH and 2×20 mL of ether, resulted in 204 mg (79%) of the title compound as a brown solid. ¹H NMR (300 MHz, CDCl₃) δ 1.08 (6H, t), 2.30 (6H, d), 2.56-2.75 (6H, m), 3.50 (2H, m), 5.25 (2H, s), 5.32 (2H, s), 6.95 (1H, s), 7.65 (2H, s), 7.86 (1H, s), 13.10 (1H, s). MS m/z: 529 [M+1]^{+·}.

### Example III-70

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4-methylpiperazin-1-yl)ethyl)-1H-pyrrole-3-carboxamide

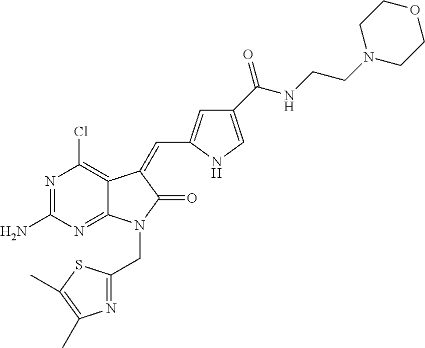

The title compound was prepared in 66% yield using the same protocols for the synthesis of Example-69 with the 2-morpholinoethanamine replacing N1,N1-diethylethane-1,2-diamine. ¹H NMR (400 MHz, DMSO-d₆) δ 2.19 (3H, s), 2.26 (3H, s), 2.43 (6H, m), 3.30 (2H, m), 3.57 (4H, t), 5.13 (2H, s), 7.38 (3H, s), 7.75 (1H, s), 7.76 (1H, s), 8.01 (1H, t), 12.61 (1H, s). MS m/z: 543 [M+1]^{+·}.

### Example III-71

### (Z)-N-(2-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-1H-imidazol-4-yl)isobutyramide

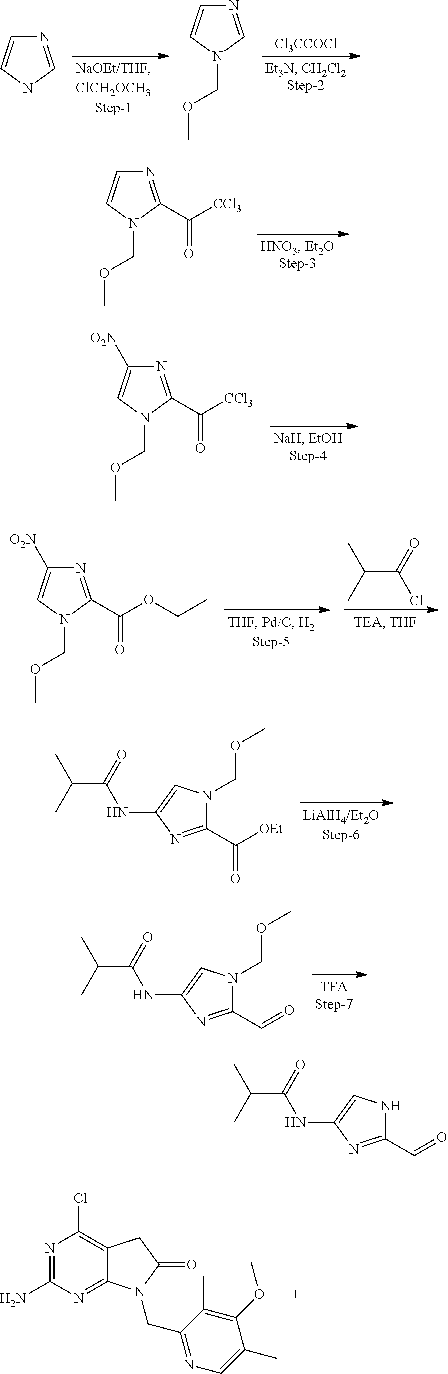

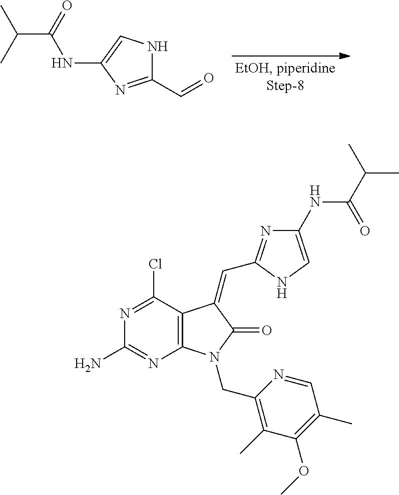

### Step-1:1-(Methoxymethyl)-1H-imidazole

To a solution of 1H-imidazole (40.8 g, 600.00 mmol) in EtOH (1200 mL) under nitrogen was added a solution of sodium ethanolate (40.8 g, 600.00 mmol) in THF (1200 mL). The resulting solution was stirred for 1 hr at room temperature and concentrated under vacuum. To the residue was added chloro(methoxy)methane (53.4 g, 667.50 mmol) and the resulting solution was stirred for overnight at room temperature. The solids were filtered. The filtrate was concentrated and distilled under reduced pressure, resulted in 40 g (58%) of 1-(methoxymethyl)-1H-imidazole as white oil.

### Step-2: 2,2,2-Trichloro-1-(1-(methoxymethyl)-1H-imidazol-2-yl)ethanone

To a solution of 1-(methoxymethyl)-1H-imidazole (52 g, 464.29 mmol) in DCM (1000 mL) under N₂ was added 2,2,2-trichloroacetyl chloride (92 g, 511.11 mmol) dropwise. The resulting solution was stirred for 30 minutes at room temperature. To it was added Et₃N (51 g, 504.00 mmol) dropwise and the resulting solution was stirred for 2 hours at room temperature. The reaction mixture was concentrated and the residue was purified on a silica gel column with ethyl acetate/petroleum ether (1:20), resulted in 73 g (61%) of 2,2,2-trichloro-1-(1-(methoxymethyl)-1H-imidazol-2-yl)ethanone as yellow oil.

### Step-3: 2,2,2-Trichloro-1-(1-(methoxymethyl)-4-nitro-1H-imidazol-2-yl)ethanone

To above product (73 g, 285.16 mmol) in ethoxyethane (486 mL) was added HNO₃ (121.6 mL) dropwise temperature kept at −45° C. The resulting solution was stirred for 5 hr at room temperature. The reaction was then quenched by the addition of 1000 mL of water/ice and extracted with 3×1000 mL of dichloromethane. Combined organic layers were washed with 3×3000 mL of H₂O and 3×2000 mL of brine and dried over anhydrous magnesium sulfate. The resulting solution was concentrated under vacuum, resulted in 50 g (55%) of 2,2,2-trichloro-1-(1-(methoxymethyl)-4-nitro-1H-imidazol-2-yl)ethanone as red oil.

### Step-4: Ethyl 1-(methoxymethyl)-4-nitro-1H-imidazole-2-carboxylate

To a solution of 2,2,2-trichloro-1-(1-(methoxymethyl)-4-nitro-1H-imidazol-2-yl)ethanone (50 g, 166.11 mmol) in EtOH (500 mL) under nitrogen was added NaH (1 g, 41.67 mmol). The mixture was stirred for 3 hr at room temperature. The reaction was worked up, resulted in 19 g (49%) of ethyl 1-(methoxymethyl)-4-nitro-1H-imidazole-2-carboxylate as a yellow solid.

### Step-5: Ethyl 4-isobutyramido-1-(methoxymethyl)-1H-imidazole-2-carboxylate

A solution of ethyl 1-(methoxymethyl)-4-nitro-1H-imidazole-2-carboxylate (6 g, 26.20 mmol) in THF (40 mL) was added with Pd/C (0.5 g). The mixture was stirred under a hydrogen atmosphere for 4 hr at room temperature. The solids were filtered out and washed with 2×20 mL of THF. Combined organic layers were mixed with TEA (4 g, 39.60 mmol) followed by the addition of a solution of isobutyryl chloride (3.3 g, 30.99 mmol,) in THF (10 mL) dropwise at 10° C. The resulting solution was stirred overnight at room temperature. The resulting mixture was concentrated under vacuum and extracted with 2×50 mL of ethyl acetate. Combined organic layers were dried over anhydrous magnesium sulfate, concentrated under vacuum, and the residue was purified on a silica gel column with ethyl acetate/petroleum ether (1:10-1:4) to afford 5 g (71%) of ethyl 4-isobutyramido-1-(methoxymethyl)-1H-imidazole-2-carboxylate as a white solid.

### Step-6: N-(2-Formyl-1-(methoxymethyl)-1H-imidazol-4-yl)isobutyramide

To a mixture of LiAlH₄ (740 mg, 19.47 mmol) in Et₂O (30 mL)was added a solution of ethyl 4-isobutyramido-1-(methoxymethyl)-1H-imidazole-2-carboxylate (2.65 g, 9.85 mmol) in Et₂O (20 mL) dropwise at −10° C. and then added MnO₂ (8.4 g, 96.55 mmol). The resulting mixture was stirred for 2 hr at −10° C. and then heated to reflux for overnight. After it was cooled to room temperature, the solids were filtered out and washed with 3×50 mL of ethyl acetate. The combined filtrates were concentrated under vacuum and the residue was purified on a silica gel column with ethyl acetate/petroleum ether (1:5), resulted in 1.5 g (68%) of N-(2-formyl-1-(methoxymethyl)-1H-imidazol-4-yl)isobutyramide as a white solid.

### Step-7: N-(2-Formyl-1H-imidazol-4-yl)isobutyramide

A solution of ethyl 4-isobutyramido-1-(methoxymethyl)-1H-imidazole-2-carboxylate (1.2 g, 5.33 mmol) in TFA (15 mL) was refluxed for overnight. The resulting mixture was concentrated under vacuum and the residue was purified on a silica gel column with petroleum ether/ethyl acetate (1:10), resulted in 0.4 g (41%) of N-(2-formyl-1H-imidazol-4-yl)isobutyramide as a white solid.

### Step-8: (Z)-N-(2-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-1H-imidazol-4-yl)isobutyramide

To a solution of above product (120 mg, 0.66 mmol) in EtOH (8 mL) was added 2-amino-4-chloro-7-((4-methoxy-3,5-dimethylpyridin-2-yl)methyl)-5H-pyrrolo[2,3-d]pyrimidin-6(7H)-one (230 mg, 0.69 mmol) and piperidine (1 mg). The resulting solution was stirred for 6 hr at room temperature. The solids were collected by filtration and washed with 2×10 mL of EtOH and 2×10 mL of Et₂O, resulted in 200 mg (61%) of the title compound as a red solid. NMR (400 MHz, CDCl₃) δ 1.22-1.27 (6H, m), 2.22 (3H, s), 2.37 (3H, s), 2.56 (1H, m), 3.80 (3H, s), 5.10 (2H, s), 5.29 (2H, s), 7.65 (1H, s), 7.92 (2H, s), 8.13 (1H, s), 13.67 (1H, s). MS m/z: 497 [M+1]^{+·}.

### Example III-72

### (Z)-N-(2-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-1-(methoxymethyl)-1H-imidazol-4-yl)isobutyramide

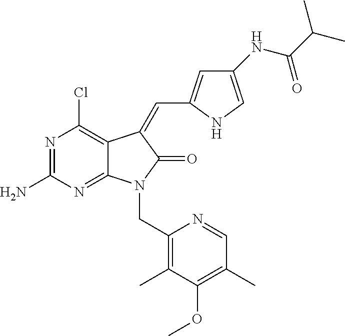

The title compound was prepared using the same protocols for the synthesis of example-71 with the corresponding aldehyde replacing N-(2-formyl-1H-imidazol-4-yl)isobutyramide. NMR (400 MHz, DMSO-d₆) δ 1.05 (6H, d), 2.14 (3H, s), 2.25 (3H, s), 2.64 (1H, m), 3.17 (3H, s), 3.73 (3H, s), 4.89 (2H, s), 5.28 (2H, s), 7.42 (2H, s), 7.53 (2H, d), 8.01 (1H, s), 10.36 (1H, s). MS m/z: 497 [M+1]^{+·}.

### Example III-73

### (Z)-N-(2-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-1H-imidazol-4-yl)cyclopentanecarboxamide

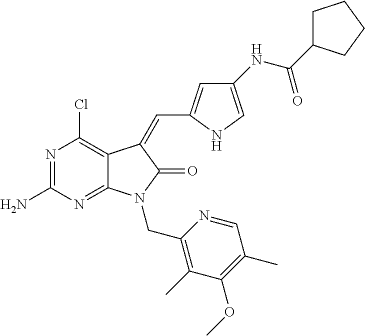

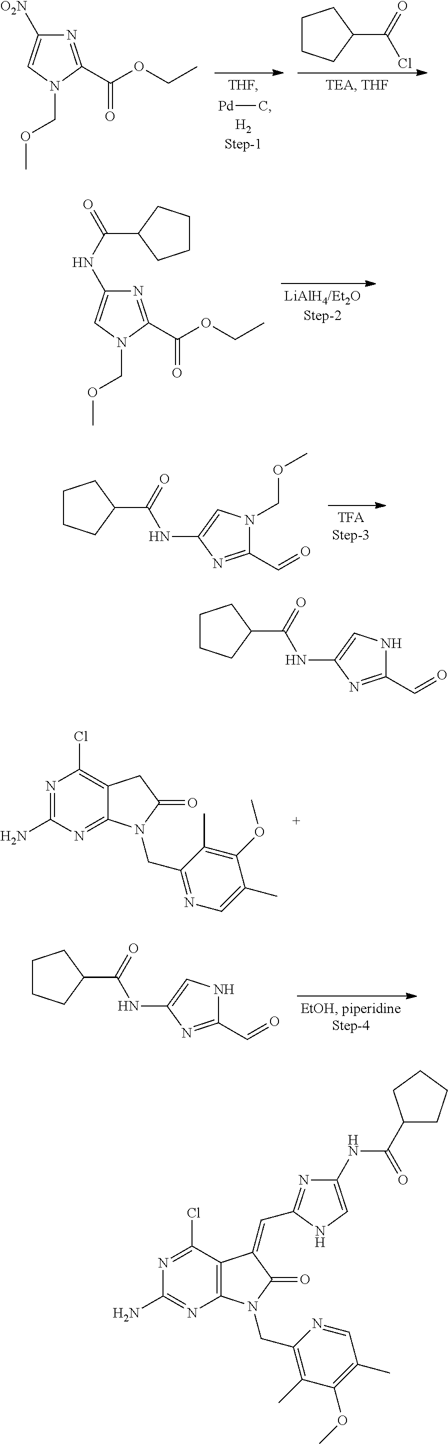

### Step-1: Ethyl 4-(cyclopentanecarboxamido)-1-(methoxymethyl)-1H-imidazole-2-carboxylate

To a solution of ethyl 1-(methoxymethyl)-4-nitro-1H-imidazole-2-carboxylate (3 g, 13.10 mmol) in THF (20 mL) under N₂ was added Pd/C (0.5 g). To this was introduced hydrogen and the mixture was stirred for 3 hrs at room temperature. The solids were filtered out and washed with 10 mL of THF. To the combined filtrates was added TEA (2.6 g, 25.74 mmol) and cyclopentanecarbonyl chloride (2.6 g, 19.55 mmol) in dropwise at 0° C. The resulting solution was stirred for overnight at room temperature. The resulting mixture was concentrated under vacuum and the residue was dissolved in 150 mL of ethyl acetate. The resulting solution was washed with 2×100 mL of water and 30 mL of brine. The mixture was dried over anhydrous magnesium sulfate and concentrated under vacuum. The residue was purified on a silica gel column with ethyl acetate/petroleum ether (1:5) to give 3.3 g (85%) of ethyl 4-(cyclopentanecarboxamido)-1-(methoxymethyl)-1H-imidazole-2-carboxylate as white oil.

### Step-2: N-(2-Formyl-1-(methoxymethyl)-1H-imidazol-4-yl)cyclopentanecarboxamide

To a solution of LiAlH₄ (770 mg, 20.26 mmol) in THF (20 mL) under N₂ was added a solution of ethyl 4-(cyclopentanecarboxamido)-1-(methoxymethyl)-1H-imidazole-2-carboxylate (3 g, 10.16 mmol) in THF (10 mL) in dropwise at −20° C. This was followed by the addition of a solution of MnO₂ (6.2 g, 71.26 mmol) in EtOAc (30 mL). The resulting solution was stirred for 2 hrs at −20° C. and was heated to reflux for overnight. The reaction was then quenched by the addition of 20 mL of ethyl acetate and the solids were filtered out. The resulting mixture was extracted with 2×20 mL of ethyl acetate and the organic layers combined. The mixture was dried over anhydrous magnesium sulfate and concentrated under vacuum. The residue was purified on a silica gel column with ethyl acetate/petroleum ether (1:5), resulted in 1.9 g (74%) of N-(2-formyl-1-(methoxymethyl)-1H-imidazol-4-yl)cyclopentanecarboxamide as a white solid.

### Step-3: N-(2-Formyl-1H-imidazol-4-yl)cyclopentanecarboxamide

A mixture of N-(2-formyl-1-(methoxymethyl)-1H-imidazol-4-yl)cyclopentanecarboxamide (100 mg, 0.40 mmol) and 2,2,2-trifluoroacetic acid (1 mL) under N₂ was refluxed for 6 hrs. The resulting mixture was concentrated under vacuum and the residue was purified on a silica gel column with ethyl acetate/petroleum ether (1:5). The product was used in next step.

### Step-4: (Z)-N-(2-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-1H-imidazol-4-yl)cyclopentanecarboxamide

To a solution of 2-amino-4-chloro-7-((4-methoxy-3,5-dimethylpyridin-2-yl)methyl)-5H-pyrrolo[2,3-d]pyrimidin-6(7H)-one (110 mg, 0.33 mmol) in EtOH (10 mL) was added N-(2-formyl-1H-imidazol-4-yl)cyclopentanecarboxamide (71 mg, 0.34 mmol) and piperidine (a drop). The resulting solution was stirred for 5 hrs at room temperature. The solids were collected by filtration, resulted in 160 mg (93%) of the title compound as a yellow solid. ¹H NMR (400 MHz, CDCl₃) δ, 1.64 (2H, m), 1.78 (2H, m), 1.80-2.00 (4H, m), 2.20 (3H, s), 2.35 (3H, s), 2.73 (1H, m), 3.78 (3H, s), 4.70 (4H, s), 5.09 (2H, s), 7.654 (1H, s), 7.91 (1H, s), 8.07 (1H, s). MS m/z: 532 [M+1]^{+·}.

### Example III-74

### (Z)-2-Amino-4-chloro-7-((4-methoxy-3,5-dimethylpyridin-2-yl)methyl)-5-((4-(morpholinomethyl)-1H-imidazol-2-yl)methylene)-5H-pyrrolo[2,3-d]pyrimidin-6(7H)-one

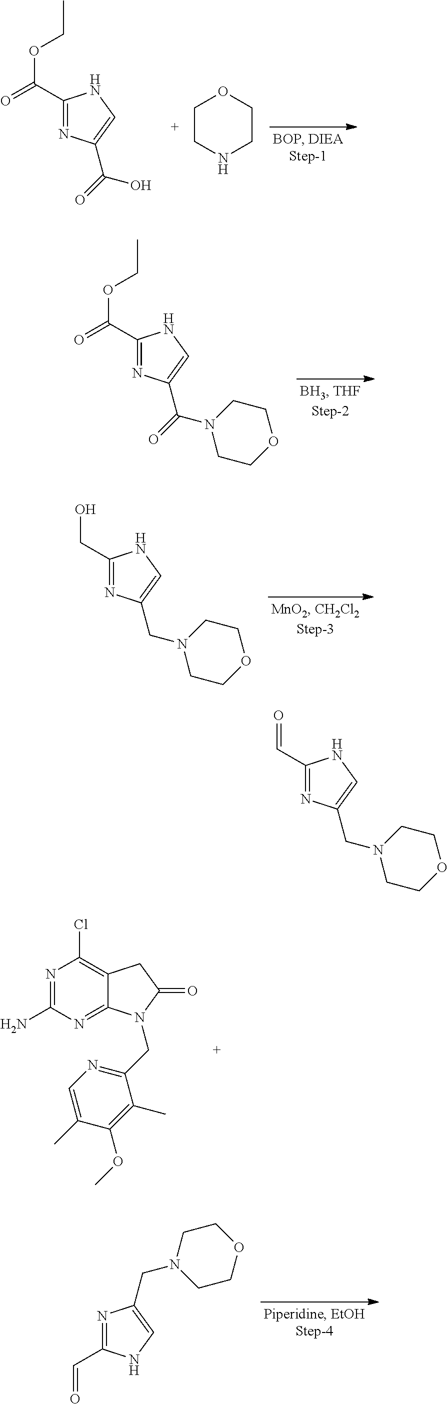

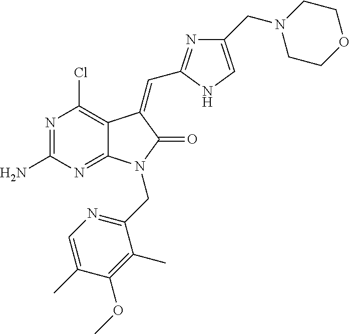

### Step-1: Ethyl 5-(morpholine-4-carbonyl)-1H-imidazole-2-carboxylate

A mixture of 2-(ethoxycarbonyl)-1H-imidazole-5-carboxylic acid (500 mg, 2.70 mmol), morpholine (275 mg, 3.16 mmol), Bop (1325 mg, 3.00 mmol), and DIEA (500 mg, 3.88 mmol) was stirred for 12 hr at room temperature. The reaction mixture was treated with 1×100 mL of H₂O. The resulting solution was extracted with 4×200 mL of dichloromethane. The combined organic layers were washed with 100 mL of brine and dried over anhydrous sodium sulfate. After the solvent was evaporated, the residue was purified on a silica gel column with dichloromethane/methanol (50:1), resulted in 0.5 g (73%) of ethyl 5-(morpholine-4-carbonyl)-1H-imidazole-2-carboxylate as a white solid.

### Step-2: (5-(Morpholinomethyl)-1H-imidazol-2-yl)methanol

A solution of ethyl 5-(morpholine-4-carbonyl)-1H-imidazole-2-carboxylate (700 mg, 2.76 mmol), borane (7 mL), and THF (10 mL) was stirred for 12 hr at room temperature. The reaction was treated with 20 mL of MeOH. The resulting mixture was concentrated under vacuum. The residue was purified on a silica gel column with dichloromethane/methanol (20:1), resulted in 0.3 g (55%) of (5-(morpholinomethyl)-1H-imidazol-2-yl)methanol as white oil.

### Step-3: 5-(Morpholinomethyl)-1H-imidazole-2-carbaldehyde

A mixture of (5-(morpholinomethyl)-1H-imidazol-2-yl)methanol (300 mg, 1.52 mmol), MnO₂ (900 mg, 10.34 mmol), and CH₂Cl₂ (50 mL) was stirred for 12 hr at room temperature. The solids were filtered out and the resulting mixture was concentrated under vacuum to give 160 mg (54%) of 5-(morpholinomethyl)-1H-imidazole-2-carbaldehyde as white oil.

### Step-4: (Z)-2-Amino-4-chloro-7-((4-methoxy-3,5-dimethylpyridin-2-yl)methyl)-5-((4-(morpholinomethyl)-1H-imidazol-2-yl)methylene)-5H-pyrrolo[2,3-d]pyrimidin-6(7H)-one

A mixture of 2-amino-4-chloro-7-((4-methoxy-3,5-dimethylpyridin-2-yl)methyl)-5H-pyrrolo[2,3-d]pyrimidin-6(7H)-one (220 mg, 0.66 mmol), 5-(morpholinomethyl)-1H-imidazole-2-carbaldehyde (123 mg, 0.63 mmol), and a drop of piperidine was stirred for 12 hr at room temperature. The solid was filtered, resulted in 0.245 g (73%) of the title compound as a yellow solid. ¹H NMR (300 MHz, CD₃COCD₃) S 2.18 (3H, s), 2.32-2.50 (7H, m), 3.59 (6H, m), 3.82 (3H, s), 5.11 (2H, d), 6.61 (2H, s), 7.29 (2H, d), 7.91 (1H, s), 7.98 (1H, s), 13.50 (1H, s). MS m/z: 511 [M+1]^{+·}.

### Example III-75

### (Z)-N-(2-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-1H-imidazol-4-yl)tetrahydro-2H-pyran-4-carboxamide

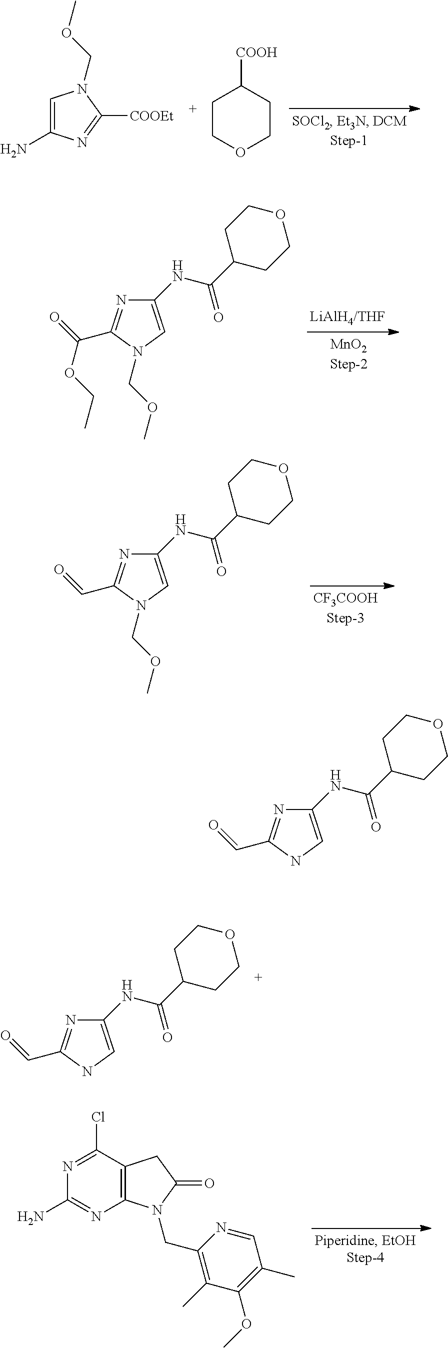

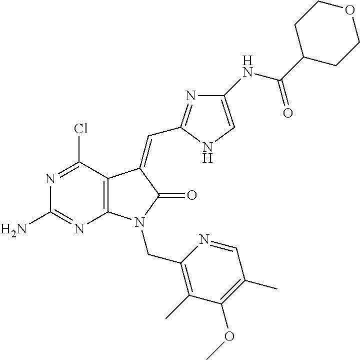

### Step-1: Ethyl 1-(methoxymethyl)-4-(tetrahydro-2H-pyran-4-carboxamido)-1H-imidazole-2-carboxylate

To 2.7 g of tetrahydro-2H-pyran-4-carboxylic acid (20.75 mmol) was added sulfuryl dichloride (27 mL), ethyl 4-amino-1-(methoxymethyl)-1H-imidazole-2-carboxylate (2.6 g, 13.05 mmol), triethylamine (5.2 g, 51.38 mmol), and CH₂Cl₂ (30 mL). The resulting solution was refluxed for 2 hr and was continued at room temperature for 12 hr with stirring. The reaction was then quenched by the addition of 20 mL of ice/salt. The organic layer was washed with 2×20 mL of brine, dried over anhydrous magnesium sulfate, and concentrated under vacuum. The residue was purified on a silica gel column with ethyl acetate/petroleum ether (1:10), resulted in 3.5 g (53%) of ethyl 1-(methoxymethyl)-4-(tetrahydro-2H-pyran-4-carboxamido)-1H-imidazole-2-carboxylate as a white solid.

### Step-2: N-(2-Formyl-1-(methoxymethyl)-1H-imidazol-4-yl)-tetrahydro-2H-pyran-4-carboxamide

To a mixture of LiAlH₄ (730 mg, 19.21 mmol) in THF (20 mL) was added a solution of ethyl 1-(methoxymethyl)-4-(tetrahydro-2H-pyran-4-carboxamido)-1H-imidazole-2-carboxylate (3 g, 9.64 mmol) in THF (10 mL) dropwise and followed by the addition of a solution of MnO₂ (5.9 g, 67.82 mmol) in EtOAc (30 mL). During the process, the reaction temperature was kept at −20° C. The resulting mixture was stirred for 2 hr at −20° C. and for overnight at 0° C. The reaction was then treated with 20 mL of EA. The solids were filtered out and filtrate was washed with 2×20 mL of EA, dried over anhydrous magnesium sulfate, and concentrated under vacuum. The residue was purified on a silica gel column with ethyl acetate/petroleum ether (1:5), resulted in 2 g (78%) of N-(2-formyl-1-(methoxymethyl)-1H-imidazol-4-yl)-tetrahydro-2H-pyran-4-carboxamide as a white solid.

### Step-3: N-(2-Formyl-1H-imidazol-4-yl)-tetrahydro-2H-pyran-4-carboxamide

A mixture of N-(2-formyl-1-(methoxymethyl)-1H-imidazol-4-yl)-tetrahydro-2H-pyran-4-carboxamide (1000 mg, 3.74 mmol) and 2,2,2-trifluoroacetic acid (1 mL) was refluxed for 6 hr under nitrogen. The resulting mixture was concentrated under vacuum. The residue was purified on a silica gel column with ethyl acetate/petroleum ether (1:5), resulted in 0.3 g (36%) of N-(2-formyl-1H-imidazol-4-yl)-tetrahydro-2H-pyran-4-carboxamide as a light red solid.

### Step-4: (Z)-N-(2-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-1H-imidazol-4-yl)tetrahydro-2H-pyran-4-carboxamide

To a solution of 2-amino-4-chloro-7-((4-methoxy-3,5-dimethylpyridin-2-yl)methyl)-5H-pyrrolo[2,3-d]pyrimidin-6(7H)-one (173 mg, 0.52 mmol) in EtOH (10 mL) was added N-(2-formyl-1H-imidazol-4-yl)-tetrahydro-2H-pyran-4-carboxamide (120 mg, 0.54 mmol) and piperidine (a drop). The resulting solution was stirred for 5 hr at room temperature. The solids were collected by filtration, resulted in 230 mg (82%) of the title compound as a yellow solid. ¹H NMR (400 MHz, CDCl₃) δ 1.88 (4H, m), 2.22 (3H, s), 2.37 (3H, s), 2.53 (1H, m), 3.46 (2H, m), 3.80 (3H, s), 4.06 (2H, d), 5.11 (2H, s), 5.29 (2H, s), 7.65 (1H, s), 7.93 (2H, m), 8.13 (1H, s), 13.68 (1H, s). MS m/z: 539 [M+1]^{+·}.

### Example III-76

### (Z)-2-(2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-isopropyl-1H-imidazole-4-carboxamide

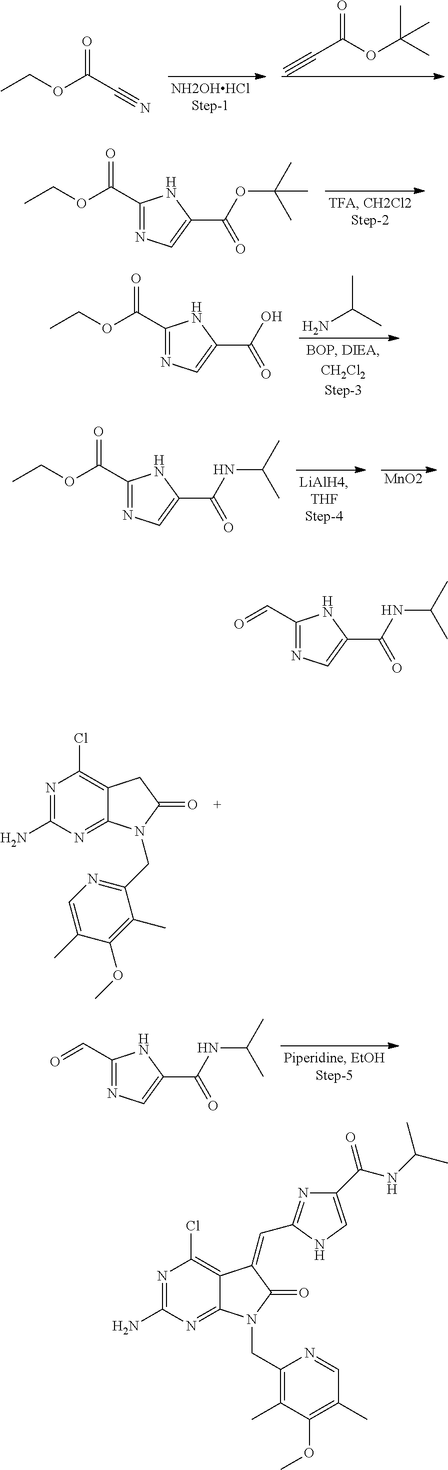

### Step-1: 5-tert-Butyl 2-ethyl 1H-imidazole-2,5-dicarboxylate

To ethyl cyanoformate (10 g, 101.01 mmol) was added NH₂OH.HCl (8.4 g, 121.74 mmol), K₂CO₃ (19.9 g, 144.20 mmol), and CH₂Cl₂/H₂O (100 mL). The resulting mixture was stirred for 12 hr at room temperature. The solids were filtered out and the filtrated was concentrated under vacuum. The residue was applied onto a silica gel column with ethyl acetate/petroleum ether (1:1), resulted in 6 g (45%) of (Z)-ethyl 2-amino-2-(hydroxyimino)acetate as a white solid. To a mixture of (Z)-ethyl 2-amino-2-(hydroxyimino)acetate (1 g, 7.58 mmol) and tert-butyl propiolate (940 mg, 7.46 mmol) was added Et₃N (765 mg, 7.57 mmol) and toluene (50 mL). The resulting solution was stirred at 140° C. for 1 min. The resulting mixture was washed with 50 mL of H₂O and then was extracted with 3×50 mL of dichloromethane. The organic layers were combined and washed with 20 mL of brine, then dried over anhydrous sodium sulfate. Evaporated the solvents and the residue was purified onto a silica gel column with ethyl acetate/petroleum ether (1:1), resulted in 1.1 g (61%) of 5-tert-butyl 2-ethyl 1H-imidazole-2,5-dicarboxylate as a yellow solid.

### Step-2: 2-(Ethoxycarbonyl)-1H-imidazole-5-carboxylic acid

Into a 100-mL round-bottom flask, was placed 5-tert-butyl 2-ethyl 1H-imidazole-2,5-dicarboxylate (2 g, 8.33 mmol). To this was added CF₃COOH (10 mL) in CH₂Cl₂ (7 mL). After the resulting solution was stirred for 12 hr at room temperature, it was concentrated under vacuum. The residue was treated with 30 mL of CH₃OH and the solids were collected by filtration, resulted in 2 g (97%) of 2-(ethoxycarbonyl)-1H-imidazole-5-carboxylic acid as a yellow solid.

### Step-3: Ethyl 5-(isopropylcarbamoyl)-1H-imidazole-2-carboxylate

To a mixture of 2-(ethoxycarbonyl)-1H-imidazole-5-carboxylic acid (600 mg, 3.24 mmol) and propan-2-amine (230 mg, 3.90 mmol) was added BOP (1.58 g, 3.57 mmol) at 0° C. and DIEA (500 mg, 3.88 mmol). The resulting solution was stirred for 12 hr at room temperature and then concentrated to dryness under vacuum. The residue was purified onto a silica gel column with dichloromethane/methanol (20:1), resulted in 0.6 g (82%) of ethyl 5-(isopropylcarbamoyl)-1H-imidazole-2-carboxylate as a white solid.

### Step-4: 2-Formyl-N-isopropyl-1H-imidazole-5-carboxamide

To ethyl 5-(isopropylcarbamoyl)-1H-imidazole-2-carboxylate (800 mg, 3.54 mmol) in THF (50 mL) was added LiAlH₄ (800 mg, 21.05 mmol) in several batches and the resulting solution was stirred for 4 hrs at room temperature. The reaction was then quenched by the addition of 4 mL of water. The solids were filtered out. The solvent was evaporated and the residue was purified onto a silica gel column with dichloromethane/methanol (5:1), resulted in 0.45 g (69%) of 2-(hydroxymethyl)-N-isopropyl-1H-imidazole-5-carboxamide as a white solid. To it (450 mg, 2.45 mmol) was added MnO₂ (4 g, 45.98 mmol) and CH₂Cl₂ (50 ml). The resulting mixture was stirred for 24 hr at room temperature. The solids were filtered out and the filtrate was concentrated under vacuum and the residue was purified onto a silica gel column with dichloromethane/methanol (10:1), resulted in 0.15 g (34%) of 2-formyl-N-isopropyl-1H-imidazole-5-carboxamide as a white solid.

### Step-5: (Z)-242-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-isopropyl-1H-imidazole-4-carboxamide

To a mixture of 2-amino-4-chloro-7-((4-methoxy-3,5-dimethylpyridin-2-yl)methyl)-5H-pyrrolo[2,3-d]pyrimidin-6(7H)-one (220 mg, 0.66 mmol) and 2-formyl-N-isopropyl-1H-imidazole-5-carboxamide (200 mg, 1.10 mmol) in ethanol (2 mL) was added piperidine (a drop). The resulting mixture was stirred for 12 hrs at room temperature. The solids were collected by filtration, resulted in 0.083 g (34%) of the title compound as a yellow solid. ¹H NMR (400 MHz, DMSO-d₆) δ 1.16 (6H, d), 2.14 (3H, s), 2.29 (3H, s), 3.75 (3H, s), 4.09 (1H, m), 5.04 (2H, s), 7.54 (2H, s), 7.78 (1H, s), 7.82 (1H, s), 7.90 (1H, d), 8.02 (1H, s), 13.42 (1H, s). MS m/z: 497 [M+1]^{+·}.

### Example III-77

### (Z)-2-(2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-isobutyl-1H-imidazole-4-carboxamide

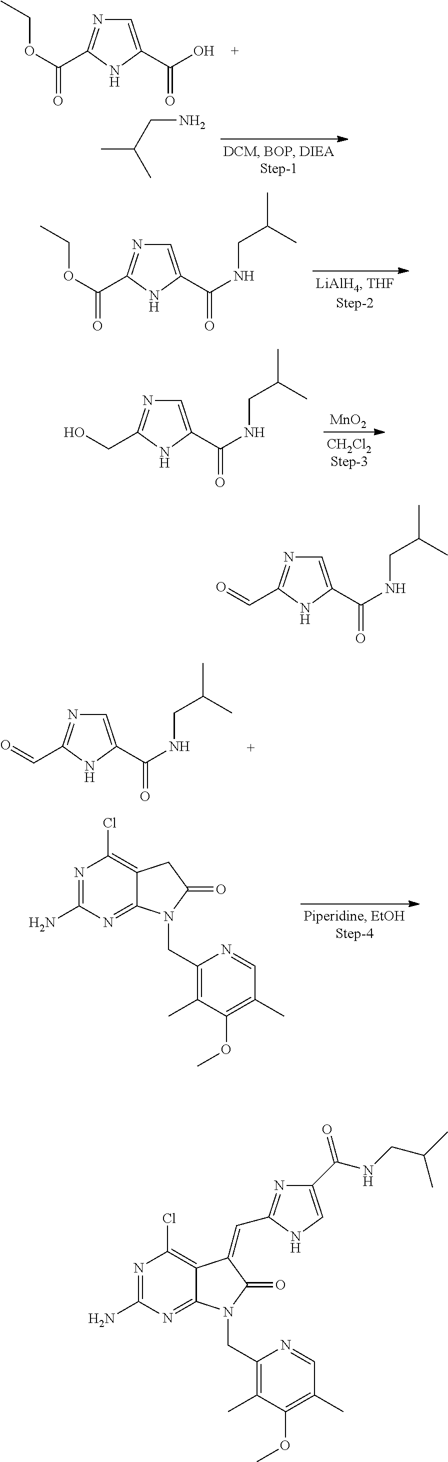

### Step-1: 2-(Ethoxycarbonyl)-1H-imidazole-4-carboxylic acid

To a solution of 2-(ethoxycarbonyl)-1H-imidazole-4-carboxylic acid (100 mg, 0.54 mmol) in DCM (10 mL) at 0° C. was added 2-methylpropan-1-amine (40 mg, 0.55 mmol), BOP (288 mg, 0.65 mmol), and DIEA (84 mg, 0.65 mmol). The resulting solution was allowed stirred for 3 hr at room temperature. The resulting mixture was concentrated under vacuum and the residue was purified onto a silica gel column with dichloromethane/methanol (20:1), resulted in 100 mg (77%) of ethyl 4-(isobutylcarbamoyl)-1H-imidazole-2-carboxylate as a yellow solid.

### Step-2: 2-(Hydroxymethyl)-N-isobutyl-1H-imidazole-5-carboxamide

Into a 50-mL 3-necked round-bottom flask, was placed a solution of ethyl 5-(isobutylcarbamoyl)-1H-imidazole-2-carboxylate (50 mg, 0.21 mmol) in THF (10 mL). To it was added LiAlH₄ (10 mg, 0.26 mmol). The resulting mixture was stirred for 10 min at room temperature. The solids were filtered out and the filtrate was concentrated under vacuum. The residue was purified onto a silica gel column with dichloromethane/methanol (20:1), resulted in 10 mg (24%) of 2-(hydroxymethyl)-N-isobutyl-1H-imidazole-5-carboxamide as yellow oil.

### Step-3: 2-Formyl-N-isobutyl-1H-imidazole-5-carboxamide

To a solution of 2-(hydroxymethyl)-N-isobutyl-1H-imidazole-5-carboxamide (200 mg, 1.02 mmol) in CH₂Cl₂ (20 mL) was added MnO₂ (1000 mg, 11.49 mmol). The mixture was stirred for overnight at room temperature. The solids were filtered out and the filtrate was concentrated under vacuum, resulted in 100 mg (51%) of 2-formyl-N-isobutyl-1H-imidazole-5-carboxamide as yellow oil.

### Step-4: (Z)-2-(2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-isobutyl-1H-imidazole-4-carboxamide

To a mixture of 2-amino-4-chloro-7-((5-methoxy-4,6-dimethylpyridin-2-yl)methyl)-5H-pyrrolo[2,3-d]pyrimidin-6(7H)-one (340 mg, 1.02 mmol) in EtOH (10 mL) was added 2-formyl-N-isobutyl-1H-imidazole-4-carboxamide (200 mg, 1.03 mmol) and piperidine (5 mg). The resulting solution was stirred for 3 hours at room temperature. The resulting mixture was concentrated under vacuum and the residue was purified onto a silica gel column with ethyl acetate/dichloromethane (1:1), resulted in 63 mg (12%) of the title compound as a yellow solid. ¹H NMR (300 MHz, DMSO-d₆) δ 0.86 (6H, d), 1.84 (1H, m), 2.14 (3H, s), 2.29 (3H, s), 3.06 (2H, t), 3.75 (3H, s), 5.05 (2H, s), 6.83 (1H, s), 7.56 (2H, s), 7.77 (1H, s), 7.83 (1H, s), 8.02 (1H, s), 13.43 (1H, s). MS m/z: 511 [M+1]+·.

### Example III-78

### (Z)-2-(2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(4-hydroxycyclohexyl)-1H-imidazole-4-carboxamide

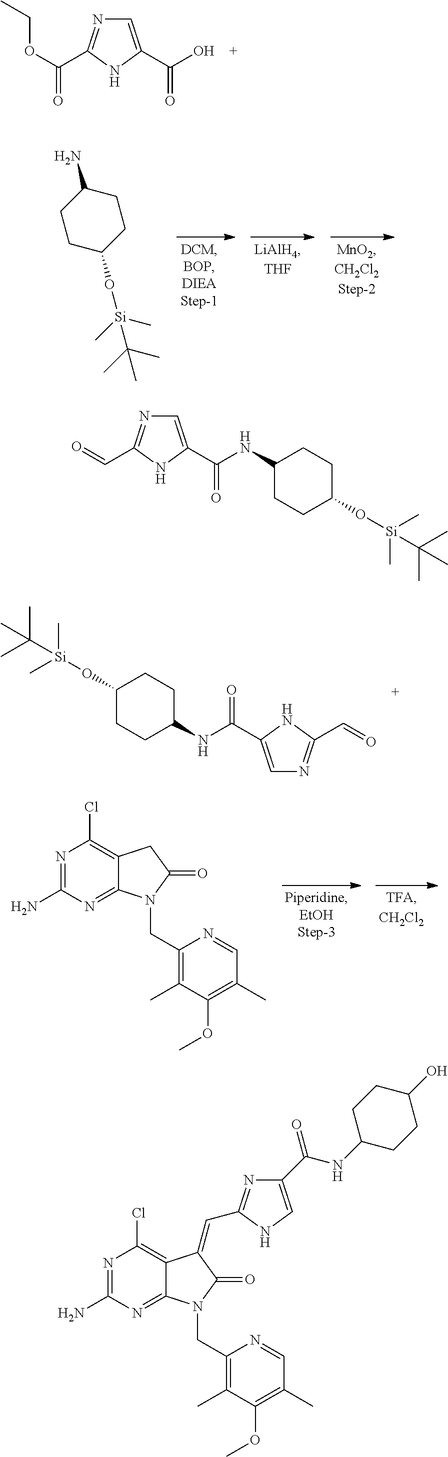

### Step-1: Ethyl 4-((1r,4r)-4-(tert-butyldimethylsilyloxy)cyclohexylcarbamoyl)-1H-imidazole-2-carboxylate

To a solution of (1r, 4r)-4-(tert-butyldimethylsilyloxy)cyclohexanamine (1.4 g, 6.11 mmol) in DCM (20 mL) was added 2-(ethoxycarbonyl)-1H-imidazole-4-carboxylic acid (1 g, 5.43 mmol), BOP (2.9 g, 6.56 mmol) and DIEA (840 mg, 6.51 mmol). The resulting solution was stirred for 3 hr at room temperature and then concentrated under vacuum. The residue was purified onto a silica gel column with dichloromethane/methanol (20:1), resulted in 1.9 g (89%) of ethyl 4-((1r, 4r)-4-(tert-butyldimethylsilyloxy)cyclohexylcarbamoyl)-1H-imidazole-2-carboxylate as a yellow solid.

### Step-2: N-((1 s, 4s)-4-(tert-Butyldimethylsilyloxy)cyclohexyl)-2-formyl-1H-imidazole-4-carboxamide

To a mixture of ethyl 4-(((1s,4s)-4-(tert-butyldimethylsilyloxy)cyclohexyl)carbamoyl)-1H-imidazole-2-carboxylate (160 mg, 0.41 mmol) in THF (20 mL) was added LiAlH₄ (160 mg, 4.21 mmol). The reaction was stirred for 30 min at room temperature and then quenched by the addition of 10 mL of water. The resulting solution was extracted with 4×10 mL of ethyl acetate and the organic layers combined and dried over anhydrous sodium sulfate and concentrated under vacuum. This resulted in 100 mg (70%) of N-((1 s,4s)-4-(tert-butyldimethylsilyloxy)cyclohexyl)-2-(hydroxymethyl)-1H-imidazole-4-carboxamide as yellow oil. Into a 100-mL round-bottom flask, was placed N-((1s,4s)-4-(tert-butyldimethylsilyloxy)cyclohexyl)-2-(hydroxymethyl)-1H-imidazole-4-carboxamide (140 mg, 0.40 mmol) in CH₂Cl₂ (20 mL). To this was added MnO₂ (282 mg, 3.24 mmol). The resulting solution stirred for 12 min at room temperature. The solids were filtered out. The filtrate was concentrated under vacuum. The residue was purified onto a silica gel column with ethyl acetate/petroleum ether (1:1), resulted in 100 mg (72%) of N-((1s,4s)-4-(tert-butyldimethylsilyloxy)cyclohexyl)-2-formyl-1H-imidazole-4-carboxamide as a yellow solid.

### Step-3: (Z)-2-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(4-hydroxycyclohexyl)-1H-imidazole-4-carboxamide

A mixture of N-((1s,4s)-4-(tert-butyldimethylsilyloxy)cyclohexyl)-2-formyl-1H-imidazole-4-carboxamide (430 mg, 1.23 mmol), piperidine (a drop), and 2-amino-4-chloro-7-((5-methoxy-4,6-dimethylpyridin-2-yl)methyl)-5H-pyrrolo[2,3-d]pyrimidin-6(7H)-one (388.5 mg, 1.17 mmol) in EtOH (20 mL)was stirred for 12 hr at room temperature. The solids were collected by filtration, resulted in 500 mg (61%) of 2-((E)-(2-amino-4-chloro-7-((4-methoxy-3,5-dimethylpyridin-2-yl)methyl)-6-oxo-6,7-dihydropyrrolo[2,3-d]pyrimidin-5-ylidene) methyl)-N-((1s,4s)-4-(tert-butyldimethylsilyloxy)cyclohexyl)-1H-imidazole-4-carboxamide as a yellow solid. To it (500 mg, 0.75 mmol) in THF (10 mL) was added H₂O (10 mL) and CF₃COOH (225 g, 3.75 mol). The resulting solution was stirred for 30 min at room temperature and concentrated under vacuum. The product was precipitated by the addition of diethyl ether and collected by filtration, resulted in 0.311 g (74%) of the title compound as a yellow solid. ¹H NMR (300 MHz, DMSO-d₆) δ 1.24 (2H, m), 1.43 (2H, m), 1.81 (4H, m), 2.19 (3H, s), 2.31 (3H, s), 3.39 (1H, m), 3.72 (1H, m), 3.79 (3H, s), 5.08 (2H, s), 7.62 (2H, s), 7.77 (1H, s), 7.90 (1H, s), 8.02-8.15 (2H, m). MS m/z: 553 [M+1]^{+·}.

### Example III-79

### (Z)-2-Amino-4-chloro-5-((4-(4-hydroxypiperidine-1-carbonyl)-1H-imidazol-2-yl)methylene)-7-((4-methoxy-3,5-dimethylpyridin-2-yl)methyl)-5H-pyrrolo[2,3-d]pyrimidin-6(7H)-one

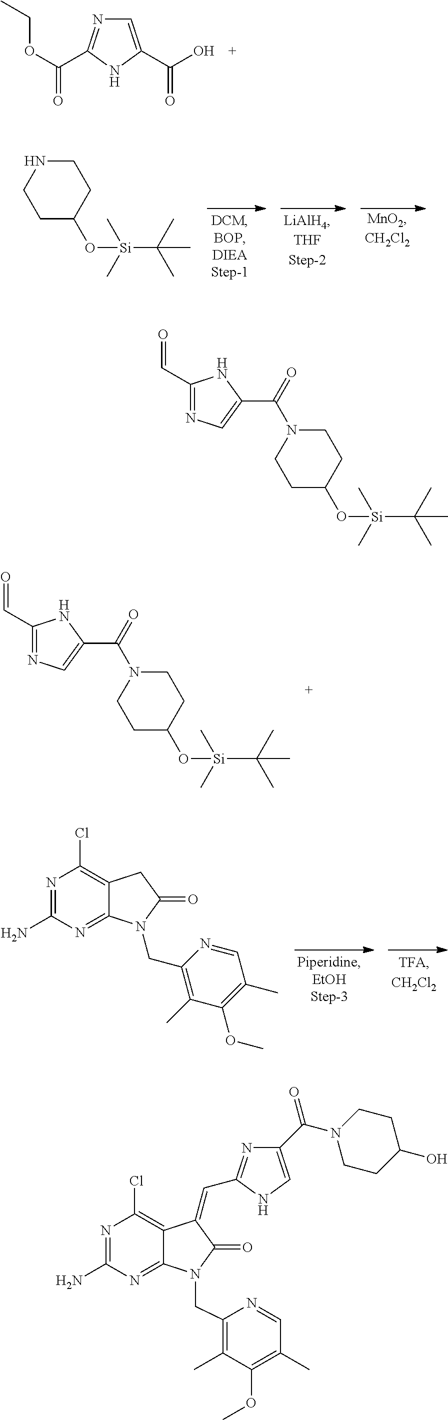

### Step-1: Ethyl 5-(4-(tert-butyldimethylsilyloxy)piperidine-1-carbonyl)-1H-imidazole-2-carboxylate

To a mixture of 2-(ethoxycarbonyl)-1H-imidazole-5-carboxylic acid (400 mg, 2.16 mmol) and 4-(tert-butyldimethylsilyloxy)piperidine (510 mg, 2.36 mmol) was added Bop (1.15 g, 2.60 mmol), DIEA (335 mg, 2.60 mmol), and CH₂Cl₂ (60 mL). The resulting solution was stirred for 12 hr at room temperature and then treated with 100 mL of H₂O. The resulting solution was extracted with 3×50 mL of dichloromethane. The organic layers were combined, washed with 100 mL of brine, dried over anhydrous sodium sulfate, and concentrated to dryness. The residue was purified onto a silica gel column with dichloromethane/methanol (50:1), resulted in 0.8 g (97%) of ethyl 5-(4-(tert-butyldimethylsilyloxy)piperidine-1-carbonyl)-1H-imidazole-2-carboxylate as a white solid.

### Step-2: 5-(4-(tert-Butyldimethylsilyloxy)piperidine-1-carbonyl)-1H-imidazole-2-carbaldehyde

To a solution of ethyl 5-(4-(tert-butyldimethylsilyloxy)piperidine-1-carbonyl)-1H-imidazole-2-carboxylate (800 mg, 2.09 mmol) THF (60 mL) was added LiAlH₄ (160 mg, 4.21 mmol). The resulting mixture was stirred for 8 hr at room temperature and then quenched by the addition of 0.5 mL of H₂O. The solids were filtered out and the filtrate was concentrated under vacuum, resulted in 0.46 g (67%) of (5-((4-(tert-butyldimethylsilyloxy)piperidin-1-yl)methyl)-1H-imidazol-2-yl)methanol as yellow oil. To it (0.46 g, 1.35 mmol) in CH₂Cl₂ (50 mL) was added MnO₂ (1 g, 11.49 mmol, 8.50 equiv). The resulting mixture was stirred for 12 hr at room temperature. The solids were filtered out and filtrate was concentrated under vacuum. The residue was purified onto a silica gel column with dichloromethane/methanol (15:1), resulted in 0.23 g (50%) of 5-(4-(tert-butyldimethylsilyloxy)piperidine-1-carbonyl)-1H-imidazole-2-carbaldehyde as yellow oil.

### Step-3: (Z)-2-Amino-4-chloro-5-((4-(4-hydroxypiperidine-1-carbonyl)-1H-imidazol-2-yl)methylene)-7-((4-methoxy-3,5-dimethylpyridin-2-yl)methyl)-5H-pyrrolo[2,3-d]pyrimidin-6(7H)-one

A mixture of 2-amino-4-chloro-7-((4-methoxy-3,5-dimethylpyridin-2-yl)methyl)-5H-pyrrolo[2,3-d]pyrimidin-6(7H)-one (200 mg, 0.60 mmol), 5-(4-(tert-butyldimethylsilyloxy)piperidine-1-carbonyl)-1H-imidazole-2-carbaldehyde (220 mg, 0.65 mmol), and piperidine (a drop) in EtOH (2 mL) was stirred for 12 hr at room temperature. The solids were collected by filtration, resulted in 188 mg (48%) of (Z)-2-amino-5-((4-(4-(tert-butyldimethylsilyloxy)piperidine-1-carbonyl)-1H-imidazol-2-yl)methylene)-4-chloro-7-((4-methoxy-3,5-dimethylpyridin-2-yl)methyl)-5H-pyrrolo[2,3-d]pyrimidin-6(7H)-one as a yellow solid. To the solid (180 mg, 0.29 mmol) was added 10 mL of THF:H₂O:CF₃COOH (1:1:3). The resulting solution was stirred for 30 min at room temperature and concentrated under vacuum. The residue was treated with 20 mL of C₂H₅OH. The solids were collected by filtration, resulted in 107 mg (73%) of the title compound as a yellow solid. ¹H NMR (300 MHz, DMSO-d₆) δ, 1.36 (2H, m), 1.79 (2H, m), 2.19 (3H, s), 2.30 (3H, s), 3.19 (1H, m), 3.59 (1H, m), 3.75 (1H, m), 3.80 (3H, s), 4.03 (1H, m), 4.44 (2H, m), 5.08 (2H, s), 7.57 (2H, s), 7.71 (1H, s), 7.83 (1H, s), 8.10 (1H, s). MS m/z: 539 [M+1]^{+·}.

### Example III-80

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(diethylamino)ethyl)-1H-pyrrole-2-carboxamide

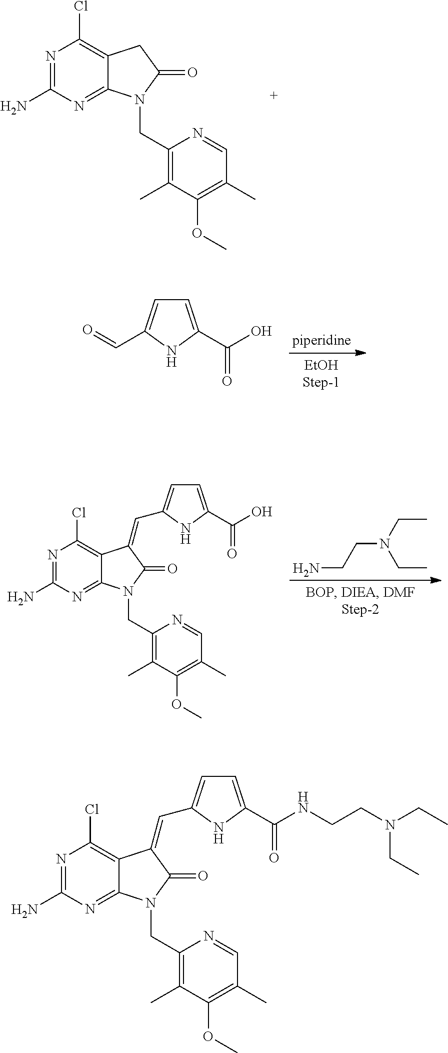

### Step-1: (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-1H-pyrrole-2-carboxylic acid

To the mixture of 2-amino-4-chloro-7-((4-methoxy-3,5-dimethylpyridin-2-yl)methyl)-5H-pyrrolo[2,3-d]pyrimidin-6(7H)-one (400 mg, 1.2 mmol) in EtOH (70 ml) was added 5-formyl-1H-pyrrole-2-carboxylic acid (167 mg, 1.2 mmol) and a drop of piperidine. The resulting solution was stirred for 8 hr at 60° C. The reaction mixture was filtered and the solid was washed with cold EtOH, and then dried under vacuum to afford (Z)-5-((2-amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-1H-pyrrole-2-carboxylic acid as a brown solid (400 mg, 73%).

### Step-2: (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(diethylamino)ethyl)-1H-pyrrole-2-carboxamide

To a mixture of above product (0.54 g, 1.12 mmol) in 5 ml DMF, HOBt (0.34 g, 2 mmol), EDC (0.43 g. 2.2 mmol), TEA (0.32 g, 3.4 mmol). After the mixture was stirred for 1 h, N1,N1-diethylethane-1,2-diamine (0.25 g, 2.2 mmol) was added and the mixture was stirred under N₂ at RT for overnight. Water was added to the reaction and the solution was exacted by EtOAc (100 ml×2). The combined organic layers were washed by saturated solution of NaHCO₃ (2×20 mL) and dried by Na₂SO₄. It was concentrated and the residue was purified using TLC to give the title compound as a brown solid (0.18 g, 27%). ¹H NMR (300 MHz, CDCl₃) δ 1.03 (6H, t), 2.18 (3H, S), 2.34 (3H, s), 2.56-2.66 (6H, m), 3.48 (2H, m), 3.77 (3H, 3H), 5.08 (2H, s), 5.26 (2H, s), 6.65 (2H, d), 6.87 (1H, s), 7.82 (1H, S), 7.02 (1H, s), 12.43 (1H, s). MS m/z: 553 [M+1]+·.

### Example III-81

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-morpholinoethyl)-1H-pyrrole-2-carboxamide

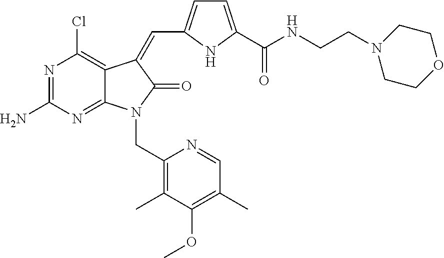

The title compound was prepared in 25% yield using the same protocols for the synthesis of example-80 with the corresponding amine replacing N1,N1-diethylethane-1,2-diamine. ¹H NMR (300 MHz, CDCl₃) δ 2.19 (3H, s), 2.35 (3H, s), 2.4-2.7 (7H, m), 3.55 (2H, b), 3.74-3.78 (7H, m), 5.09 (2H, s), 5.13 (2H, s), 6.72 (2H, d), 7.87 (1H, s), 8.02 (1H, s), 13.5 (1H, s). MS m/z: 567 [M+1]^{+·}.

### Example III-82

### (S,Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2,3-dihydroxypropyl)-1H-pyrrole-2-carboxamide

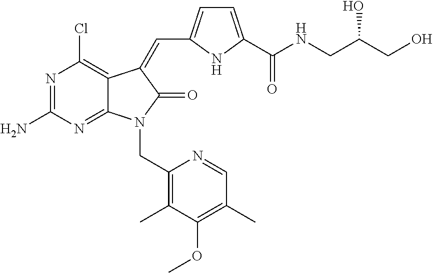

The title compound was prepared in 19% yield using the same protocols for the synthesis of example-80 with the corresponding amine replacing N1,N1-diethylethane-1,2-diamine. ¹H NMR (300 MHz, DMSO-d₆) δ 2.13 (3H, s), 2.29 (3H, m), 3.20 (2H, m), 3.65 (1H, m), 3.74 (3H, s), 4.54 (1H, m), 4.79 (1H, b), 5.03 (2H, s), 6.95 (1H, s), 7.10 (1H, s), 7.25 (2H, s), 7.78 (1H, s), 7.98 (1H, s), 8.40 (1H, t), 13.05 (1H, s). MS m/z: 528 [M+1]^{+·}.

### Example III-83

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4-methylpiperazin-1-yl)ethyl)-1H-pyrrole-2-carboxamide

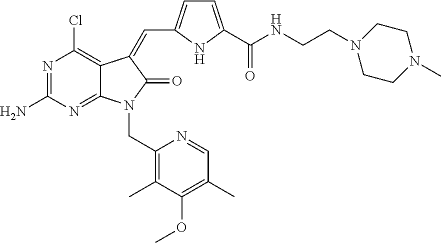

The title compound was prepared in 31% yield using the same protocols for the synthesis of example-80 with the corresponding amine replacing N1,N1-diethylethane-1,2-diamine. ¹H NMR (300 MHz, DMSO-d₆) δ 2.13 (3H, s), 2.15 (3H, s), 2.2-2.4 (12H, m), 3.74 (3H, s), 5.03 (2H, s), 6.94 (2H, d), 7.21 (2H, s), 7.77 (1H, s), 7.98 (1H, b), 8.39 (1H, s), 13.07 (1H, s). MS m/z: 580 [M+1]^{+·}.

### Example III-84

### (Z)-2-Amino-4-chloro-5-((5-(4-(2-hydroxyethyl)piperazine-1-carbonyl)-1H-pyrrol-2-yl)methylene)-7-((4-methoxy-3,5-dimethylpyridin-2-yl)methyl)-5H-pyrrolo[2,3-d]pyrimidin-6(7H)-one

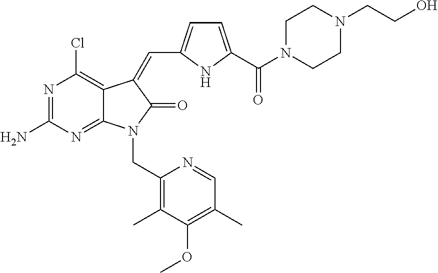

The title compound was prepared in 27% yield using the same protocols for the synthesis of example-80 with the corresponding amine replacing N1,N1-diethylethane-1,2-diamine. ¹H NMR (300 MHz, DMSO-d₆) δ 2.13 (3H, s), 2.28 (1H, m), 2.3-2.4 (6H, m), 2.48 (2H, q), 3.66 (4H, b), 4.41 (1H, t), 5.02 (2H, s), 6.70 (1H, t), 6.98 (1H, s), 7.27 (2H, s), 7.78 (1H, s), 7.98 (1H, s), 13.08 (1H, s). MS m/z: 567 [M+1]^{+·}.

### Example III-85

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4-hydroxypiperidin-1-yl)ethyl)-1H-pyrrole-2-carboxamide

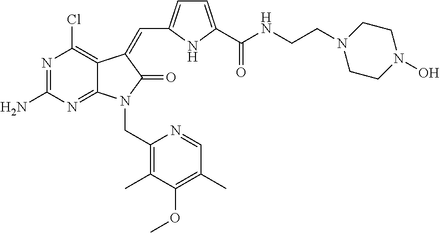

The title compound was prepared in 19% yield using the same protocols for the synthesis of example-80 with the corresponding amine replacing N1,N1-diethylethane-1,2-diamine. ¹H NMR (300 MHz, DMSO-d₆) δ 1.6-2.0 (4H, m), 2.14 (3H, s), 2.29 (3H, s), 2.9-3.3 (5H, m), 3.5-3.7 (4H, m), 3.74 (3H, s), 5.03 (2H, s), 6.98 (1H, s), 7.10 (s, 1H), 7.28 (2H, m), 7.76 (1H, s), 7.98 (1H, s), 8.92 (1H, d), 10.07 (1H, b), 13.11 (1H, s). MS m/z: 581 [M+1]^{+·}.

### Example III-86

### (Z)-2-Amino-4-chloro-545-(3-(diethylamino)propyl)-1H-pyrrol-2-yl)methylene)-7-((4-methoxy-3,5-dimethylpyridin-2-yl)methyl)-5H-pyrrolo[2,3-d]pyrimidin-6(7H)-one

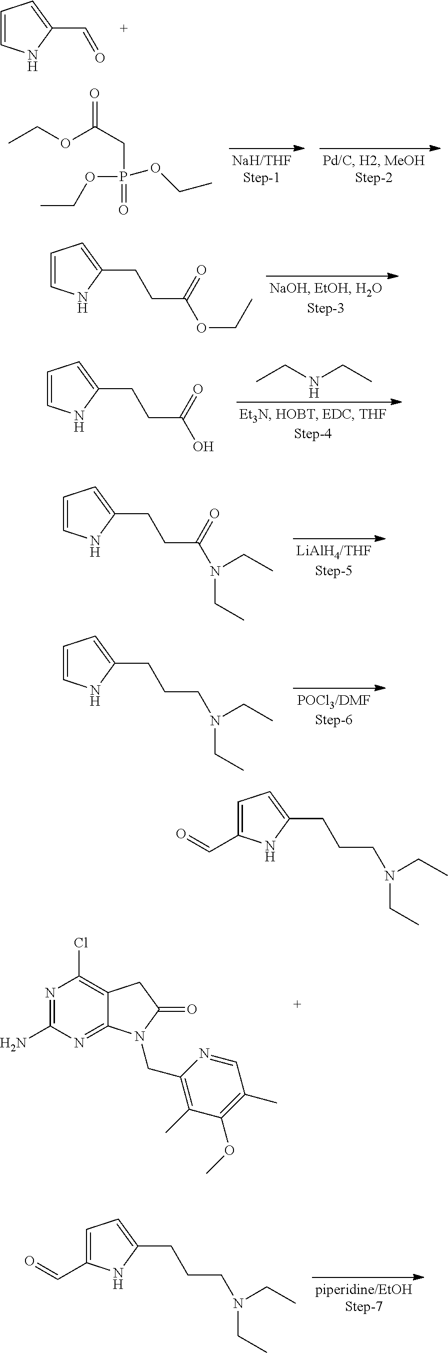

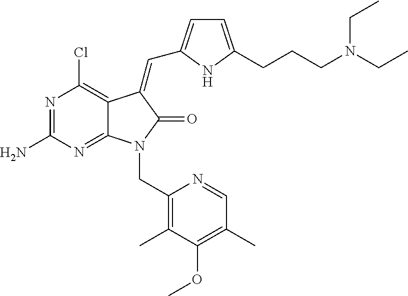

### Step-1: Ethyl 3-(1H-pyrrol-2-yl)acrylate

To a solution of ethyl 2-(diethoxyphosphoryl)acetate (5.2 g, 23.19 mmol) in 20 mL of THF under N₂ was added NaH (840 mg, 35.00 mmol) and 1H-pyrrole-2-carbaldehyde (2.0 g, 21.03 mmol) with stirring at 0° C. The resulting solution was stirred for 0.5 hr at 0° C. The reaction was treated with 10% of NH₄Cl/H₂O (200 mL) and extracted with 2×50 mL of ethyl acetate. The organic layers were combined, dried over anhydrous magnesium sulfate, and concentrated under vacuum. The residue was purified using a silica gel column with ethyl acetate/petroleum ether (1:20), resulted in 1 g (27%) of ethyl 3-(1H-pyrrol-2-yl)acrylate as a white solid.

### Step-2: Ethyl 3-(1H-pyrrol-2-yl)propanoate

A mixture of (E)-ethyl 3-(1H-pyrrol-2-yl)acrylate (8 g, 48.43 mmol) in MeOH (50 mL) and Pd/C (0.8 g) was stirred under H₂ at room temperature for overnight. The solids were collected by filtration. The resulting mixture was concentrated under vacuum, resulted in 8 g (94%) of ethyl 3-(1H-pyrrol-2-yl)propanoate as yellow oil.

### Step-3: 3-(1H-Pyrrol-2-yl)propanoic acid

To a solution of ethyl 3-(1H-pyrrol-2-yl) propanoate (8 g, 47.85 mmol) in EtOH/H₂O (80/40 mL) was added NaOH (3.8 g, 95.00 mmol) in portions at 0-5° C. The resulting solution was stirred for 5 hr at room temperature, monitored by LCMS, and then concentrated under vacuum. After the pH value of the solution was adjusted to 3-4 with HCl (10%), it was extracted with 3×100 mL of ethyl acetate. The combined organic layers were dried over anhydrous magnesium sulfate and concentrated under vacuum, resulted in 7 g (95%) of 3-(1H-pyrrol-2-yl)propanoic acid as a white solid.

### Step-4: N,N-Diethyl-3-(1H-pyrrol-2-yl)propanamide

To a mixture of 3-(1H-pyrrol-2-yl)propanoic acid (5.0 g, 35.92 mmol) in THF (100 mL) and diethylamine (3.0 g, 41.04 mmol) at 0° C. was added HOBT (7.25 g, 53.66 mmol), triethylamine (6.5 g, 64.23 mmol), and EDC (8.25 g, 43.04 mmol). The resulting solution was stirred for overnight at room temperature. The reaction was treated with 50 mL of water, extracted with 3×100 mL of ethyl acetate. The combined organic layers were washed with 2×50 mL of brine, dried over anhydrous magnesium sulfate, and concentrated under vacuum. The residue was purified onto a silica gel column with dichloromethane/methanol (20:1), resulted in 5.0 g (70%) of N,N-diethyl-3-(1H-pyrrol-2-yl)propanamide as yellow oil. ¹H NMR (400 MHz, CDCl₃) δ 1.14 (6H, m), 2.63 (2H, m), 2.97 (2H, m), 3.28 (2H, m), 3.42 (2H, m), 5.90 (s1H, s), 6.08 (1H, s), 6.66 (1H, s), 9.29 (1H, s), MS m/z: 195 [M+1]^{+·}.

### Step-5: N,N-Diethyl-3-(1H-pyrrol-2-yl)propan-1-amine

To a solution of N,N-diethyl-3-(1H-pyrrol-2-yl)propanamide (3.0 g, 15.44 mmol) in THF (30 mL) under N₂ was added LiAlH₄ (3.0 g, 78.95 mmol). The mixture was stirred for 2 hr at room temperature and then treated with 20 mL of ethyl acetate and 20 mL of water. The solids were filtered out and the filtrate was extracted with 3×100 mL of ethyl acetate. The organic layers were combined, dried over anhydrous magnesium sulfate, and concentrated under vacuum. The residue was purified onto a silica gel column with dichloromethane/methanol (10:1), resulted in 2.1 g (72%) of N,N-diethyl-3-(1H-pyrrol-2-yl)propan-1-amine as brown oil. NMR (400 MHz, CDCl₃) δ 1.05 (6H, m), 1.80 (2H, m), 2.48˜2.68 (6H, m), 2.71 (2H, m), 5.88 (1H, s), 6.12 (1H, s), 6.64 (1H, s), 9.66 (1H, s), MS m/z: 181 [M+1]+·.

### Step-6: 5-(3-(Diethylamino)propyl)-1H-pyrrole-2-carbaldehyde

A mixture of DMF (2.9 g, 33.33 mmol) and POCl₃ (1.85 g, 12.25 mmol) at 0° C. was added a solution of N,N-diethyl-3-(1H-pyrrol-2-yl)propan-1-amine (2 g, 11.11 mmol) in DMF (20 mL) dropwise. The resulting solution was stirred for 2 hr at room temperature. The reaction was then quenched by the addition of 50 mL of water and the pH value of the solution was adjusted to 11 with NaOH (1 N). The resulting solution was extracted with 4×30 mL of dichloromethane. The organic layers were combined and dried over anhydrous sodium sulfate. The residue was purified onto a silica gel column with dichloromethane/methanol (50:1-10:1), resulted in 1 g (41%) of 5-(3-(diethylamino)propyl)-1H-pyrrole-2-carbaldehyde as yellow oil.

### Step-7: (Z)-2-Amino-4-chloro-5-((5-(3-(diethylamino)propyl)-1H-pyrrol-2-yl)methylene)-7-((4-methoxy-3,5-dimethylpyridin-2-yl)methyl)-5H-pyrrolo[2,3-d]pyrimidin-6(7H)-one

To a mixture of 2-amino-4-chloro-7-((4-methoxy-3,5-dimethylpyridin-2-yl)methyl)-5H-pyrrolo[2,3-d]pyrimidin-6(7H)-one (334 mg, 1.00 mmol) and 5-(3-(diethylamino)propyl)-1H-pyrrole-2-carbaldehyde (230 mg, 1.11 mmol) in EtOH (30 mL) was added piperidine (a drop). The resulting solution was stirred for overnight at room temperature. The resulting mixture was concentrated under vacuum. The residue was purified onto a silica gel column with dichloromethane/methanol (50:1˜30:1), resulted in 200 mg (38%) of the title compound a brown solid. ¹H NMR (400 MHz, CDCl₃) δ 1.06 (6H, t), 1.92 (2H, m), 2.21 (3H, s), 2.37 (3H, s), 2.60 (6H, m), 2.74 (2H, t), 3.79 (3H, s), 4.99 (2H, s), 5.11 (2H, s), 6.18 (1H, s), 6.75 (1H, s), 7.88 (1H, s), 8.09 (1H, s), 12.86 (1H, s). MS m/z: 524 [M+1]^{+·}.

### Example III-87

### (Z)-3-(5-42-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-1H-pyrrol-2-yl)-N,N-diethylpropanamide

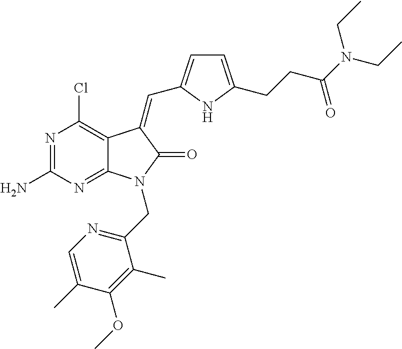

The title compounds were prepared in 77% yield using the same protocols for the synthesis of example-1 with the corresponding aldehydes replacing 1H-pyrrole-2-carbaldehyde. ¹H NMR (400 MHz, DMSO-d₆) δ 0.94 (3H, t), 1.01 (3H, t), 2.14 (3H, s), 2.29 (3H, s), 2.63 (2H, t), 2.91 (2H, t), 3.23 (4H, m), 3.75 (3H, s), 5.03 (2H, s), 6.18 (1H, t), 6.90 (1H, s), 7.06 (2H, s), 7.72 (1H, s), 7.99 (1H, s), 12.62 (1H, s). MS m/z: 538 [M+1]^{+·}.

### Example III-88

### (Z)-Ethyl 3-(5-((2-amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-1H-pyrrol-2-yl)propanoate

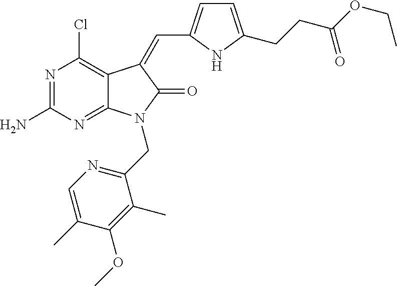

The title compounds were prepared in 73% yield using the same protocols for the synthesis of example-1 with the corresponding aldehydes replacing 1H-pyrrole-2-carbaldehyde. NMR (400 MHz, DMSO-d₆) δ 1.11 (3H, t), 2.14 (3H, s), 2.29 (3H, s), 2.65 (2H, t), 2.93 (2H, t), 3.75 (3H, s), 4.02 (2H, m), 5.04 (2H, s), 6.18 (1H, t), 6.91 (1H, s), 7.08 (2H, s), 7.73 (1H, s), 7.99 (11I, s), 12.66 (1H, s). MS m/z: 511 [M+1]^{+·}.

### Example III-89

### 2-Amino-7-((4-methoxy-3,5-dimethylpyridin-2-yl)methyl)-4-(trifluoromethyl)-5H-pyrrolo[2,3-d]pyrimidin-6(7H)-one

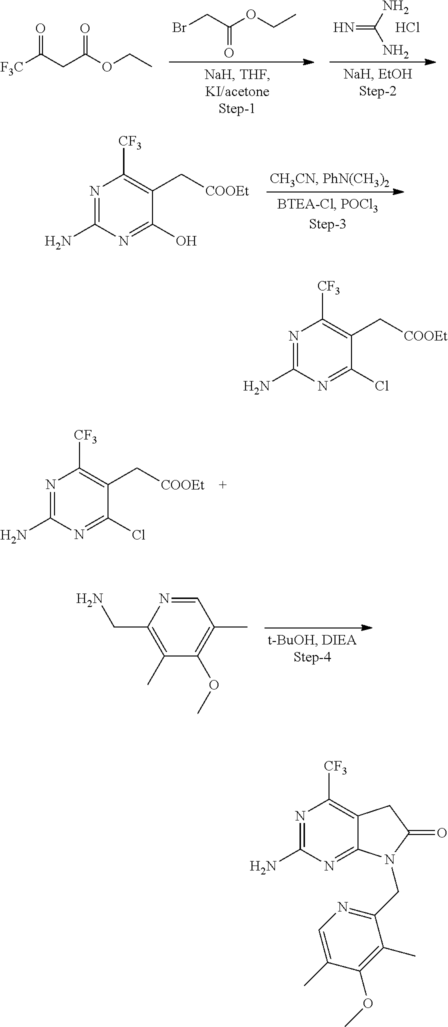

### Step-1: Diethyl 2-(2,2,2-trifluoroacetyl)succinate

To a mixture of NaH (8.6 g, 215.00 mmol, 60%) in THF (120 mL) was added ethyl 4,4,4-trifluoro-3-oxobutanoate (40 g, 217.39 mmol) dropwise at 0° C. The resulting mixture was stirred for 1 hr at room temperature and then concentrated under vacuum. The residue was treated with KI (3.6 g, 21.69 mmol) and 320 mL of acetone, to which was added at 0° C. ethyl 2-bromoacetate (40 g, 239.52 mmol) dropwise with stirring. The reaction mixture was heated to reflux for 36 hrs and then quenched by the addition of 500 mL of ice water. The resulting solution was extracted with 3×200 mL of ethyl acetate. The organic layers were combined, dried over Na₂SO₄, and concentrated under vacuum, resulted in 32 g (55%) of diethyl 2-(2,2,2-trifluoroacetyl)succinate as yellow oil.

### Step-2: Ethyl 2-(2-amino-4-hydroxy-6-(trifluromethyl)pyrimidin-5-yl)acetate

To a solution of NaH (15 g, 375.00 mmol, 60%) in EtOH (250 mL) was added guanidine hydrochloride (35 g, 368.42 mmol) in several batches at 0° C. After 30 minutes, the solids were filtered out. To the filtrate was added a solution of diethyl 2-(2,2,2-trifluoroacetyl)succinate (100 g, 370.37 mmol) in EtOH (100 mL). The resulting solution was refluxed for 3 hr. The mixture was concentrated under vacuum and was treated with 500 mL of ice water. The solids were collected by filtration, resulted in 80 g (82%) of ethyl 2-(2-amino-4-hydroxy-6-(trifluoromethyl)pyrimidin-5-yl)acetate as a yellow solid.

### Step-3: Ethyl 2-(2-amino-4-chloro-6-(trifluoromethyl)pyrimidin-5-yl)acetate

To a solution of ethyl 2-(2-amino-4-hydroxy-6-(trifluoromethyl)pyrimidin-5-yl)acetate (10 g, 37.72 mmol) in CH₃CN (mL) was added PhN(CH₃)₂ (9.13 g, 60.07 mmol), BTEA-Cl (43.13 g, 190.00 mmol), and POCl₃ (28.88 g, 190.00 mmol). The resulting solution was stirred for 2 hr at 60° C. After the reaction mixture was cooled, the solids were collected by filtration, resulted in 7.2 g (67%) of ethyl 2-(2-amino-4-chloro-6-(trifluoromethyl)pyrimidin-5-yl)acetate as a white solid.

### Step-4: 2-Amino-7-((4-methoxy-3,5-dimethylpyridin-2-yl)methyl)-4-(trifluoromethyl)-5H-pyrrolo[2,3-d]pyrimidin-6(7H)-one

To a solution of ethyl 2-(2-amino-4-chloro-6-(trifluoromethyl)pyrimidin-5-yl)acetate (5 g, 17.63 mmol) in t-BuOH (50 mL) under N₂ was added (4-methoxy-3,5-dimethylpyridin-2-yl)methanamine (5.81 g, 34.96 mmol) and DIEA (6.84 g, 53.02 mmol). The resulting solution was stirred for 12 hr at 100° C. After the reaction was cooled, the solids were collected by filtration, resulted in 3.7 g (57%) of the title compound as a white solid. ¹H NMR (300 MHz, CDCl₃) δ 2.22 (3H, s), 2.33 (3H, s), 3.71 (2H, t), 3.79 (3H, s), 4.97 (2H, s), 5.23 (2H, s), 8.10 (1H, s). MS m/z: 368 [M+1]^{+·}.

### Example III-90

### (Z)-5-((2-Amino-7-((4-methoxy-3,5-dimethylpyridin-2-yl)methyl)-6-oxo-4-(trifluoromethyl)-6,7-dihydro-5H-pyrrolo[2,3-d]pyrimidin-5-ylidene)methyl)-1H-pyrrole-3-carboxylic acid

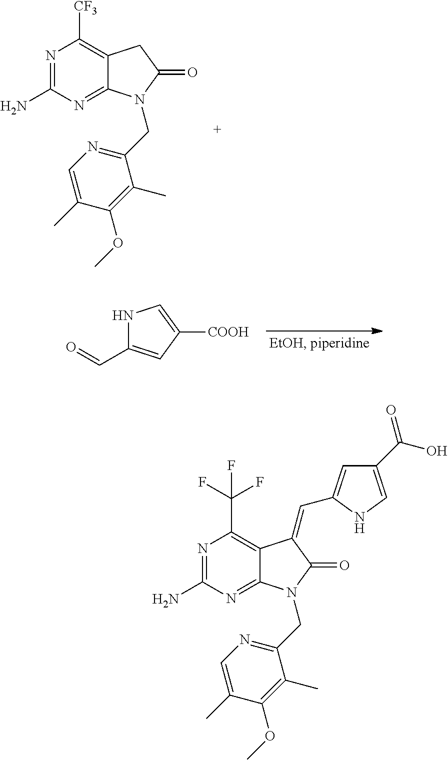

A mixture of 2-amino-7-((4-methoxy-3,5-dimethylpyridin-2-yl)methyl)-4-(trifluoromethyl)-5H-pyrrolo[2,3-d]pyrimidin-6(7H)-one (1 g, 2.72 mmol) in EtOH (20 mL), 5-formyl-1H-pyrrole-3-carboxylic acid (420 mg, 3.02 mmol), and piperidine (a drop) was stirred for 12 hr at room temperature. The solids were filtered and washed with 3×10 mL of Et₂O, resulted in 1.3 g (95%) of the title compound as a yellow solid. ¹H NMR (300 MHz, DMSO-d₆) δ 2.14 (3H, s), 2.30 (3H, s), 3.75 (1H, s), 5.06 (2H, s), 7.09 (1H, s), 7.38 (3H, s), 7.76 (1H, t), 8.01 (1H, s), 12.21 (1H, s, br), 12.90 (1H, s). MS m/z: 489 [M+1]^{+·}.

### Example III-91

### (Z)-5-((2-Amino-7-((4-methoxy-3,5-dimethylpyridin-2-yl)methyl)-6-oxo-4-(trifluoromethyl)-6,7-dihydro-5H-pyrrolo[2,3-d]pyrimidin-5-ylidene)methyl)-N-(2-(diethylamino)ethyl)-1H-pyrrole-3-carboxamide

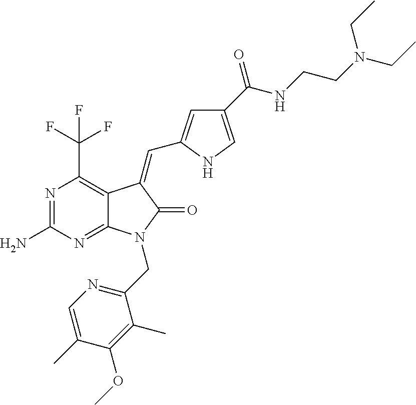

The title compound was prepared in 13% yield using the same protocols for the synthesis of example-16. ¹H NMR (400 MHz, DMSO-d₆) δ 1.08 (6H, t), 2.14 (3H, s), 2.30 (3H, s), 2.84 (6H, m), 3.39 (2H, m), 3.75 (3H, s), 5.07 (2H, s), 7.30-7.38 (4H, m), 7.75 (1H, s), 8.00 (1H, s), 8.31 (1H, m), 12.79 (1H, s). MS m/z: 587 [M+1]^{+·}.

### Example III-92

### (Z)-5-((2-Amino-7-((4-methoxy-3,5-dimethylpyridin-2-yl)methyl)-6-oxo-4-(trifluoromethyl)-6,7-dihydro-5H-pyrrolo[2,3-d]pyrimidin-5-ylidene)methyl)-2,4-dimethyl-N-(2-(pyrrolidin-1-yl)ethyl)-1H-pyrrole-3-carboxamide

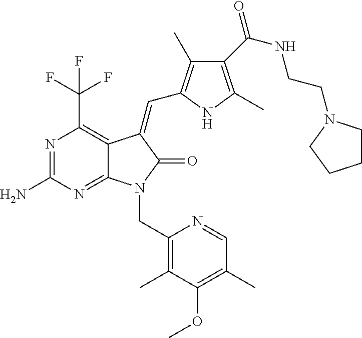

The title compounds were prepared using the same protocols for the synthesis of example-90. ¹H NMR (400 MHz, DMSO-d₆) δ 1.91 (4H, b), 2.14 (3H, s), 2.29 (4H, m), 2.35 (3H, s), 2.45 (3H, s), 3.22 (6H, m), 3.51 (2H, m), 3.75 (3H, s), 5.07 (2H, s), 7.23 (2H, s), 7.44 (1H, s), 7.80 (1H, t), 7.99 (1H, s), 12.90 (1H, s).

### Example III-93

### (Z)-2-Amino-4-chloro-7-((4-methoxy-3,5-dimethylpyridin-2-yl)methyl)-5-((4-(2-morpholinoacetyl)-1H-pyrrol-2-yl)methylene)-5H-pyrrolo[2,3-d]pyrimidin-6(7H)-one

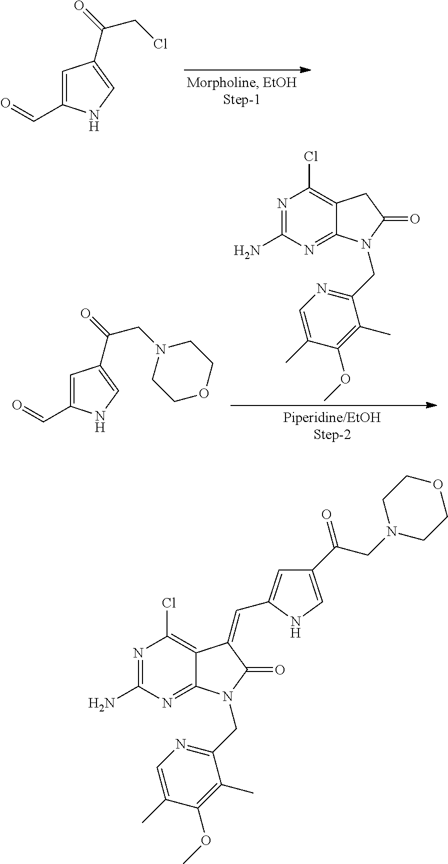

### Step-1: 4-(2-Morpholinoacetyl)-1H-pyrrole-2-carbaldehyde

To a solution of 4-(2-chloroacetyl)-1H-pyrrole-2-carbaldehyde (200 mg, 1.17 mmol) in EtOH (30 mL) was added morpholine (153 g, 1.76 mol). The resulting solution was refluxed for overnight. The resulting mixture was concentrated under vacuum and treated with 30 mL of EtOAc. After the resulting mixture was washed with 3×20 mL of H₂O and 20 mL of NaHCO₃, it was dried over anhydrous sodium sulfate and concentrated under vacuum, resulted in 200 mg (77%) of 4-(2-morpholinoacetyl)-1H-pyrrole-2-carbaldehyde as brown oil.

### Step-2: (Z)-2-Amino-4-chloro-7-((4-methoxy-3,5-dimethylpyridin-2-yl)methyl)-5-((4-(2-morpholinoacetyl)-1H-pyrrol-2-yl)methylene)-5H-pyrrolo[2,3-d]pyrimidin-6(7H)-one

To a mixture of 2-amino-4-chloro-7-((4-methoxy-3,5-dimethylpyridin-2-yl)methyl)-5H-pyrrolo[2,3-d]pyrimidin-6(7H)-one (255 mg, 0.77 mmol) in EtOH (20 mL) was added 4-(2-morpholinoacetyl)-1H-pyrrole-2-carbaldehyde (170 g, 765.77 mmol) and piperidine (a drop). The resulting mixture was stirred for overnight at room temperature. The solids were collected by filtration and washed with 3×30 mL of EtOH and 2×20 mL of ethyl acetate, resulted in 300 mg (69%) of the title compound as a yellow solid. ¹H NMR (300 MHz, DMSO-d₆) δ 2.14 (3H, s), 2.29 (3H, s), 2.48 (4H, m), 3.58 (6H, m), 3.75 (3H, s), 5.03 (2H, s), 7.27 (2H, s), 7.46 (1H, s), 7.80 (1H, s), 8.01 (2H, m), 12.85 (1H, s). MS m/z: 538 [M+1]^{+·}.

### Example III-94

### (Z)-2-Amino-4-chloro-7-((4-methoxy-3,5-dimethylpyridin-2-yl)methyl)-5-(4-(2-(4-(trifluoromethyl)piperidin-1-yl)acetyl)-1H-pyrrol-2-yl)methylene)-5H-pyrrolo[2,3-d]pyrimidin-6(7H)-one

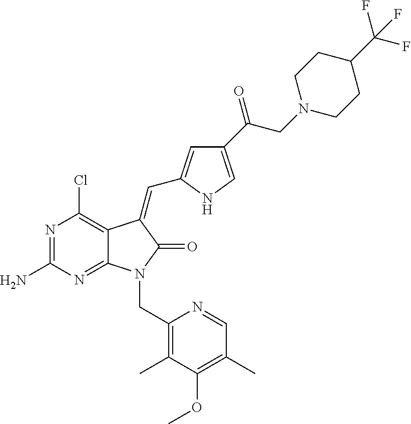

The title compound was prepared in 86% yield using the same protocols for the synthesis of example-93 with the corresponding amine replacing morpholine. ¹H NMR (400 MHz, DMSO-d₆) δ 1.46 (2H, m), 1.49 (2H, d), 2.1-2.3 (9H, m), 2.95 (2H, d), 3.56 (2H, s), 3.73 (3H, s), 5.02 (2H, s), 7.26 (2H, s), 7.43 (1H, s), 7.79 (1H, s), 8.0 (1H, s), 8.02 (1H, s), 12.83 (1H, s). MS m/z: [M+1]^{+·}.

### Example III-95

### (Z)-2-Amino-4-chloro-5-((4-(2-(diethylamino)acetyl)-1H-pyrrol-2-yl)methylene)-7-((4-methoxy-3,5-dimethylpyridin-2-yl)methyl)-5H-pyrrolo[2,3-d]pyrimidin-6(7H)-one

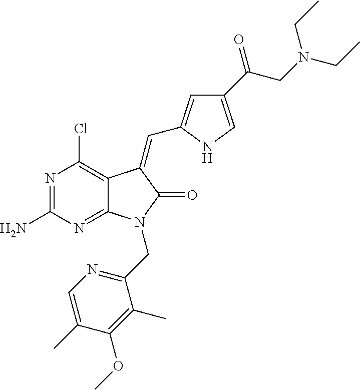

The title compound was prepared in 42% yield using the same protocols for the synthesis of example-93 with the corresponding amine replacing morpholine. ¹H NMR (300 MHz, DMSO-d₆) δ 0.97 (6H, t), 2.14 (3H, s), 2.29 (3H, s), 2.54 (4H, m), 3.60 (2H, s), 3.74 (3H, s), 5.02 (2H, s), 7.26 (2H, s), 7.44 (1H, s), 7.79 (1H, s), 8.01 (2H, m), 12.83 (1H, s). MS m/z: 524 [M+1]^{+·}.

### Example III-96

### (Z)-2-Amino-4-chloro-7-((4-methoxy-3,5-dimethylpyridin-2-yl)methyl)-5-((4-(2-morpholinoacetyl)-1H-pyrrol-2-yl)methylene)-5H-pyrrolo[2,3-d]pyrimidin-6(7H)-one

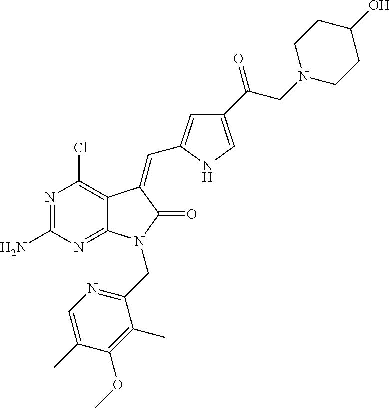

The title compound was prepared in 80% yield using the same protocols for the synthesis of example-93 with the corresponding amine replacing morpholine. NMR (400 MHz, DMSO-d₆) δ 1.68 (2H, m), 1.41 (2H, m), 2.17 (4H, m), 2.28 (4H, m), 2.72 (2H, m), 3.42 (2H, m), 3.74 (3H, s), 4.54 (2H, b), 5.02 (2H, s), 7.26 (3H, m), 7.43 (1H, s), 7.79 (1H, s), 8.00 (2H, d), 12.83 (1H, s). MS m/z: 562 [M+1]^{+·}.

### Example III-97

### (Z)-2-Amino-4-chloro-5-((4-(2-(4,4-difluoropiperidin-1-yl)acetyl)-1H-pyrrol-2-yl)methylene)-7-((4-methoxy-3,5-dimethylpyridin-2-yl)methyl)-5H-pyrrolo[2,3-d]pyrimidin-6(7H)-one

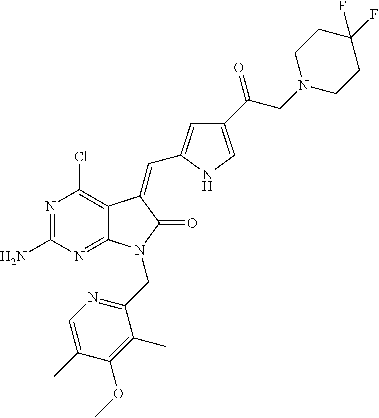

The title compound was prepared in 67% yield using the same protocols for the synthesis of example-93 with the corresponding amine replacing morpholine. ¹H NMR (400 MHz, DMSO-d₆) δ 1.96 (4H, m), 2.14 (3H, s), 2.29 (3H, s), 2.64 (4H, t), 3.68 (2H, s), 3.74 (3H, s), 5.03 (2H, s), 7.25 (2H, s), 7.45 (1H, b), 7.81 (1H, s), 8.02 (2H, m), 12.80 (1H, s). MS m/z: [M+1]^{+·}.

### Example III-98

### (Z)-2-Amino-4-chloro-5-((4-(2-(3,3-difluoropyrrolidin-1-yl)acetyl)-1H-pyrrol-2-yl)methylene)-7-((4-methoxy-3,5-dimethylpyridin-2-yl)methyl)-5H-pyrrolo[2,3-d]pyrimidin-6(7H)-one

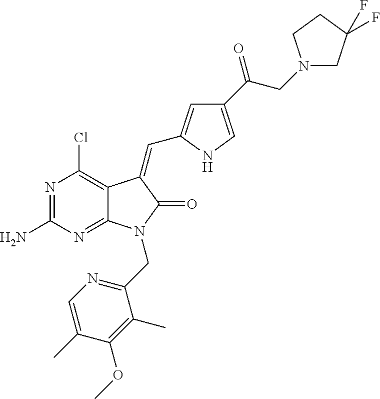

The title compound was prepared in 66% yield using the same protocols for the synthesis of example-93 with the corresponding amine replacing morpholine. ¹H NMR (400 MHz, DMSO-d₆) δ 1.65 (2H, m), 1.88 (2H, m), 2.14 (3H, s), 2.29 (3H, s), 2.85 (2H, t), 3.72 (2H, s), 3.74 (3H, s), 5.03 (2H, s), 7.25 (2H, s), 7.44 (1H, d), 7.79 (1H, s), 7.96 (1H, m), 8.01 (1H, s), 12.80 (1H, s).

### Example III-99

### (Z)-2-Amino-4-chloro-5-((4-(2-(2-(diethylamino)ethoxy)acetyl)-1H-pyrrol-2-yl)methylene)-7-((4-methoxy-3,5-dimethylpyridin-2-yl)methyl)-5H-pyrrolo[2,3-d]pyrimidin-6(7H)-one

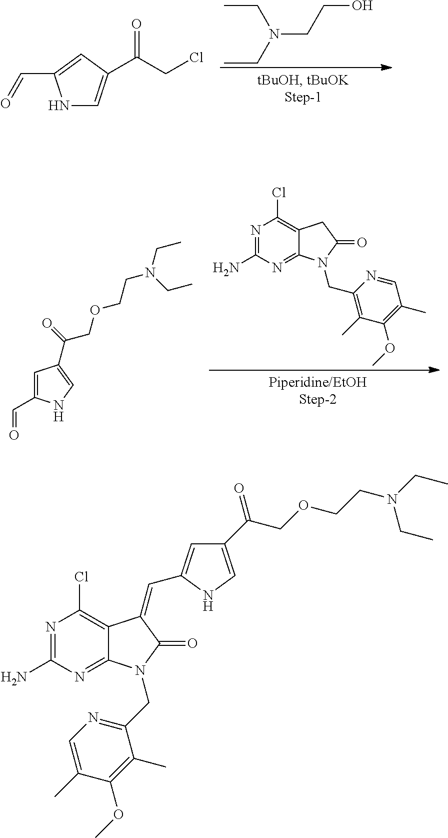

### Step-1: 4-(2-(2-(Diethylamino)ethoxy)acetyl)-1H-pyrrole-2-carbaldehyde

To a mixture of potassium 2-methylpropan-2-olate (1.96 g, 17.50 mmol) and 2-(diethylamino)ethanol (10 mL) was added 4-(2-chloroacetyl)-1H-pyrrole-2-carbaldehyde (1 g, 5.85 mmol). The resulting solution was stirred for 1 hr at 60° C. The reaction was then quenched by the addition of 10 mL of water. After the resulting solution was extracted with 3×20 mL of ethyl acetate, the organic layers were combined and concentrated under vacuum, resulted in 200 mg (14%) of 4-(2-(2-(diethylamino)ethoxy)acetyl)-1H-pyrrole-2-carbaldehyde as yellow oil.

### Step-2: (Z)-2-Amino-4-chloro-5-((4-(2-(2-(diethylamino)ethoxy)acetyl)-1H-pyrrol-2-yl)methylene)-7-((4-methoxy-3,5-dimethylpyridin-2-yl)methyl)-5H-pyrrolo[2,3-d]pyrimidin-6(7H)-one

A mixture of 2-amino-4-chloro-7-((4-methoxy-3,5-dimethylpyridin-2-yl)methyl)-5H-pyrrolo[2,3-d]pyrimidin-6(7H)-one (300 mg, 0.90 mmol), 4-(2-(2-(diethylamino)ethoxy)acetyl)-1H-pyrrole-2-carbaldehyde (360 mg, 1.43 mmol), and piperidine (a drop) in EtOH (20 mL) was stirred for overnight at room temperature. The solids were collected by filtration and washed with 10 mL of EtOH, resulted in 385.9 mg (72%) of the title compound as a yellow solid. ¹H NMR (400 MHz, CDCl₃) δ 1.05 (6H, t), 2.21 (3H, s), 2.36 (3H, s), 2.61 (4H, m), 2.75 (2H, t), 3.67 (2H, t), 3.79 (3H, s), 4.51 (2H, s), 5.09 (2H, s), 5.18 (2H, s), 7.24 (1H, s), 7.81 (1H, m), 7.90 (1H, s), 8.08 (1H, s), 13.29 (1H, s). MS m/z: 568 [M+1]^{+·}.

### Example III-100

### (Z)-2-Amino-4-chloro-7-((4-methoxy-3,5-dimethylpyridin-2-yl)methyl)-5-(4-(2-(2-morpholinoethoxy)acetyl)-1H-pyrrol-2-yl)methylene)-5H-pyrrolo[2,3-d]pyrimidin-6(7H)-one

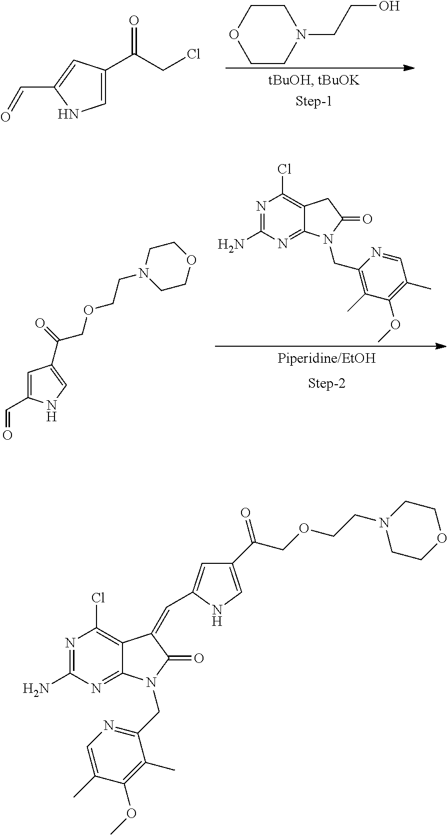

### Step-1

### 4-(2-(2-Morpholinoethoxy)acetyl)-1H-pyrrole-2-carbaldehyde

After a mixture of 2-morpholinoethanol (10 mL) and potassium 2-methylpropan-2-olate (1.96 g, 17.50 mmol) was stirred for 10 mins, to it was added 4-(2-chloroacetyl)-1H-pyrrole-2-carbaldehyde (1 g, 5.85 mmol). The resulting solution was stirred for 1 hr at room temperature and treated with 20 mL of ethyl acetate. The solids were filtered out and washed with 3×20 mL of water. The filtrates were extracted with ethyl acetate. The organic layer was dried over anhydrous sodium sulfate and concentrated under vacuum, resulted in 0.5 g (32%) of 4-(2-(2-morpholinoethoxy)acetyl)-1H-pyrrole-2-carbaldehyde as yellow oil.

### Step-2: (Z)-2-Amino-4-chloro-7-((4-methoxy-3,5-dimethylpyridin-2-yl)methyl)-5-((4-(2-(2-morpholinoethoxy)acetyl)-1H-pyrrol-2-yl)methylene)-5H-pyrrolo[2,3-d]pyrimidin-6(7H)-one

To a mixture of 2-amino-4-chloro-7-((4-methoxy-3,5-dimethylpyridin-2-yl)methyl)-5H-pyrrolo[2,3-d]pyrimidin-6(7H)-one (350 mg, 1.05 mol, 1.00 equiv) in ethanol (20 mol) was added 4-(2-(2-morpholinoethoxy)acetyl)-1H-pyrrole-2-carbaldehyde (699 mg, 2.63 mol) and piperidine (a drop). The resulting mixture was stirred for 12 hr at room temperature. The solids were collected by filtration, resulted in 320 mg (52%) of the title compound as a yellow solid. ¹H NMR (400 MHz, CDCl₃) δ 2.21 (3H, s), 2.37 (3H, s), 2.55-2.78 (6H, m), 3.79 (9H, m), 4.54 (2H, s), 5.10 (2H, s), 5.15 (2H, s), 7.21 (1H, s), 7.78 (1H, d), 7.90 (1H, s), 8.08 (1H, s), 13.33 (1H, s). MS m/z: 582 [M+1]^{+·}.

### Example III-101

### (Z)-2-Amino-4-chloro-7-((4-methoxy-3,5-dimethylpyridin-2-yl)methyl)-5-β4-(2-(2-(pyrrolidin-1-yl)ethoxy)acetyl)-1H-pyrrol-2-yl)methylene)-5H-pyrrolo[2,3-d]pyrimidin-6(7H)-one

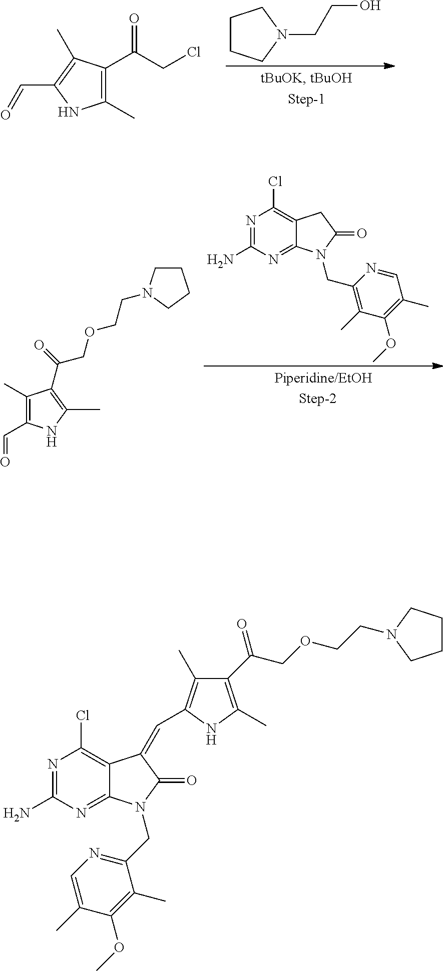

### Step-1: 3,5-Dimethyl-4-(2-(2-(pyrrolidin-1-yl)ethoxy)acetyl)-1H-pyrrole-2-carbaldehyde

To 2-(pyrrolidin-1-yl)ethanol (1.24 g, 10.78 mmol) was added t-BuOH (10 mL) and then t-BuOK (1.21 g, 10.80 mmol). The mixture was stirred for 10 min. To it was added 4-(2-chloroacetyl)-3,5-dimethyl-1H-pyrrole-2-carbaldehyde (1 g, 5.03 mmol) at 0° C. The resulting solution was stirred for 2 hr at room temperature and then quenched by the addition of 10 mL of water. A solution of HCl (IN) was employed to adjust the pH to 7-8. The resulting mixture was concentrated under vacuum. The residue was purified onto a silica gel column with dichloromethane/methanol (20:1), resulted in 1.3 g (93%) of 3,5-dimethyl-4-(2-(2-(pyrrolidin-1-yl)ethoxy)acetyl)-1H-pyrrole-2-carbaldehyde as a yellow solid.

### Step-2: (Z)-2-Amino-4-chloro-7-((4-methoxy-3,5-dimethylpyridin-2-yl)methyl)-5-((4-(2-(2-(pyrrolidin-1-yl)ethoxy)acetyl)-1H-pyrrol-2-yl)methylene)-5H-pyrrolo[2,3-d]pyrimidin-6(7H)-one

To 2-amino-4-chloro-7-((4-methoxy-3,5-dimethylpyridin-2-yl)methyl)-5H-pyrrolo[2,3-d]pyrimidin-6(7H)-one (400 mg, 1.20 mmol) was added 3,5-dimethyl-4-(2-(2-(pyrrolidin-1-yl)ethoxy)acetyl)-1H-pyrrole-2-carbaldehyde (390 mg, 1.40 mmol), piperidine (a drop), and EtOH (30 mL). The resulting mixture stirred for 12 hr at 80° C. and then concentrated under vacuum. The residue was purified onto an alumina column with dichloromethane/methanol (50:1-20:1), resulted in 0.232 g (33%) of the title compound as a yellow solid. ¹H NMR (400 MHz, DMSO-d₆) δ 1.65 (4H, m), 1.85-2.21 (4H, m), 2.14 (3H, s), 2.28 (3H, s), 2.43 (3H, s), 2.53 (3H, s), 2.62 (2H, t), 3.59 (2H, t), 3.75 (3H, s), 4.50 (2H, s), 5.03 (2H, s), 7.19 (2H, s), 7.82 (1H, s), 7.99 (1H, s), 13.03 (1H, s). MS m/z: 594 [M+1]^{+·}.

### Example III-102

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(diethylamino)ethyl)-2,4-dimethyl-1H-pyrrole-3-carboxamide

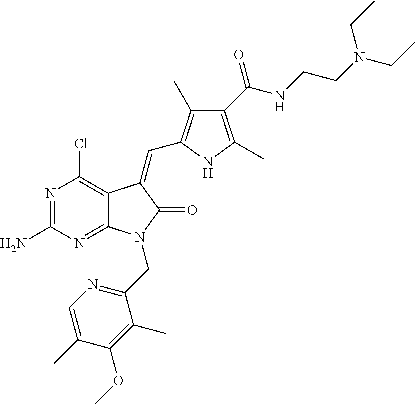

A mixture of N-(2-(diethylamino)ethyl)-5-formyl-2,4-dimethyl-1H-pyrrole-3-carboxamide (250 mg, 0.94 mmol, 1.05 equiv), 2-amino-4-chloro-7-((4-methoxy-3,5-dimethylpyridin-2-yl)methyl)-5H-pyrrolo[2,3-d]pyrimidin-6(7H)-one (300 mg, 0.90 mmol), and piperidine (a drop) in EtOH (20 ml) was stirred for overnight at room temperature. The solids were collected by filtration and washed with 2×30 mL of EtOH, resulted in 250 mg (48%) of the title compound as a yellow solid. ¹H NMR (400 MHz, CDCl₃) δ 1.03 (6H, t), 2.19 (3H, s), 2.35 (3H, s), 2.46 (3H, s), 2.54 (3H, s), 2.57 (4H, m), 2.66 (2H, m), 3.49 (2H, d), 3.76 (3H, s), 5.06 (2H, s), 5.08 (2H, s), 6.50 (1H, s), 7.96 (1H, s), 8.06 (1H, s), 12.94 (1H, s). MS m/z: 581 [M+1]^{+·}.

### Example III-103

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2,4-dimethyl-N-(2-morpholinoethyl)-1H-pyrrole-3-carboxamide

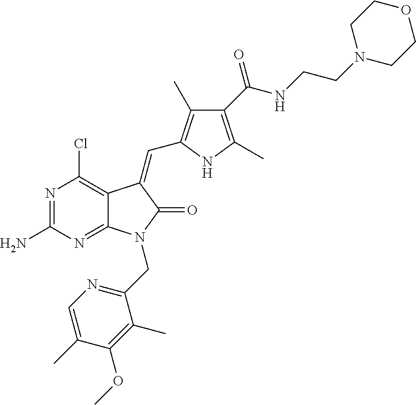

The title compound was prepared using the same protocols for the synthesis of example-102 with the corresponding aldehyde. ¹H NMR (400 MHz, DMSO-d₆) δ 2.14 (3H, s), 2.28 (3H, s), 2.34 (3H, s), 2.4-2.5 (9H, m), 3.36 (2H, m), 3.57 (4H, b), 3.74 (3H, s), 5.03 (2H, s), 7.09 (2H, s), 7.51 (1H, t), 7.77 (1H, s), 7.99 (1H, s), 12.77 (1H, s).

### Example III-104

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2,4-dimethyl-N-(2-(pyrrolidin-1-yl)ethyl)-1H-pyrrole-3-carboxamide

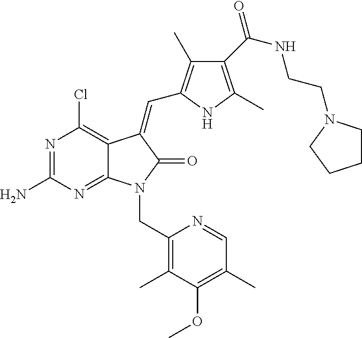

The title compound was prepared using the same protocols for the synthesis of example-102 with the corresponding aldehyde. ¹H NMR (400 MHz, DMSO-d₆) δ 2.14 (3H, s), 2.28 (4H, m), 2.39 (3H, s), 2.90 (2H, m), 3.74 (3H, s), 5.03 (2H, s), 7.26 (3H, m), 7.80 (1H, s), 8.0 (1H, s), 12.75 (1H, s). MS m/z: 479 [M+1]^{+·}.

### Example III-105

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2,4-dimethyl-N-(2-(piperidin-1-yl)ethyl)-1H-pyrrole-3-carboxamide

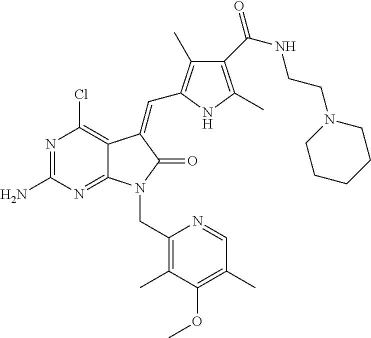

The title compound was prepared using the same protocols for the synthesis of example-102 with the corresponding aldehyde. ¹H NMR (400 MHz, DMSO-d₆) δ 1.39 (2H, m), 1.49 (4H, m), 2.14 (3H, s), 2.28 (3H, s), 2.34-2.44 (11H, m), 3.74 (3H, s), 5.02 (2H, s), 7.09 (2H, s), 7.46 (1H, t), 7.77 (1H, s), 7.98 (1H, s), 12.77 (1H, s).

### Example III-106

### 5-[2-Amino-4-chloro-7-(4-methoxy-3,5-dimethyl-pyridin-2-ylmethyl)-6-oxo-6,7-dihydro-pyrrolo[2,3-d]pyrimidin-(5Z)-ylidenemethyl]-2,4-dimethyl-1H-pyrrole-3-carboxylic acid [2-(1,1-dioxo-thiomorpholin-4-yl)-ethyl]-amide

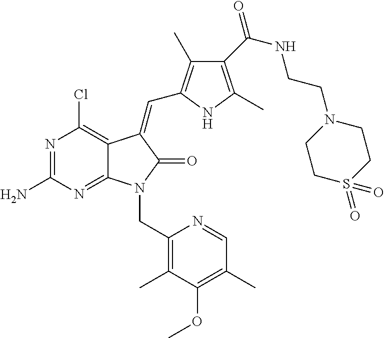

The title compound was prepared using the same protocols for the synthesis of example-102 with the corresponding aldehyde. ¹H NMR (400 MHz, DMSO-d₆) δ 2.14 (3H, s), 2.28 (3H, s), 2.34 (3H, s), 2.41 (3H, s), 2.52 (2H, m), 2.63 (2H, t), 2.95 (4H, m), 3.06 (4H, m), 3.74 (3H, s), 5.03 (2H, s), 7.09 (2H, s), 7.54 (1H, t), 7.78 (1H, s), 7.99 (t, t), 12.77 (1H, s).

### Example III-107

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4-hydroxypiperidin-1-yl)ethyl)-2,4-dimethyl-1H-pyrrole-3-carboxamide

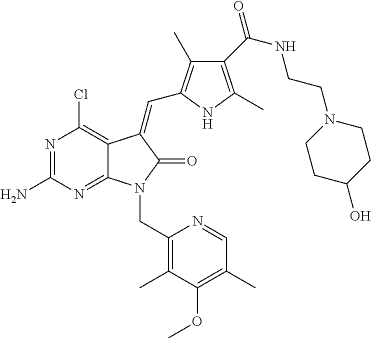

The title compound was prepared using the same protocols for the synthesis of example-102 with the corresponding aldehyde. ¹H NMR (400 MHz, DMSO-d₆) δ 1.37 (2H, m), 1.65-1.76 (3H, m), 2.06 (2H, t), 2.14 (3H, s), 2.28 (3H, s), 2.33 (3H, s), 2.35-2.45 (5H, m), 2.74 (2H, m), 3.43 (1H, m), 3.60 (1H, m), 4.52 (1H, d), 5.01 (2H, s), 7.08 (1H, s), 7.45 (1H, t), 7.76 (1H, s), 7.98 (1H, s), 12.76 (1H, s). MS m/z: [M+1]^{+·}.

### Example III-108

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4,4-difluoropiperidin-1-yl)ethyl)-2,4-dimethyl-1H-pyrrole-3-carboxamide

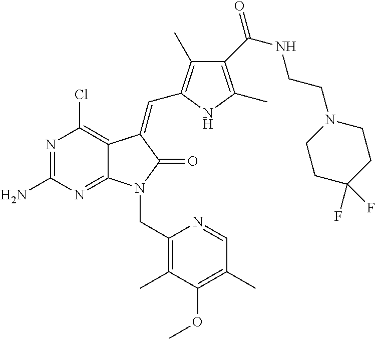

The title compound was prepared using the same protocols for the synthesis of example-102 with the corresponding aldehyde. ¹H NMR (400 MHz, DMSO-d₆) δ 1.94 (4H, m), 2.14 (3H, s), 2.28 (3H, s), 2.33 (3H, s), 2.40 (3H, s), 2.54 (4H, m), 2.34 (4H, m), 3.74 (3H, s), 5.03 (2H, s), 7.09 (2H, s), 7.52 (1H, t), 7.77 (1H, s), 7.98 (1H, s), 12.77 (1H, s).

### Example III-109

### (Z)-N-(2-(4-Acetylpiperazin-1-yl)ethyl)-5-((2-amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2,4-dimethyl-1H-pyrrole-3-carboxamide

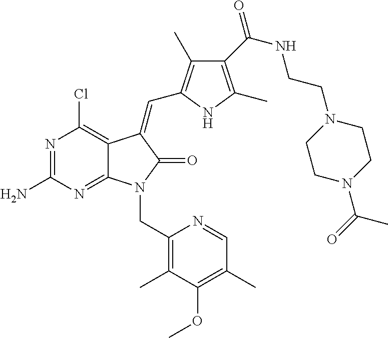

The title compound was prepared using the same protocols for the synthesis of example-102 with the corresponding aldehyde. ¹H NMR (400 MHz, DMSO-d₆) δ 1.97 (3H, s), 2.14 (3H, s), 2.28 (3H, s), 2.3-2.5 (12H, m), 3.2-5.5 (6H, m), 3.74 (3H, s), 5.01 (2H, s), 7.09 (2H, s), 7.52 (1H, t), 7.56 (1H, s), 7.98 (1H, s), 12.76 (1H, s).

### Example III-110

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(3-hydroxypiperidin-1-yl)ethyl)-2,4-dimethyl-1H-pyrrole-3-carboxamide

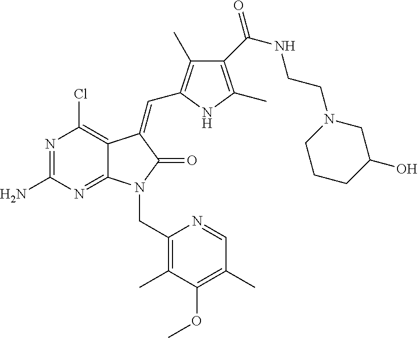

The title compound was prepared using the same protocols for the synthesis of example-102 with the corresponding aldehyde. ¹H NMR (400 MHz, DMSO-d₆) δ 1.08 (1H, m), 1.38 (1H, m), 1.62 (1H, m), 1.72-1.94 (3H, m), 2.14 (3H, s), 2.28 (3H, s), 2.32 (3H, s), 2.39-2.48 (3H, m), 2.67 (1H, m), 2.85 (1H, m), 3.30 (1H, m), 3.45 (1H, m), 3.61 (1H, m), 3.74 (3H, s), 4.57 (s, d), 5.03 (2H, s), 7.09 (2H, s), 7.46 (1H, t), 7.76 (1H, s), 7.98 (1H, s), 12.76 (1H, s).

### Example III-111

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4-isopropylpiperazin-1-yl)ethyl)-2,4-dimethyl-1H-pyrrole-3-carboxamide

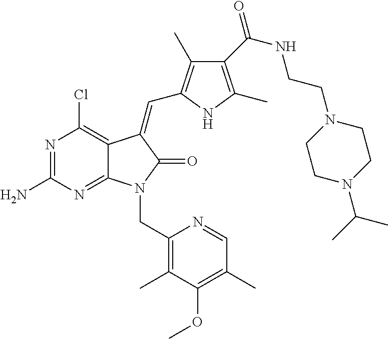

The title compound was prepared using the same protocols for the synthesis of example-102 with the corresponding aldehyde. ¹H NMR (400 MHz, DMSO-d₆) δ 0.95 (6H, d), 2.14 (3H, s), 2.28 (3H, s), 2.32 (3H, s), 2.4-2.5 (11H, m), 2.55 (1H, m), 3.30 (4H, m), 3.74 (3H, s), 5.01 (2H, s), 7.09 (2H, s), 7.47 (1H, s), 7.75 (1H, s), 7.98 (1H, s), 12.76 (1H, s).

### Example III-112

### (S,Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-hydroxy-3-morpholinopropyl)-2,4-dimethyl-1H-pyrrole-3-carboxamide

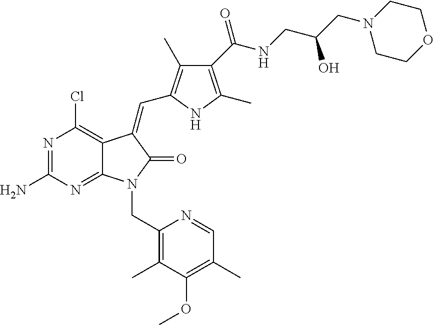

The title compound was prepared using the same protocols for the synthesis of example-102 with the corresponding aldehyde. ¹H NMR (400 MHz, DMSO-d₆) δ 2.14 (3H, s), 2.28 (3H, s), 2.34 (3H, s), 2.42 (3H, s), 3.23 (8H, m), 3.65 (4H, m), 3.74 (3H, s), 3.92 (1H, b), 5.03 (2H, s), 7.11 (2H, s), 7.63 (1H, t), 7.77 (1H, s), 7.98 (1H, s), 12.79 (1H, s).

### Example III-113

### (Z)-tert-Butyl 4-(2-(5-((2-amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2,4-dimethyl-1H-pyrrole-3-carboxamido)ethyl)piperazine-1-carboxylate

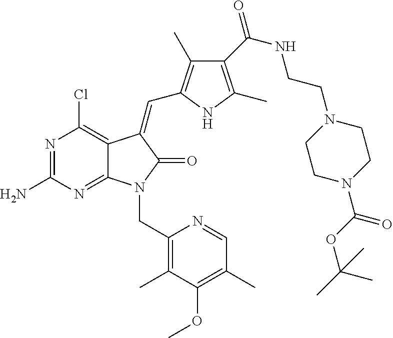

The title compound was prepared using the same protocols for the synthesis of example-102 with the corresponding aldehyde. ¹H NMR (400 MHz, DMSO-d₆) δ 1.40 (9H, s), 2.14 (3H, s), 2.28 (3H, s), 2.34 (3H, s), 2.42 (3H, s), 2.51 (6H, m), 3.31 (6H, b), 3.74 (3H, s), 5.03 (2H, s), 7.11 (2H, s), 7.56 (1H, b), 7.77 (1H, s), 7.98 (1H, s), 12.80 (1H, s).

### Example III-114

### (Z)-tert-Butyl 4-(3-(5-((2-amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2,4-dimethyl-1H-pyrrole-3-carboxamido)-2-hydroxypropyl)piperazine-1-carboxylate

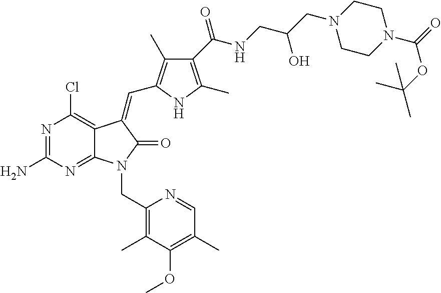

The title compound was prepared using the same protocols for the synthesis of example-102 with the corresponding aldehyde. ¹H NMR (400 MHz, DMSO-d₆) δ 1.39 (9H, s), 2.14 (3H, s), 2.28 (3H, s), 2.33 (3H, s), 2.41 (3H, s), 3.2-3.4 (14H, m), 3.74 (3H, s), 3.84 (1H, b), 5.03 (2H, s), 7.10 (2H, s), 7.58 (1H, b), 7.77 (1H, s), 7.98 (1H, s), 12.78 (1H, s).

### Example III-115

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2,4-dimethyl-N-(2-morpholinoethyl)-1H-pyrrole-3-carboxamide

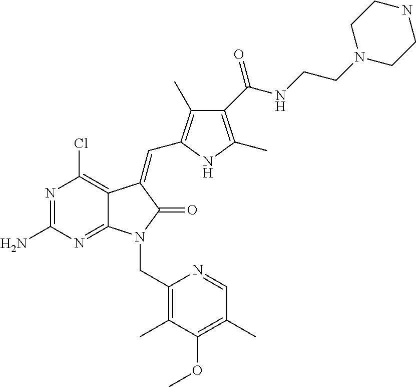

The title compound was prepared using the same protocols for the synthesis of example-102 with the corresponding aldehyde. ¹H NMR (400 MHz, DMSO-d₆) δ 2.14 (3H, s), 2.2-2.5 (17H, m), 2.65 (4H, m), 3.14 (1H, m), 3.74 (3H, s), 5.02 (2H, s), 7.09 (2H, s), 7.55 (1H, t), 7.76 (1H, s), 7.98 (1H, s), 12.77 (1H, s).

### Example III-116

### (Z)-5-((2-Amino-4-chloro-6-oxo-7-((4-(trifluoromethyl)pyridin-2-yl)methyl)-6,7-dihydro-5H-pyrrolo[2,3-d]pyrimidin-5-ylidene)methyl)-2,4-dimethyl-N-(2-(pyrrolidin-1-yl)ethyl)-1H-pyrrole-3-carboxamide

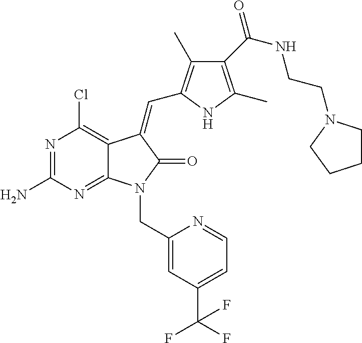

The title compound was prepared using the same protocols for the synthesis of example-66 with the corresponding aldehyde. ¹H NMR (400 MHz, DMSO-d₆) δ 1.24 (4H, m), 1.87 (3H, s), 2.03 (3H, s), 2.33 (4H, m), 2.77 (2H, m), 3.40 (2H, m), 5.19 (2H, s), 7.23 (3H, m), 7.70 (1H, s), 7.87 (1H, s), 7.91 (1H, t), 8.63 (1H, s), 12.79 (1H, s). MS m/z: 589 [M+1]^{+·}.

### Example III-117

### (Z)-5-((2-Amino-4-chloro-7-((4-methoxy-3,5-dimethylpyridin-2-yl)methyl)-6oxo-6,7-dihydro-5H-pyrrolo[2,3-d]pyrimidin-5-ylidene)methyl)-N-(2-(diethylamino)ethyl)-4-methyl-1H-pyrrole-3-carboxamide

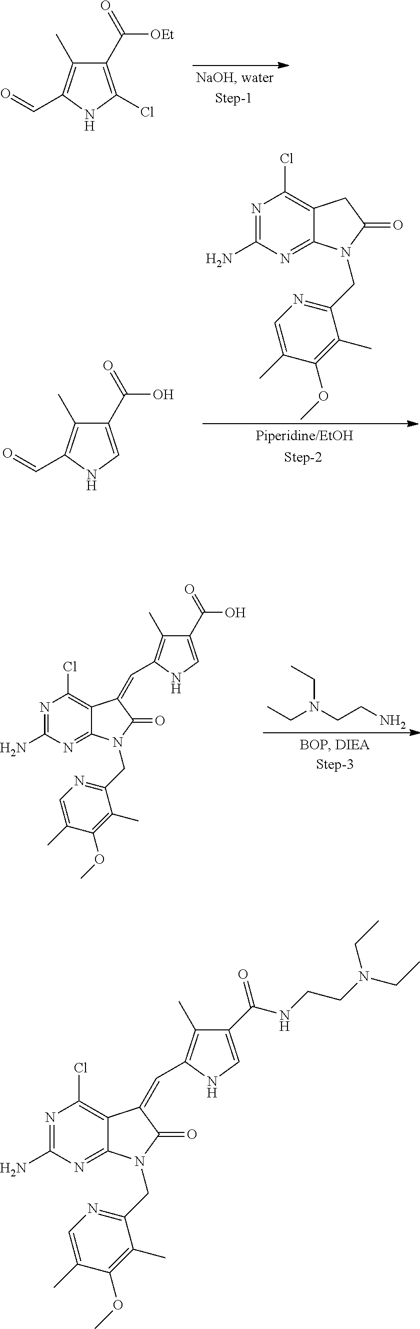

### Step-1: 5-Formyl-4-methyl-1H-pyrrole-3-carboxylic acid

To a solution of ethyl 5-formyl-4-methyl-1H-pyrrole-3-carboxylate (3 g, 15.75 mmol) in 50 mL of MeOH was added 10 mL of 2N NaOH. The resulting solution was refluxed for 3 hr. The pH value of the solution was adjusted to 2 with HCl (36%). The solids were collected by filtration, resulted in 2 g (82%) of 5-formyl-4-methyl-1H-pyrrole-3-carboxylic acid as a white solid.

### Step-2: (Z)-5-((2-Amino-4-chloro-7-((4-methoxy-3,5-dimethylpyridin-2-yl)methyl)-6-oxo-6,7-dihydropyrrolo[2,3-d]pyrimidin-5-ylidene)methyl)-4-methyl-1H-pyrrole-3-carboxylic acid

To a solution of 2-amino-4-chloro-7-((4-methoxy-3,5-dimethylpyridin-2-yl)methyl)-5H-pyrrolo[2,3-d]pyrimidin-6(7H)-one (1.2 g, 3.60 mmol) in EtOH (20 mL) under N₂ was added 5-formyl-4-methyl-1H-pyrrole-3-carboxylic acid (600 mg, 3.92 mmol) and a drop of piperidine. The resulting mixture was stirred at 50° C. for 12 hours and cooled to 5° C. The solids were collected by filtration, resulted in 1.4 g (83%) of (Z)-5-((2-amino-4-chloro-7-((4-methoxy-3,5-dimethylpyridin-2-yl)methyl)-6-oxo-6,7-dihydropyrrolo[2,3-d]pyrimidin-5-ylidene)methyl)-4-methyl-1H-pyrrole-3-carboxylic acid as a yellow solid.

### Step-3: (Z)-5-((2-Amino-4-chloro-7-((4-methoxy-3,5-dimethylpyridin-2-yl)methyl-6oxo-6,7-dihydro-5H-pyrrolo[2,3-d]pyrimidin-5-ylidene)methyl)-N-(2-diethylamino)ethyl)-4-methyl-1H-pyrrole-3-carboxamide

To a solution of (Z)-5-((2-amino-4-chloro-7-((4-methoxy-3,5-dimethylpyridin-2-yl)methyl)-6-oxo-6,7-dihydropyrrolo[2,3-d]pyrimidin-5-ylidene)methyl)-4-methyl-1H-pyrrole-3-carboxylic acid (400 mg, 0.85 mmol) in DMF (20 mL) was added BOP (455.3 mg, 1.03 mmol), DIEA (142 mg, 1.10 mmol), and N1,N1-diethylethane-1,2-diamine (141.5 mg, 1.22 mmol). The resulting solution was stirred for an hour at room temperature and treated with 20 mL of water. The solids were collected by filtration, resulted in 260 mg (54%) of the title compound as a yellow solid. ¹H NMR (400 MHz, DMSO-d₆) δ 1.09 (6H, t), 2.14 (3H, s), 2.29 (3H, s), 2.46 (3H, s), 2.90 (6H, m), 3.35 (2H, m), 3.74 (3H, s), 5.03 (2H, s), 7.20 (2H, s), 7.73 (1H, d, J=2.5 Hz), 7.83 (1H, s), 8.00 (2H, m), 12.80 (1H, s). MS m/z: 567 [M+1]^{+·}.

### Example III-118

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-4-methyl-N-(2-morpholinoethyl)-1H-pyrrole-3-carboxamide

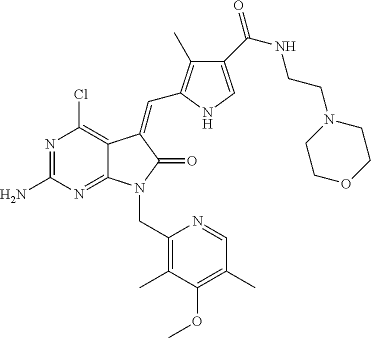

The title compound was prepared in 42% yield using the same protocols for the synthesis of example-117 with the corresponding amine replacing N1,N1-diethylethane-1,2-diamine. NMR (400 MHz, DMSO-d₆) δ 2.14 (3H, s), 2.29 (3H, s), 2.42 (6H, m), 2.45 (3H, s), 3.29 (2H, m), 3.57 (4H, t), 3.74 (3H, s), 5.03 (2H, s), 7.17 (2H, s), 7.72 (1H, d), 7.83 (1H, s), 7.85 (1H, t), 8.00 (1H, s), 12.79 (1H, s). MS m/z: 581 [M+1]^{+·}.

### Example III-119

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-4-methyl-N-(2-(pyrrolidin-1-yl)ethyl)-1H-pyrrole-3-carboxamide

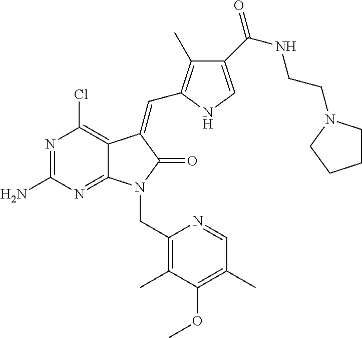

The title compound was prepared in 87% yield using the same protocols for the synthesis of example-102 with the corresponding aldehyde. ¹H NMR (400 MHz, DMSO-d₆) 1.68 (4H, m), 2.14 (3H, s), 2.29 (3H, s), 2.45 (3H, s), 2.48 (6H, m), 3.29 (2H, m), 3.74 (3H, s), 5.04 (2H, s), 7.18 (2H, s), 7.73 (1H, d, J=3.5 Hz), 7.83 (1H, s), 7.88 (1H, t), 8.00 (1H, s), 12.79 (1H, s). MS m/z: 565 [M+1]^{+·}.

### Example III-120

### (2)-5-((2-Amino-4-chloro-7-((4-methoxypyridin-2-yl)methyl)-6-oxo-6,7-dihydro-5H-pyrrolo[2,3-d]pyrimidin-5-ylidene)methyl)-2,4-dimethyl-N-(2-(pyrrolidin-1-yl)ethyl)-1H-pyrrole-3-carboxamide

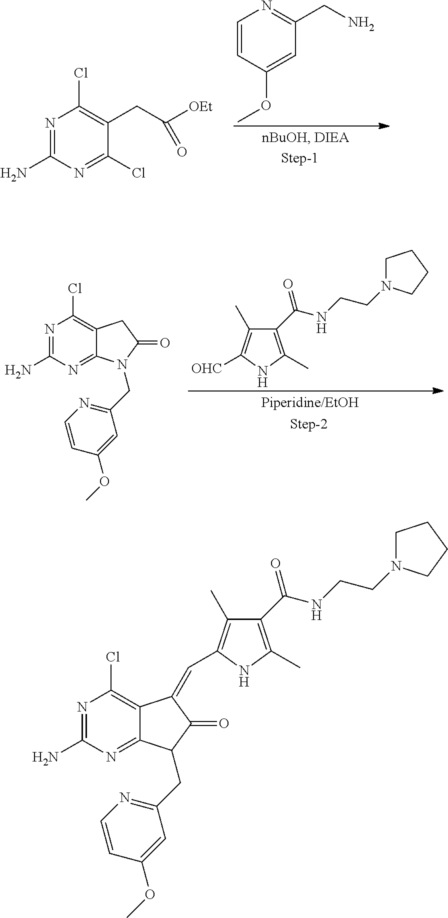

### Step-1: 2-Amino-4-chloro-7-((4-methoxypyridin-2-yl)methy)-5H-pyrrolo[2,3-d]pyrimidin-6(7H)-one

To a solution of ethyl 2-(2-amino-4,6-dichloropyrimidin-5-yl)acetate (1.8 g, 7.23 mmol) in t-BuOH (20 mL) was added (4-methoxypyridin-2-yl)methanamine (1.4 g, 10.14 mmol) was added DIEA (2.8 g, 21.71 mmol). The resulting solution was refluxed for overnight and then concentrated under vacuum. The residue was purified onto a silica gel column with ethyl acetate/petroleum ether (1:20-1:5), resulted in 1 g (37%) of ethyl 2-(2-amino-4-chloro-6-((4-methoxypyridin-2-yl)methylamino)pyrimidin-5-yl)acetate as a brown solid. To it (1 g, 2.71 mmol) was added n-BuOH (30 mL) and the resulting solution was heated to reflux for 5 hr. After the reaction was cooled to room temperature with a water/ice bath, the solids were collected by filtration, resulted in 0.6 g (71%) of 2-amino-4-chloro-7-((4-methoxypyridin-2-yl)methyl)-5H-pyrrolo[2,3-d]pyrimidin-6(7H)-one as a white solid.

### Step-2: (Z)-5-((2-Amino-4-chloro-7-((4-methoxypyridin-2-yl)methyl)-6-oxo-6,7-dihydro-5H-pyrrolo[2,3-d]pyrimidin-5-ylidene)methyl)-2,4-dimethyl-N-(2-(pyrrolidin-1-yl)ethyl)-1H-pyrrole-3-carboxamide

A mixture of 5-formyl-2,4-dimethyl-N-(2-(pyrrolidin-1-yl)ethyl)-1H-pyrrole-3-carboxamide (440 mg, 1.67 mmol) and 2-amino-4-chloro-7-((4-methoxypyridin-2-yl)methyl)-5H-pyrrolo[2,3-d]pyrimidin-6(7H)-one (460 mg, 1.51 mmol) in EtOH (30 mL) was added piperidine (a drop). The resulting solution was refluxed for overnight. The resulting mixture was concentrated under vacuum. The residue was purified onto a silica gel column with dichloromethane/methanol (20:1), resulted in 210.7 mg (25%) of the title compound as a yellow solid. ¹H NMR (400 MHz, DMSO-d₆) δ 1.68 (4H, m), 1.86-2.15 (6H, m), 2.32 (3H, s), 2.41 (3H, s), 2.56 (2H, m), 3.81 (3H, s), 5.05 (2H, s), 6.88 (2H, m), 7.16 (2H, s), 7.57 (1H, t), 7.78 (1H, s), 8.25 (1H, m), 12.73 (1H, s). MS m/z: 551 [M+1]^{+·}.

### Example III-121

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2-chloro-4-methyl-N-(2-(pyrrolidin-1-yl)ethyl)-1H-pyrrole-3-carboxamide

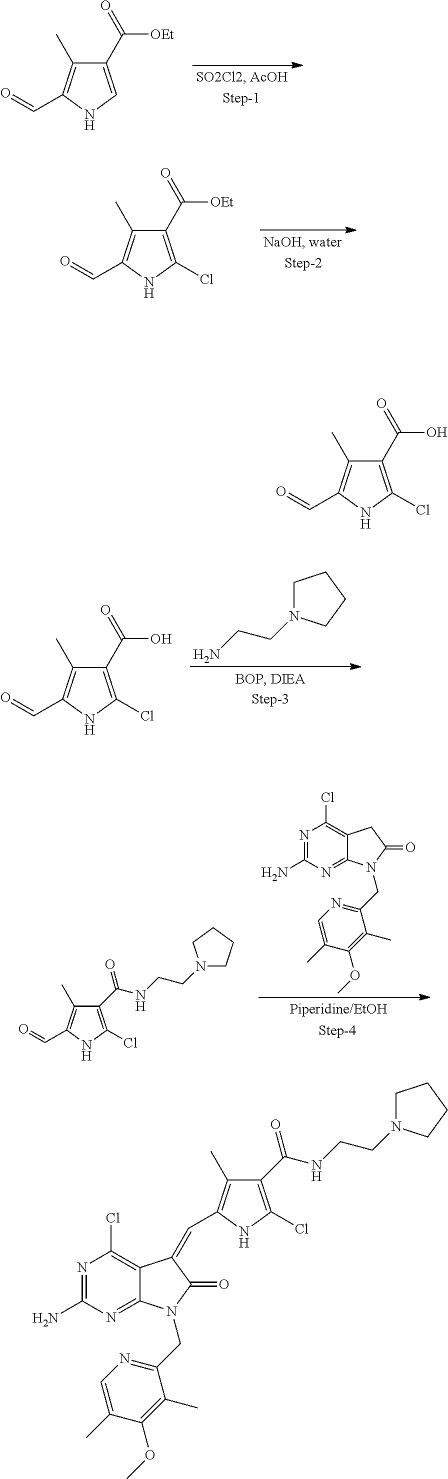

### Step-1: Ethyl 2-chloro-5-formyl-4-methyl-1H-pyrrole-3-carboxylate

To ethyl 5-formyl-4-methyl-1H-pyrrole-3-carboxylate (3.4 g, 18.78 mmol) was added a solution of SO₂Cl₂ (1.78 g, 13.19 mmol) in CH₃COOH (7.6 mL) dropwise at room temperature and followed by addition of CH₃COOH (15.2 mL). The resulting solution was stirred for 10 min at room temperature and then quenched by the addition of 100 mL of Na₂CO₃ (20% solution). The solids were collected by filtration, dried in air, and dissolved in EtOH. The solution was heated at 80° C. and treated with a solution of Na₂CO₃ (5.1 g) in H₂O (83 mL). After the filtration, the pH value of the filtrate was adjusted to 7 with CH₃COOH. The solids were filtered and washed with 20 mL of H₂O and dried, resulted in 2.0 g (52%) of ethyl 2-chloro-5-formyl-4-methyl-1H-pyrrole-3-carboxylate as a white solid.

### Step-2: 2-Chloro-5-formyl-4-methyl-1H-pyrrole-3-carboxylic acid

A mixture of ethyl 2-chloro-5-formyl-4-methyl-1H-pyrrole-3-carboxylate (650 mg, 3.02 mmol) and NaOH (3 N, 20 mL) was stirred at 80° C. for 2 hr. After it was cooled to room temperature with a water/ice bath, the pH value of the solution was adjusted to ˜3 with HCl (5 N). The solids were collected by filtration, resulted in 0.5 g (88%) of 2-chloro-5-formyl-4-methyl-1H-pyrrole-3-carboxylic acid as a gray solid.

### Step-3: 2-Chloro-5-formyl-4-methyl-N-(2-(pyrrolidin-1-yl)ethyl)-1H-pyrrole-3-carboxamide

A mixture of 2-chloro-5-formyl-4-methyl-1H-pyrrole-3-carboxylic acid (880 mg, 4.71 mmol) was added 2-(pyrrolidin-1-yl)ethanamine (590 mg, 5.18 mmol), BOP (2.5 g, 5.66 mmol), and DIEA (910 mg, 7.05 mmol, 1.50 equiv) in CH₂Cl₂ (20 mL) was stirred for 6 hr at room temperature. The reaction was then treated with 10 mL of water and concentrated under vacuum. The residue was purified onto a silica gel column with dichloromethane/methanol (20:1). This resulted in 0.85 g (64%) of 2-chloro-5-formyl-4-methyl-N-(2-(pyrrolidin-1-yl)ethyl)-1H-pyrrole-3-carboxamide as a yellow solid

### Step-4: (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2-chloro-4-methyl-N-(2-(pyrrolidin-1-yl)ethyl)-1H-pyrrole-3-carboxamide

To a mixture of 2-amino-4-chloro-7-((4-methoxy-3,5-dimethylpyridin-2-yl)methyl)-5H-pyrrolo[2,3-d]pyrimidin-6(7H)-one (460 mg, 1.38 mmol) and 2-chloro-5-formyl-4-methyl-N-(2-(pyrrolidin-1-yl)ethyl)-1H-pyrrole-3-carboxamide (400 mg, 1.41 mmol) was added piperidine (a drop) and EtOH (20 mL). It was allowed stirred at 50° C. for 12 hr and was cooled to room temperature. The solids were collected by filtration and washed with 30 mL of EtOH, resulted in 355 mg (43%) of the title compound as a yellow solid. ¹H NMR (400 MHz, DMSO-d₆) δ 1.85 (4H, m), 2.14 (3H, s), 2.29 (3H, s), 2.36 (3H, s), 3.03 (6H, m), 3.48 (2H, m), 3.75 (3H, s), 5.05 (2H, s), 7.28 (2H, s), 7.76 (1H, s), 8.01 (2H, m), 13.48 (1H, s). MS m/z: 599 [M+1]^{+·}.

### Example III-122

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4-methylpiperazin-1-yl)ethyl)-1H-pyrrole-3-carboxamide

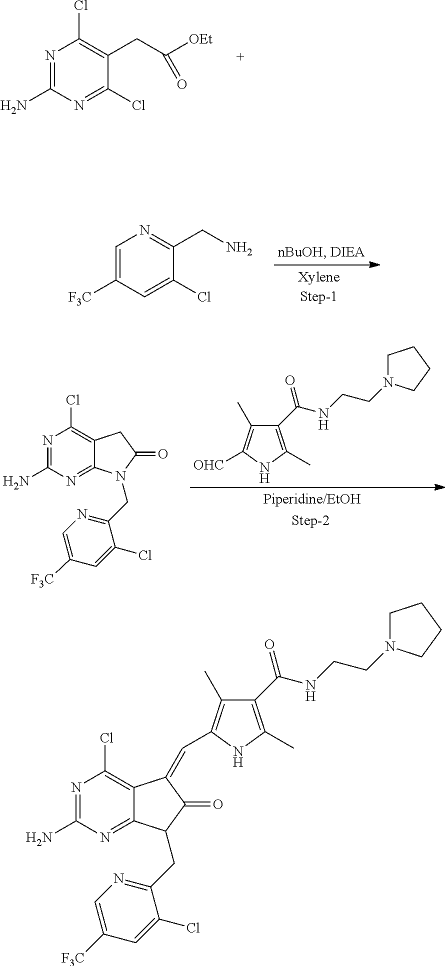

### Step-1: 2-Amino-4-chloro-7-((3-chloro-5-(trifluoromethyl)pyridin-2-yl)methyl)-5H-pyrrolo[2,3-d]pyrimidin-6(7H)-one

To ethyl 2-(2-amino-4,6-dichloropyrimidin-5-yl)acetate (1.05 g, 4.20 mmol) under N₂ was added (3-chloro-5-(trifluoromethyl)pyridin-2-yl)methanamine (1.10 g, 5.22 mmol), t-BuOH (10.0 mL), and DIEA (2.02 g, 15.63 mmol). The resulting solution was stirred for overnight at 80-90° C. The resulting solution was treated with ethyl acetate/H₂O and extracted with 3×20 mL of ethyl acetate. The combined organic layers were dried over anhydrous magnesium sulfate and concentrated under vacuum, resulted in 0.5 g (crude) of ethyl 2-(2-amino-4-chloro-6-((3-chloro-5-(trifluoromethyl)pyridin-2-yl)methylamino)pyrimidin-5-1)acetate as a white solid. To ethyl 2-(2-amino-4-chloro-6-((3-chloro-5-(trifluoromethyl)pyridin-2-yl)methylamino)pyrimidin-5-yl)acetate (600 mg, 1.41 mmol) under N₂ was added xylene (10 mL) and 4-methylbenzenesulfonic acid (0.3 g). The resulting solution was stirred for overnight at 90-100° C. and treated with 30 mL of H₂O/ice. The resulting mixture was extracted with 3×30 mL of ethyl acetate, the organic layers combined and dried over Na₂SO₄, then concentrated under vacuum, resulted in 0.3 g (56%) of 2-amino-4-chloro-7-((3-chloro-5-(trifluoromethyl)pyridin-2-yl)methyl)-5H-pyrrolo[2,3-d]pyrimidin-6(7H)-one as a yellow solid.

### Step-2: (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-N-(2-(4-methylpiperazin-1-yl)ethyl)-1H-pyrrole-3-carboxamide

To a mixture of 2-amino-4-chloro-7-((3-chloro-4-(trifluoromethyl)pyridin-2-yl)methyl)-5H-pyrrolo[2,3-d]pyrimidin-6(7H)-one (120 mg, 0.32 mmol) and 5-formyl-2,4-dimethyl-N-(2-(pyrrolidin-1-yl)ethyl)-1H-pyrrole-3-carboxamide (100 mg, 0.38 mmol) in EtOH (20 mL) was added piperidine (a drop). The resulting mixture was stirred for 12 hr at 50° C. The solids were collected by filtration and washed with 2×20 mL of EtOH, resulted in 165 mg (83%) of the title compound as a yellow solid. ¹H NMR (400 MHz, DMSO-d₆) δ 1.69 (4H, m), 2.33 (3H, s), 2.40 (3H, s), 2.53 (4H, m), 2.57 (2H, m), 3.31 (2H, m), 5.28 (2H, s), 7.17 (2H, s), 7.59 (1H, t), 7.80 (1H, s), 8.56 (1H, s), 8.84 (1H, s). MS m/z: 623 [M+1]^{+·}.

### Example III-123

### (Z)-5-((2-Amino-4-chloro-7-((4-methoxy-3,5-dimethylpyridin-2-yl)methyl)-6-oxo-6,7-dihydro-5H-pyrrolo[2,3-d]pyrimidin-5-ylidene)methyl)-2-methyl-N-(2-(pyrrolidin-1-yl)ethyl)-4-(trifluoromethyl)-1H-pyrrole-3-carboxamide

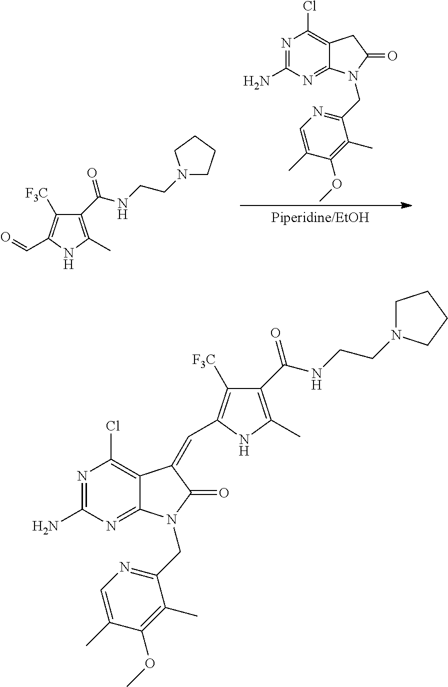

To a solution of N-(2-(diethylamino)ethyl)-5-formyl-2-methyl-4-(trifluoromethyl)-1H-pyrrole-3-carboxamide (200 mg, 0.63 mmol) in EtOH (20 mL) was added 2-amino-4-chloro-7-((4-methoxy-3,5-dimethylpyridin-2-yl)methyl)-5H-pyrrolo[2,3-d]pyrimidin-6(7H)-one (210 mg, 0.63 mmol) and piperidine (2 mg). The resulting mixture was stirred for overnight at room temperature. The solids were collected by filtration, resulted in 300 mg (57%) of the title compound as a yellow solid. ¹H NMR (400 MHz, DMSO-d₆) δ 0.98 (6H, m), 2.14 (3H, s), 2.28 (3H, s), 2.33 (3H, s), 2.53 (6H, m), 3.28 (2H, m), 3.75 (3H, s), 5.03 (2H, s), 7.11 (2H, s), 7.46 (1H, t), 7.78 (1H, s), 8.00 (2H, m), 12.78 (1H, s). MS m/z: 635 [M+1]^{+·}. (Z)-5-((2-amino-4-chloro-7-((4-methoxy-3,5-dimethylpyridin-2-yl)methyl)-6-oxo-6,7-dihydro-5H-pyrrolo[2,3-d]pyrimidine-5-ylidene)methyl)-N-(2-(pyrrolidin-2-yl)ethyl)-1H-pyrrole-3-carboxamide.

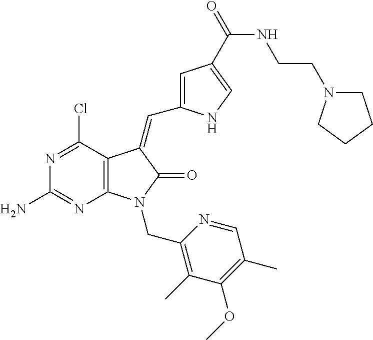

Additional exemplary compounds of formula (III) are shown above.

### Exemplary Compounds of Formula (IV)

### Example IV-1

### 2-amino-4-chloro-8-((4-methoxy-3,5-dimethylpyridin-2-yl)methyl)-7,8-dihydropyrido[2,3-d]pyrimidin-5(6H)-one

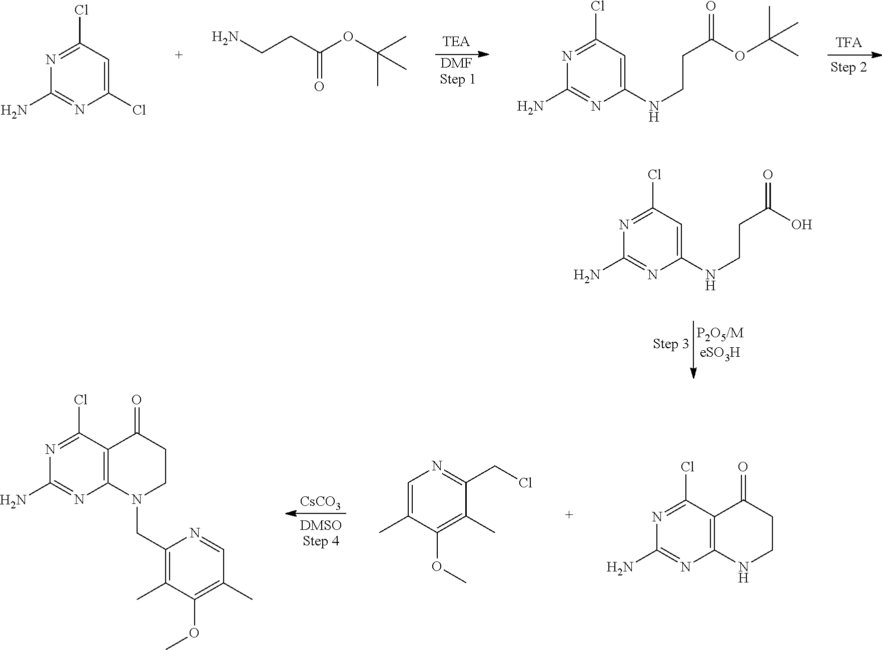

### Step 1: tert-Butyl 3-(2-amino-6-chloropyrimidin-4-ylamino)propanoate

To a stirred solution of 2-amino-4,6-dichloropyrimidine (4.48 g, 27.3 mmol) and β-alanine tert-butyl ester hydrochloride (4.97 g, 27.4 mmol) in DMF (30 mL) was added triethylamine (4.0 mL, 28.7 mmol). The resulting mixture was stirred at 60° C. for 16 h. The reaction mixture was cooled to room temperature, and then diluted with ethyl acetate (120 mL) and saturated NH₄Cl solution (100 mL). The organic phase was separated, and the aqueous phase was extracted with ethyl acetate (30 mL×2). The combined organic phase was washed with brine (40 mL×3), dried over MgSO₄, filtered through a layer of silica gel to remove the yellow color, and then concentrated on a rotary evaporator to dryness. The residue was triturated with hexanes (15 mL). The product was collected by filtration, washed with hexanes (10 mL×2), and dried in vacuo to afford tert-Butyl 3-(2-amino-6-chloropyrimidin-4-ylamino)propanoate as a light-yellow solid (5.2 g, yield 70%).

¹H NMR (CDCl₃) δ (ppm) 5.79 (1H, s), 5.26 (1H, s, br), 4.85 (2H, s, br), 3.55 (2H, m), 2.50 (2H, t, J=6.0 Hz), 1.46 (9H, s).

### Step 2: 3-(2-Amino-6-chloropyrimidin-4-ylamino)propanoic acid

tert-Butyl 3-(2-amino-6-chloropyrimidin-4-ylamino)propanoate (5.1 g, 18.7 mmol) was dissolved in trifluoroacetic acid (60 mL). The resulting solution was stirred at room temperature for 1 h, and then concentrated on a rotary evaporator to dryness. The residue was re-dissolved in trifluoroacetic acid (50 mL). The solution was allowed to stand at room temperature for 1 h, and then concentrated to dryness. Water (30 mL) was added to the residue. The resulting mixture was stirred at 0° C. and neutralized carefully with 1 N NaOH solution to pH 3-4. The precipitated product was collected by filtration, washed with cold water (8 mL×2), and dried in vacuo to yield 3-(2-amino-6-chloropyrimidin-4-ylamino)propanoic acid as a light-yellow solid (3.6 g, yield 89%).

¹H NMR (DMSO-d₆) δ (ppm) 12.21 (1H, s, br), 7.22 (1H, s, br), 6.44 (2H, s, br), 5.76 (1H, s), 3.41 (2H, m), 2.47 (2H, t, J=6.8 Hz).

### Step 3: 2-Amino-4-chloro-7,8-dihydropyrido[2,3-d]pyrimidin-5(6H)-one

A solution of 3-(2-amino-6-chloropyrimidin-4-ylamino)propanoic acid (3.5 g, 16.2 mmol) in Eaton's reagent (P₂O₅, 7.5 wt. % in methanesulfonic acid) (47 mL) was stirred at 75° C. for 3 h. The solution was cooled to room temperature, and then poured into a mixture of crushed ice (300 g) and ammonium hydroxide (50 mL). Ethyl acetate (300 mL) was added. The resulting mixture was stirred at 0° C., and the pH was adjusted with ammonium hydroxide to 6. The mixture was filtered, and the filter cake was washed with ethyl acetate (15 mL×2). The organic phase of the filtrate was separated, and the aqueous phase was extracted with ethyl acetate repeatedly (50 mL×12). The combined organic phase was washed with saturated NaHCO₃ solution (150 mL) and brine (150 mL×2), dried over MgSO₄, and then concentrated on a rotary evaporator to dryness to afford 2-amino-4-chloro-7,8-dihydropyrido[2,3-d]pyrimidin-5(6H)-one an off-white solid (370 mg, yield 12%). The crude product was used for the next step without further purification.

¹H NMR (DMSO-d₆) δ (ppm) 7.89 (1H, s), 7.22 (1H, s, br), 7.16 (1H, s, br), 3.38 (2H, m), 2.47 (2H, t, J=6.8 Hz).

### Step 4: 2-amino-4-chloro-8-((4-methoxy-3,5-dimethylpyridin-2-yl)methyl)-7,8-dihydropyrido[2,3-d]pyrimidin-5(6H)-one

A suspension of 2-amino-4-chloro-7,8-dihydropyrido[2,3-d]pyrimidin-5(6H)-one (370 mg, 1.86 mmol), 2-chloromethyl-4-methoxy-3,5-dimethylpyridine hydrochloride (621 mg, 2.80 mmol) and cesium carbonate (1.52 g, 4.66 mmol) in anhydrous DMSO (6 mL) was stirred at room temperature under a nitrogen atmosphere for 3 h. The mixture was diluted with ethyl acetate (30 mL) and saturated NH₄Cl solution (20 mL). The organic phase was separated, and the aqueous phase was extracted with ethyl acetate (10 mL×2). The combined organic phase was washed with brine (10 mL×3), dried over MgSO₄, filtered through a layer of silica gel to remove the yellow color, and then concentrated on a rotary evaporator to dryness. The residue was diluted with ether (5 mL). The resulting mixture was allowed to stand at room temperature overnight. The crystallized product was collected by filtration, washed with a mixture of ether and hexanes (v/v 1:1, 3 mL×2), and dried in vacuo to afford 2-amino-4-chloro-8-((4-methoxy-3,5-dimethylpyridin-2-yl)methyl)-7,8-dihydropyrido[2,3-d]pyrimidin-5(6H)-one as a white solid (376 mg, yield 58%).

¹H NMR (CDCl₃) δ (ppm) 8.17 (1H, s), 5.22 (2H, s, br), 4.95 (2H, s), 3.77 (3H, s), 3.61 (2H, t, J=7.0 Hz), 2.65 (2H, t, J=7.0 Hz), 2.26 (3H, s), 2.25 (3H, s).

### Example IV-2

### 2-Amino-4-chloro-6-(cyclopentyl(hydroxy)methyl)-8-((4-methoxy-3,5-dimethylpyridin-2-yl)methyl)-7,8-dihydropyrido[2,3-d]pyrimidin-5(6H)-one

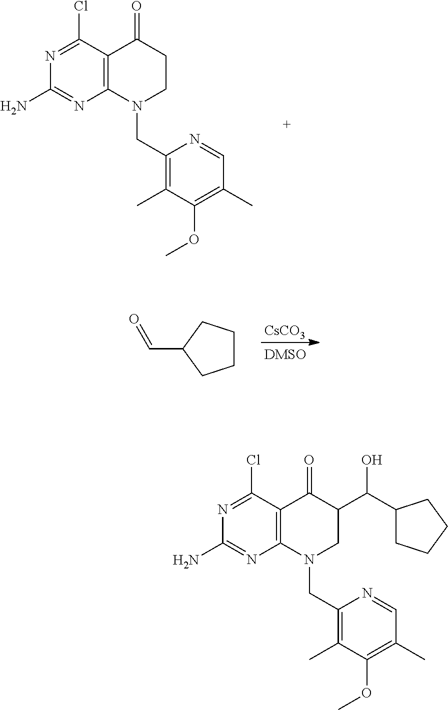

To a stirred suspension of 2-amino-4-chloro-8-((4-methoxy-3,5-dimethylpyridin-2-yl)methyl)-7,8-dihydropyrido[2,3-d]pyrimidin-5(6H)-one (50 mg, 0.14 mmol) and cesium carbonate (141 mg, 0.43 mmol) in anhydrous DMSO (3 mL) was added cyclopentanecarboxaldehyde (154 μL, 1.44 mmol). The resulting mixture was stirred at room temperature under a nitrogen atmosphere for 4 h. The mixture was diluted with ethyl acetate (15 mL) and saturated NH₄Cl solution (10 mL). The organic phase was separated, and the aqueous phase was extracted with ethyl acetate (5 mL×2). The combined organic phase was washed with brine (5 mL×3), dried over MgSO₄, and then concentrated on a rotary evaporator to dryness. The residue was purified by flash chromatography (silica gel, ethyl acetate/hexanes v/v from 0 to 100%). A white solid was obtained (34 mg, yield 53%).

1H NMR (CDCl₃) δ (ppm) 8.14 (1H, s), 6.49 (1H, s, br), 5.51 (1H, d, J=16.0 Hz), 5.09 (2H, s, br), 4.11 (1H, d, J=16.0 Hz), 4.02 (1H, dd, J=13.0, 3.8 Hz), 3.81-3.73 (6H, m), 2.60 (1H, m), 2.34 (3H, s), 2.27-2.22 (5H, m), 2.00 (1H, m), 1.85 (1H, m), 1.62 (1H, m), 1.55 (1H, m), 1.37 (1H, m), 1.28 (1H, m).

### Example IV-4

### 2-Amino-4-chloro-6-(1-hydroxy-2-methylpropyl)-8-((4-methoxy-3,5-dimethylpyridin-2-yl)methyl)-7,8-dihydropyrido[2,3-d]pyrimidin-5(6H)-one

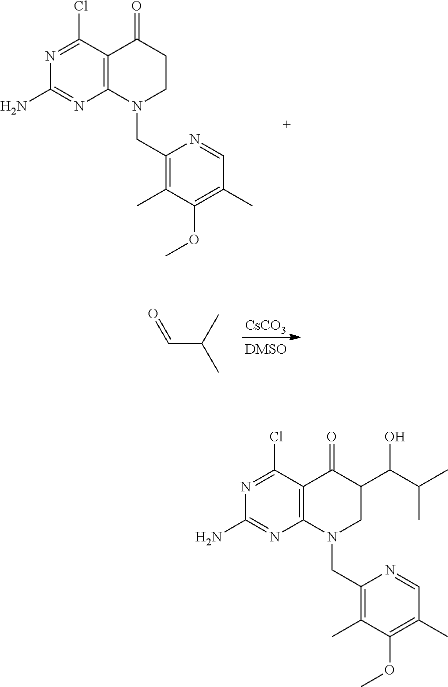

To a stirred suspension of 2-amino-4-chloro-8-((4-methoxy-3,5-dimethylpyridin-2-yl)methyl)-7,8-dihydropyrido[2,3-d]pyrimidin-5(6H)-one (72 mg, 0.21 mmol) and cesium carbonate (216 mg, 0.66 mmol) in anhydrous DMSO (5 mL) was added isobutyraldehyde (202 μL, 2.21 mmol). The resulting mixture was stirred at room temperature under a nitrogen atmosphere for 2.5 h. The mixture was diluted with ethyl acetate (20 mL) and saturated NH₄Cl solution (20 mL). The organic phase was separated, and the aqueous phase was extracted with ethyl acetate (10 mL×2). The combined organic phase was washed with brine (10 mL×3), dried over MgSO₄, and then concentrated on a rotary evaporator to dryness. The residue was purified by flash chromatography (silica gel, ethyl acetate/hexanes v/v from 0 to 100%). A white solid was obtained (50 mg, yield 58%).

¹H NMR (CDCl₃) δ (ppm) 8.15 (1H, s), 6.51 (1H, s, br), 5.52 (1H, d, J=16.4 Hz), 5.13 (2H, s, br), 4.08 (1H, d, J=16.4 Hz), 4.03 (1H, dd, J=13.2, 3.2 Hz), 3.80 (3H, s), 3.77 (1H, m), 3.66 (1H, m), 2.69 (1H, m), 2.35 (3H, s), 2.26 (3H, s), 1.75 (1H, m), 1.03 (3H, d, J=6.8 Hz), 0.94 (3H, d, J=6.8 Hz).

### Example IV-6

### 2-Amino-4-chloro-6-isobutyl-8-((4-methoxy-3,5-dimethylpyridin-2-yl)methyl)-7,8-dihydropyrido[2,3-d]pyrimidin-5(6H)-one

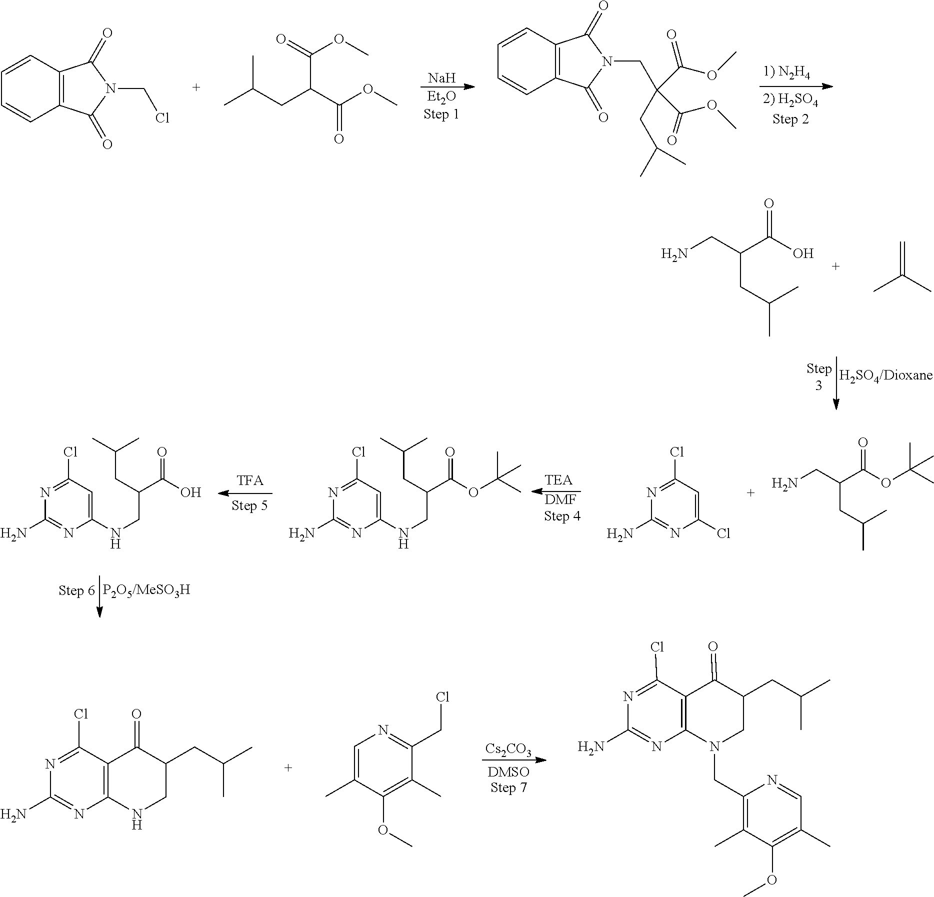

### Step 1: Dimethyl 2-((1,3-dioxoisoindolin-2-yl)methyl)-2-isobutylmalonate

To a stirred suspension of sodium hydride (60% in mineral oil, 2.4 g, 60.0 mmol) in anhydrous ether (170 mL) was added dropwise dimethyl isobutylmalonate (10.0 mL, 53.8 mmol) over 10 min. The resulting mixture was stirred at room temperature under a nitrogen atmosphere for 3 h, and was then chilled in an ice-bath. To the stirred cold solution was added N-(chloromethyl)phthalimide (10.1 g, 51.6 mmol) in one portion. The mixture was allowed to warm to room temperature slowly, and was stirred at room temperature for 18 h, followed by refluxing for 1 h. The reaction mixture was cooled to room temperature, and cold 1 N HCl (215 mL) was added. The organic phase was separated, and the aqueous phase was extracted with ether (40 mL). The combined organic phase was washed with brine (40 mL×2), dried over MgSO₄, and then concentrated on a rotary evaporator to dryness. The residue was triturated with hexanes (60 mL). The product was collected by filtration, washed with hexanes (30 mL×2), and dried in vacuo to afford dimethyl 2-((1,3-dioxoisoindolin-2-yl)methyl)-2-isobutylmalonate as a white solid (15.3 g, yield 85%).

¹H NMR (CDCl₃) δ (ppm) 7.84 (2H, q, J=2.8 Hz), 7.72 (2H, q, J=2.8 Hz), 4.30 (2H, s), 3.78 (6H, s), 2.02 (1H, m), 1.80 (2H, d, J=6.4 Hz), 0.86 (6H, d, J=6.8 Hz).

### Step 2: 2-(aminomethyl)-4-methylpentanoic acid

To a stirred solution of dimethyl 2-((1,3-dioxoisoindolin-2-yl)methyl)-2-isobutylmalonate (6.0 g, 17.3 mmol) in methanol (150 mL) was added dropwise hydrazine hydrate (1.4 mL, 44.9 mmol). The resulting solution was stirred at room temperature for 0.5 h, and 5% HCl (60 mL) was added. The resulting mixture was stirred at room temperature overnight. A white precipitate formed. The mixture was concentrated on a rotary evaporator to remove most of methanol, and then filtered. The filter cake was washed with water (10 mL×3). The filtrate was washed with ether (30 mL×3). The organic phase was discarded. The aqueous phase was basified with 10 N NaOH solution to pH 10, and then extracted with ether (30 mL×3). The combined organic phase was washed with brine (30 mL×2), dried over MgSO₄, and then concentrated to dryness. The oily residue was dissolved in 6.5% H₂SO₄ (30 mL). The resulting solution was refluxed for 16 h, and then concentrated on a rotary evaporator to one third volume. The solution was basified carefully with ammonium hydroxide to pH 6 under magnetic stirring. The resulting mixture was allowed to stand at 4° C. overnight. The crystallized product was collected by filtration, washed with cold water (2 mL×3) and ether (5 mL×2), and dried in vacuo to afford 2-(aminomethyl)-4-methylpentanoic acid as a white solid (0.69 g, yield 28%).

¹H NMR (D₂O) δ (ppm) 3.08 (2H, m), 2.61 (1H, m), 1.65-1.50 (2H, m), 1.33 (1H, 0.92 (3H, d, J=6.8 Hz), 0.91 (3H, d, J=6.8 Hz).

### Step 3: tert-Butyl 2-(aminomethyl)-4-methylpentanoate

To a cold solution of 2-(aminomethyl)-4-methylpentanoic acid (685 mg, 4.7 mmol) in a mixture of 1,4-dioxane (20 mL) and concentrated H₂SO₄ (2.0 mL, 36.8 mmol) in a 120 mL pressure reaction vessel was added liquid 2-methylpropene (20 mL). The reaction vessel was sealed. The resulting solution was stirred at room temperature for 24 h, and was then poured into a stirred cold mixture of ether (100 mL) and 1 N NaOH solution (100 mL). The organic phase was separated, and the aqueous phase was extracted with ether (20 mL×3). The combined organic phase was washed with brine (40 mL×2), dried over MgSO₄, and then concentrated on a rotary evaporator to dryness. The residue was further dried under high vacuum to afford tert-butyl 2-(aminomethyl)-4-methylpentanoate as a colorless oil was obtained (474 mg, yield 50%).

¹H NMR (CDCl₃) δ (ppm) 2.90-2.71 (2H, m), 2.40 (1H, m), 1.64-1.47 (2H, m), 1.46 (9H, s), 1.33 (2H, s, br), 1.20 (1H, m), 0.92 (3H, d, J=6.8 Hz), 0.90 (3H, d, J=6.8 Hz).

### Step 4: tert-Butyl 2-((2-amino-6-chloropyrimidin-4-ylamino)methyl)-4-methylpentanoate

To a stirred solution of 2-amino-4,6-dichloropyrimidine (383 mg, 2.34 mmol) and tert-butyl 2-(aminomethyl)-4-methylpentanoate (474 mg, 2.35 mmol) in DMF (4 mL) was added triethylamine (349 μL, 2.50 mmol). The resulting mixture was stirred at 60° C. for 16 h. The reaction mixture was cooled to room temperature, and then diluted with ethyl acetate (25 mL) and saturated NH₄Cl solution (20 mL). The organic phase was separated, and the aqueous phase was extracted with ethyl acetate (10 mL×2). The combined organic phase was washed with brine (10 mL×3), dried over MgSO₄, filtered through a layer of silica gel to remove the yellow color, and then concentrated on a rotary evaporator to dryness to afford tert-butyl 2-((2-amino-6-chloropyrimidin-4-ylamino)methyl)-4-methylpentanoate as a light-yellow solid (763 mg, yield 99%). The crude product was used for the next step without further purification.

¹H NMR (CDCl₃) δ (ppm) 5.78 (1H, s), 5.12 (1H, s, br), 4.82 (2H, s), 3.40 (2H, m), 2.62 (1H, m), 1.71-1.50 (2H, m), 1.44 (9H, s), 1.26 (1H, m), 0.93 (6H, d, J=6.4 Hz).

### Step 5: 2-((2-Amino-6-chloropyrimidin-4-ylamino)methyl)-4-methylpentanoic acid

Tert-Butyl 2-((2-amino-6-chloropyrimidin-4-ylamino)methyl)-4-methylpentanoate (763 mg, 2.32 mmol) was dissolved in trifluoroacetic acid (8 mL). The resulting solution was stirred at room temperature for 1 h, and then concentrated on a rotary evaporator to dryness. The residue was re-dissolved in trifluoroacetic acid (8 mL). The solution was allowed to stand at room temperature for 1 h, and then concentrated to dryness. Water (10 mL) was added to the residue. The resulting mixture was stirred at 0° C. and neutralized carefully with 1 N NaOH solution to pH 3-4. The precipitated product was collected by filtration, washed with cold water (2 mL×2), and dried in vacuo to afford 242-Amino-6-chloropyrimidin-4-ylamino)methyl)-4-methylpentanoic acid as a light-yellow solid (633 mg, yield 100%). The crude product was used for the next step without further purification.

¹H NMR (DMSO-d₆) δ (ppm) 12.2 (1H, s, br), 7.54 (1H, s, br), 6.67 (2H, s, br), 5.85 (1H, s), 3.37 (2H, m), 2.61 (1H, m), 1.57 (1H, m), 1.43 (1H, m), 1.25 (1H, m), 0.87 (3H, d, J=6.8 Hz), 0.86 (3H, d, J=6.8 Hz).

### Step 6: 2-Amino-4-chloro-6-isobutyl-7,8-dihydropyrido[2,3-d]pyrimidin-5(6H)-one

A solution of 2-((2-amino-6-chloropyrimidin-4-ylamino)methyl)-4-methylpentanoic acid (630 mg, 2.3 mmol) in Eaton's reagent (P₂O₅, 7.5 wt. % in methanesulfonic acid) (6.5 mL) was stirred at 75° C. for 3 h. The solution was cooled to room temperature, and then poured into a mixture of crushed ice (40 g) and ammonium hydroxide (7 mL). Ethyl acetate (30 mL) was added. The resulting mixture was stirred at 0° C., and the pH was adjusted with ammonium hydroxide to 6. The mixture was filtered, and the filter cake was washed with ethyl acetate (5 mL×2). The organic phase of the filtrate was separated, and the aqueous phase was extracted with ethyl acetate repeatedly (15 mL×3). The combined organic phase was washed with saturated NaHCO₃ solution (30 mL) and brine (30 mL×2), dried over MgSO₄, and then concentrated on a rotary evaporator to dryness to afford 2-amino-4-chloro-6-isobutyl-7,8-dihydropyrido[2,3-d]pyrimidin-5(6H)-one as an off-white solid (107 mg, yield 18%). The crude product was used for the next step without further purification.

¹H NMR (DMSO-d₆) δ (ppm) 7.93 (1H, s), 7.24 (1H, s, br), 7.14 (1H, s, br), 3.42 (1H, m), 3.10 (1H, m), 2.40 (1H, m), 1.54 (2H, m), 1.16 (1H, m), 0.89 (3H, d, J=6.4 Hz), 0.83 (3H, d, J=6.4 Hz).

### Step 7: 2-Amino-4-chloro-6-isobutyl-8-((4-methoxy-3,5-dimethylpyridin-2-yl)methyl)-7,8-dihydropyrido[2,3-d]pyrimidin-5(6H)-one

A suspension of 2-amino-4-chloro-6-isobutyl-7,8-dihydropyrido[2,3-d]pyrimidin-5(6H)-one (104 mg, 0.41 mmol), 2-chloromethyl-4-methoxy-3,5-dimethylpyridine hydrochloride (140 mg, 0.63 mmol) and cesium carbonate (342 mg, 10.5 mmol) in anhydrous DMSO (3 mL) was stirred at room temperature under a nitrogen atmosphere for 3 h. The mixture was diluted with ethyl acetate (15 mL) and saturated NH₄Cl solution (10 mL). The organic phase was separated, and the aqueous phase was extracted with ethyl acetate (5 mL×2). The combined organic phase was washed with brine (5 mL×3), dried over MgSO₄, filtered through a layer of silica gel to remove the yellow color, and then concentrated on a rotary evaporator to dryness. The residue was purified by flash chromatography (silica gel, ethyl acetate/hexanes v/v from 0 to 100%) to yield 2-amino-4-chloro-6-isobutyl-8-((4-methoxy-3,5-dimethylpyridin-2-yl)methyl)-7,8-dihydropyrido[2,3-d]pyrimidin-5(6H)-one as a white solid (51 mg, yield 31%).

¹H NMR (CDCl₃) δ (ppm) 8.18 (1H, s), 5.16 (2H, s, br), 5.00 (1H, d, J=15.0 Hz), 4.92 (1H, d, J=15.0 Hz), 3.77 (3H, s), 3.59 (1H, dd, J=13.2, 5.2 Hz), 3.33 (1H, dd, J=13.2, 8.4 Hz), 2.59 (1H, m), 2.56 (6H, s), 1.64 (1H, m), 1.42 (1H, m), 1.11 (1H, m), 0.84 (3H, d, J=6.4 Hz), 0.80 (3H, d, J=6.4 Hz).

Additional exemplary compounds of formula (IV) are shown above.

The following compounds, among others, can be prepared using the similar protocols described above:
2-Amino-4-chloro-6-(cyclopentylmethyl)-8-((4-methoxy-3,5-dimethylpyridin-2-yl)methyl)-7,8-dihydropyrido[2,3-d]pyrimidin-5(6H)-one
2-Amino-4-chloro-6-(cyclohexylmethyl)-8-((4-methoxy-3,5-dimethylpyridin-2-yl)methyl)-7,8-dihydropyrido[2,3-d]pyrimidin-5(6H)-one
2-Amino-4-chloro-8-((4-methoxy-3,5-dimethylpyridin-2-yl)methyl)-6-((tetrahydro-2H-pyran-4-yl)methyl)-7,8-dihydropyrido[2,3-d]pyrimidin-5(6H)-one
2-Amino-4-chloro-6-isopentyl-8-((4-methoxy-3,5-dimethylpyridin-2-yl)methyl)-7,8-dihydropyrido[2,3-d]pyrimidin-5(6H)-one
2-Amino-4-chloro-6-(cyclopropylmethyl)-8-((4-methoxy-3,5-dimethylpyridin-2-yl)methyl)-7,8-dihydropyrido[2,3-d]pyrimidin-5(6H)-one
2-Amino-4-chloro-6-isopropyl-8-((4-methoxy-3,5-dimethylpyridin-2-yl)methyl)-7,8-dihydropyrido[2,3-d]pyrimidin-5(6H)-one
2-Amino-4-chloro-6-cyclopentyl-8-((4-methoxy-3,5-dimethylpyridin-2-yl)methyl)-7,8-dihydropyrido[2,3-d]pyrimidin-5(6H)-one
2-Amino-4-chloro-6-cyclohexyl-8-((4-methoxy-3,5-dimethylpyridin-2-yl)methyl)-7,8-dihydropyrido[2,3-d]pyrimidin-5(6H)-one
2-Amino-4-chloro-8-((4-methoxy-3,5-dimethylpyridin-2-yl)methyl)-6-(tetrahydro-2H-pyran-4-yl)-7,8-dihydropyrido[2,3-d]pyrimidin-5(6H)-one
2-Amino-4-chloro-8-((4-methoxy-3,5-dimethylpyridin-2-yl)methyl)-6-propyl-7,8-dihydropyrido[2,3-d]pyrimidin-5(6H)-one
2-Amino-4-chloro-6-ethyl-8-((4-methoxy-3,5-dimethylpyridin-2-yl)methyl)-7,8-dihydropyrido[2,3-d]pyrimidin-5(6H)-one
2-Amino-4-chloro-8-((4-methoxy-3,5-dimethylpyridin-2-yl)methyl)-6-methyl-7,8-dihydropyrido[2,3-d]pyrimidin-5(6H)-one
6-Allyl-2-amino-4-chloro-8-((4-methoxy-3,5-dimethylpyridin-2-yl)methyl)-7,8-dihydropyrido[2,3-d]pyrimidin-5(6H)-one
2-Amino-4-chloro-8-((4-methoxy-3,5-dimethylpyridin-2-yl)methyl)-6-(prop-2-ynyl)-7,8-dihydropyrido[2,3-d]pyrimidin-5(6H)-one
2-Amino-6-benzyl-4-chloro-8-((4-methoxy-3,5-dimethylpyridin-2-yl)methyl)-7,8-dihydropyrido[2,3-d]pyrimidin-5(6H)-one

### Exemplary Compounds of Formula (V)

### Example V-1

### (E)-2-Amino-4-chloro-6-(cyclopentylmethylene)-8-((4-methoxy-3,5-dimethylpyridin-2-yl)methyl)-7,8-dihydropyrido[2,3-d]pyrimidin-5(6H)-one

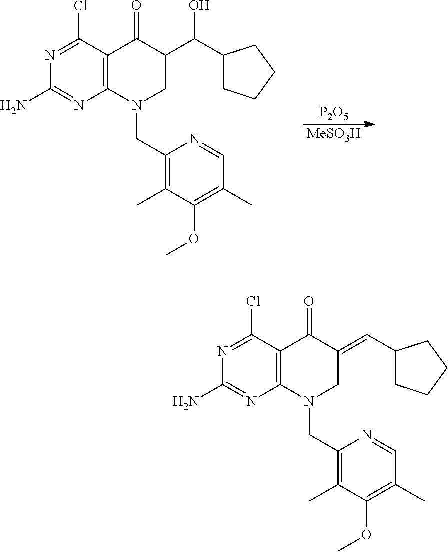

2-Amino-4-chloro-6-(cyclopentyl(hydroxy)methyl)-8-((4-methoxy-3,5-dimethylpyridin-2-yl)methyl)-7,8-dihydropyrido[2,3-d]pyrimidin-5(6H)-one (24 mg, 0.054 mmol) was dissolved in Eaton's reagent (P₂O₅, 7.5 wt. % in methanesulfonic acid) (1 mL). The resulting solution was allowed to stand at room temperature for 0.5 h, and was then poured into a mixture of crushed ice (10 g) and ammonium hydroxide (1 mL). Ethyl acetate (10 mL) was added. The resulting mixture was stirred at 0° C., and the pH was adjusted carefully with 1 N NaOH solution to 6. The organic phase was separated, and the aqueous phase was extracted with ethyl acetate (5 mL×2). The combined organic phase was washed with brine (5 mL×3), dried over MgSO₄, and then concentrated on a rotary evaporator to dryness. The residue was purified by flash chromatography (silica gel, ethyl acetate/hexanes v/v from 0 to 100%). A white solid was obtained (5 mg, yield 22%).

1H NMR (CDCl₃) S (ppm) 8.21 (1H, s), 6.72 (1H, dt, J=10.0, 2.0 Hz), 5.22 (2H, s, br), 4.96 (2H, s), 4.31 (2H, d, J=2.0 Hz), 3.78 (3H, s), 2.44 (1H, m), 2.27 (3H, s), 2.24 (3H, s), 1.75-1.61 (4H, m), 1.60-1.51 (2H, m), 1.37-1.24 (2H, m).

### Example V-2

### (E)-2-Amino-4-chloro-8-((4-methoxy-3,5-dimethylpyridin-2-yl)methyl)-6-(2-methylpropylidene)-7,8-dihydropyrido[2,3-d]pyrimidin-5(6H)-one

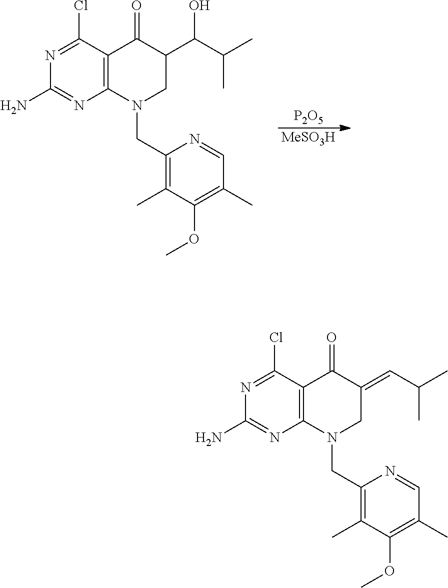

2-Amino-4-chloro-6-(1-hydroxy-2-methylpropyl)-8-((4-methoxy-3,5-dimethylpyridin-2-yl)methyl)-7,8-dihydropyrido[2,3-d]pyrimidin-5(6H)-one (39 mg, 0.093 mmol) was dissolved in Eaton's reagent (P₂O₅, 7.5 wt. % in methanesulfonic acid) (1 mL). The resulting solution was allowed to stand at room temperature for 0.5 h, and was then poured into a mixture of crushed ice (10 g) and ammonium hydroxide (1 mL). Ethyl acetate (10 mL) was added. The resulting mixture was stirred at 0° C., and the pH was adjusted carefully with 1 N NaOH solution to 6. The organic phase was separated, and the aqueous phase was extracted with ethyl acetate (5 mL×2). The combined organic phase was washed with brine (5 mL×3), dried over MgSO₄, and then concentrated on a rotary evaporator to dryness. The residue was purified by flash chromatography (silica gel, ethyl acetate/hexanes v/v from 0 to 100%). A white solid was obtained (24 mg, yield 64%).

1H NMR (CDCl₃) δ (ppm) 8.18 (1H, s), 6.60 (1H, dt, J=11.2, 2.4 Hz), 5.19 (2H, s, br), 4.96 (2H, s), 4.29 (2H, d, J=2.4 Hz), 3.76 (3H, s), 2.34 (1H, m), 2.25 (3H, s), 2.24 (3H, s), 0.94 (6H, d, J=7.6 Hz).

Additional exemplary compounds of formula (V) are shown above.

The following compounds, among others, can be prepared using the similar protocols described above:
(E)-2-Amino-4-chloro-6-(cyclohexylmethylene)-8-((4-methoxy-3,5-dimethylpyridin-2-yl)methyl)-7,8-dihydropyrido[2,3-d]pyrimidin-5(6H)-one
(E)-2-Amino-4-chloro-8-((4-methoxy-3,5-dimethylpyridin-2-yl)methyl)-6-((tetrahydro-2H-pyran-4-yl)methylene)-7,8-dihydropyrido[2,3-d]pyrimidin-5(6H)-one
(E)-2-Amino-4-chloro-8-((4-methoxy-3,5-dimethylpyridin-2-yl)methyl)-6-(3-methylbutylidene)-7,8-dihydropyrido[2,3-d]pyrimidin-5(6H)-one
(E)-2-Amino-4-chloro-6-(cyclopropylmethylene)-8-((4-methoxy-3,5-dimethylpyridin-2-yl)methyl)-7,8-dihydropyrido[2,3-d]pyrimidin-5(6H)-one
(E)-2-Amino-4-chloro-8-((4-methoxy-3,5-dimethylpyridin-2-yl)methyl)-6-propylidene-7,8-dihydropyrido[2,3-d]pyrimidin-5(6H)-one
(E)-2-Amino-6-benzylidene-4-chloro-8-((4-methoxy-3,5-dimethylpyridin-2-yl)methyl)-7,8-dihydropyrido[2,3-d]pyrimidin-5(6H)-one

Compounds of formula (VI) can be prepared in methods analogous to those used for preparation of compounds of formula (IV), using methods within the knowledge of persons of ordinary skill in the art.

Additional exemplary compounds of formula (VI) are shown above.

### Exemplary Compounds of Formula (VII)

### Example VII-1

### 2-Amino-4-chloro-8-((4-methoxy-3,5-dimethylpyridin-2-yl)methyl)-7,8-dihydropyrido[2,3-d]pyrimidin-6(5H)-one

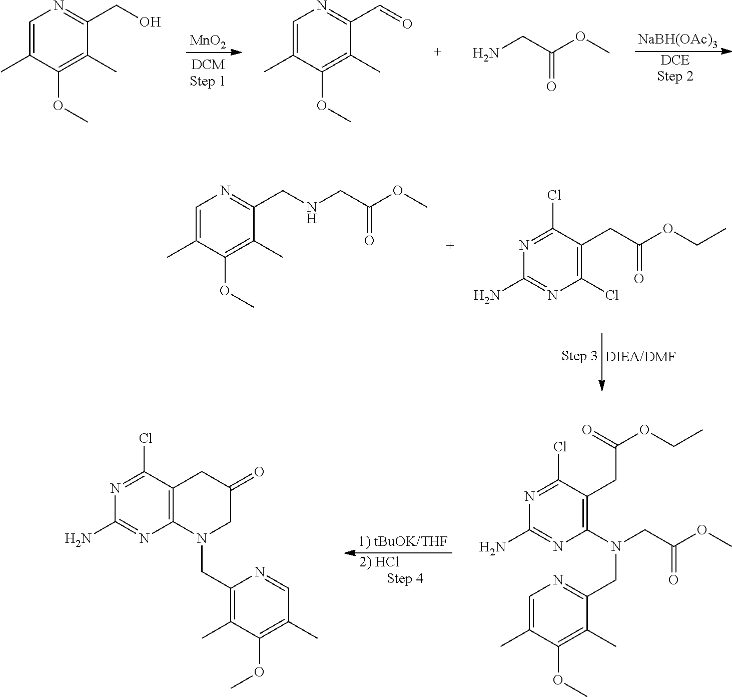

### Step 1: 4-Methoxy-3,5-dimethylpicolinaldehyde

To a stirred solution of (4-methoxy-3,5-dimethylpyridin-2-yl)methanol (9.6 g, 57.4 mmol) in chloroform (450 mL) was added activated MnO2 (60.0 g, 690 mmol) in batches at room temperature over 10 min. The resulting mixture was stirred at room temperature for 18 h, and was then filtered through Celite. The filter cake was washed with chloroform (75 mL×2). The filtrate was concentrated on a rotary evaporator to dryness. The oily residue was further dried under high vacuum to afford 4-methoxy-3,5-dimethylpicolinaldehyde as a light-pink oil (9.1 g, yield 96%), which crystallized upon storage at −20° C.

1H NMR (CDCl₃) δ (ppm) 10.15 (1H, s), 8.46 (1H, s), 3.80 (3H, s), 2.58 (3H, s), 2.35 (3H, s).

### Step 2: Methyl 2-((4-methoxy-3,5-dimethylpyridin-2-yl)methylamino)acetate

A suspension of glycine methyl ester hydrochloride (1.8 g, 14.3 mmol), 4-methoxy-3,5-dimethylpicolinaldehyde (2.15 g, 13.0 mmol) and triethylamine (2.2 mL, 15.8 mmol) in 1,2-dichloroethane (45 mL) was stirred at room temperature for 10 min, and sodium triacetoxyborohydride (3.86 g, 18.2 mmol) was added in one portion. The resulting mixture was stirred at room temperature under a nitrogen atmosphere for 1.5 h. Saturated NaHCO₃ solution (50 mL) was added. The organic phase was separated, and the aqueous phase was extracted with dichloromethane (15 mL×2). The combined organic phase was washed with water (15 mL×2), dried over MgSO₄, and concentrated on a rotary evaporator to dryness. The residue was further dried under high vacuum to afford methyl 2-((4-methoxy-3,5-dimethylpyridin-2-yl)methylamino)acetate as a light-yellow oil (2.25 g, yield 73%).

1H NMR (CDCl₃) δ (ppm) 8.20 (1H, s), 3.88 (2H, s), 3.75 (3H, s), 3.73 (3H, s), 3.54 (2H, s), 2.69 (1H, s, br), 2.24 (3H, s), 2.23 (3H, s).

### Step 3: Ethyl 2-(2-amino-4-chloro-6-((2-methoxy-2-oxoethyl)((4-methoxy-3,5-dimethylpyridin-2-yl)methyl)amino)pyrimidin-5-yl)acetate

To a stirred solution of ethyl 2-(2-amino-4,6-dichloropyrimidin-5-yl)acetate (2.35 g, 9.4 mmol) and methyl 2-((4-methoxy-3,5-dimethylpyridin-2-yl)methylamino)acetate (2.25 g, 9.4 mmol) in DMF (15 mL) was added N,N-diisopropylethylamine (1.73 mL, 9.9 mmol). The resulting mixture was stirred at 90° C. for 16 h. The reaction mixture was cooled to room temperature, and then diluted with ethyl acetate (100 mL) and saturated NH₄Cl solution (60 mL). The organic phase was separated, and the aqueous phase was extracted with ethyl acetate (20 mL×2). The combined organic phase was washed with brine (40 mL×3), dried over MgSO₄, and then concentrated on a rotary evaporator to a small volume (ca. 10 mL). The obtained suspension was filtered, and the filter cake was washed with ethyl acetate (3 mL×2). The filtrate was concentrated to dryness. The residue was purified by flash chromatography (silica gel, ethyl acetate/hexanes v/v from 0 to 100%) to yield ethyl 2-(2-amino-4-chloro-6-((2-methoxy-2-oxoethyl)((4-methoxy-3,5-dimethylpyridin-2-yl)methyl)amino)pyrimidin-5-yl)acetate as a light-brown solid (566 mg, yield 13%).

1H NMR (CDCl₃) δ (ppm) 8.24 (1H, s), 4.80 (2H, s), 4.75 (2H, s), 4.12 (2H, q, J=8.0 Hz), 4.10 (2H, s), 3.76 (3H, s), 3.71 (3H, s), 3.51 (2H, s), 2.26 (3H,$), 2.10 (3H, s), 1.19 (3H, t, J=8.0 Hz).

### Step 4: 2-Amino-4-chloro-8-((4-methoxy-3,5-dimethylpyridin-2-yl)methyl)-7,8-dihydropyrido[2,3-d]pyrimidin-6(5H)-one

To a stirred solution of ethyl 2-(2-amino-4-chloro-6-((2-methoxy-2-oxoethyl)((4-methoxy-3,5-dimethylpyridin-2-yl)methyl)amino)pyrimidin-5-yl)acetate (452 mg, 1.0 mmol) in anhydrous tetrahydrofuran (3 mL) at 0° C. under a nitrogen atmosphere was added potassium tert-butoxide solution (1.0 M in tetrahydrofuran, 1.2 mL, 1.2 mmol). The resulting solution was stirred at room temperature under a nitrogen atmosphere for 3 h, and 6 N HCl (3 mL) was added. The resulting mixture was stirred at 65° C. for 24 h. The reaction mixture was then cooled to room temperature, and diluted with water (25 mL) and ethyl acetate (20 mL). The mixture was basified carefully with 1 N NaOH solution to pH 6 under magnetic stirring. The organic phase was separated, and the aqueous phase was extracted with ethyl acetate (10 mL×2). The combined organic phase was washed with brine (15 mL×2), dried over MgSO₄, and then concentrated on a rotary evaporator to dryness. The residue was purified by flash chromatography (silica gel, ethyl acetate/hexanes v/v from 0 to 100%) to yield 2-Amino-4-chloro-8-((4-methoxy-3,5-dimethylpyridin-2-yl)methyl)-7,8-dihydropyrido[2,3-d]pyrimidin-6(5H)-one as a white solid (23 mg, yield 7%).

1H NMR (CDCl₃) δ (ppm) 8.15 (1H, s), 4.87 (2H, s), 4.76 (2H, s), 4.02 (2H, s), 3.77 (3H, s), 3.58 (2H, s), 2.24 (6H, s).

Additional exemplary compounds of formula (VII) are shown above.

The following compounds, among others, can be prepared using the similar protocols described above:
2-Amino-4-chloro-8-((4-methoxy-3,5-dimethylpyridin-2-yl)methyl)-5-methyl-7,8-dihydropyrido[2,3-d]pyrimidin-6(5H)-one
2-Amino-4-chloro-5-ethyl-8-((4-methoxy-3,5-dimethylpyridin-2-yl)methyl)-7,8-dihydropyrido[2,3-d]pyrimidin-6(5H)-one
2-Amino-4-chloro-5-isopropyl-8-((4-methoxy-3,5-dimethylpyridin-2-yl)methyl)-7,8-dihydropyrido[2,3-d]pyrimidin-6(5H)-one
2-Amino-4-chloro-8-((4-methoxy-3,5-dimethylpyridin-2-yl)methyl)-5,5-dimethyl-7,8-dihydropyrido[2,3-d]pyrimidin-6(5H)-one
2′-Amino-4′-chloro-8′-((4-methoxy-3,5-dimethylpyridin-2-yl)methyl)-7′,8′-dihydro-6′H-spiro[cyclopentane-1,5′-pyrido[2,3-d]pyrimidin]-6′-one
2′-Amino-4′-chloro-8′-((4-methoxy-3,5-dimethylpyridin-2-yl)methyl)-7′,8′-dihydro-6′H-spiro[cyclohexane-1,5′-pyrido[2,3-d]pyrimidin]-6′-one
2-Amino-4-chloro-5,5-diethyl-8-((4-methoxy-3,5-dimethylpyridin-2-yl)methyl)-7,8-dihydropyrido[2,3-d]pyrimidin-6(5H)-one
2-Amino-4-chloro-5-isobutyl-8-((4-methoxy-3,5-dimethylpyridin-2-yl)methyl)-7,8-dihydropyrido[2,3-d]pyrimidin-6(5H)-one

### Example VIII-1

### Methyl 2-amino-4-chloro-8-((4-methoxy-3,5-dimethylpyridin-2-yl)methyl)-7,8-dihydropyrido[2,3-d]pyrimidine-6-carboxylate

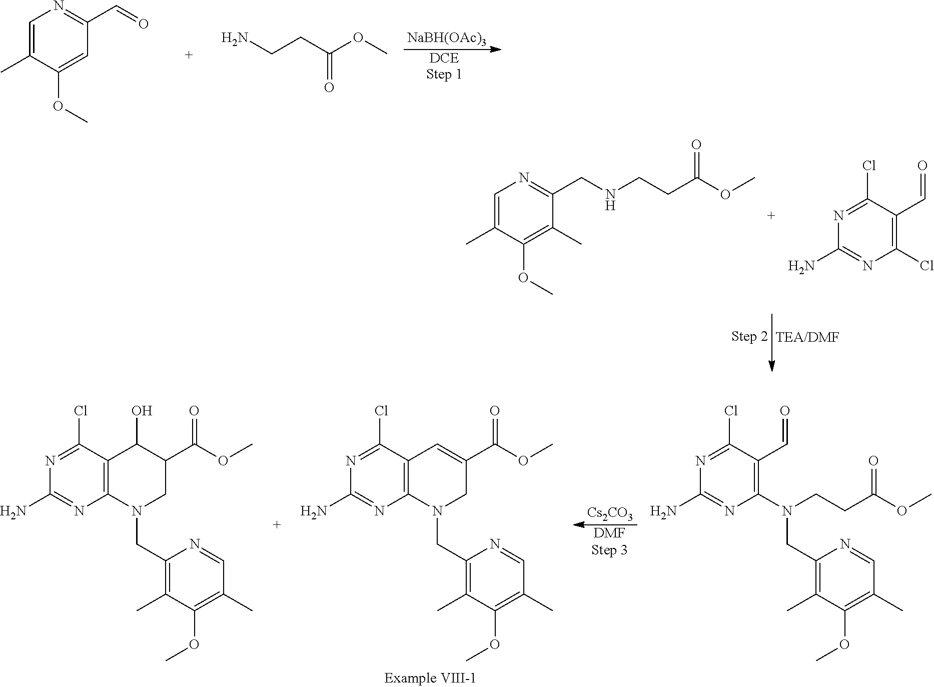

### Step 1: Methyl 3-((4-methoxy-3,5-dimethylpyridin-2-yl)methylamino)propanoate

A suspension of β-alanine methyl ester hydrochloride (2.00 g, 14.3 mmol), 4-methoxy-3,5-dimethylpicolinaldehyde (2.15 g, 13.0 mmol) and triethylamine (2.2 mL, 15.8 mmol) in 1,2-dichloroethane (45 mL) was stirred at room temperature for 10 min, and sodium triacetoxyborohydride (3.86 g, 18.2 mmol) was added in one portion. The resulting mixture was stirred at room temperature under a nitrogen atmosphere for 1.5 h. Saturated NaHCO₃ solution (50 mL) was added. The organic phase was separated, and the aqueous phase was extracted with dichloromethane (15 mL×2). The combined organic phase was washed with water (15 mL×2), dried over MgSO₄, and concentrated on a rotary evaporator to dryness. The residue was further dried under high vacuum to afford methyl 3-((4-methoxy-3,5-dimethylpyridin-2-yl)methylamino)propanoate as a light-yellow oil (2.3 g, yield 70%).

### Step 2: Methyl 3-((2-amino-6-chloro-5-formylpyrimidin-4-yl)((4-methoxy-3,5-dimethylpyridin-2-yl)methyl)amino)propanoate

To a stirred solution of 2-amino-4,6-dichloropyrimidine-5-carboxaldehyde (760 mg, 3.96 mmol) and methyl 3-((4-methoxy-3,5-dimethylpyridin-2-yl)methylamino)propanoate (1.01 g, 4.00 mmol) in DMF (6 mL) was added triethylamine (560 μL, 4.02 mmol). The resulting mixture was stirred at room temperature for 2 h, and then diluted with chloroform (60 mL), saturated NH₄Cl solution (25 mL) and water (15 mL). The organic phase was separated, and the aqueous phase was extracted with chloroform (20 mL×2). The combined organic phase was washed with water (20 mL×3), dried over MgSO₄, and then concentrated on a rotary evaporator to a small volume (ca. 5 mL). The obtained suspension was diluted with a mixture of ether and hexanes (v/v 1:1, 15 mL). The resulting mixture was allowed to stand at room temperature overnight. The product was collected by filtration, washed with hexanes (5 mL×2), and dried in vacuo to afford Methyl 3-((2-amino-6-chloro-5-formylpyrimidin-4-yl)((4-methoxy-3,5-dimethylpyridin-2-yl)methyl)amino)propanoate as a white solid (1.4 g, yield 87%).

¹H NMR (DMSO-d₆) δ (ppm) 9.82 (1H, s), 8.13 (1H, s), 7.58 (1H, s, br), 7.43 (1H, s, br), 4.70 (2H, s), 3.70 (3H, s), 3.54 (2H, t, J=6.8 Hz), 3.53 (3H, s), 2.71 (2H, t, J=6.8 Hz), 2.18 (3H, s), 2.01 (3H, s).

### Step 3: Methyl 2-amino-4-chloro-5-hydroxy-8-((4-methoxy-3,5-dimethylpyridin-2-yl)methyl)-5,6,7,8-tetrahydropyrido[2,3-d]pyrimidine-6-carboxylate and methyl 2-amino-4-chloro-8-((4-methoxy-3,5-dimethylpyridin-2-yl)methyl)-7,8-dihydropyrido[2,3-d]pyrimidine-6-carboxylate

A suspension of methyl 3-((2-amino-6-chloro-5-formylpyrimidin-4-yl)((4-methoxy-3,5-dimethylpyridin-2-yl)methyl)amino)propanoate (816 mg, 2.0 mmol) and cesium carbonate (977 mg, 3.0 mmol) in DMF (10 mL) was stirred at room temperature under a nitrogen atmosphere for 30 h. The reaction mixture was diluted with chloroform (60 mL), saturated NH₄Cl solution (30 mL) and water (10 mL). The organic phase was separated, and the aqueous phase was extracted with chloroform (20 mL×2). The combined organic phase was washed with water (20 mL×3), dried over MgSO₄, and then concentrated on a rotary evaporator to dryness. The residue was purified by flash chromatography (silica gel, ethyl acetate/hexanes v/v from 0 to 100%) to afford methyl 2-amino-4-chloro-5-hydroxy-8-((4-methoxy-3,5-dimethylpyridin-2-yl)methyl)-5,6,7,8-tetrahydropyrido[2,3-d]pyrimidine-6-carboxylate as a white solid (153 mg, yield 7%) and methyl 2-amino-4-chloro-8-((4-methoxy-3,5-dimethylpyridin-2-yl)methyl)-7,8-dihydropyrido[2,3-d]pyrimidine-6-carboxylate as a light-yellow solid

Methyl 2-amino-4-chloro-5-hydroxy-8-((4-methoxy-3,5-dimethylpyridin-2-yl)methyl)-5,6,7,8-tetrahydropyrido[2,3-d]pyrimidine-6-carboxylate: ¹H NMR (DMSO-d₆) 6 (ppm) 8.14 (1H, s), 6.39 (2H, s, br), 5.34 (1H, d, J=5.2 Hz), 5.00 (2H, m), 4.81 (1H, d, J=15.2 Hz), 3.71 (3H, s), 3.63 (3H, s), 3.61 (1H, m), 3.26 (1H, m), 2.84 (1H, m), 2.18 (6H, s).

Methyl 2-amino-4-chloro-8-((4-methoxy-3,5-dimethylpyridin-2-yl)methyl)-7,8-dihydropyrido[2,3-d]pyrimidine-6-carboxylate: ¹H NMR (DMSO-d₆) δ (ppm) 8.17 (1H, s), 7.34 (1H, s), 6.95 (2H, s, br), 4.80 (2H, s), 4.37 (2H, s), 3.72 (3H, s), 3.67 (3H, s), 2.18 (6H, s).

### Biological Activity

Most of the compounds synthesized were evaluated for their ability to induce degradation of the HSP90 client protein Her2 in SKBR-3 cells. Selected compounds were evaluated in a fluorescence polarization (FP) assay for their affinity to HSP90. Compounds with IC₅₀ values less than 2.0 μM in the Her2 degradation assay were then further evaluated for their inhibitory activity against the growth of several human tumor cell lines in culture, including MCF-7, BT474, SKBR-3, and HT29.

### Her-2 Degradation Assay

SKBr-3 cells grown in McCoy's 5a Medium supplemented with 10% fetal bovine serum (FBS) were plated into 12-well plates at 50% confluency. Increasing concentrations of inhibitors were added 24 hours later (cells are 65-70% confluent) and incubated overnight for 18 hours. The wells were washed with 2 ml of phosphate buffered saline (PBS), and 50 ul of trypsin was added to each well. After trypsinization was complete, 150 ul of FBS was added to each well. Then 200 ul of cells were transferred to 96-well plates and plates were centrifuged at 1200 rpm for 2 minutes using a tabletop centrifuge. The cells were washed once with 250 ul PBS before being lysed with 100 ul of 50 mM Tris (pH 7.4), 0.1% NP-40 lysis buffer. Protein concentrations of clarified lysates were determined using the BCA Assay (Calbiochem) according to the manufacturer's instructions. 2 ug of each lysate was analyzed in the human sp 185 Her-2 ELISA (Bender Medsystems) according to the manufacturer's instructions. ELISA plates were read at 450 nm using a multiwell plate spectrophotometer. Background was determined by measuring the Abs 450 nm of wells with no lysate added. The background value was subtracted from all values. Absorbance 450 nm values for each inhibitor were plotted against the log 10 inhibitor concentration, and IC50 values were calculated using a nonlinear least-square curve fitting program in Prism 5.

### MTS Assay (Cytotoxicity)

Cells were seeded in 96-well plates at 2000 cells/well in a final culture volume of 100 ul for 24 hours before the addition of 25 ul of increasing concentrations of inhibitors that was incubated for 5 days. Viable cell number was determined using the CellTiter 96 AQueous One Solution Cell Proliferation Assay (Promega). Plates were read at 490 nm absorbance 2-4 hours after addition of the MTS reagent using a multiwell plate spectrophotometer. Background was determined by measuring the Abs 490 nm of cell culture medium and MTS in the absence of cells. The background value was subtracted from all values. Absorbance 490 nm values for each inhibitor were plotted against the log 10 inhibitor concentration, and IC₅₀ values were calculated using a nonlinear least-square curve fitting program in Prism 5.

### Bioactivity of Compounds of the Invention

Compounds of the invention are found to be effective inhibitors of HSP90 activity and of Her-2 degradation, typically have IC₅₀ values in the sub-micromolar range. Compounds of the invention also inhibit the growth of human tumor cells including MCF-7, SKBR-3, BT474, and HT-29 with GI₅₀ values less than 2 μM. Table 1 shows IC₅₀ values for HSP90 and Her2 degradation for a series of compounds of formula (I).

TABLE 1
In vitro biological activity of series I toward HSP90 in a FP
and Her2 degradation assay

| | | | Her2 |
|---|---|---|---|
| | | FP assay | degradation |
| Series | R | IC₅₀, μM^{a} | IC₅₀, μM^{a} |
| I-2 | H | 0.88 | 0.57 |
| I-18 | — | na | >10 |
| I-19 | — | na | >5 |
| I-20 | — | >5 | >5 |
| I-5 | CH₃ | 0.96 | 0.93 |
| I-3 | (CH₃)₂CH(CH₂)₃— | 0.90 | 1.91 |
| I-7 | (pyrrolidinel-yl)-(CH₂)₂— | 0.66 | 0.51 |
| I-10 | (morpholino-1-yl)-(CH₂)₃— | na | 1.55 |
| I-4 | (CH₃)₂CH— | 0.07 | 0.05 |
| I-6 | (CH₃)₂CHCH₂— | 0.05 | 0.04 |
| I-14 | cyclopentyl | na | 0.11 |
| I-15 | (CH₃)₂N(CH₂)₂NHCOCH(CH₃)— | na | 0.10 |
| I-16 | cyclopentyl | na | 0.02 |
| I-17 | (morpholin-1-yl)(CH₂)₂NHCO— | na | 1.38 |

^{a}The IC₅₀ values are means of three independent experiments (na = not tested).

TABLE 2
Inhibition of Hsp90 binding and activity by 4, 5, 7-substituted 2-amino-8-((4-
methoxy-3,5-dimethylpyridin-2-yl)methyl)-7,8-dihydropteridin-6(5H)-ones

| | | | | FP assay | Her2 degradation |
|---|---|---|---|---|---|
| ID | R₁ | R₂ | R₃ | IC₅₀, μM^{a} | IC₅₀, μM^{a} |
| I-6 | Cl | (CH₃)₂CHCH₂— | H | 0.05 | 0.04 |
| I-23 | OCH₃ | (CH₃)₂CHCH₂— | H | na | 1.07 |
| I-24 | CH₃ | (CH₃)₂CHCH₂— | H | na | 0.98 |
| I-25 | Cl | H | (S)—CH₂CH₂OH | 0.64 | 0.28 |
| I-26 | Cl | H | (R)—CH₃— | 0.40 | 0.35 |
| I-27 | Cl | H | (S)—CH₃— | na | 0.43 |
| I-28 | Cl | H | (S)—(CH₃)₂CH— | 2.14 | 0.65 |
| I-29 | Cl | H | (S)—(CH₃)₂CHCH₂— | na | 0.37 |
| I-30 | Cl | (CH₃)₂CHCH₂— | (S)—CH₃— | na | >5 |
| I-31 | Cl | (CH₃)₂CHCH₂— | (S)—(CH₃)₂CH— | na | >5 |
| I-32 | Cl | (CH₃)₂CH— | (S)—(CH₃)₂CH— | 0.08 | 0.06 |

^{a}The IC₅₀ values are means of three independent experiments (na = not tested).

TABLE 3
Inhibition of Hsp90 binding and activity by 8-(aryl-methyl)
substituted 2-amino-4-chloro-5-isobutyl-7,8-dihydropteridin-6(5H)-one

| | | | | FP assay | Her2 degradation |
|---|---|---|---|---|---|
| ID | R1 | R2 | R3 | IC₅₀, μM^{a} | IC₅₀, μM^{a} |
| If | CH₃ | OCH₃ | CH₃ | 0.05 | 0.03 |
| I-33 | CH₃ | Cl | CH₃ | na | 0.59 |
| I-34 | CH₃ | Br | CH₃ | 0.06 | 0.10 |
| I-35 | OCH₃ | OCH₃ | H | na | 0.16 |
| I-36 | — | — | — | na | >5 |

^{a}The IC₅₀ values are means of three independent experiments (na = not tested).

TABLE 4
Growth inhibition of tumor cell lines and human mammary
epithelial cell in vitro

| | MCF-7 | HT29 | SKBR-3 | BT474 | hMEpiC |
|---|---|---|---|---|---|
| ID | IC₅₀, μM^{a} | IC₅₀, μM^{a} | IC₅₀, μM^{a} | IC₅₀, μM^{a} | IC₅₀, μM^{a} |
| I-2 | 1.49 | 1.18 | 0.39 | 0.65 | na |
| I-5 | 2.19 | 1.47 | 0.46 | 0.95 | na |
| I-3 | 3.11 | 2.26 | 0.97 | 1.65 | na |
| I-7 | 1.32 | 1.08 | 0.30 | 0.52 | na |
| I-10 | 2.59 | 2.95 | 1.11 | 1.81 | na |
| I-4 | 0.05 | 0.06 | 0.02 | 0.03 | 0.32 |
| I-6 | 0.07 | 0.04 | 0.01 | 0.02 | 0.13 |
| I-14 | 0.82 | 0.33 | 0.12 | 0.29 | na |
| I-15 | 0.12 | 0.10 | 0.03 | 0.06 | na |
| I-16 | 2.14 | 1.20 | >5 | >10 | na |
| I-17 | 0.04 | 0.02 | 0.005 | 0.01 | na |
| I-26 | 0.79 | 0.47 | 0.22 | 0.41 | na |
| I-27 | 0.94 | 0.66 | 0.25 | 0.47 | na |
| I-28 | 1.60 | 1.06 | 0.43 | 0.76 | na |
| I-29 | 0.93 | 0.69 | 0.23 | 0.49 | na |
| I-30 | 0.04 | 0.09 | 0.005 | 0.02 | na |
| I-33 | 0.47 | 0.28 | 0.11 | 0.24 | na |
| I-34 | 0.28 | 0.14 | 0.06 | 0.13 | 0.18 |
| I-35 | 0.10 | 0.04 | 0.01 | 0.03 | 0.24 |

^{a}The IC₅₀ values are means of three independent experiments (na = not tested).

TABLE 5
Biological data for series III

| | Her2 | | | | |
|---|---|---|---|---|---|
| Example | Degradation | Growth Inhibition (IC₅₀, μM) | | | |
| Number | IC₅₀, μM | MCF-7 | HT29 | SKBR-3 | BT474 |
| III-1 | 0.09 | 0.09 | 0.03 | 0.09 | 0.03 |
| III-2 | 1.67 | 3.65 | 1.21 | 0.96 | 1.02 |
| III-3 | 0.09 | 0.1 | 0.07 | 0.04 | 0.06 |
| III-4 | 0.34 | 0.53 | 0.12 | 0.16 | 0.19 |
| III-5 | 0.48 | 0.96 | 0.56 | 0.23 | 0.21 |
| III-7 | 6.57 | 5.43 | 2.01 | 5.75 | 4.71 |
| III-8 | 0.15 | 0.81 | 1.31 | 0.15 | 0.47 |
| III-16 | 0.03 | 0.1 | 0.05 | 0.02 | 0.01 |
| III-18 | 0.36 | >0.5 | 0.11 | 0.03 | 0.03 |
| III-19 | 0.01 | 0.07 | 0.01 | 0.001 | 0.0007 |
| III-10 | 0.28 | 1.27 | 0.48 | 0.17 | 0.32 |
| III-21 | 0.06 | 0.15 | 0.06 | 0.004 | 0.01 |
| III-22 | 0.08 | 0.4 | 0.36 | 0.08 | 0.11 |
| III-52 | 0.06 | 0.19 | 0.24 | 0.14 | 0.41 |
| III-11 | 0.19 | 1.38 | 0.38 | 0.14 | 0.43 |
| III-57 | 0.49 | 0.73 | 0.58 | 0.17 | 0.52 |
| III-71 | 0.05 | 0.37 | nt | 0.03 | 0.08 |
| III-72 | 0.06 | 0.36 | nt | 0.09 | 0.07 |
| III-59 | 0.1 | 1.7 | 0.32 | 0.06 | 0.16 |
| III-75 | 0.09 | 0.16 | 0.05 | 0.007 | 0.06 |
| III-26 | 0.005 | 0.03 | 0.0006 | <0.0002 | 0.001 |
| III-27 | 0.13 | 0.33 | 0.13 | 0.05 | 0.08 |
| III-28 | 0.11 | 0.89 | 0.05 | 0.04 | 0.02 |
| III-29 | 0.07 | 0.41 | nt | 0.02 | 0.03 |
| III-30 | 0.05 | 0.68 | 0.21 | 0.02 | 0.07 |
| III-102 | 0.04 | 21.3 | 0.03 | 0.01 | 0.07 |
| III-33 | 0.04 | 0.11 | 0.02 | 0.0006 | 0.003 |
| III-34 | 0.02 | 0.17 | 0.02 | 0.01 | 0.04 |
| III-94 | 0.08 | 0.39 | 0.35 | 0.09 | 0.79 |
| III-36 | 0.008 | 0.41 | nt | 0.0009 | 0.003 |
| III-93 | 0.02 | 0.13 | nt | 0.01 | 0.06 |
| III-86 | 0.31 | 1.37 | nt | 1.13 | 0.76 |
| III-95 | 0.01 | 0.71 | nt | 0.002 | 0.02 |
| III-69 | 0.01 | 0.77 | nt | 0.01 | 0.06 |
| III-103 | 0.07 | 0.81 | nt | 0.04 | 0.11 |
| III-104 | 0.01 | 5.88 | nt | 0.01 | 0.19 |
| III-105 | 0.07 | >1 | nt | 0.1 | 1.28 |
| III-106 | 0.04 | 0.24 | nt | 0.04 | 0.09 |
| III-107 | 0.02 | 0.23 | nt | 0.005 | 0.02 |
| III-70 | 0.04 | 0.32 | nt | 0.02 | 0.08 |

Compounds active in the Her2 degradation assay were further evaluated for their ability to inhibit human tumor cell growth in vitro (Table 4).

Using the synthetic procedures provided herein, it is within ordinary skill to prepare any compounds of the invention. Using the knowledge of the person of ordinary skill combined with the above cited references and methods for evaluation of HSP90 inhibitory bioactivity, the person of ordinary skill in the art can evaluate any compound so prepared for its effectiveness in inhibiting HSP90, and for effectiveness in cell-based bioassays indicative of HPS90 inhibition in vivo. Accordingly, the full scope of the claims provided below are enabled by the disclosure herein.

It is within ordinary skill to evaluate any compound disclosed and claimed herein for effectiveness in inhibition of HSP90 and in the various cellular assays using the procedures described above. Accordingly, the person of ordinary skill can prepare and evaluate any of the claimed compounds without undue experimentation.

Any compound found to be an effective inhibitor of HSP90 can likewise be tested in animal models and in human clinical studies using the skill and experience of the investigator to guide the selection of dosages and treatment regimens.

### REFERENCES

1. Maloney, A.; Workman, P. Expert Opin. Biol. Ther. 2002, 2, 3.
2. Pearl, L. H.; Prodromou, C. Annu. Rev. Biochem. 2006, 75, 271.
3. Whitesell, L.; Lindquist, S. L. Nat. Rev. Cancer 2005, 5, 761.
4. Weigel, B. J.; Blaney, S. M.; Reid, J. M.; Safgren, S. L.; Bagatell, R.; Kersey, J.; Neglia, J. P.; Ivy, S. P.; Ingle, A. M.; Whitesell, L.; Gilbertson, R. J.; Krailo, M.; Ames, M.; Adamson, P. C. Clin. Cancer Res. 2007, 13, 1789.
5. Ronne, E. A.; Kondagunta, G. V.; Ishill, N.; Sweeney, S. M.; DeLuca, J. K.; Schwartz, L.; Bacik, J.; Motzer, R. J. Invest. New Drugs 2006, 24, 543.
6. McCollum, A. K.; TenEyck, C. J.; Stensgard, B.; Morlan, B. W.; Ballman, K. V.; Jenkins, R. B.; Toft, D. O.; Erlichman, C. Cancer Res. 2008, 68(18), 7419.
7. Kasibhatla, S. R.; Hong, K.; Biamonte, M. A.; Busch, D. J.; Karjian, P. L.; Sensintaffar, J. L.; Kamal, A.; Lough, R. E.; Brekken, J.; Lundgren, K.; Crecko, R.; Timony, G. A.; Ran, Y.; Mansfield, R.; Fritz, L. C.; Ulm, E.; Burrows, F. J.; Boehm, M. F. J. Med. Chem. 2007, 50, 2767.
8. Chandarlapaty, S.; Sawai, A.; Ye, Q.; Scott, A.; Silinski, M; Huang, K.; Fadden, P.; Partdrige, J.; Hall, S.; Steed, P.; Norton, L.; Rosen, N.; Solit, D. B. Clin. Cancer Res. 2008, 14(1), 240.
9. Brough, P. A.; Aherne, W.; Barril, X.; Borgognoni, J.; Boxall, K.; Cansfield, J. E.; Cheung, K. J.; Collins, I.; Davies, N. G. M.; Drysdale, M. J.; Dymock, B.; Eccles, S. A.; Finch, H.; Fink, A.; Hayes, A.; Howes, R.; Hubbard, R. E.; James, K.; Jordan, A. M.; Lockie, A.; Martins, V.; Massey, A.; Matthews, T. P.; McDonald, E.; Northfield, C. J.; Pearl, L. H.; Prodromou, C.; Ray, S.; Raynaud, F. I.; Roughley, S. D.; Sharp, S. Y.; Surgenor, A.; Walmsley, D. L.; Webb, P.; Wood, M.; Workman, P.; Wright, L. J. Med. Chem. 2008, 51, 196.
10. Stebbins, C. E.; Russo, A. A.; Schneider, C.; Rosen, N.; Hartl, F. U.; Pavletich, N. P. Cell 1997, 89, 239.
11. Barril, X.; Beswick, M. C.; Collier, A.; Drysdale, M. J.; Dymock, B. W.; Fink, A.; Grant, K.; Howes, R.; Jordan, A. M.; Massey, A.; Surgenor, A.; Wayne, J.; Workman, P.; Wright, L. Bioorg. Med. Chem. Lett. 2006, 16(9), 2543.
12. Barta, T. E.; Veal, J. M.; Rice, J. W.; Partridge, J. M.; Fadden, R. P.; Ma, W.; Jenks, M.; Geng, L.; Hanson, G. J.; Huang, K. H.; Barabasz, A. F.; Foley, B. E.; Otto, J.; Hall, S. E. Bioorg. Med. Chem. Lett. 2008, 18, 3517.
13. Denny, W. A.; Dobrusin, E. M.; Kramer, J. B.; MC Namara, D. J.; Rewcastle, G. W.; Showalter, H. D. H., Toogood, P. L. PCT Pat. Appl., WO200119825, 2001.
14. Ohmori, J.; Kubota, H.; Shimizu-Sasamata, M.; Okada, M.; Samamoto, S. J. Med. Chem. 1996, 39(6), 1331.
15. Cushman, M.; Jin, G.; Illarionov, B.; Fischer, M.; Ladenstein, R.; Bacher, A. J. Org. Chem. 2005, 70(20), 8162.
16. Makara, G.; Ewing, W.; Ma, Y.; Wintner, E. J. Org. Chem. 2001, 66(17), 5783.
17. Kasibhatla, S. R.; Hong, K.; Biamonte, M. A.; Busch, D. J.; Karjian, P. L.; Sensintaffar, J. L.; Kamal, A.; Lough, R. E.; Brekken, J.; Lundgren, K.; Grecko, R.; Timony, G. A.; Ran, Y.; Mansfield, R.; Fritz, L. C.; Ulm, E.; Burrows, F. J.; Boehm, M. F. J. Med. Chem. 2007, 50(12), 2726.

1. A compound of formula (III): 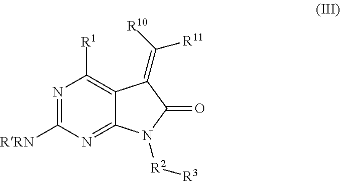
wherein
R and R′ are independently at each occurrence H, or (C₁-C₈)alkyl wherein any alkyl is optionally mono- or independently pluri-substituted with OR, halo, cyano, OC(O)R, OC(O)OR, CO₂R, NRR′, C(O)NRR′, N(R)C(O)R′, oxo, OC(O)NRR′, SR, S(O)R, S(O)₂R, S(O)₂OR, or SO₂NRR′ alkyl, acyl, alkoxy, cycloalkyl, heterocyclyl, aryl, or heteroaryl, wherein any alkyl, acyl, alkoxy, cycloalkyl, heterocyclyl, aryl, or heteroaryl is substituted with 0-4 Z; wherein any carbon atom of the alkyl, acyl, alkoxy, cycloalkyl, heterocyclyl, aryl, or heteroaryl, can be replaced by a heteroatom selected from the group consisting of NR, O, S, S(O), and S(O)₂; or, R and R′ can together with a nitrogen atom to which they are bonded form a heterocyclic or heteroaryl ring, wherein the heterocyclic or heteroaryl ring can further comprise 1-3 O, N, S, S(O), or S(O)₂ heteroatoms therewithin and is substituted with 0-4 Z groups wherein Z comprises independently at each occurrence (C₁-C₆)alkyl, (C₂-C₆)alkenyl, (C₂-C₆)alkynyl, halo, haloalkyl, cyano, hydroxy, oxo, alkoxy, NRR′, SR, CO₂R, nitro, SO₂R, SOR, acyl, haloacyl, N(R)C(O)R′, or CONRR′, wherein any alkyl, alkenyl, alkynyl, haloalkyl, alkoxy, R or R′ can be substituted with 0-4 Z;
R¹ comprises H, halo, NRR′, or hydroxyl; or comprises (C₁-C₆)alkyl, haloalkyl, cycloalkyl, or heterocyclyl, any of which can be optionally substituted by oxo, CO₂R, heterocylyl, aryl, OR, NRR′, or SR;
R² comprises (C(R″)₂)ₙ wherein n is 0-3, wherein R″ is independently at each occurrence H, alkyl, halo, haloalkyl, OR, or NRR′, or two R″ together with a carbon atom to which they are bonded comprise a carbonyl;
R³ comprises alkyl, cycloalkyl, heterocyclyl, aryl, or heteroaryl, wherein any alkyl, cycloalkyl, heterocyclyl, aryl, or heteroaryl is substituted with 0-5 Z groups;
R¹⁰ and R¹¹ are each independently H, alkyl, aryl, cycloalkyl, heterocyclyl, or heteroaryl, wherein any alkyl, aryl, cycloalkyl, heterocyclyl, or heteroaryl is substituted with 0-4 Z; wherein any aryl, cycloalkyl, heterocyclyl, or heteroaryl can be fused to a five to seven-membered carbocyclic or heterocyclic ring which is substituted with 0-4 Z; or, R¹⁰ and R¹¹ together with a carbon atom to which they are bonded form an optionally substituted carbocyclic or heterocyclic ring which is substituted with 0-4 Z; wherein a double bond marked Z, E can be in either a Z or an E configuration, or a mixture thereof;
or any salt, solvate, hydrate, tautomer, stereoisomer, or prodrug thereof.

2.-27. (canceled)

28. The compound of claim 1 wherein R³ comprises a substituted pyridyl moiety.

29. The compound of claim 28 wherein R²-R³ comprises a 3,5-dimethyl-4-methoxypyrid-2-ylmethyl moiety.

30. The compound of claim 1 wherein R¹ is halo.

31. The compound of claim 30 wherein R¹ is chloro.

32. The compound of claim 1 wherein R¹⁰ is H and R¹¹ is a substituted or unsubstituted heteroaryl group.

33. The compound of claim 1 wherein R¹¹ comprises a pyrrole or imidazole ring.

34. The compound of claim 1, comprising any of: 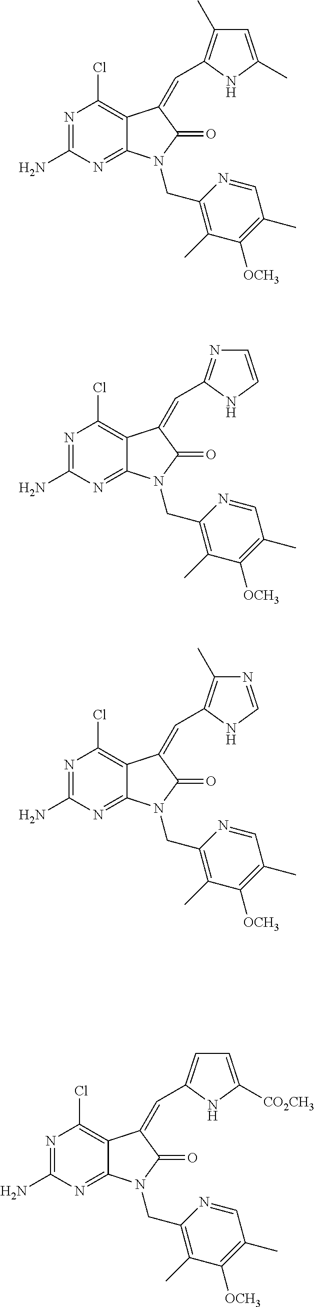 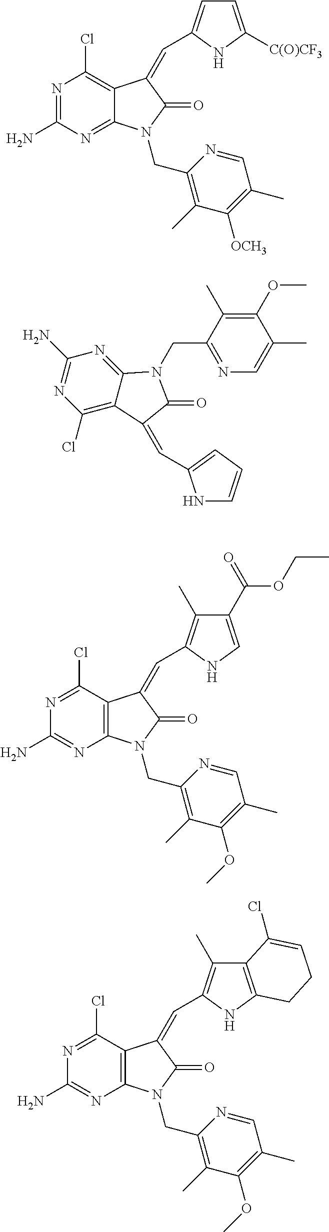 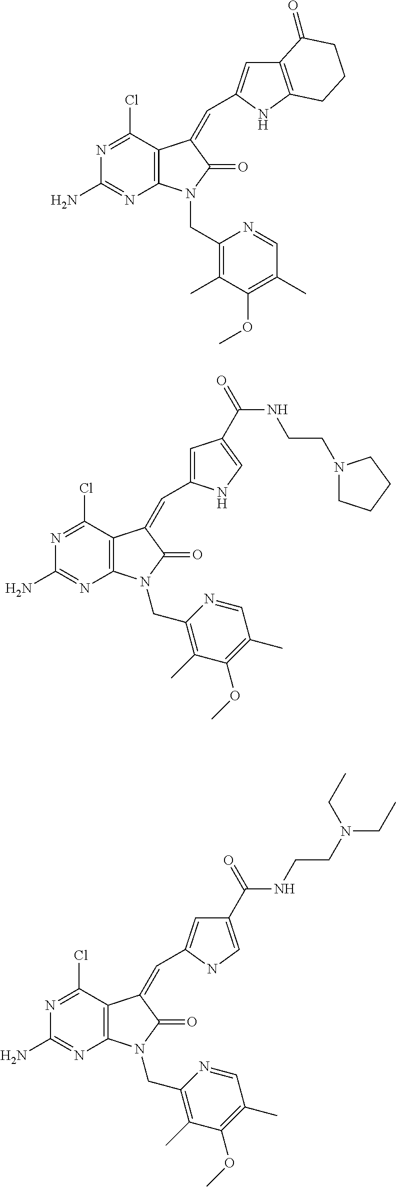 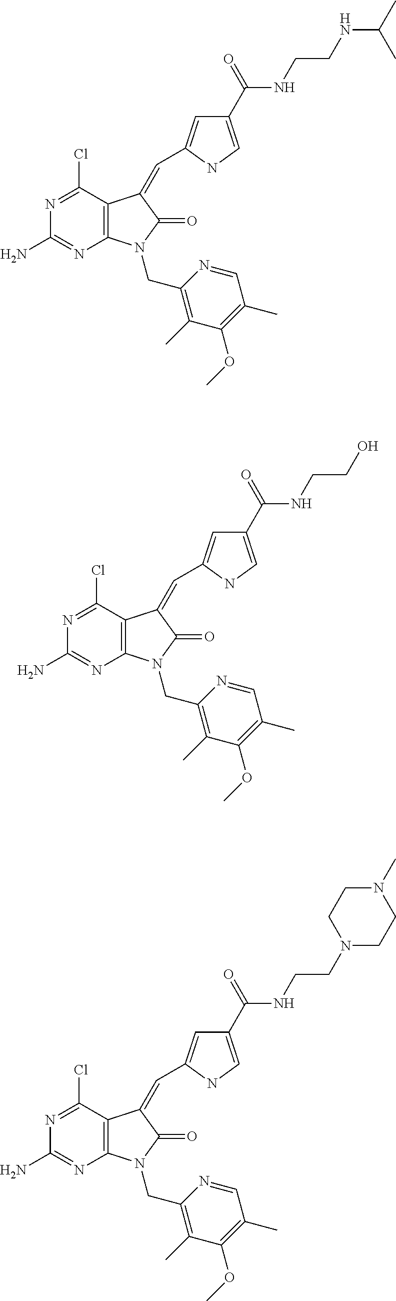 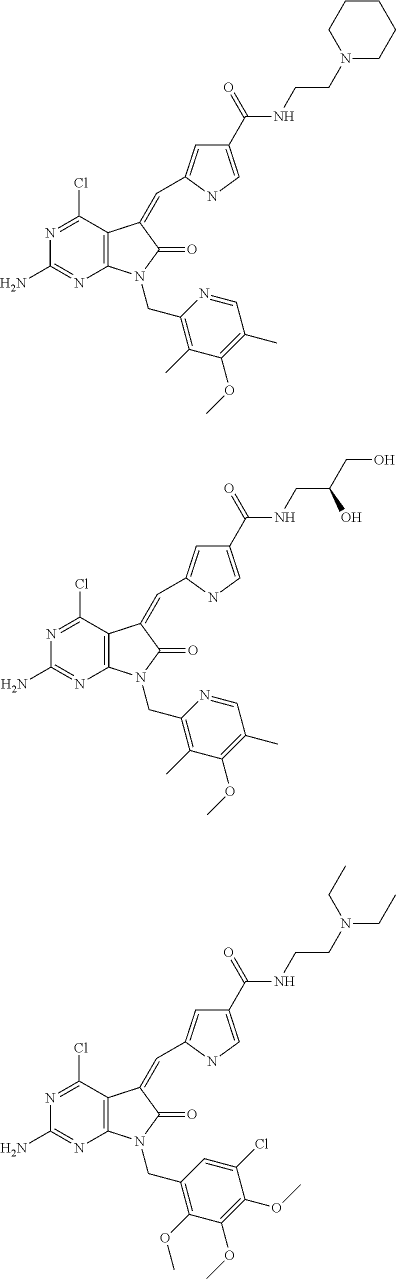 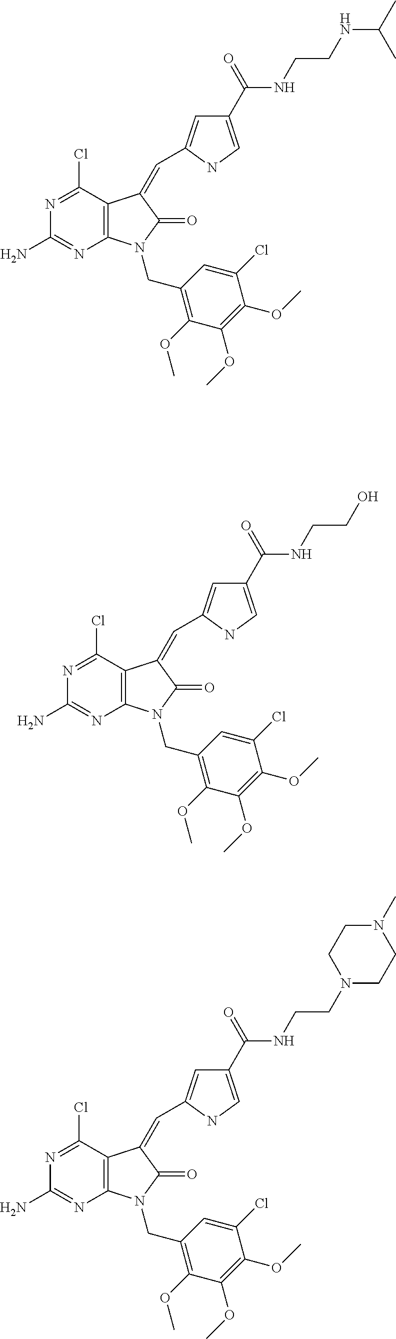 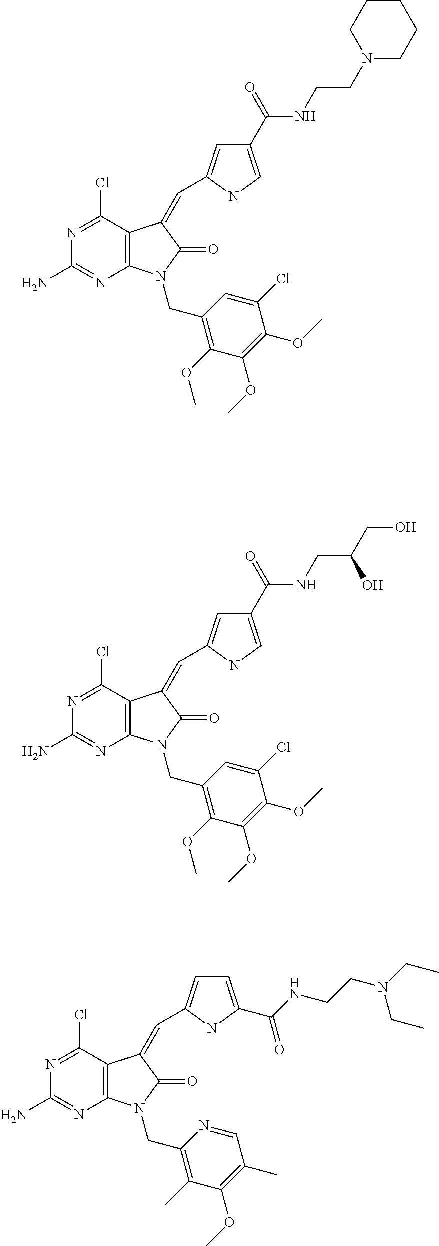 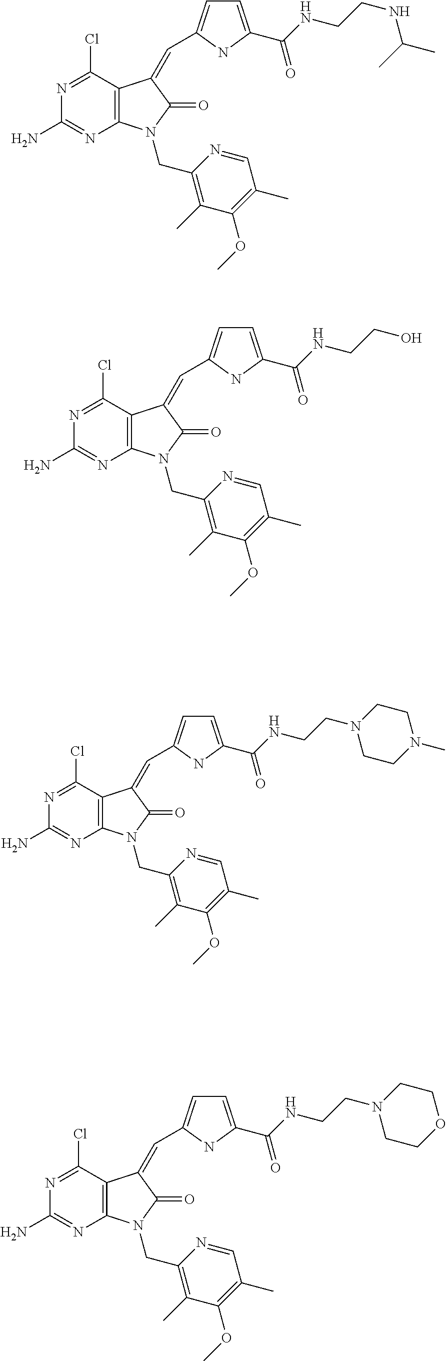 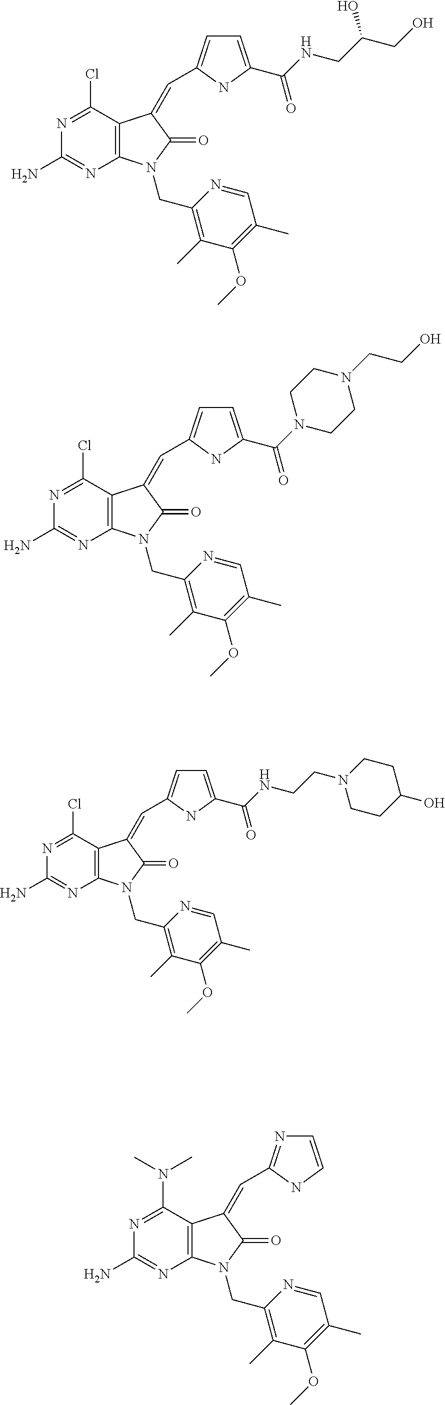 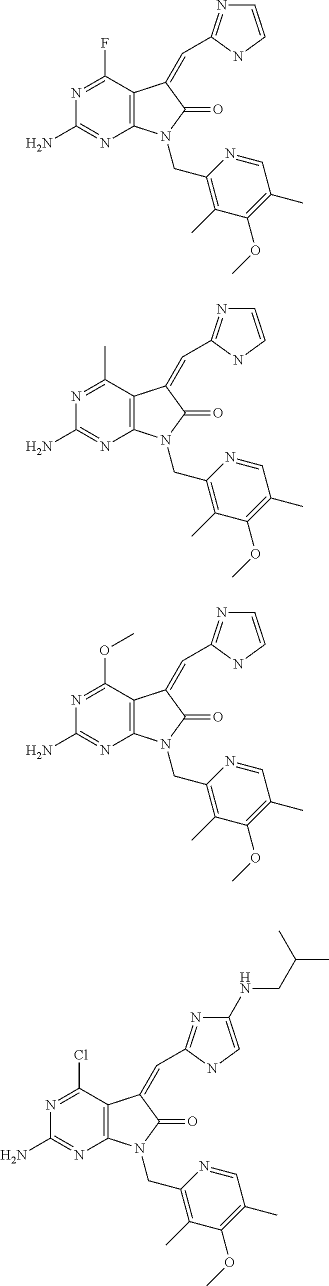 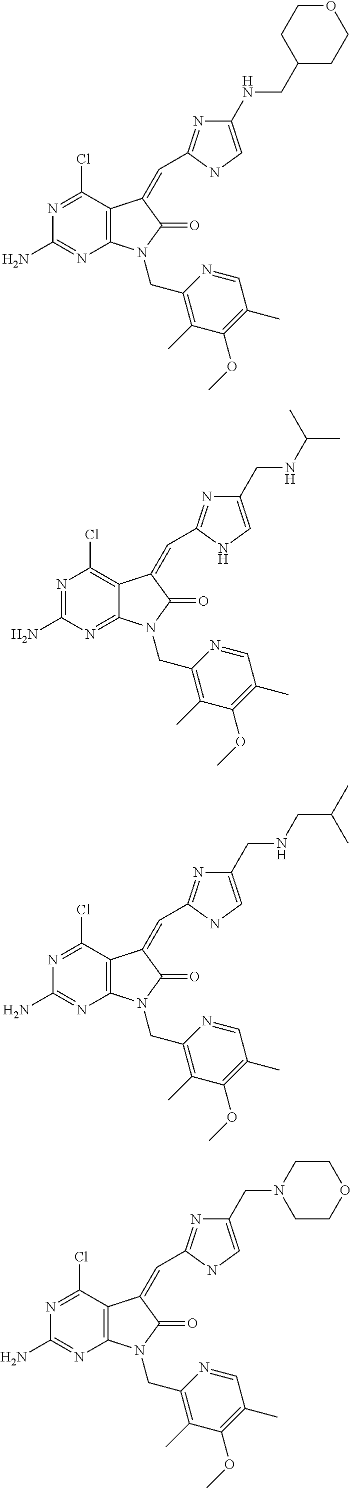 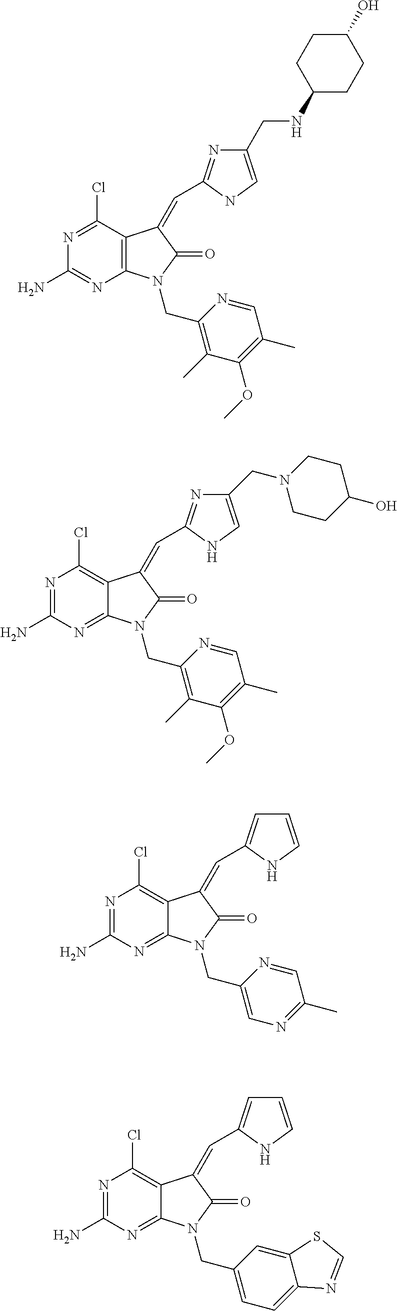 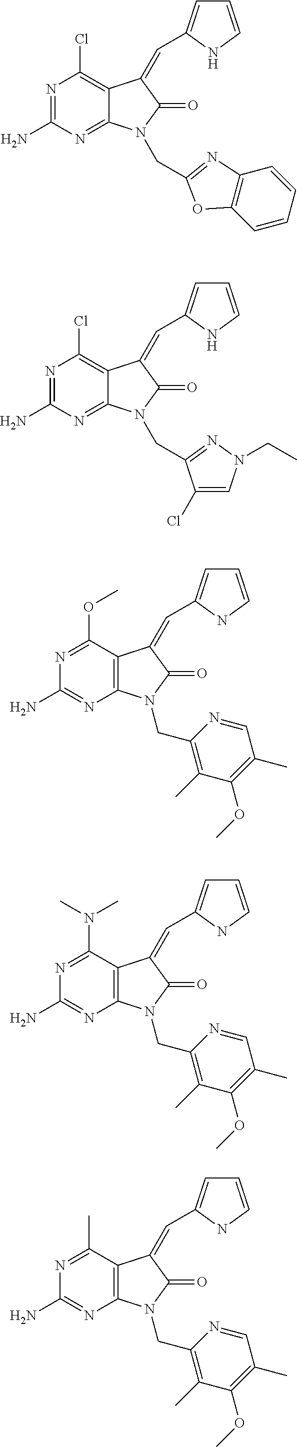 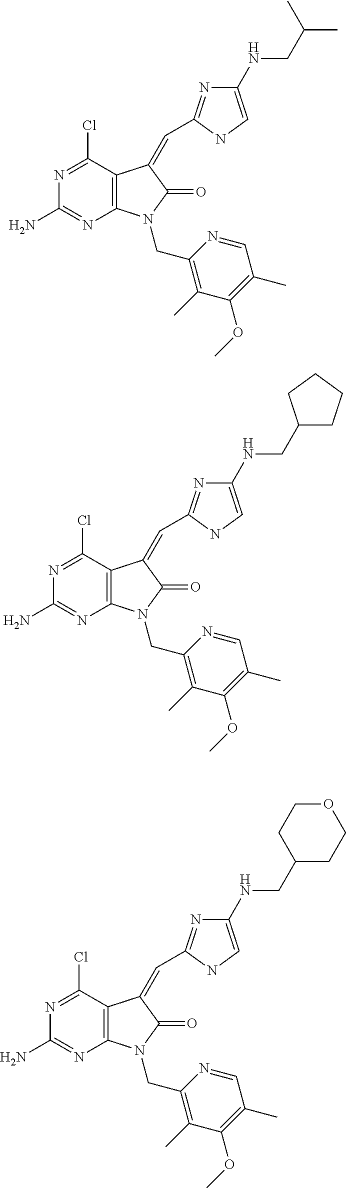 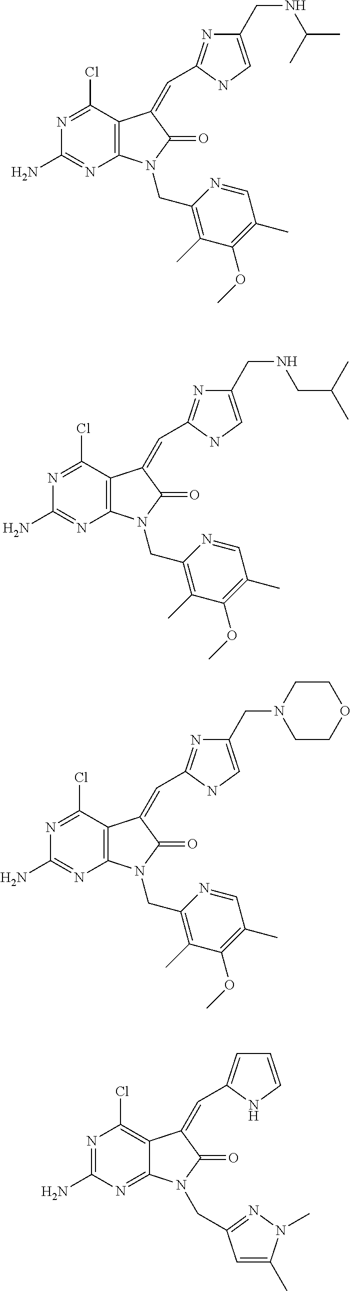 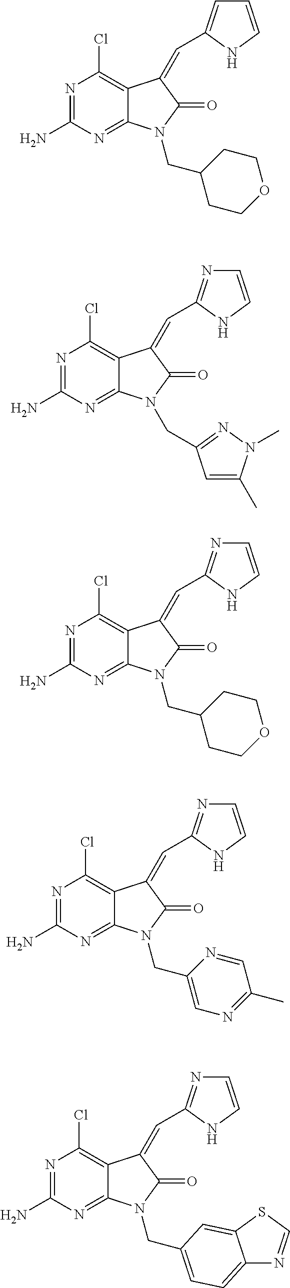 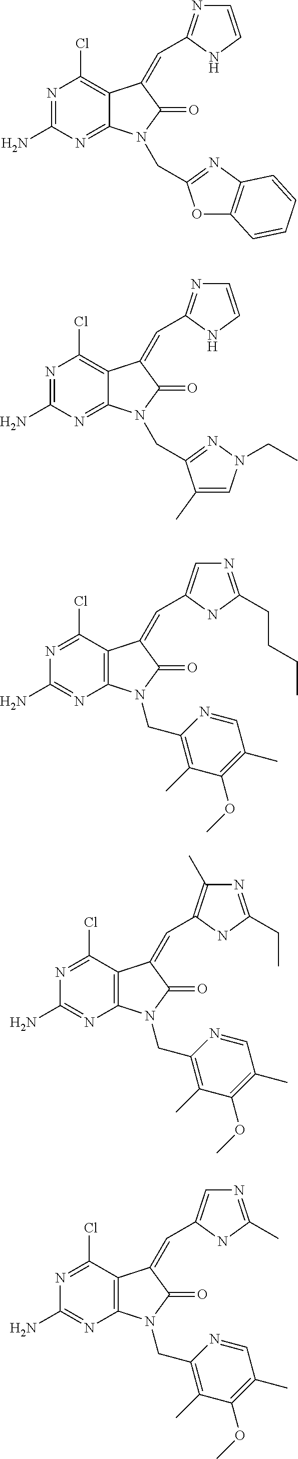 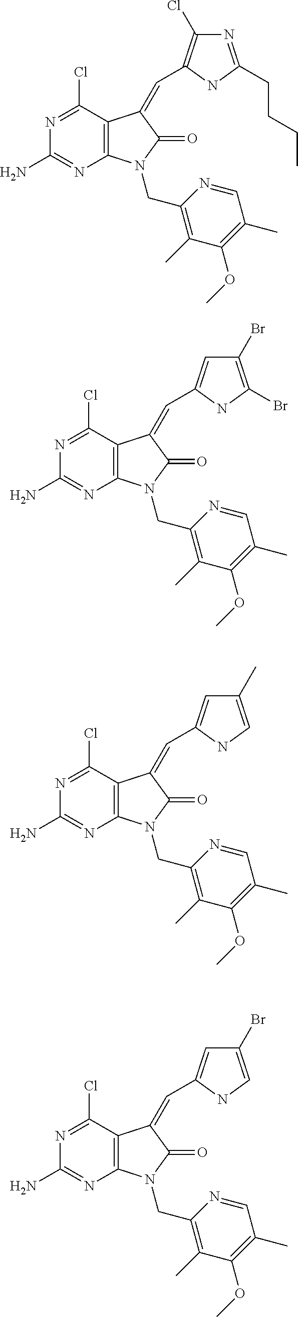 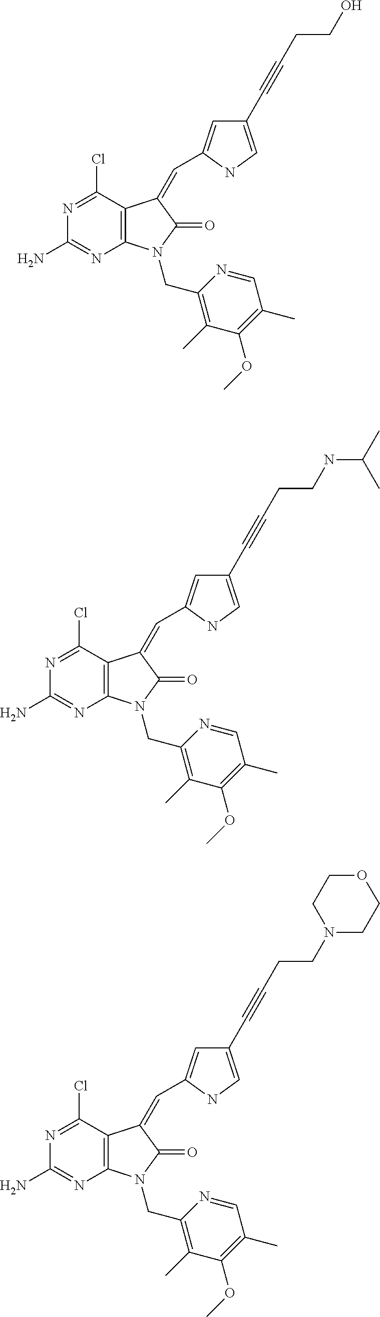 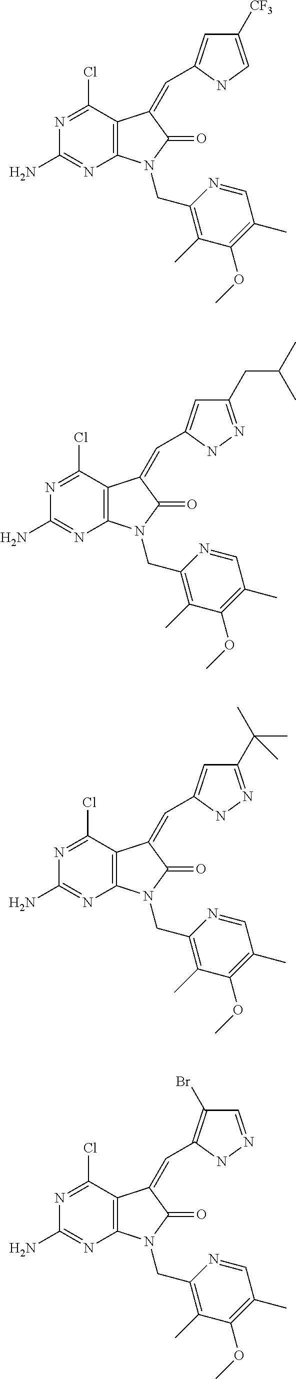 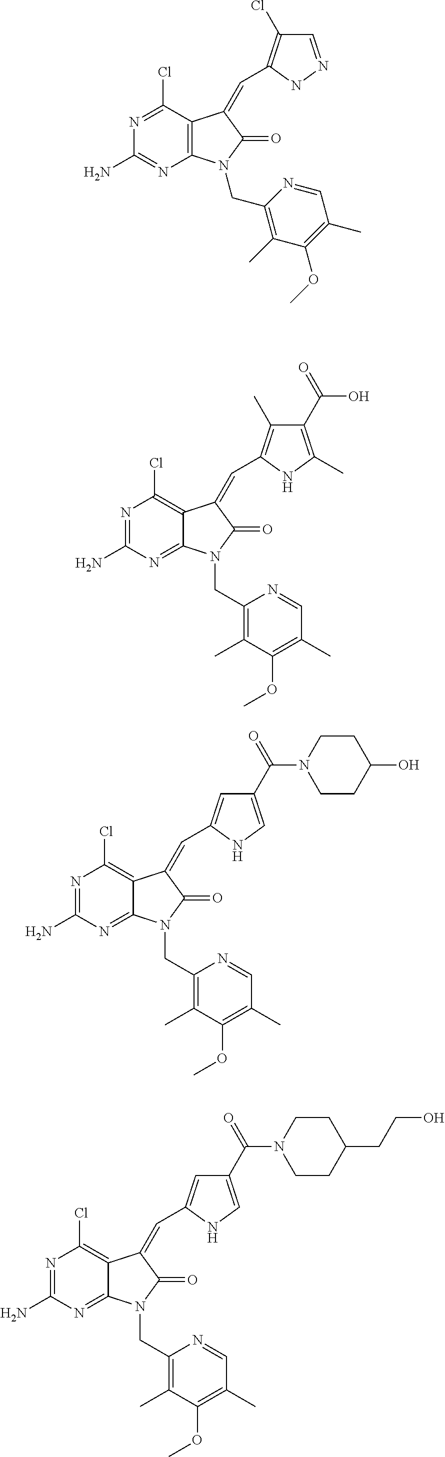 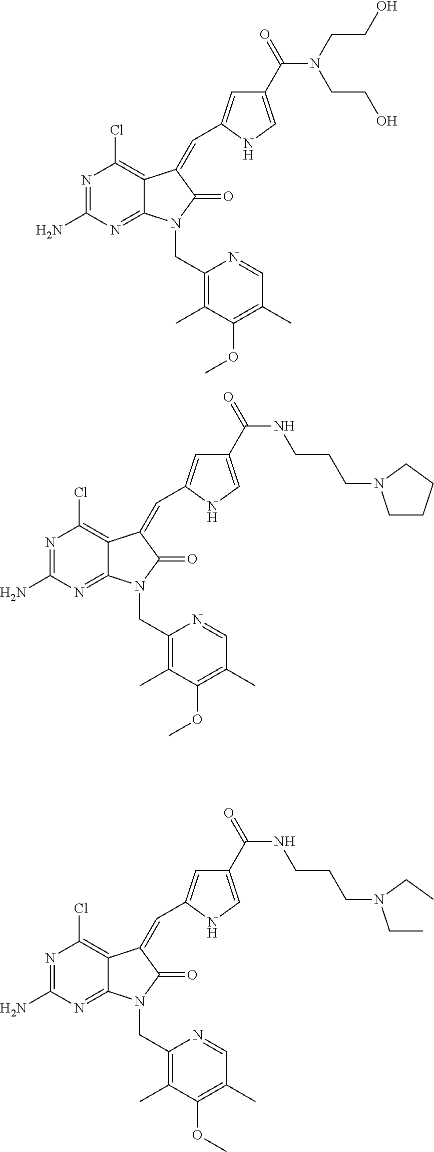 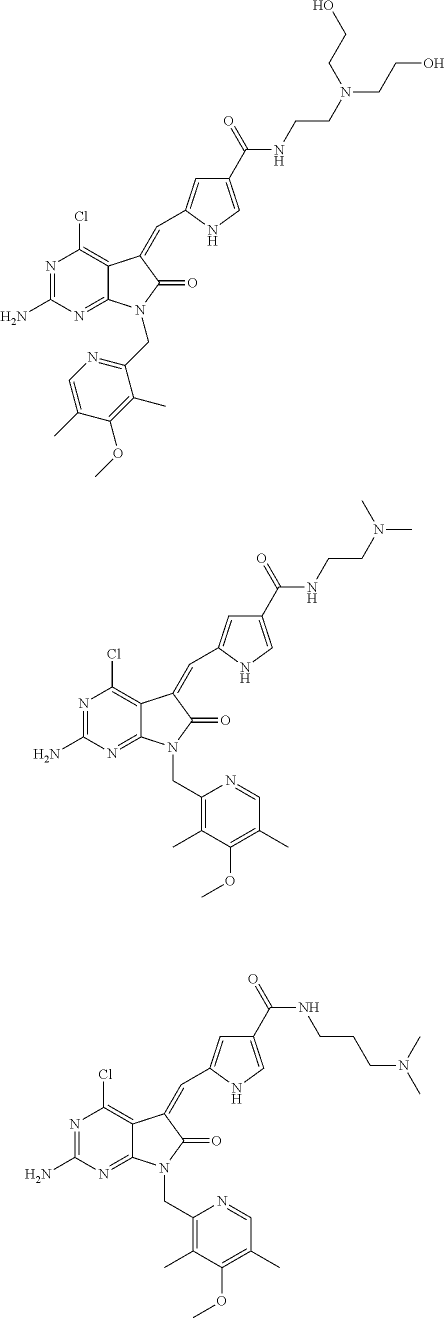 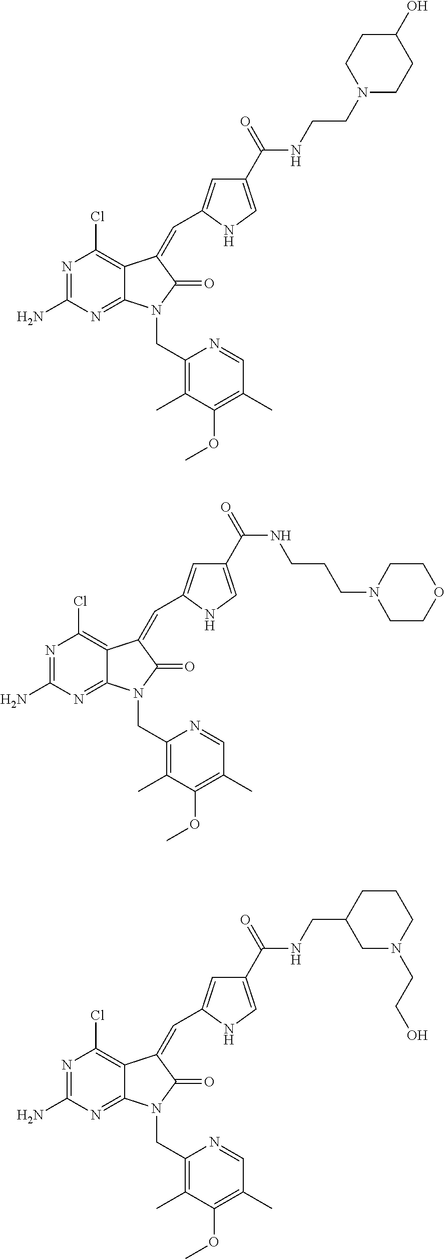 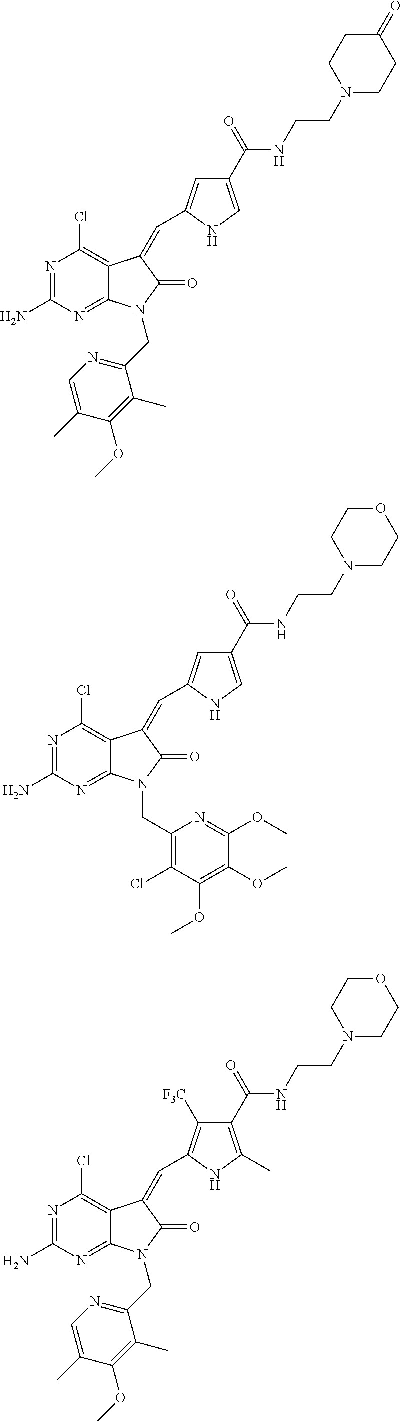 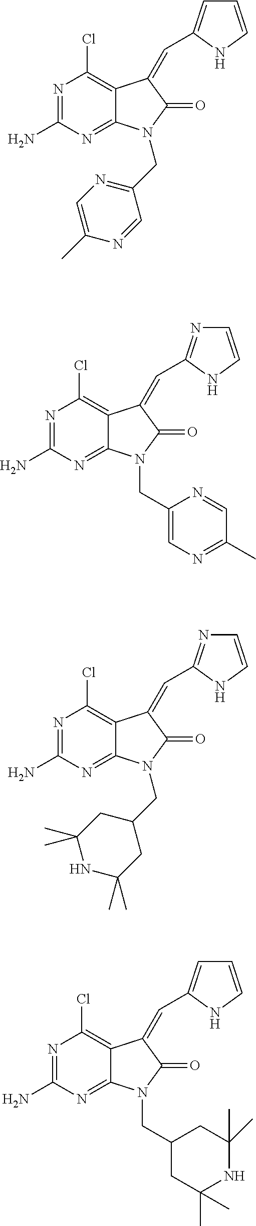 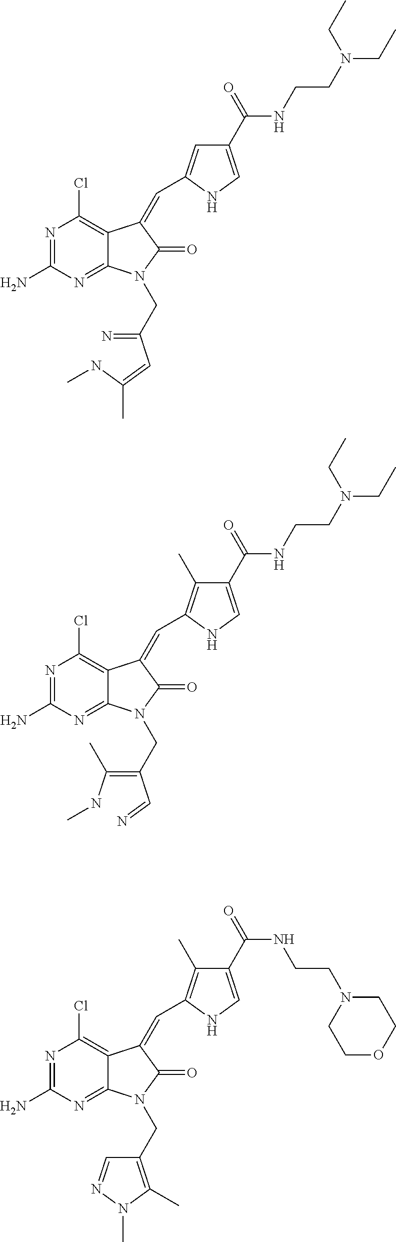 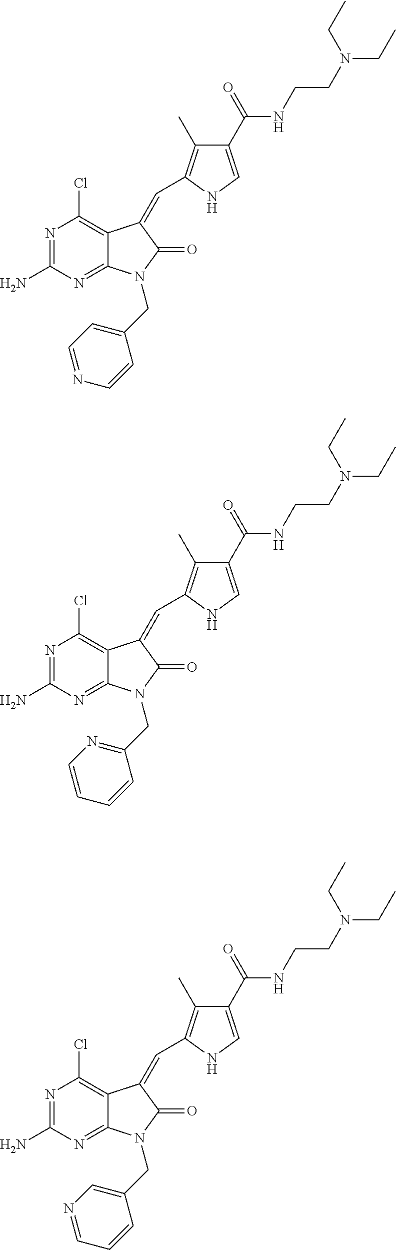 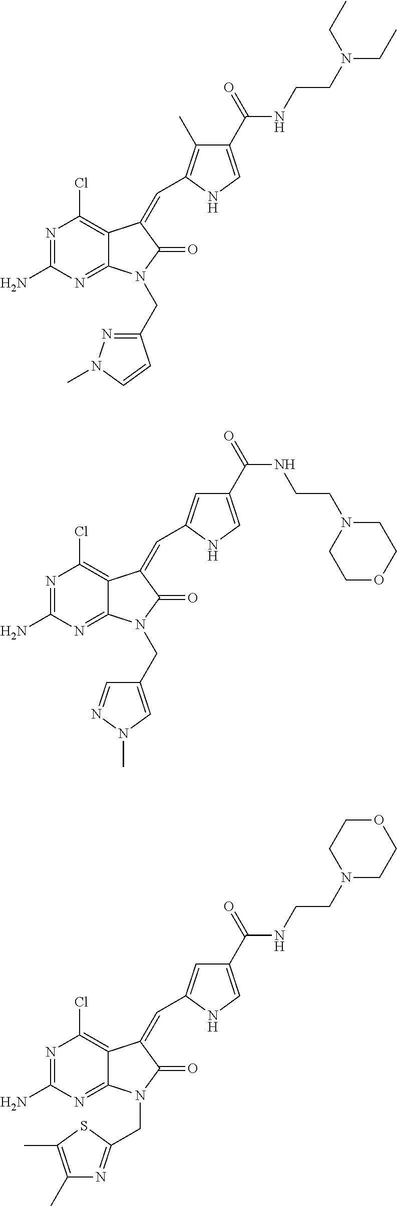 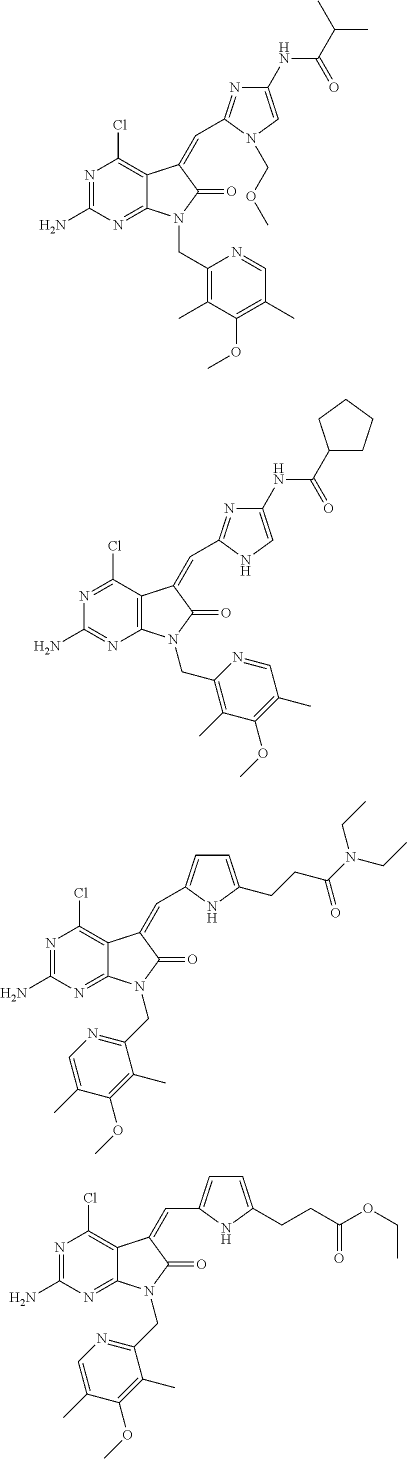 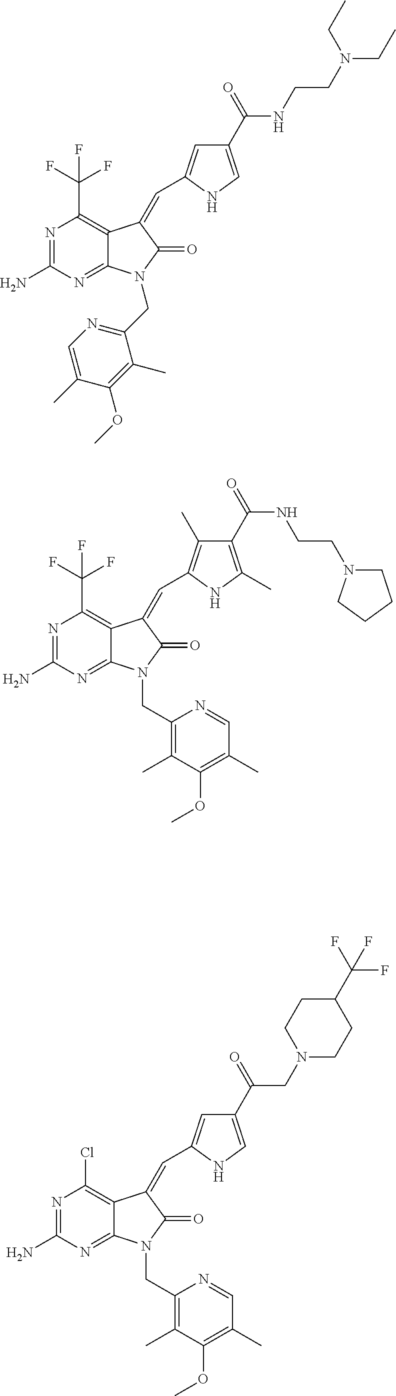 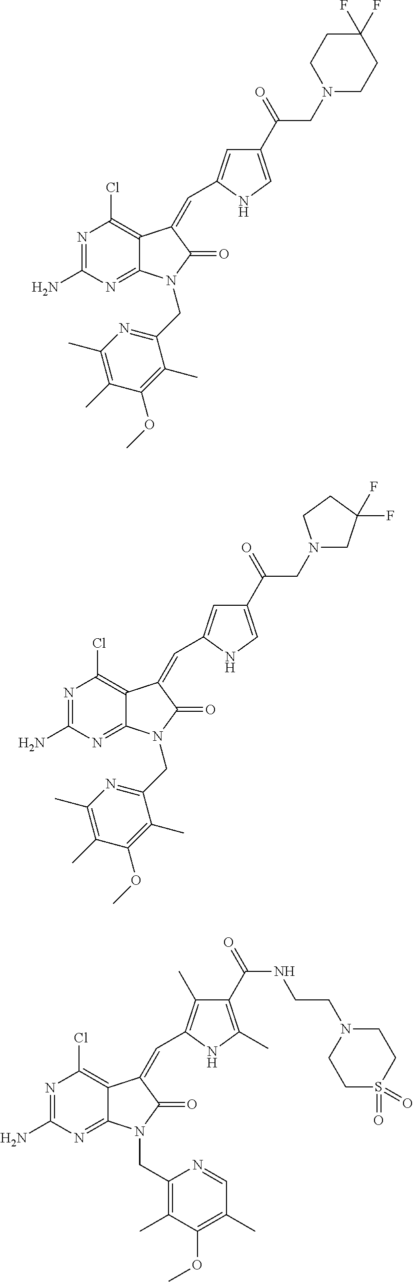 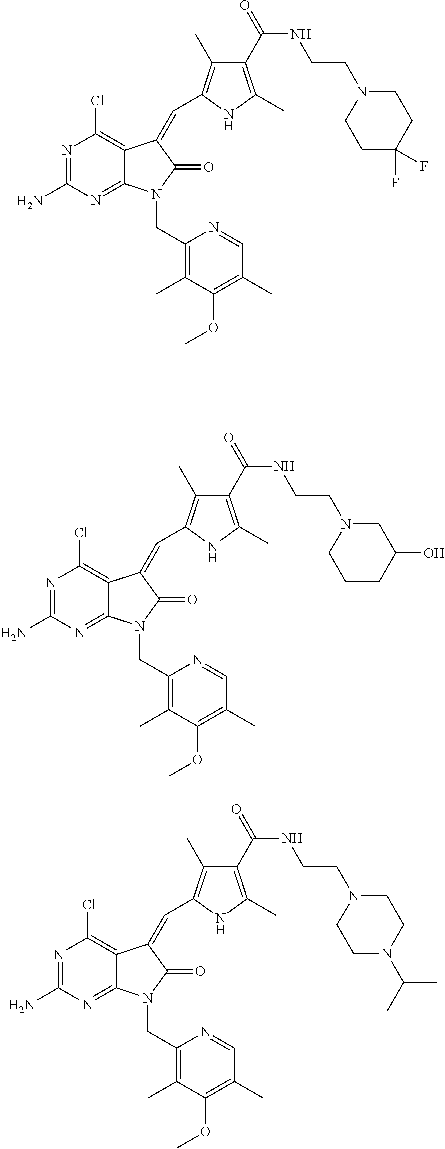 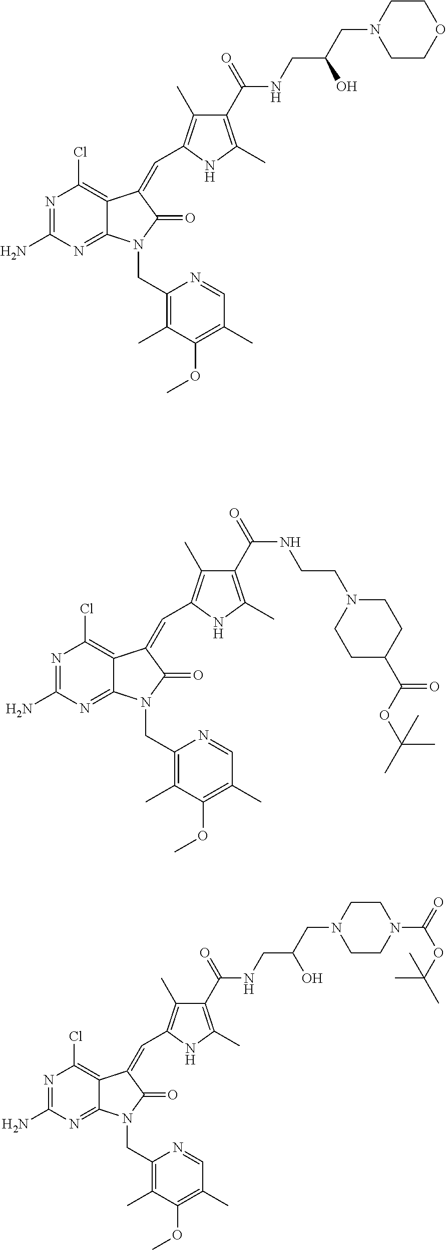 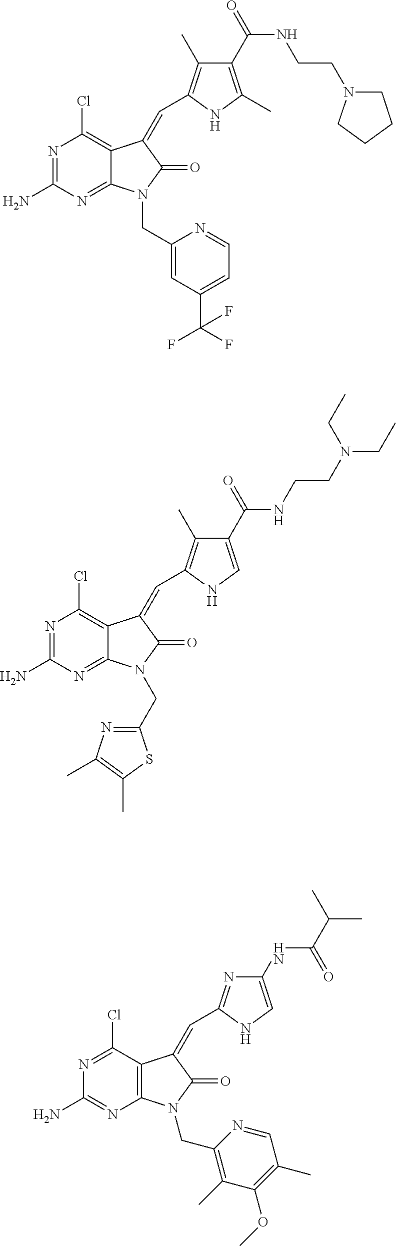 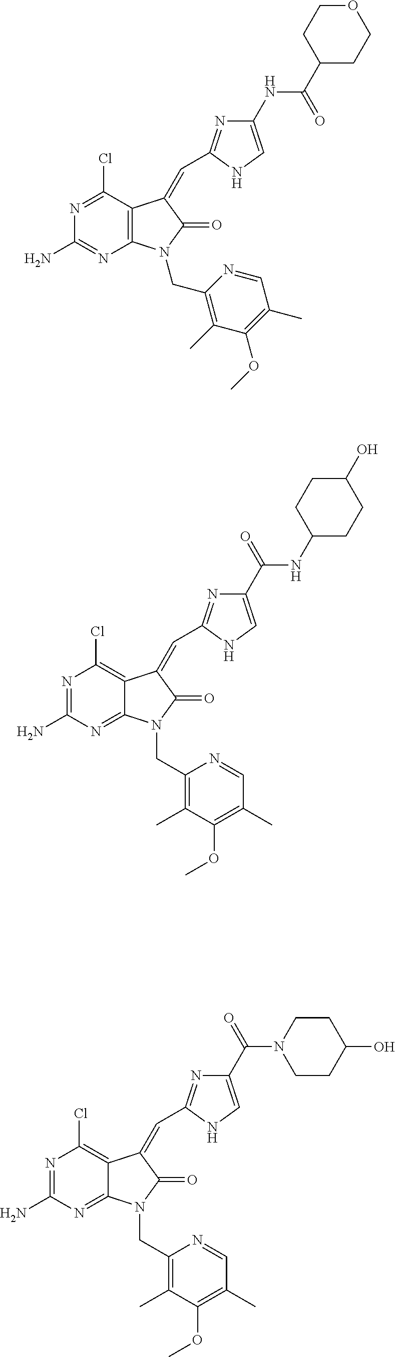 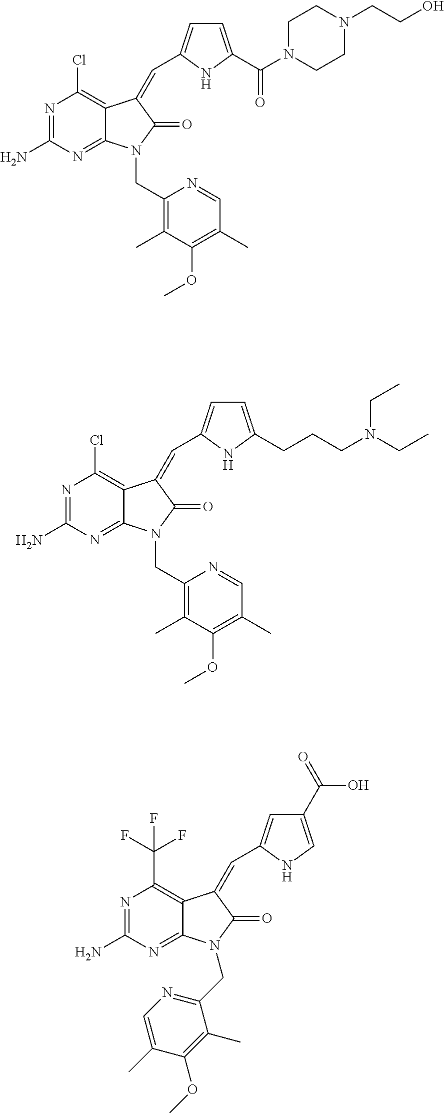 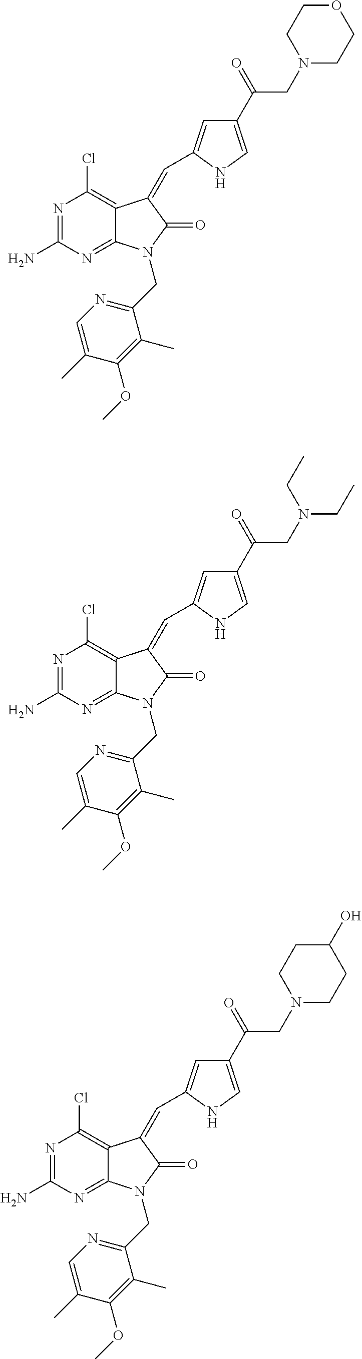 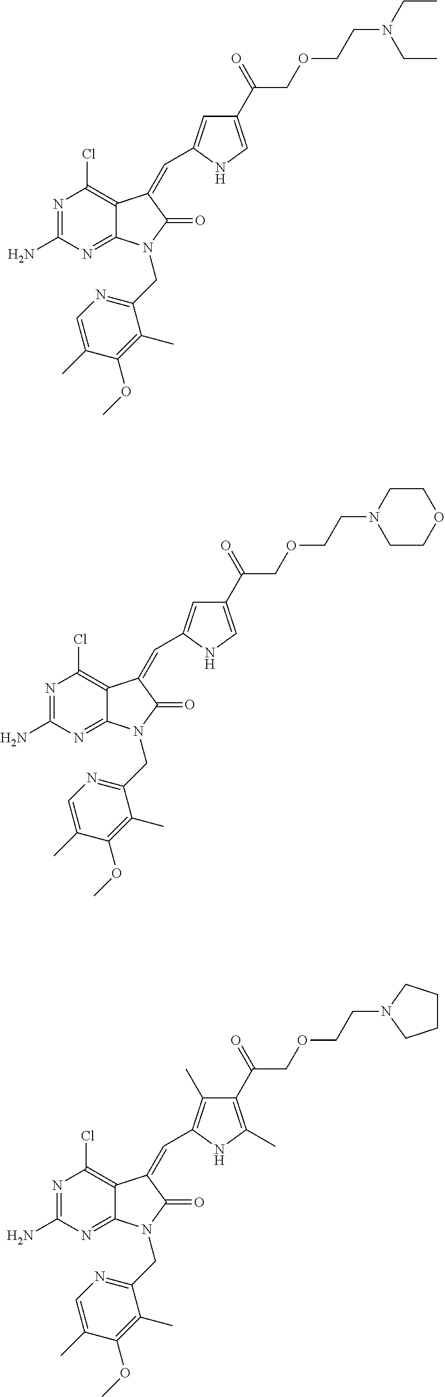 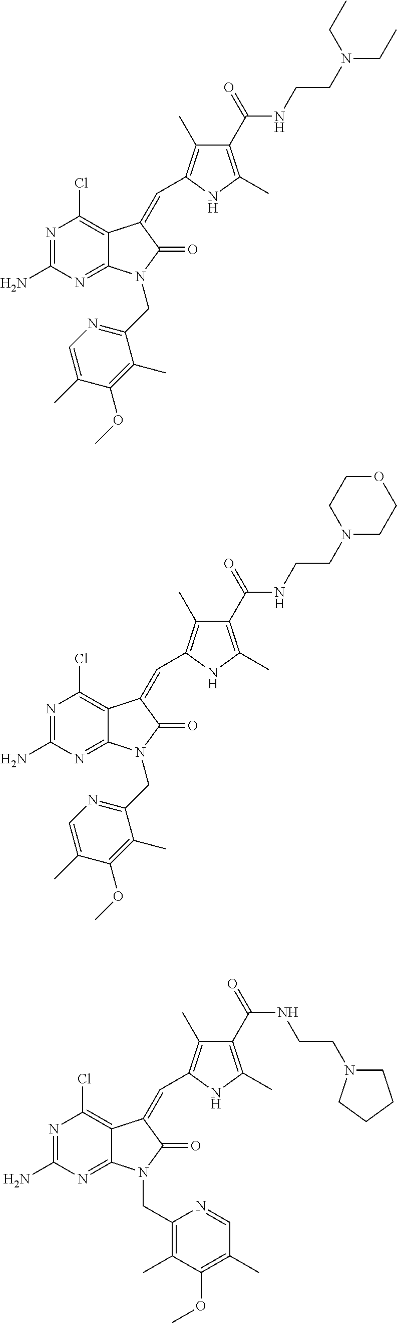 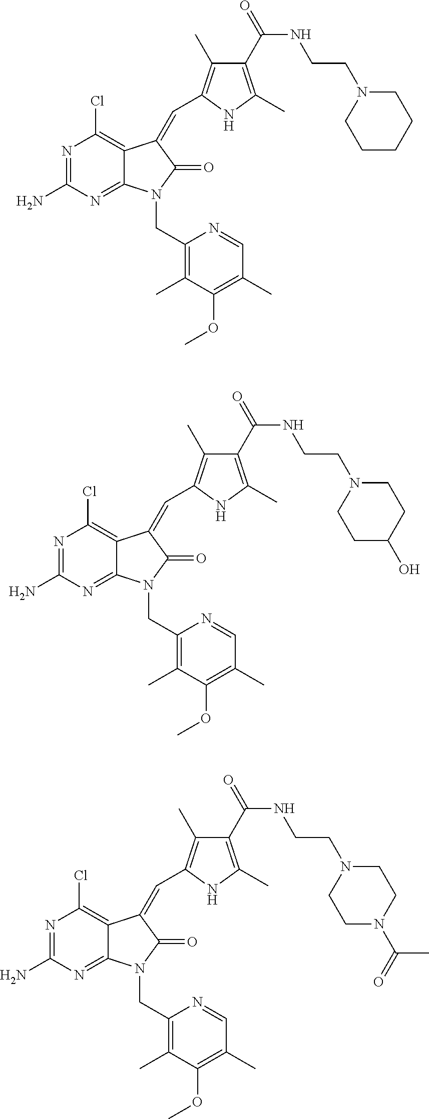 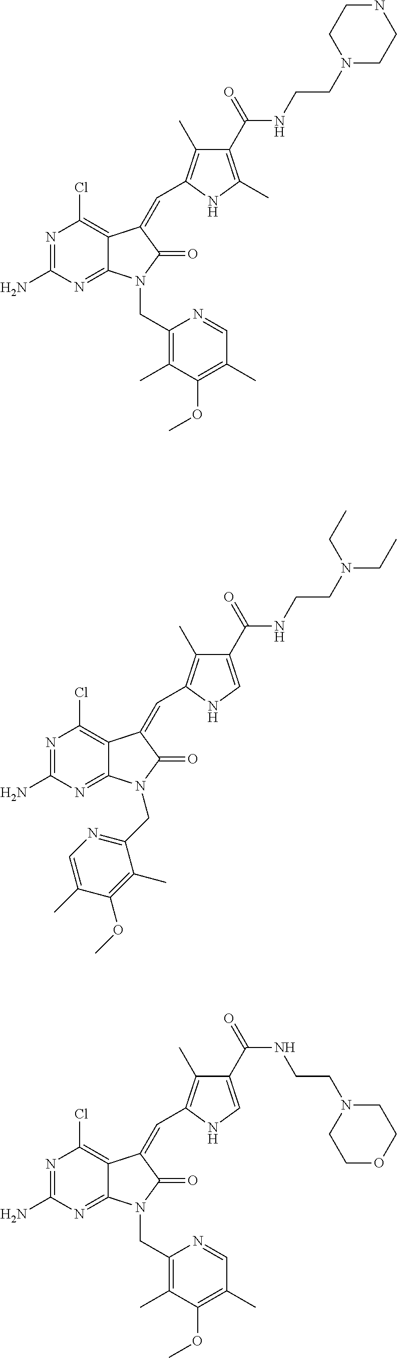 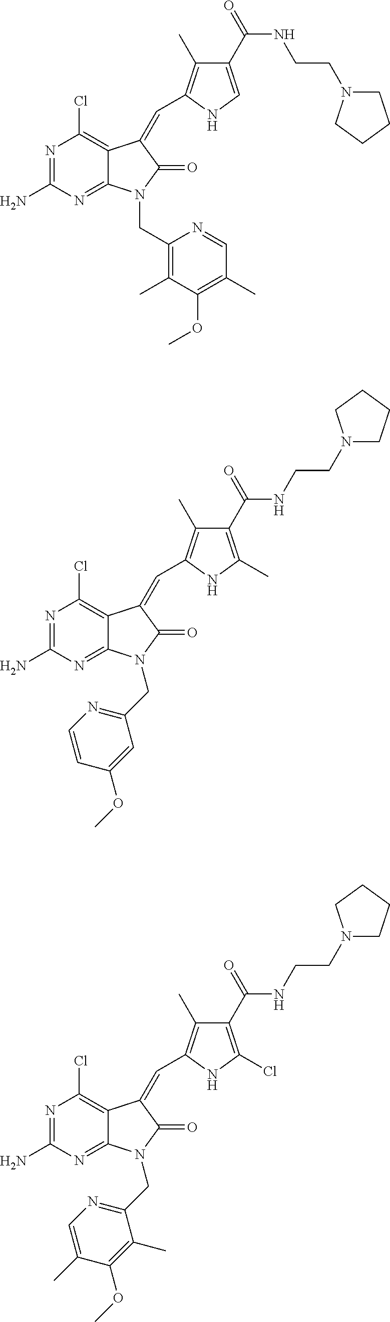 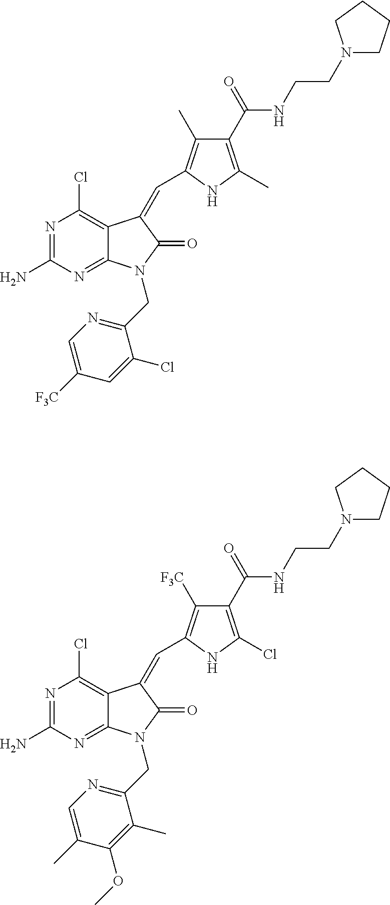
or any salt, solvate, hydrate, tautomer, stereoisomer, or prodrug thereof.

35.-68. (canceled)

69. A pharmaceutical composition comprising the compound of claim 1 and a suitable excipient.

70. A pharmaceutical combination comprising the compound of claim 1 and a second medicament.

71. A pharmaceutical composition comprising the combination of claim 70 and a suitable excipient.

72. The combination of claim 70 wherein the second medicament is adapted for treatment of cancer, a neurodegenerative disease, or Alzheimer's disease.

73.-74. (canceled)

75. A method of synthesis of a compound of formula (III): 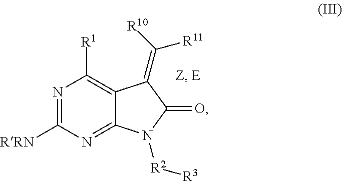
wherein R, R¹, R², R³, R¹⁰ and R¹¹ are as defined in claim 1, the method comprising contacting a compound of formula (IIIa): 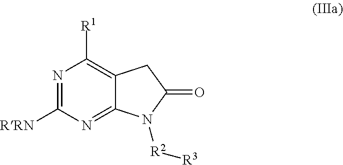
and R¹⁰—C(O)—R¹¹ under conditions suitable to bring about the formation of the compound of formula (III), wherein a double bond marked Z, E can be in either a Z or an E configuration, or a mixture thereof.

76.-79. (canceled)

80. A method of treatment of a malcondition in a patient wherein inhibition of Hsp90 is medically indicated comprising administering an effective amount of the compound of claim 1 to the patient in a dose, at a frequency, and for a duration of time sufficient to provide a beneficial effect to the patient.

81. The method of claim 80 wherein the malcondition comprises a cancer, a neurodegenerative disease, or Alzheimers disease.
